(19) United States
(12) Patent Application Publication
(10) Pub. No.: US 2023/0357162 A1
(43) Pub. Date: Nov. 9, 2023

(54) COMPOUNDS, COMPOSITIONS AND METHODS

(71) Applicant: Denali Therapeutics Inc., South San Francisco, CA (US)

(72) Inventors: Alex L. Bagdasarian, South San Francisco, CA (US); Robert A. Craig, II, South San Francisco, CA (US); Javier de Vicente Fidalgo, Foster City, CA (US); Anthony A. Estrada, Danville, CA (US); Brian M. Fox, Redwood City, CA (US); Cheng Hu, San Mateo, CA (US); Benjamin J. Huffman, Brisbane, CA (US); Katrina W. Lexa, San Francisco, CA (US); Lizanne G. Nilewski, Pacifica, CA (US); Maksim Osipov, Redwood City, CA (US); Arun Thottumkara, San Francisco, CA (US)

(21) Appl. No.: 18/041,539

(22) PCT Filed: Aug. 13, 2021
(86) PCT No.: PCT/US2021/045924

§ 371 Date: Feb. 13, 2023

Related U.S. Application Data
(60) Provisional application No. 63/151,600, filed on Feb. 19, 2021.
(60) Provisional application No. 63/066,074, filed on Aug. 14, 2020.

(51) Int. Cl. C07D 237/32; C07D 401/12; C07D 403/12; C07D 405/14; C07D 471/04; C07D 405/12; C07D 487/04; C07D 413/12

(52) U.S. Cl. CPC C07D 237/32; C07D 401/12; C07D 403/12; C07D 405/12; C07D 405/14; C07D 413/12; C07D 471/04; C07D 487/04

(57) ABSTRACT

The present disclosure relates generally to small molecule modulators of NLR Family Pyrin Domain Containing 3 (NL-RP3), or a pharmaceutically acceptable salt, isotopically enriched analog, stereoisomer, mixture of stereoisomers, or prodrug thereof, methods of making and intermediates thereof, and methods of using thereof.

This application claims the benefit under 35 U.S.C. §119(e) to U.S. Provisional Application Nos. 63/066,074, filed Aug. 14, 2020, and 63/151,600, filed Feb. 19, 2021, each of which is incorporated by reference in its entirety.

### FIELD

The present disclosure relates generally to small molecule modulators of NLR Family Pyrin Domain Containing 3 (NLRP3), and their use as therapeutic agents.

### BACKGROUND

Inhibition of NLRP3 activation has been shown to result in potent therapeutic effects in animal models of inflammatory diseases. Modulators of NLRP3, inhibitors in particular, have broad therapeutic potential in a wide array of auto-inflammatory and chronic inflammatory diseases that either require better treatment options or for which no adequate therapies exist. Therapies targeting NLRP3-dependent cytokines are already approved for therapeutic use; however, they have notable disadvantages relative to direct NLRP3 antagonists. There remains a strong impetus for the discovery and clinical development of molecules that antagonize NLRP3.

### DESCRIPTION

Provided herein are compounds, or a pharmaceutically acceptable salt, isotopically enriched analog, stereoisomer, mixture of stereoisomers, or prodrug thereof, that are useful in treating and/or preventing diseases mediated, at least in part, by NLRP3.

In some embodiments, provided are compounds that modulate the activity of NLRP3. In some embodiments, the compounds inhibit the activation of NLRP3.

In another embodiment, provided is a pharmaceutical composition comprising a compound as described herein, or a pharmaceutically acceptable salt, isotopically enriched analog, stereoisomer, mixture of stereoisomers, or prodrug thereof, and a pharmaceutically acceptable carrier.

In another embodiment, provided is a method for treating a disease or condition mediated, at least in part, by NLRP3, the method comprising administering an effective amount of the pharmaceutical composition comprising a compound as described herein, or a pharmaceutically acceptable salt, isotopically enriched analog, stereoisomer, mixture of stereoisomers, or prodrug thereof.

In another embodiment, provided is a method for treating a disease or condition mediated, at least in part, by TNF-α, the method comprising administering an effective amount of the pharmaceutical composition comprising a compound as described herein, or a pharmaceutically acceptable salt, isotopically enriched analog, stereoisomer, mixture of stereoisomers, or prodrug thereof. In some embodiments the administration is to a subject resistant to treatment with an anti-TNF-α agent. In some embodiments, the disease is a gut disease or condition. In some embodiments, the disease or condition is inflammatory bowel disease, Crohn’s disease, or ulcerative colitis.

The disclosure also provides compositions, including pharmaceutical compositions, kits that include the compounds, or a pharmaceutically acceptable salt, isotopically enriched analog, stereoisomer, mixture of stereoisomers, or prodrug thereof, methods of using (or administering) and making the compounds, or a pharmaceutically acceptable salt, isotopically enriched analog, stereoisomer, mixture of stereoisomers, or prodrug thereof, and intermediates thereof.

The disclosure further provides compounds, or a pharmaceutically acceptable salt, isotopically enriched analog, stereoisomer, mixture of stereoisomers, or prodrug thereof, or compositions thereof for use in a method of treating a disease, disorder, or condition that is mediated, at least in part, by NLRP3.

Moreover, the disclosure provides uses of the compounds, or a pharmaceutically acceptable salt, isotopically enriched analog, stereoisomer, mixture of stereoisomers, or prodrug thereof, or compositions thereof in the manufacture of a medicament for the treatment of a disease, disorder, or condition that is mediated, at least in part, by NLRP3.

The description herein sets forth exemplary embodiments of the present technology. It should be recognized, however, that such description is not intended as a limitation on the scope of the present disclosure but is instead provided as a description of exemplary embodiments.

### 1. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ_ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ± 10%. In other embodiments, the term “about” includes the indicated amount ± 5%. In certain other embodiments, the term “about” includes the indicated amount ± 1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl) or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, a divalent heteroaryl group, etc., may also be referred to as an “alkylene” group or an “alkylenyl” group (for example, methylenyl, ethylenyl, and propylenyl), an “arylene” group or an “arylenyl” group (for example, phenylenyl or napthylenyl, or quinolinyl for heteroarylene), respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O-”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Alkoxyalkyl” refers to the group “alkyl-O-alkyl”.

“Alkylthio” refers to the group “alkyl-S-”. “Alkylsulfinyl” refers to the group “alkyl-S(O)-”. “Alkylsulfonyl” refers to the group “alkyl-S(O)₂-”. “Alkylsulfonylalkyl” refers to -alkyl-S(O)₂-alkyl.

“Acyl” refers to a group -C(O)R^{Y}, wherein R^{y} is hydrogen, alkyl, alkenyl, alkynyl, cycloalkyl, heterocyclyl, aryl, heteroalkyl, or heteroaryl; each of which may be optionally substituted, as defined herein. Examples of acyl include, e.g., formyl, acetyl, cyclohexylcarbonyl, cyclohexylmethyl-carbonyl, and benzoyl.

“Amido” refers to both a “C-amido” group which refers to the group -C(O)NR^{y}R^{Z} and an “N-amido” group which refers to the group -NR^{y}C(O)R^{Z}, wherein R^{y} and R² are independently hydrogen, alkyl, alkenyl, alkynyl, cycloalkyl, heterocyclyl, aryl, heteroalkyl, or heteroaryl; each of which may be optionally substituted, as defined herein, or R^{y} and R² are taken together to form a cycloalkyl or heterocyclyl; each of which may be optionally substituted, as defined herein.

“Amino” refers to the group -NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Amidino” refers to -C(NR^{y})(NR^{z}₂), wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Arylalkyl” or “Aralkyl” refers to the group “aryl-alkyl-”.

“Carbamoyl” refers to both an “O-carbamoyl” group which refers to the group -O-C(O)NR^{y}R^{z} and an “N-carbamoyl” group which refers to the group -NR^{y}C(O)OR^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Carboxyl ester” or “ester” refer to both -OC(O)R^{X} and -C(O)OR^{x}, wherein R^{x} is alkyl, alkenyl, alkynyl, cycloalkyl, heterocyclyl, aryl, heteroalkyl, or heteroaryl; each of which may be optionally substituted, as defined herein.

“Cyanoalkyl” refers to refers to an alkyl group as defined above, wherein one or more (e.g., 1 or 2) hydrogen atoms are replaced by a cyano (—CN) group.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₂ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Cycloalkylalkyl” refers to the group “cycloalkyl-alkyl-”.

“Imino” refers to a group -C(NR^{y})R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Imido” refers to a group -C(O)NR^{y}C(O)R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Halogen” or “halo” refers to atoms occupying group VIIA of the periodic table, such as fluoro, chloro, bromo, or iodo.

“Haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6 or 1 to 3) hydrogen atoms are replaced by a halogen.

“Haloalkoxyalkyl” refers to an alkoxyalkyl group as defined above, wherein one or more (e.g., 1 to 6 or 1 to 3) hydrogen atoms are replaced by a halogen.

“Hydroxyalkyl” refers to an alkyl group as defined above, wherein one or more (e.g., 1 to 6 or 1 to 3) hydrogen atoms are replaced by a hydroxy group.

“Heteroalkyl” refers to an alkyl group in which one or more of the carbon atoms (and any associated hydrogen atoms), excluding any terminal carbon atom(s), are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, -NR^{Y}-, —O—, —S—, —S(O)—, —S(O)₂—, and the like, wherein R^{y} is hydrogen, alkyl, alkenyl, alkynyl, cycloalkyl, heterocyclyl, aryl, heteroalkyl, or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃,—CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.), and amines (e.g., -CH_{Z}NR^{y}CH₃, -CH(CH₃)NR^{Y}CH₃, -CH_{Z}CH_{Z}NR^{y}CH₃, -CH_{Z}CH_{Z}NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkyl includes 2 to 10 carbon atoms, 2 to 8 carbon atoms, or 2 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) or N-oxide (—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂₋₁₂ heterocyclyl), 2 to 10 ring carbon atoms (i.e., C₂₋₁₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃₋₈ heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Heterocyclylalkyl” refers to the group “heterocyclyl-alkyl-.”

“Oxime” refers to the group -CR^{y}(=NOH) wherein R^{y} is hydrogen, alkyl, alkenyl, alkynyl, cycloalkyl, heterocyclyl, aryl, heteroalkyl, or heteroaryl; each of which may be optionally substituted, as defined herein.

“Sulfonyl” refers to the group -S(O)₂R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfonyl are methylsulfonyl, ethylsulfonyl, phenylsulfonyl, and toluenesulfonyl.

“Sulfinyl” refers to the group -S(O)R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfinyl are methylsulfinyl, ethylsulfinyl, phenylsulfinyl, and toluenesulfinyl.

“Sulfonamido” refers to the groups -SO₂NR^{y}R^{z} and -NR^{y}SO₂R^{z}, where R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5 or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5 or 1 to 3) hydrogen atom is replaced by a bond to a non-hydrogen atom such as, but not limited to alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanadino, halo, haloalkyl, haloalkoxy, hydroxyalkyl, heteroalkyl, heteroaryl, heteroarylalkyl, heterocyclyl, heterocyclylalkyl, —NHNH₂, ═NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide, or -Si(R^{y})₃, wherein each R^{y} is independently hydrogen, alkyl, alkenyl, alkynyl, heteroalkyl, cycloalkyl, aryl, heteroaryl, or heterocyclyl.

In certain embodiments, “substituted” includes any of the above alkyl, alkenyl, alkynyl, cycloalkyl, heterocyclyl, aryl, or heteroaryl groups in which one or more (e.g., 1 to 5 or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, -NR^{g}R^{h}, -NR^{g}C(O)R^{h}, -NR^{g}C(O)NR^{g}R^{h}, -NR^{g}C(O)OR^{h}, -NR^{g}S(O)₁₋₂R^{h}, -C(O)R^{g}, -C(O)OR^{g}, -OC(O)OR^{g}, -OC(O)R^{g}, -C(O)NR^{g}R^{h}, -OC(O)NR^{g}R^{h}, -OR^{g}, -SR⁸, -S(O)R^{g}, -S(O)₂R^{g}, -OS(O)₁₋₂R^{g}, -S(O)₁₋₂OR^{g}, -NR^{g}S(O)₁₋₂NR^{g}R^{h}, =NSO₂R⁸, =NOR^{g}, -S(O)₁₋₂NR^{g}R^{h}, —SF₅, —SCF₃, or —OCF₃. In certain embodiments, “substituted” also means any of the above groups in which one or more (e.g., 1 to 5 or 1 to 3) hydrogen atoms are replaced with -C(O)R^{g}, -C(O)OR^{g}, -C(O)NR^{g}R^{h}, -CH₂SO₂R^{g}, or -CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5 or 1 to 3) hydrogen atoms are replaced by a bond to an amino, cyano, hydroxy, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} are taken together with the atoms to which they are attached to form a heterocyclyl ring optionally substituted with oxo, halo, or alkyl optionally substituted with oxo, halo, amino, hydroxy, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to three.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵ 1, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example, by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life, reduced dosage requirements, and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino, and/or carboxyl groups, or groups similar thereto.

Provided are also a pharmaceutically acceptable salt, isotopically enriched analog, deuterated analog, stereoisomer, mixture of stereoisomers, and prodrugs of the compounds described herein. “Pharmaceutically acceptable” or “physiologically acceptable” refer to compounds, salts, compositions, dosage forms, and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri- cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds described herein, or their pharmaceutically acceptable salts include an asymmetric center and may thus give rise to enantiomers, diastereomers, and other stereoisomeric forms that may be defined, in terms of absolute stereochemistry, as (R)- or (S)— or, as (D)— or (L)— for amino acids. The present disclosure is meant to include all such possible isomers, as well as their racemic and optically pure forms. Optically active (+) and (-), (R)- and (S)—, or (D)— and (L)— isomers may be prepared using chiral synthons or chiral reagents, or resolved using conventional techniques, for example, chromatography and/or fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers, or mixtures thereof, and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds described herein, and the like. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

### 2. Compounds

Provided herein are compounds that are modulators of NLRP3. In certain embodiments, provided is a compound of Formula I:

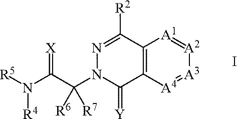

or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)_{2,} -NR¹¹C(O)N(R¹¹)₂, -C(O)N(R¹¹)₂, -NR¹¹C(0)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, -NO₂, —SF₅, -OR¹¹, -C(O)R¹², -C(O)OR¹¹, -SR¹¹, -NR¹¹S(O)₀₋₂-R¹¹, -NR¹¹S(O)₀₋₂N(R¹¹)₂, -NR¹¹C(O)N(R¹¹)₂,-NR¹¹C(O)OR¹¹, -OC(O)R¹¹, -OC(O)N(R¹¹)₂, halo, or cyano; wherein the C₁₋₆ alkyl, C₂₋₆ alkenyl, C₂₋₆ alkynyl, or C₁₋₆ haloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five z^{la};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)2, -NR¹¹C(O)N(R¹¹)₂, -C(O)N(R¹¹)₂, -NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)R¹², -C(O)OR¹¹, -NR¹¹S(O)₀₋₂-R¹¹, -NR¹¹S(O)₀₋₂N(R¹¹)₂, -NR¹¹C(O)N(R¹¹)₂, -NR¹¹C(O)R¹¹, -OC(O)N(R¹¹)₂, or -NR¹¹C(O)OR¹¹;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
R¹² is C₁₋₆ alkyl, C₂₋₆ alkenyl, C₂₋₆ alkynyl, or C₁₋₆ haloalkyl;
each Z¹ª is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)₀₋₂R¹³, -NR¹³S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂, -NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b}_{;}
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, -N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, —C(O)—, —C(O)O—, —C(O)NH—, -C(O)N(C₁₋₆ alkyl)-, -C(O)N(C₂₋₆ alkenyl)-, -C(O)N(C₂₋₆ alkynyl)-, -C(O)N(C₁₋₆ haloalkyl)-, -C(O)N(C₃₋₁₀ cycloalkyl)-, -C(O)N(heterocyclyl)-, -C(O)N(aryl)-, -C(O)N(heteroaryl)-, -NHC(O)-, -NHC(O)O-, -NHC(O)NH-, -NHS(O)-, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

In certain embodiments, when one of R⁴ and R⁵ is hydrogen, the other of R⁴ and R⁵ is not C₃-alkyl substituted with an optionally substituted piperazinyl ring.

In certain embodiments, when R² is unsubstituted C₁₋₆ alkyl, or unsubstituted C₂₋₆ alkenyl and one R¹ is unsubstituted C₁₋₆ alkyl, unsubstituted C₂₋₆ alkenyl, unsubstituted C₅₋₇ cycloalkyl, unsubstituted C₁₋₆ alkoxy, halo, benzyl, or hydroxy; then:
R⁴ and R⁵ are not independently hydrogen, unsubstituted C₁₋₆ alkyl, unsubstituted C₂₋₆ alkenyl, unsubstituted C₅₋₇ cycloalkyl, unsubstituted aryl, or aryl substituted with one Z¹; and
R⁴ and R⁵, together with the nitrogen to which they are attached, are not unsubstituted piperidinyl, unsubstituted morpholinyl, or piperazinyl substituted with C₁₋₆ alkyl or aryl.

In certain embodiments, when R² is -CH₂-C(O)OR¹¹; then R⁴ and R⁵, together with the nitrogen to which they are attached, are not unsubstituted morpholinyl.

In certain embodiments, provided is a compound of Formula I:
or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, -NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, —NO₂, —SF₅, -OR¹¹, -C(O)R¹², -C(O)OR¹¹, -SR¹¹, -NR¹¹S(O)₀₋₂-R¹¹, -NR¹¹S(O)₀₋₂N(R¹¹)₂, -NR¹¹C(O)N(R¹¹)₂, -NR¹¹C(O)OR¹¹, -OC(O)R¹¹, -OC(O)N(R¹¹)₂, halo, or cyano; wherein the C₁₋₆ alkyl, C₂₋₆ alkenyl, C₂₋₆ alkynyl, or C₁₋₆ haloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{1a};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, -NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)OR¹¹, -NR¹¹S(O)₀₋₂-R¹¹, -NR¹¹S(O)₀₋₂N(R¹¹)₂, -NR¹¹C(O)N(R¹¹)₂, -NR¹¹C(O)R¹¹, -OC(O)N(R¹¹)₂, or -NR¹¹C(O)OR¹¹;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
R¹² is C₁₋₆ alkyl, C₂₋₆ alkenyl, C₂₋₆ alkynyl, or C₁₋₆ haloalkyl;
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)₀₋₂R¹³, -NR¹³S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂, -NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, -N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, —C(O)—, —C(O)O—, —C(O)NH—, -C(O)N(C₁₋₆ alkyl)-, -C(O)N(C₂₋₆ alkenyl)-, -C(O)N(C₂₋₆ alkynyl)-, -C(O)N(C₁₋₆ haloalkyl)-, -C(O)N(C₃₋₁₀ cycloalkyl)-, -C(O)N(heterocyclyl)-, -C(O)N(aryl)-, -C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

In certain embodiments, provided is a compound of Formula I, or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, -NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₃₋₁₀ cycloalkyl; wherein the C₃₋₁₀ cycloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹;
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{1a};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, -NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)OR¹¹, -NR¹¹S(O)₀₋₂-R¹¹, -NR¹¹S(O)₀₋₂N(R¹¹)₂, -NR¹¹C(O)N(R¹¹)₂, -NR¹¹C(O)R¹¹, -OC(O)N(R¹¹)₂, or -NR¹¹C(O)OR¹¹;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)₀₋₂R¹³, -NR¹³S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂, -NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, -N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, —C(O)—, —C(O)O—, —C(O)NH—, -C(O)N(C₁₋₆ alkyl)-, -C(O)N(C₂₋₆ alkenyl)-, -C(O)N(C₂₋₆ alkynyl)-, -C(O)N(C₁₋₆ haloalkyl)-, -C(O)N(C₃₋₁₀ cycloalkyl)-, -C(O)N(heterocyclyl)-, -C(O)N(aryl)-, -C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

In certain embodiments of Formula I, X is O. In certain embodiments of Formula I, Y is O. In certain embodiments, X is S. In certain embodiments of Formula I, Y is S. In certain embodiments of Formula I, X is O, and Y is S. In certain embodiments of Formula I, X is S, and Y is O. In certain embodiments of Formula I, X and Y are O. In certain embodiments of Formula I, X and Y are S.

In certain embodiments, provided is a compound of Formula II:

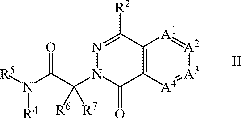

or a pharmaceutically acceptable salt, isotopically enriched analog, stereoisomer, mixture of stereoisomers, or prodrug thereof, wherein:
A¹, A², A³ and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, -NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, —NO₂, —SF₅, -OR¹¹, -C(O)R¹², -C(O)OR¹¹, -SR¹¹, -NR¹¹S(O)₀₋₂-R¹¹, -NR¹¹S(O)₀₋₂N(R¹¹)₂, -NR¹¹C(O)N(R¹¹)₂, -NR¹¹C(O)OR¹¹, -OC(O)R¹¹, -OC(O)N(R¹¹)₂, halo, or cyano; wherein the C₁₋₆ alkyl, C₂₋₆ alkenyl, C₂₋₆ alkynyl, or C₁₋₆ haloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹;
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{1a};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, -NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)R¹², -C(O)OR¹¹, -NR¹¹S(O)₀₋₂-R¹¹, -NR¹¹S(O)₀₋₂N(R¹¹)₂, -NR¹¹C(O)N(R¹¹)₂, -NR¹¹C(O)R¹¹, -OC(O)N(R¹¹)₂, or -NR¹¹C(O)OR¹¹;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
R¹² is C₁₋₆ alkyl, C₂₋₆ alkenyl, C₂₋₆ alkynyl, or C₁₋₆ haloalkyl; wherein each C₁₋₆ alkyl, C₂₋₆ alkenyl, C₂₋₆ alkynyl, or C₁₋₆ haloalkyl of R¹² is independently optionally substituted with one to five Z²;
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)₀₋₂R¹³, -NR¹³S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂, -NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, -N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, —C(O)—, —C(O)O—, —C(O)NH—, -C(O)N(C₁₋₆ alkyl)-, -C(O)N(C₂₋₆ alkenyl)-, -C(O)N(C₂₋₆ alkynyl)-, -C(O)N(C₁₋₆ haloalkyl)-, -C(O)N(C₃₋₁₀ cycloalkyl)-, -C(O)N(heterocyclyl)-, -C(O)N(aryl)-, -C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

In certain embodiments, when one of R⁴ and R⁵ is hydrogen, the other of R⁴ and R⁵ is not C₃-alkyl substituted with an optionally substituted piperazinyl ring.

In certain embodiments, when R² is unsubstituted C₁₋₆ alkyl, or unsubstituted C₂₋₆ alkenyl and one R¹ is unsubstituted C₁₋₆ alkyl, unsubstituted C₂₋₆ alkenyl, unsubstituted C₅₋₇ cycloalkyl, unsubstituted C₁₋₆ alkoxy, halo, benzyl, or hydroxy; then:
R⁴ and R⁵ are not independently hydrogen, unsubstituted C₁₋₆ alkyl, unsubstituted C₂₋₆ alkenyl, unsubstituted C₅₋₇ cycloalkyl, unsubstituted aryl, or aryl substituted with one Z¹; and
R⁴ and R⁵, together with the nitrogen to which they are attached, are not unsubstituted piperidinyl, unsubstituted morpholinyl, or piperazinyl substituted with C₁₋₆ alkyl or aryl.

In certain embodiments, when R² is -CH₂-C(O)OR¹¹; then R⁴ and R⁵, together with the nitrogen to which they are attached, are not unsubstituted morpholinyl.

In certain embodiments, provided is a compound of Formula II, or a pharmaceutically acceptable salt, isotopically enriched analog, stereoisomer, mixture of stereoisomers, or prodrug thereof, wherein:
A¹, A², A³ and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, -NR¹¹C(O)R¹¹, -OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, —NO₂, —SF₅, -OR¹¹, -C(O)R¹², -C(O)OR¹¹, -SR¹¹, -NR¹¹S(O)₀₋₂-R¹¹, -NR¹¹S(O)₀₋₂N(R¹¹)₂, -NR¹¹C(O)N(R¹¹)₂, -NR¹¹C(O)OR¹¹, -OC(O)R¹¹, -OC(O)N(R¹¹)₂, halo, or cyano; wherein the C₁₋₆ alkyl, C₂₋₆ alkenyl, C₂₋₆ alkynyl, or C₁₋₆haloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{1a};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, -NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)OR¹¹, -NR¹¹S(O)₀₋₂-R¹¹, -NR¹¹S(O)₀₋₂N(R¹¹)₂, -NR¹¹C(O)N(R¹¹)₂, -NR¹¹C(O)R¹¹, -OC(O)N(R¹¹)₂, or -NR¹¹C(O)OR¹¹;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)₀₋₂R¹³, -NR¹³S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂, -NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
R¹² is C₁₋₆ alkyl, C₂₋₆ alkenyl, C₂₋₆ alkynyl, or C₁₋₆ haloalkyl;
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆alkyl)-, -N(C₂₋₆ alkenyl)-, -N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, —C(O)—, —C(O)O—, —C(O)NH—, -C(O)N(C₁₋₆ alkyl)-, -C(O)N(C₂₋₆ alkenyl)-, -C(O)N(C₂₋₆ alkynyl)-, -C(O)N(C₁₋₆ haloalkyl)-, -C(O)N(C₃₋₁₀ cycloalkyl)-, -C(O)N(heterocyclyl)-, -C(O)N(aryl)-, -C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

In certain embodiments, each of A¹, A², A³, and A⁴ is independently CH or CR¹; provided at least one of A¹, A², A³, and A⁴ is CR¹.

In certain embodiments, one of A¹, A², A³, and A⁴ is N; one of A¹, A², A³, and A⁴ is CR¹; and the remaining A¹, A², A³, and A⁴ are independently CH or CR¹.

In certain embodiments, two of A¹, A², A³, and A⁴ are N; one of A¹, A², A³, and A⁴ is CR¹; and the remaining A¹, A², A³, and A⁴ is CH or CR¹.

In certain embodiments, A² is CR¹ and A¹, A³ and A⁴ are each independently N, CH, or CR¹.

In certain embodiments, A³ is CR¹ and A¹, A², and A⁴ are each independently N, CH, or CR¹.

In certain embodiments, each R¹ is independently halo, cyano, C₁₋₆ alkyl, C₃₋₁₀ cycloalkyl, -N(R¹¹)₂, -OR¹¹, or -SR¹¹; wherein each C₁₋₆ alkyl or C₃₋₁₀ cycloalkyl is independently optionally substituted with one to eight Z¹. In certain embodiments, each R¹ is independently halo, cyano, C₁₋₆ alkyl, C₃₋₁₀ cycloalkyl, -OR¹¹, or -SR¹¹; wherein each C₁₋₆ alkyl or C₃₋₁₀ cycloalkyl is independently optionally substituted with one to eight Z¹. In certain embodiments, each R¹ is independently halo, cyano, C₁₋₆ alkyl, C₃₋₁₀ cycloalkyl, -OR¹¹, or -SR¹¹; wherein each C₁₋₆ alkyl is independently optionally substituted with one to eight Z¹.

In certain embodiments, each R¹ is independently halo, cyano, C₁₋₆ alkyl, C₁₋₆ alkoxy, C₁₋₆ haloalkyl, C₁₋₆ haloalkoxy, -N(R¹¹)₂, -SR¹¹, or C₃₋₁₀ cycloalkyl. In certain embodiments, each R¹ is independently halo, cyano, C₁₋₆ alkyl, C₁₋₆ alkoxy, C₁₋₆ haloalkyl, C₁₋₆ haloalkoxy, -SR¹¹, or C₃₋₁₀ cycloalkyl. In certain embodiments, each R¹ is independently halo, cyano, C₁₋₆ alkyl, C₁₋₆ alkoxy, or C₁₋₆ haloalkyl. In certain embodiments, each R¹ is independently halo or C₁₋₆ alkyl.

In certain embodiments, each R¹ is independently fluoro, chloro, bromo, iodo, —CH₃, —CHF₂, —CF₃, —OCH₃, —OCHF₂, —OCF₃, —N(CH₃)₂, —S—CH₃, 1,1,1-trifluoropropan-2-yl, cyclopropyl, or cyclobutyl.

In certain embodiments, each R¹ is independently fluoro, chloro, bromo, iodo, —CH₃, —CHF₂, —CF₃, —OCHF₂, —OCF₃, 1,1,1-trifluoropropan-2-yl, —S—CH₃, or cyclopropyl. In certain embodiments, each R¹ is independently fluoro, bromo, or —CH₃.

In certain embodiments, R⁴ is hydrogen.

In certain embodiments, R⁶ is hydrogen or C₁₋₆ alkyl. In certain embodiments, R⁶ is hydrogen.

In certain embodiments, R⁷ is hydrogen.

In certain embodiments, R⁶ is hydrogen, and R⁷ is hydrogen.

In certain embodiments, R⁴ is hydrogen, R⁶ is hydrogen, and R⁷ is hydrogen.

In certain embodiments, R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹.

In certain embodiments, R⁵ is C₁₋₆ alkyl, C₃₋₁₀ cycloalkyl, heterocyclyl, aryl, or heteroaryl; wherein the C₃₋₁₀ cycloalkyl, heterocyclyl, or heteroaryl is independently optionally substituted with one to five Z¹.

In certain embodiments, R⁴ and R⁵ together form a heterocyclyl ring optionally substituted with one to eight Z¹.

In certain embodiments, R⁵ is C₃₋₁₀ cycloalkyl, heterocyclyl, or heteroaryl; wherein the C₃₋₁₀ cycloalkyl, heterocyclyl, or heteroaryl is independently optionally substituted with one to five Z¹.

In certain embodiments, R⁵ is C₃₋₁₀ cycloalkyl optionally substituted with one to five Z¹. In certain embodiments, R⁵ is heterocyclyl optionally substituted with one to five Z¹. In certain embodiments, R⁵ is heteroaryl optionally substituted with one to five Z¹.

In certain embodiments, R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five halo, -OR¹¹, -C(O)OR¹¹, cyano, C₁₋₆ alkyl, C₁₋₆ haloalkyl, C₃₋₁₀ cycloalkyl,heterocyclyl, aryl, or heteroaryl.

In certain embodiments, R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted with one to five halo, -OR¹¹, -C(O)OR¹¹, cyano, C₁₋₆ alkyl, C₁₋₆ haloalkyl, C₃₋₁₀ cycloalkyl, heterocyclyl, aryl, or heteroaryl.

In certain embodiments, R⁴ is hydrogen, and R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted with one to five halo, -OR¹¹, -C(O)OR¹¹, cyano, C₁₋₆ alkyl, C₁₋₆ haloalkyl, C₃₋₁₀ cycloalkyl, heterocyclyl, aryl, or heteroaryl.

In certain embodiments, R⁴ is hydrogen, R⁶ is hydrogen, R⁷ is hydrogen, and R⁵ is C₃₋₁₀ cycloalkyl, heterocyclyl, aryl, or heteroaryl; wherein the C₃₋₁₀ cycloalkyl, heterocyclyl, aryl, or heteroaryl is independently optionally substituted with one to five halo, -OR¹¹, -C(O)OR¹¹, cyano, C₁₋₆ alkyl, C₁₋₆ haloalkyl, C₃₋₁₀ cycloalkyl, heterocyclyl, aryl, or heteroaryl.

In certain embodiments, R⁵ is C₃₋₁₀ cycloalkyl, heterocyclyl, or heteroaryl; wherein the C₃₋₁₀ cycloalkyl, heterocyclyl, or heteroaryl is independently optionally substituted with one to five halo, hydroxy, C₁₋₆ alkyl, C₁₋₆ haloalkyl, C₃₋₁₀ cycloalkyl, or -C(O)OR¹¹.

In certain embodiments, R⁵ is C₃₋₁₀ cycloalkyl optionally substituted with one to five halo, -OR¹¹, -C(O)OR¹¹, cyano, C₁₋₆ alkyl, C₁₋₆ haloalkyl, C₃₋₁₀ cycloalkyl, heterocyclyl, aryl, or heteroaryl. In certain embodiments, R⁵ is heterocyclyl optionally substituted with one to five halo, -OR¹¹, -C(O)OR¹¹, cyano, C₁₋₆ alkyl, C₁₋₆ haloalkyl, C₃₋₁₀ cycloalkyl, heterocyclyl, aryl, or heteroaryl. In certain embodiments, R⁵ is heteroaryl optionally substituted with one to five halo, -OR¹¹, -C(O)OR¹¹, cyano, C₁₋₆ alkyl, C₁₋₆ haloalkyl, C₃₋₁₀ cycloalkyl, heterocyclyl, aryl, or heteroaryl.

In certain embodiments, R⁵ is 5-fluoropyrimidin-4-yl, 1-ethylpiperidin-3-yl, 1-cyclobutylpiperidin-3-yl, 3-hydroxy-3-methylcyclobutyl, 3-fluoropyridin-4-yl, tert-butyl 3,3-difluoropiperidine-1-carboxylate-5-yl, 5,5-difluoropiperidin-3-yl, 1-ethyl-5,5-difluoropiperidin-3-yl, 5-fluoropyrimidin-2-yl, pyrimidin-2-yl, 5-cyano-3-fluoropyridin-2-yl, 6-methoxypyridin-3-yl, 6-chloropyridin-3-yl, 5-fluoro-2-methylpyrimidin-4-yl, pyrimidin-4-yl, 2-(trifluoromethyl)pyrimidin-4-yl, 3-fluoropyridin-2-yl, 6-chloro-3-fluoropyridin-2-yl, 3,5-difluoropyridin-4-yl, 3,5-difluoropyridin-2-yl, 3,6-difluoropyridin-2-yl, 3-fluoro-5-(trifluoromethyl)pyridin-2-yl, 3,3-difluorocyclopentyl, 2-methyltetrahydro-2H-pyran-4-yl, 4-methyltetrahydro-2H-pyran-4-yl, 6-(trifluoromethyl)pyridin-3-yl, 2,2-difluorocyclopentyl, 5,6,7,8-tetrahydro-[1,2,4]triazolo[4,3-a]pyridin-6-yl, 1-cyclobutyl-1H-pyrazol-4-yl, 1-methyl-1H-pyrazol-5-yl, 4-fluoro-1-methyl-1H-pyrazol-5-yl, 2-cyclopropylpyrimidin-4-yl, tetrahydrofuran-3-yl, tetrahydro-2H-pyran-4-yl, 2-oxaspiro[3.3]heptan-6-yl, pyridin-3-yl, pyrimidin-5-yl, (tetrahydro-2H-pyran-3-yl)methyl, (4-methylmorpholin-3-yl)methyl, 4-cyano-2-fluorophenyl, 2,2-dimethyltetrahydro-2H-pyran-4-yl, 1-methyl-1H-indazol-6-yl, 4-fluoro-1-methyl-1H-pyrazol-3-yl, 1,4-dimethyl-1H-pyrazol-5-yl, 1,3-dimethyl-1H-pyrazol-5-yl, (4-methylmorpholin-2-yl)methyl, 1-methyl-6-oxopiperidin-3-yl, 1-(oxetan-3-yl)-1H-pyrazol-5-yl, 5-chloro-3-fluoropyridin-2-yl, 1-methyl-1H-pyrazol-3-yl, pyridazin-3-yl, pyrazin-2-yl, 1-phenylethyl, (tetrahydro-2H-pyran-4-yl)methyl, (tetrahydrofuran-3-yl)methyl, 1-cyclobutylethyl, 3-methoxycyclobutyl, 1-ethylpiperidin-3-yl, 1-(tert-butoxycarbonyl)pyrrolidin-2-yl, pyrrolidin-2-ylmethyl, (1-ethylpyrrolidin-2-yl)methyl, 4-(trifluoromethyl)pyrimidin-2-yl, 5-cyclopropylpyridin-2-yl, 6-chloro-5-fluoropyridin-3-yl, 3-cyano-5-fluoropyridin-2-yl, 3-chloro-5-fluoropyridin-2-yl, 1-methyl-3-(trifluoromethyl)-1H-pyrazol-5-yl, 3-(1H-pyrazol-1-yl)cyclobutyl, 3-fluorobicyclo[1.1.1]pentan-1-yl, 5-cyanopyridin-2-yl, 5-chloropyrimidin-2-yl, pyridazin-4-yl, 5-fluoropyridin-3-yl, 1-cyclobutyl-1H-pyrazol-5-yl, 3-fluoro-5-methylpyridin-2-yl, tetrahydro-2H-pyran-3-yl, cyclobutyl, cyclobutylmethyl, (1-methyl-1H-pyrazol-5-yl)methyl, benzyl, (tetrahydrofuran-2-yl)methyl, (tetrahydro-2H-pyran-2-yl)methyl, 4-methyloxan-4-yl, 2-methyloxan-4-yl, 5-cyanopyrimidin-2-yl, 5-methylpyrimidin-2-yl, piperidin-3-yl, 1-(tert-butoxycarbonyl)piperidin-3-yl, 1-cyclopropylpiperidin-3-yl, 4-ethyl-1,4-oxazepan-6-yl; or R⁴ and R⁵ form a 3,4-dihydroquinolin-1(2H)-yl.

In certain embodiments, R⁵ is 5-fluoropyrimidin-4-yl, 1-ethylpiperidin-3-yl, 1-cyclobutylpiperidin-3-yl, 3-hydroxy-3-methylcyclobutyl, 3-fluoropyridin-4-yl, tert-butyl 3,3-difluoropiperidine-1-carboxylate-5-yl, 5,5-difluoropiperidin-3-yl, 1-ethyl-5,5-difluoropiperidin-3-yl, 5-fluoropyrimidin-2-yl, pyrimidin-2-yl, 5-cyano-3-fluoropyridin-2-yl, 6-methoxypyridin-3-yl, 6-chloropyridin-3-yl, 5-fluoro-2-methylpyrimidin-4-yl, pyrimidin-4-yl, 2-(trifluoromethyl)pyrimidin-4-yl, 3-fluoropyridin-2-yl, 6-chloro-3-fluoropyridin-2-yl, 3,5-difluoropyridin-4-yl, 3,5-difluoropyridin-2-yl, 3,6-difluoropyridin-2-yl, 3-fluoro-5-(trifluoromethyl)pyridin-2-yl, 3,3-difluorocyclopentyl, 2-methyltetrahydro-2H-pyran-4-yl, 4-methyltetrahydro-2H-pyran-4-yl, 6-(trifluoromethyl)pyridin-3-yl, 2,2-difluorocyclopentyl, 5,6,7,8-tetrahydro-[1,2,4]triazolo[4,3-a]pyridin-6-yl, 1-cyclobutyl-1H-pyrazol-4-yl, 1-methyl-1H-pyrazol-5-yl, 4-fluoro-1-methyl-1H-pyrazol-5-yl, 2-cyclopropylpyrimidin-4-yl, tetrahydrofuran-3-yl, tetrahydro-2H-pyran-4-yl, 2-oxaspiro[3.3]heptan-6-yl, pyridin-3-yl, pyrimidin-5-yl, (tetrahydro-2H-pyran-3-yl)methyl, (4-methylmorpholin-3-yl)methyl, 4-cyano-2-fluorophenyl, 2,2-dimethyltetrahydro-2H-pyran-4-yl, 1-methyl-1H-indazol-6-yl, 4-fluoro-1-methyl-1H-pyrazol-3-yl, 1,4-dimethyl-1H-pyrazol-5-yl, 1,3-dimethyl-1H-pyrazol-5-yl, (4-methylmorpholin-2-yl)methyl, 1-methyl-6-oxopiperidin-3-yl, 1-(oxetan-3-yl)-1H-pyrazol-5-yl, 5-chloro-3-fluoropyridin-2-yl, 1-methyl-1H-pyrazol-3-yl, pyridazin-3-yl, pyrazin-2-yl, 1-phenylethyl, (tetrahydro-2H-pyran-4-yl)methyl, (tetrahydrofuran-3-yl)methyl, 1-cyclobutylethyl, 3-methoxycyclobutyl, 1-ethylpiperidin-3-yl, 1-(tert-butoxycarbonyl)pyrrolidin-2-yl, pyrrolidin-2-ylmethyl, (1-ethylpyrrolidin-2-yl)methyl, 4-(trifluoromethyl)pyrimidin-2-yl, 5-cyclopropylpyridin-2-yl, 6-chloro-5-fluoropyridin-3-yl, 3-cyano-5-fluoropyridin-2-yl, 3-chloro-5-fluoropyridin-2-yl, 1-methyl-3-(trifluoromethyl)-1H-pyrazol-5-yl, 3-(1H-pyrazol-1-yl)cyclobutyl, 3-fluorobicyclo[1.1.1]pentan-1-yl, 5-cyanopyridin-2-yl, 5-chloropyrimidin-2-yl, pyridazin-4-yl, 5-fluoropyridin-3-yl, 1-cyclobutyl-1H-pyrazol-5-yl, 3-fluoro-5-methylpyridin-2-yl, tetrahydro-2H-pyran-3-yl, cyclobutyl, cyclobutylmethyl, (1-methyl-1H-pyrazol-5-yl)methyl, benzyl, (tetrahydrofuran-2-yl)methyl, (tetrahydro-2H-pyran-2-yl)methyl, 4-methyloxan-4-yl, or 2-methyloxan-4-yl; or R⁴ and R⁵ form a 3,4-dihydroquinolin-1(2H)-yl.

In certain embodiments, R⁵ is 5-fluoropyrimidin-4-yl, 1-ethylpiperidin-3-yl, 1-cyclobutylpiperidin-3-yl, 3-hydroxy-3-methylcyclobutyl, 3-fluoropyridin-4-yl, tert-butyl 3,3-difluoropiperidine-1-carboxylate-5-yl, 5,5-difluoropiperidin-3-yl, or 1-ethyl-5,5-difluoropiperidin-3-yl.

In certain embodiments, R² is C₁₋₆ alkyl, C₁₋₆ haloalkyl, -OR¹¹, -SR¹¹, or halo; wherein the C₁₋₆ alkyl is optionally substituted with one to eight Z². In certain embodiments, R² is C₁₋₆ alkyl, C₁₋₆ haloalkyl, -OR¹¹, -SR¹¹, or halo; wherein the C₁₋₆ alkyl is optionally substituted with —OH; and R¹¹ is hydrogen, C₁₋₆ alkyl, C₁₋₆ haloalkyl, C₃₋₁₀ cycloalkyl, or heterocyclyl; wherein the C₁₋₆ alkyl, C₃₋₁₀ cycloalkyl or heterocyclyl of R¹¹ is optionally substituted with one to five Z^{1a}.

In certain embodiments, R² is fluoro, bromo, chloro, —CH₃, —OCH₃, —CH₂F, —OCH₂F, —CHF₂, —OCHF₂, —CF₃, -SCH₃, 1,1-difluoroethyl, 2,2-difluoroethyl, 1-fluoroethyl, 2-fluoroethyl, 2-hydroxyethyl, 2,2,2-trifluoroethyl, 1-hydroxyethyl, 1,1,1-trifluoropropan-2-yl, 2,2,2-trifluoroethoxy, 5-fluoropyrimidin-2-yl, cyclopropyloxy, cyclobutyloxy, ethoxy, propan-2-yloxy, (3,3-dimethylcyclobutyl)oxy, (3-methylcyclobutyl)oxy, (3-methoxycyclobutyl)oxy, oxetan-3-yloxy, 2-fluoropropoxy, 2-methoxyethoxy, cyclopropyl(fluoro)methoxy, 1-cyclopropylethoxy, [1-(2,2,2-trifluoroethyl)azetidin-3-yl]oxy, (3-cyanocyclobutyl)oxy, or (3-methoxycyclobutyl)oxy.

In certain embodiments, each Z^{1a} is independently halo, cyano, -OR¹³, C₁₋₆ alkyl, or C₃₋₁₀ cycloalkyl.

In certain embodiments, R² is C₁₋₆ alkyl, C₁₋₆haloalkyl, -OR¹¹, or halo; wherein the C1-6 alkyl is optionally substituted with one to eight Z². In certain embodiments, R² is C₁₋₆ alkyl, C₁₋₆ haloalkyl, -OR¹¹, or halo; wherein the C₁₋₆ alkyl is optionally substituted with —OH. In certain embodiments, R² is C₁₋₆ alkyl, C₁₋₆ haloalkyl, -OR¹¹, or halo; wherein the C₁₋₆ alkyl is optionally substituted with —OH. In certain embodiments, R² is C₁₋₆ alkyl, C₁₋₆ haloalkyl, -OR¹¹, or halo; wherein the C₁₋₆ alkyl is optionally substituted with —OH; and each R¹¹ is independently hydrogen, C₁₋₆ alkyl, or C₁₋₆ haloalkyl. In certain embodiments, R² is C₁₋₆ alkyl, C₁₋₆ alkoxy, C₁₋₆ haloalkyl or C₁₋₆ haloalkoxy. In certain embodiments, R² is fluoro, bromo, —CH₃, —OCH₃, —CHF₂, —OCHF₂, 1,1-difluoroethyl, 2,2-difluoroethyl, 1-fluoroethyl, 2-fluoroethyl, 2-hydroxyethyl, 2,2,2-trifluoroethyl, 1-hydroxyethyl, or 1,1,1-trifluoropropan-2-yl.

In certain embodiments, R² is C₁₋₆ alkyl, C₁₋₆ haloalkyl, or -OR¹¹, wherein R¹¹ is C₁₋₆alkyl optionally substituted with one to five Z^{1a}. In certain embodiments, R² is C₁₋₆ alkyl or C₁₋₆ haloalkyl. In certain embodiments, R² is C₁₋₆ alkyl. In certain embodiments, R² is isopropyl.

In certain embodiments, R² is -OR¹¹, and R¹¹ is C₁₋₆ alkyl, C₁₋₆haloalkyl, C₃₋₁₀ cycloalkyl, or heterocyclyl; wherein the C₁₋₆ alkyl, C₃₋₁₀ cycloalkyl or heterocyclyl of R¹¹ is optionally substituted with one to five Z^{1a}.

In certain embodiments, R² is -OR¹¹, and R¹¹ is C₁₋₆ alkyl In certain embodiments, R² is -OR¹¹, and R¹¹ is C₃₋₁₀ cycloalkyl optionally substituted with one to five Z^{1a}. In certain embodiments, R² is -OR¹¹, and R¹¹ is heterocyclyl optionally substituted with one to five Z^{1a}.

In certain embodiments, each Z^{1a} is independently halo, cyano, -OR¹³, C₁₋₆ alkyl, or C₃₋ ₁₀cycloalkyl. In certain embodiments, each Z^{1a} is independently cyano, -OR¹³, C₁₋₆ alkyl, or C₃₋ ₁₀cycloalkyl.

In certain embodiments, R² is -C(R¹⁴)₂R¹⁵; each R¹⁴ and R¹⁵ are independently hydrogen, halo, C₁₋₄ alkyl, or C₁₋₄ haloalkyl. In certain embodiments, R² is -C(R¹⁴)₂R¹⁵; each R¹⁴ is independently hydrogen, halo, C₁₋₄ alkyl, or C₁₋₄ haloalkyl, and R¹⁵ is hydrogen.

In certain embodiments, R² is C₃₋₁₀ cycloalkyl optionally substituted with one to eight Z². In certain embodiments, R² is cyclopropyl optionally substituted with one to eight Z². In certain embodiments, R² is C₃₋₁₀ cycloalkyl In certain embodiments, R² is cyclopropyl.

In certain embodiments, Z^{1a} is independently halo.

In certain embodiments, each Z¹ is independently halo, hydroxy, C₁₋₆ alkyl, C₁₋₆ haloalkyl, C₃₋₁₀ cycloalkyl, or -C(O)OR¹¹.

In certain embodiments, each R¹¹ is independently hydrogen, C₁₋₆ alkyl, C₁₋₆haloalkyl, C₃₋₁₀ cycloalkyl, or heterocyclyl; wherein the C₁₋₆ alkyl, C₃₋₁₀ cycloalkyl or heterocyclyl of R¹¹ is optionally substituted with one to five Z^{1a}. In certain embodiments, each Z^{1a} is independently halo, cyano, -OR¹³, C₁₋₆ alkyl, or C₃₋₁₀ cycloalkyl.

In certain embodiments, each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl.

In certain embodiments, each R¹¹ is independently hydrogen or C₁₋₆ alkyl. In certain embodiments, each R¹¹ is hydrogen.

In certain embodiments, R¹² is C₁₋₆ alkyl, C₂₋₆ alkenyl, C₂₋₆ alkynyl, or C₁₋₆ haloalkyl.

In certain embodiments, each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl. In certain embodiments, each R¹³ is independently hydrogen or C₁₋₆ alkyl.

In certain embodiments, R² is C₁₋₆ alkyl, R⁴ is hydrogen, R⁶ is hydrogen, and R⁷ is hydrogen.

In certain embodiments, each of A¹, A², A³, and A⁴ is independently CH or CR¹; provided at least one of A¹, A², A³, and A⁴ is CR¹; each R¹ is independently halo, cyano, C₁₋₆ alkyl, C₁₋₆ alkoxy, C₁₋₆ haloalkyl, C₁₋₆ haloalkoxy, -N(R¹¹)₂, -SR¹¹, or C₃₋₁₀ cycloalkyl; R² is C₁₋₆ alkyl, C₁₋₆ haloalkyl, -SR¹¹, -OR¹¹, or halo; wherein the C₁₋₆ alkyl is optionally substituted with one to eight Z²; R⁴ is hydrogen; R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹; R⁶ is hydrogen or C₁₋₆ alkyl; and R⁷ is hydrogen.

In certain embodiments, one of A¹, A², A³, and A⁴ is N; one of A¹, A², A³, and A⁴ is CR¹; and the remaining A¹, A², A³, and A⁴ are independently CH or CR¹; each R¹ is independently halo, cyano, C₁₋₆ alkyl, C₁₋₆ alkoxy, C₁₋₆ haloalkyl, C₁₋₆haloalkoxy, -N(R¹¹)₂, -SR¹¹, or C₃₋₁₀ cycloalkyl; R² is C₁₋₆ alkyl, C₁₋₆ haloalkyl, -SR¹¹, -OR¹¹, or halo; wherein the C₁₋₆ alkyl is optionally substituted with one to eight Z²; R⁴ is hydrogen; R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹; R⁶ is hydrogen or C₁₋₆ alkyl; and R⁷ is hydrogen.

In certain embodiments, two of A¹, A², A³, and A⁴ are N; one of A¹, A², A³, and A⁴ is CR¹; and the remaining A¹, A², A³, and A⁴ is CH or CR¹; each R¹ is independently halo, cyano, C₁₋₆ alkyl, C₁₋₆ alkoxy, C₁₋₆haloalkyl, C₁₋₆ haloalkoxy, -N(R¹¹)₂, -SR¹¹, or C₃₋₁₀ cycloalkyl; R² is C₁₋₆ alkyl, C₁₋₆ haloalkyl, -SR¹¹, -OR¹¹, or halo; wherein the C₁₋₆ alkyl is optionally substituted with one to eight Z²; R⁴ is hydrogen; R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹; R⁶ is hydrogen or C₁₋₆ alkyl; and R⁷ is hydrogen.

In certain embodiments, A² is CR¹ and A¹, A³ and A⁴ are each independently N, CH, or CR¹; each R¹ is independently halo, cyano, C₁₋₆ alkyl, C₁₋₆ alkoxy, C₁₋₆ haloalkyl, C₁₋₆ haloalkoxy, -SR¹¹, -N(R¹¹)₂, or C₃₋₁₀ cycloalkyl; R² is C₁₋₆ alkyl, C₁₋₆ haloalkyl, -SR¹¹, -OR¹¹, or halo; wherein the C₁₋₆ alkyl is optionally substituted with one to eight Z²; R⁴ is hydrogen; R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹; R⁶ is hydrogen or C₁₋₆ alkyl; and R⁷ is hydrogen.

In certain embodiments, A³ is CR¹ and A¹, A², and A⁴ are each independently N, CH, or CR¹; each R¹ is independently halo, cyano, C₁₋₆ alkyl, C₁₋₆ alkoxy, C₁₋₆ haloalkyl, C₁₋₆ haloalkoxy, -SR¹¹, -N(R¹¹)₂, or C₃₋₁₀ cycloalkyl; R² is C₁₋₆ alkyl, C₁₋₆ haloalkyl, -SR¹¹, -OR¹¹, or halo; wherein the C₁₋₆ alkyl is optionally substituted with one to eight Z²; R⁴ is hydrogen; R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹; R⁶ is hydrogen or C₁₋₆ alkyl; and R⁷ is hydrogen.

In certain embodiments, each of A¹, A², A³, and A⁴ is independently CH or CR¹; provided at least one of A¹, A², A³, and A⁴ is CR¹; each R¹ is independently halo, cyano, C₁₋₆ alkyl, C₁₋₆ alkoxy, C₁₋₆ haloalkyl, C₁₋₆ haloalkoxy, -SR¹¹, or C₃₋₁₀ cycloalkyl; R² is C₁₋₆ alkyl, C₁₋₆ haloalkyl, -OR¹¹, or halo; wherein the C₁₋₆ alkyl is optionally substituted with one to eight Z²; R⁴ is hydrogen; R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹; R⁶ is hydrogen or C₁₋₆ alkyl; and R⁷ is hydrogen.

In certain embodiments, one of A¹, A², A³, and A⁴ is N; one of A¹, A², A³, and A⁴ is CR¹; and the remaining A¹, A², A³, and A⁴ are independently CH or CR¹; each R¹ is independently halo, cyano, C₁₋₆ alkyl, C₁₋₆ alkoxy, C₁₋₆ haloalkyl, C₁₋₆ haloalkoxy, -SR¹¹, or C₃₋₁₀ cycloalkyl; R² is C₁₋₆ alkyl, C₁₋₆ haloalkyl, -OR¹¹, or halo; wherein the C₁₋₆ alkyl is optionally substituted with one to eight Z²; R⁴ is hydrogen; R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹; R⁶ is hydrogen or C₁₋₆ alkyl; and R⁷ is hydrogen.

In certain embodiments, two of A¹, A², A³, and A⁴ are N; one of A¹, A², A³, and A⁴ is CR¹; and the remaining A¹, A², A³, and A⁴ is CH or CR¹; each R¹ is independently halo, cyano, C₁₋₆ alkyl, C₁₋₆ alkoxy, C₁₋₆ haloalkyl, C₁₋₆ haloalkoxy, -SR¹¹, or C₃₋₁₀ cycloalkyl; R² is C₁₋₆ alkyl, C₁₋₆ haloalkyl, -OR¹¹, or halo; wherein the C₁₋₆ alkyl is optionally substituted with one to eight Z²; R⁴ is hydrogen; R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹; R⁶ is hydrogen or C₁₋₆ alkyl; and R⁷ is hydrogen.

In certain embodiments, A² is CR¹ and A¹, A³ and A⁴ are each independently N, CH, or CR¹; each R¹ is independently halo, cyano, C₁₋₆ alkyl, C₁₋₆ alkoxy, C₁₋₆ haloalkyl, C₁₋₆ haloalkoxy, -SR¹¹, or C₃₋₁₀ cycloalkyl; R² is C₁₋₆ alkyl, C₁₋₆ haloalkyl, -OR¹¹, or halo; wherein the C₁₋₆ alkyl is optionally substituted with one to eight Z²; R⁴ is hydrogen; R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹; R⁶ is hydrogen or C₁₋₆ alkyl; and R⁷ is hydrogen.

In certain embodiments, A³ is CR¹ and A¹, A², and A⁴ are each independently N, CH, or CR¹; each R¹ is independently halo, cyano, C₁₋₆ alkyl, C₁₋₆ alkoxy, C₁₋₆ haloalkyl, C₁₋₆ haloalkoxy, -SR¹¹, or C₃₋₁₀ cycloalkyl; R² is C₁₋₆ alkyl, C₁₋₆ haloalkyl, -OR¹¹, or halo; wherein the C₁₋₆ alkyl is optionally substituted with one to eight Z²; R⁴ is hydrogen; R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or R⁴ and R⁵ together form a heterocyclyl ring optionally substituted with one to eight Z¹; R⁶ is hydrogen or C₁₋₆ alkyl; and R⁷ is hydrogen.

In certain embodiments, each of A¹, A², A³, and A⁴ is independently CH or CR¹; provided at least one of A¹, A², A³, and A⁴ is CR¹; each R¹ is independently halo, cyano, C₁₋₆ alkyl, C₁₋₆ alkoxy, or C₁₋₆ haloalkyl; R² is C₁₋₆ alkyl, C₁₋₆ haloalkyl, or -OR¹¹, wherein R¹¹ is C₁₋₆ alkyl optionally substituted with one to five Z^{1a}; R⁴ is hydrogen; R⁵ is C₃₋₁₀ cycloalkyl, heterocyclyl, or heteroaryl; wherein the C₃₋₁₀ cycloalkyl, heterocyclyl, or heteroaryl is independently optionally substituted with one to five Z¹; R⁶ is hydrogen; and R⁷ is hydrogen.

In certain embodiments, one of A¹, A², A³, and A⁴ is N; one of A¹, A², A³, and A⁴ is CR¹; and the remaining A¹, A², A³, and A⁴ are independently CH or CR¹; each R¹ is independently halo, cyano, C₁₋₆ alkyl, C₁₋₆ alkoxy, or C₁₋₆ haloalkyl; R² is C₁₋₆ alkyl, C₁₋₆ haloalkyl, or -OR¹¹, wherein R¹¹ is C₁₋₆ alkyl optionally substituted with one to five Z^{1a}; R⁴ is hydrogen; R⁵ is C₃₋₁₀ cycloalkyl, heterocyclyl, or heteroaryl; wherein the C₃₋₁₀ cycloalkyl, heterocyclyl, or heteroaryl is independently optionally substituted with one to five Z¹; R⁶ is hydrogen; and R⁷ is hydrogen.

In certain embodiments, two of A¹, A², A³, and A⁴ are N; one of A¹, A², A³, and A⁴ is CR¹; and the remaining A¹, A², A³, and A⁴ is CH or CR¹; each R¹ is independently halo, cyano, C₁₋₆ alkyl, C₁₋₆ alkoxy, or C₁₋₆ haloalkyl; R² is C₁₋₆ alkyl, C₁₋₆ haloalkyl, or -OR¹¹, wherein R¹¹ is C₁₋₆ alkyl optionally substituted with one to five Z^{1a}; R⁴ is hydrogen; R⁵ is C₃₋₁₀ cycloalkyl, heterocyclyl, or heteroaryl; wherein the C₃₋₁₀ cycloalkyl, heterocyclyl, or heteroaryl is independently optionally substituted with one to five Z¹; R⁶ is hydrogen; and R⁷ is hydrogen.

In certain embodiments, A² is CR¹ and A¹, A³ and A ⁴ are each independently N, CH, or CR¹; each R¹ is independently halo, cyano, C₁₋₆ alkyl, C₁₋₆ alkoxy, or C₁₋₆ haloalkyl; R² is C₁₋₆ alkyl, C₁₋₆ haloalkyl, or -OR¹¹, wherein R¹¹ is C₁₋₆ alkyl optionally substituted with one to five Z^{1a}; R⁴ is hydrogen; R⁵ is C₃₋₁₀ cycloalkyl, heterocyclyl, or heteroaryl; wherein the C₃₋₁₀ cycloalkyl, heterocyclyl, or heteroaryl is independently optionally substituted with one to five Z¹; R⁶ is hydrogen; and R⁷ is hydrogen.

In certain embodiments, A³ is CR¹ and A¹, A², and A⁴ are each independently N, CH, or CR¹; each R¹ is independently halo, cyano, C₁₋₆ alkyl, C₁₋₆ alkoxy, or C₁₋₆ haloalkyl; R² is C₁₋₆ alkyl, C₁₋₆ haloalkyl, or -OR¹¹, wherein R¹¹ is C₁₋₆ alkyl optionally substituted with one to five Z^{1a}; R⁴ is hydrogen; R⁵ is C₃₋₁₀ cycloalkyl, heterocyclyl, or heteroaryl; wherein the C₃₋₁₀ cycloalkyl, heterocyclyl, or heteroaryl is independently optionally substituted with one to five Z¹; R⁶ is hydrogen; and R⁷ is hydrogen.

In certain embodiments, provided is a compound selected from Table 1, or a pharmaceutically acceptable salt, isotopically enriched analog, prodrug, stereoisomer, or a mixture of stereoisomers thereof:

TABLE 1

| Ex. | Structure |
|---|---|
| 1 | 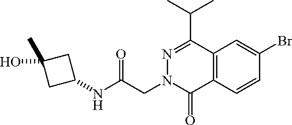 [SMILES: CC(C)c1nn(CC(=O)N[C@H]2C[C@@](C)(O)C2)c(=O)c2ccc(Br)cc12] |
| 2 | 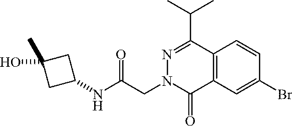 [SMILES: CC(C)c1nn(CC(=O)N[C@H]2C[C@@](C)(O)C2)c(=O)c2cc(Br)ccc12] |
| 3 | 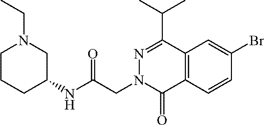 [SMILES: CCN1CCC[C@@H](NC(=O)Cn2nc(C(C)C)c3cc(Br)ccc3c2=O)C1] |
| 4 | 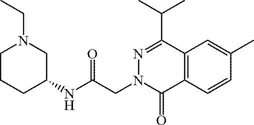 [SMILES: CCN1CCC[C@@H](NC(=O)Cn2nc(C(C)C)c3cc(C)ccc3c2=O)C1] |
| 5 | 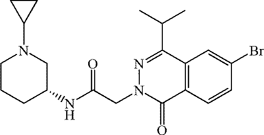 [SMILES: CC(C)c1nn(CC(=O)N[C@@H]2CCCN(C3CC3)C2)c(=O)c2ccc(Br)cc12] |
| 6 | 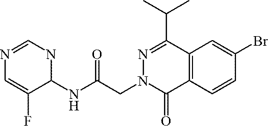 [SMILES: CC(C)c1nn(CC(=O)NC2NC=NC=C2F)c(=O)c2ccc(Br)cc12] |
| 7 | 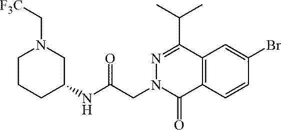 [SMILES: CC(C)c1nn(CC(=O)N[C@@H]2CCCN(CC(F)(F)F)C2)c(=O)c2ccc(Br)cc12] |
| 8 | 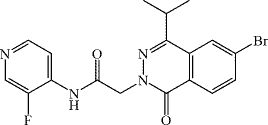 [SMILES: CC(C)c1nn(CC(=O)Nc2ccncc2F)c(=O)c2ccc(Br)cc12] |
| 9 | 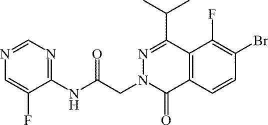 [SMILES: CC(C)c1nn(CC(=O)Nc2ncncc2F)c(=O)c2ccc(Br)c(F)c12] |
| 10 | 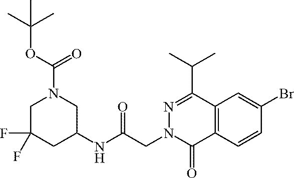 [SMILES: CC(C)c1nn(CC(=O)NC2CN(C(=O)OC(C)(C)C)CC(F)(F)C2)c(=O)c2ccc(Br)cc12] |
| 11 | 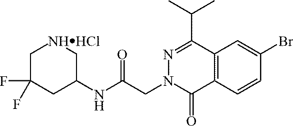 |
| 12 | 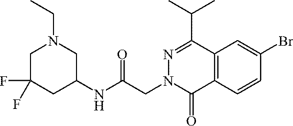 [SMILES: CCN1CC(NC(=O)Cn2nc(C(C)C)c3cc(Br)ccc3c2=O)CC(F)(F)C1] |
| 13 | 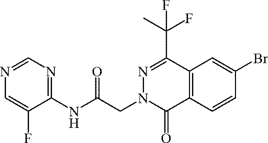 [SMILES: CC(F)(F)c1nn(CC(=O)Nc2ncncc2F)c(=O)c2ccc(Br)cc12] |
| 14 | 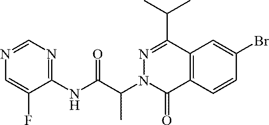 [SMILES: CC(C)c1nn(C(C)C(=O)Nc2ncncc2F)c(=O)c2ccc(Br)cc12] |
| 15 | 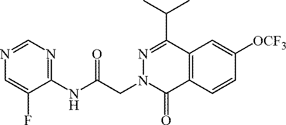 [SMILES: CC(C)c1nn(CC(=O)Nc2ncncc2F)c(=O)c2ccc(OC(F)(F)F)cc12] |
| 16 | 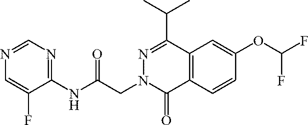 [SMILES: CC(C)c1nn(CC(=O)Nc2ncncc2F)c(=O)c2ccc(OC(F)F)cc12] |
| 17 | 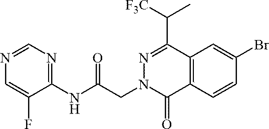 [SMILES: CC(c1nn(CC(=O)Nc2ncncc2F)c(=O)c2ccc(Br)cc12)C(F)(F)F] |
| 18 | 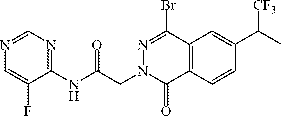 [SMILES: CC(c1ccc2c(=O)n(CC(=O)Nc3ncncc3F)nc(Br)c2c1)C(F)(F)F] |
| 19 | 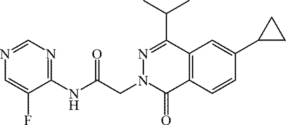 [SMILES: CC(C)c1nn(CC(=O)Nc2ncncc2F)c(=O)c2ccc(C3CC3)cc12] |
| 20 | 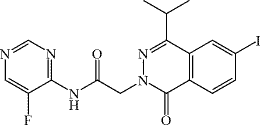 [SMILES: CC(C)c1nn(CC(=O)Nc2ncncc2F)c(=O)c2ccc(I)cc12] |
| 21 | 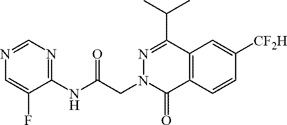 [SMILES: CC(C)c1nn(CC(=O)Nc2ncncc2F)c(=O)c2ccc(C(F)F)cc12] |
| 22 | 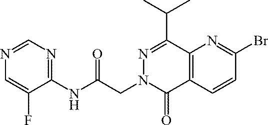 [SMILES: CC(C)c1nn(CC(=O)Nc2ncncc2F)c(=O)c2ccc(Br)nc12] |
| 23 | 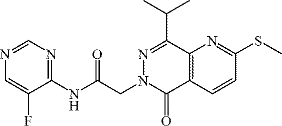 [SMILES: CSc1ccc2c(=O)n(CC(=O)Nc3ncncc3F)nc(C(C)C)c2n1] |
| 24 | 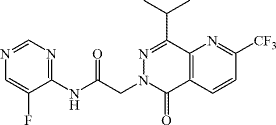 [SMILES: CC(C)c1nn(CC(=O)Nc2ncncc2F)c(=O)c2ccc(C(F)(F)F)nc12] |
| 25 | 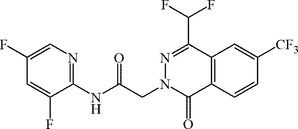 [SMILES: O=C(Cn1nc(C(F)F)c2cc(C(F)(F)F)ccc2c1=O)Nc1ncc(F)cc1F] |
| 26 | 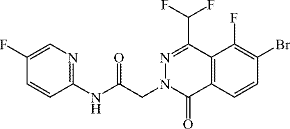 [SMILES: O=C(Cn1nc(C(F)F)c2c(F)c(Br)ccc2c1=O)Nc1ccc(F)cn1] |
| 27 | 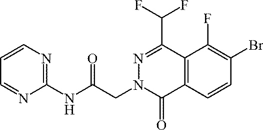 [SMILES: O=C(Cn1nc(C(F)F)c2c(F)c(Br)ccc2c1=O)Nc1ncccn1] |
| 28 | 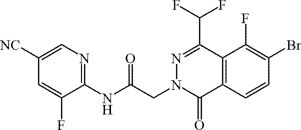 |
| 29 | 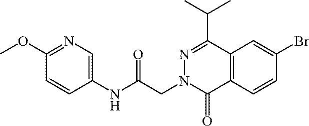 [SMILES: COc1ccc(NC(=O)Cn2nc(C(C)C)c3cc(Br)ccc3c2=O)cn1] |
| 30 | 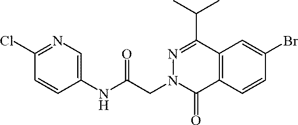 [SMILES: CC(C)c1nn(CC(=O)Nc2ccc(Cl)nc2)c(=O)c2ccc(Br)cc12] |
| 31 | 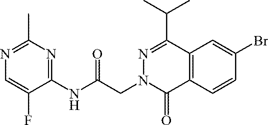 [SMILES: Cc1ncc(F)c(NC(=O)Cn2nc(C(C)C)c3cc(Br)ccc3c2=O)n1] |
| 32 | 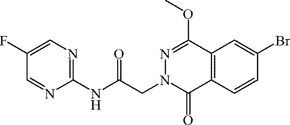 [SMILES: COc1nn(CC(=O)Nc2ncc(F)cn2)c(=O)c2ccc(Br)cc12] |
| 33 | 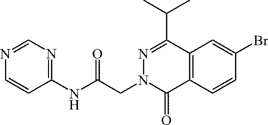 [SMILES: CC(C)c1nn(CC(=O)Nc2ccncn2)c(=O)c2ccc(Br)cc12] |
| 34 | 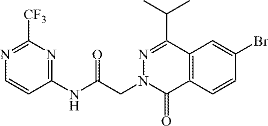 [SMILES: CC(C)c1nn(CC(=O)Nc2ccnc(C(F)(F)F)n2)c(=O)c2ccc(Br)cc12] |
| 35 | 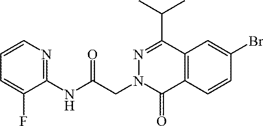 [SMILES: CC(C)c1nn(CC(=O)Nc2ncccc2F)c(=O)c2ccc(Br)cc12] |
| 36 | 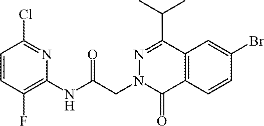 [SMILES: CC(C)c1nn(CC(=O)Nc2nc(Cl)ccc2F)c(=O)c2ccc(Br)cc12] |
| 37 | 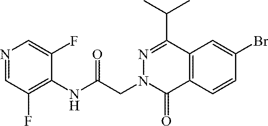 [SMILES: CC(C)c1nn(CC(=O)Nc2c(F)cncc2F)c(=O)c2ccc(Br)cc12] |
| 38 | 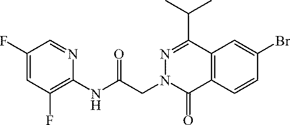 [SMILES: CC(C)c1nn(CC(=O)Nc2ncc(F)cc2F)c(=O)c2ccc(Br)cc12] |
| 39 | 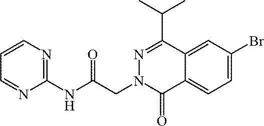 [SMILES: CC(C)c1nn(CC(=O)Nc2ncccn2)c(=O)c2ccc(Br)cc12] |
| 40 | 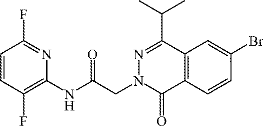 [SMILES: CC(C)c1nn(CC(=O)Nc2nc(F)ccc2F)c(=O)c2ccc(Br)cc12] |
| 41 | 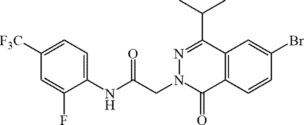 [SMILES: CC(C)c1nn(CC(=O)Nc2ccc(C(F)(F)F)cc2F)c(=O)c2ccc(Br)cc12] |
| 42 | 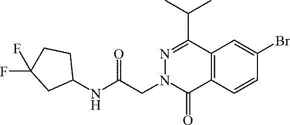 [SMILES: CC(C)c1nn(CC(=O)NC2CCC(F)(F)C2)c(=O)c2ccc(Br)cc12] |
| 43 | 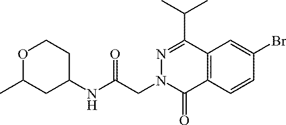 [SMILES: CC1CC(NC(=O)Cn2nc(C(C)C)c3cc(Br)ccc3c2=O)CCO1] |
| 44 | 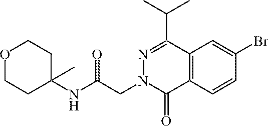 [SMILES: CC(C)c1nn(CC(=O)NC2(C)CCOCC2)c(=O)c2ccc(Br)cc12] |
| 45 | 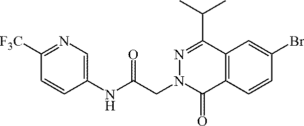 [SMILES: CC(C)c1nn(CC(=O)Nc2ccc(C(F)(F)F)nc2)c(=O)c2ccc(Br)cc12] |
| 46 | 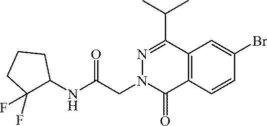 [SMILES: CC(C)c1nn(CC(=O)NC2CCCC2(F)F)c(=O)c2ccc(Br)cc12] |
| 47 | 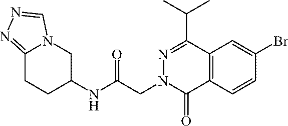 [SMILES: CC(C)c1nn(CC(=O)NC2CCc3nncn3C2)c(=O)c2ccc(Br)cc12] |
| 48 | 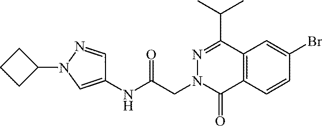 [SMILES: CC(C)c1nn(CC(=O)Nc2cnn(C3CCC3)c2)c(=O)c2ccc(Br)cc12] |
| 49 | 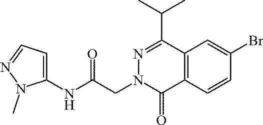 [SMILES: CC(C)c1nn(CC(=O)Nc2ccnn2C)c(=O)c2ccc(Br)cc12] |
| 50 | 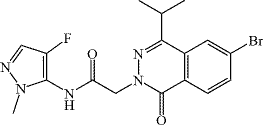 [SMILES: CC(C)c1nn(CC(=O)Nc2c(F)cnn2C)c(=O)c2ccc(Br)cc12] |
| 51 | 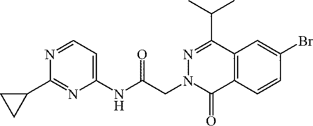 [SMILES: CC(C)c1nn(CC(=O)Nc2ccnc(C3CC3)n2)c(=O)c2ccc(Br)cc12] |
| 52 | 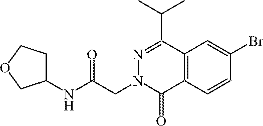 [SMILES: CC(C)c1nn(CC(=O)NC2CCOC2)c(=O)c2ccc(Br)cc12] |
| 53 | 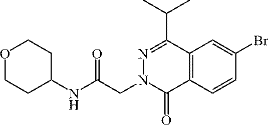 [SMILES: CC(C)c1nn(CC(=O)NC2CCOCC2)c(=O)c2ccc(Br)cc12] |
| 54 | 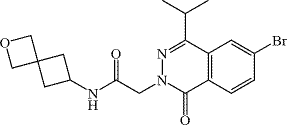 [SMILES: CC(C)c1nn(CC(=O)NC2CC3(COC3)C2)c(=O)c2ccc(Br)cc12] |
| 55 | 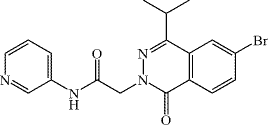 [SMILES: CC(C)c1nn(CC(=O)Nc2cccnc2)c(=O)c2ccc(Br)cc12] |
| 56 | 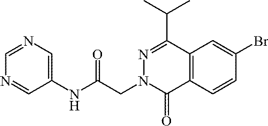 [SMILES: CC(C)c1nn(CC(=O)Nc2cncnc2)c(=O)c2ccc(Br)cc12] |
| 57 | 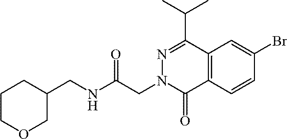 [SMILES: CC(C)c1nn(CC(=O)NCC2CCCOC2)c(=O)c2ccc(Br)cc12] |
| 58 | 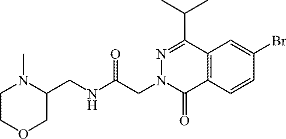 [SMILES: CC(C)c1nn(CC(=O)NCC2COCCN2C)c(=O)c2ccc(Br)cc12] |
| 59 | 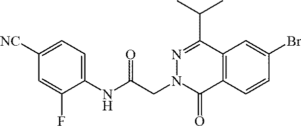 |
| 60 | 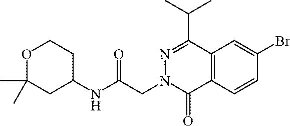 [SMILES: CC(C)c1nn(CC(=O)NC2CCOC(C)(C)C2)c(=O)c2ccc(Br)cc12] |
| 61 | 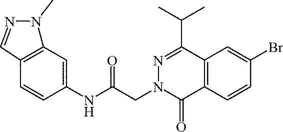 [SMILES: CC(C)c1nn(CC(=O)Nc2ccc3cnn(C)c3c2)c(=O)c2ccc(Br)cc12] |
| 62 | 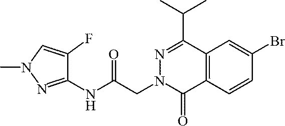 [SMILES: CC(C)c1nn(CC(=O)Nc2nn(C)cc2F)c(=O)c2ccc(Br)cc12] |
| 63 | 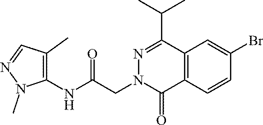 [SMILES: Cc1cnn(C)c1NC(=O)Cn1nc(C(C)C)c2cc(Br)ccc2c1=O] |
| 64 | 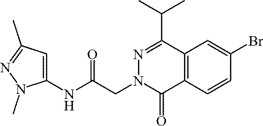 [SMILES: Cc1cc(NC(=O)Cn2nc(C(C)C)c3cc(Br)ccc3c2=O)n(C)n1] |
| 65 | 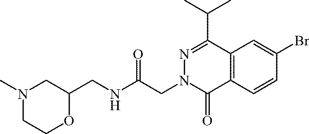 [SMILES: CC(C)c1nn(CC(=O)NCC2CN(C)CCO2)c(=O)c2ccc(Br)cc12] |
| 66 | 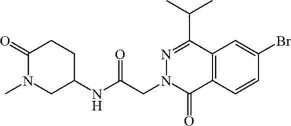 [SMILES: CC(C)c1nn(CC(=O)NC2CCC(=O)N(C)C2)c(=O)c2ccc(Br)cc12] |
| 67 | 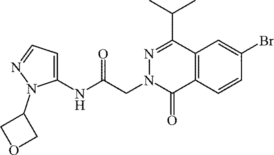 [SMILES: CC(C)c1nn(CC(=O)Nc2ccnn2C2COC2)c(=O)c2ccc(Br)cc12] |
| 68 | 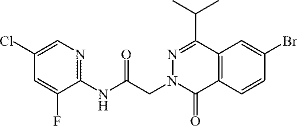 [SMILES: CC(C)c1nn(CC(=O)Nc2ncc(Cl)cc2F)c(=O)c2ccc(Br)cc12] |
| 69 | 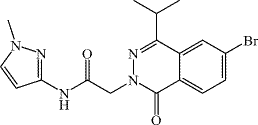 [SMILES: CC(C)c1nn(CC(=O)Nc2ccn(C)n2)c(=O)c2ccc(Br)cc12] |
| 70 | 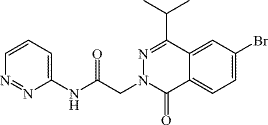 [SMILES: CC(C)c1nn(CC(=O)Nc2cccnn2)c(=O)c2ccc(Br)cc12] |
| 71 | 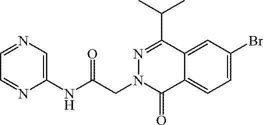 [SMILES: CC(C)c1nn(CC(=O)Nc2cnccn2)c(=O)c2ccc(Br)cc12] |
| 72 | 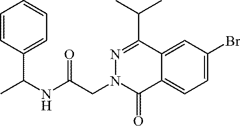 [SMILES: CC(C)c1nn(CC(=O)NC(C)c2ccccc2)c(=O)c2ccc(Br)cc12] |
| 73 | 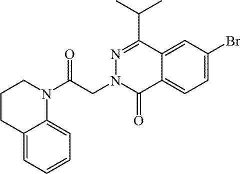 [SMILES: CC(C)c1nn(CC(=O)N2CCCc3ccccc32)c(=O)c2ccc(Br)cc12] |
| 74 | 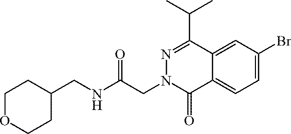 [SMILES: CC(C)c1nn(CC(=O)NCC2CCOCC2)c(=O)c2ccc(Br)cc12] |
| 75 | 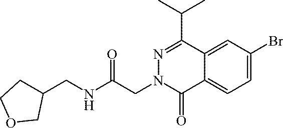 [SMILES: CC(C)c1nn(CC(=O)NCC2CCOC2)c(=O)c2ccc(Br)cc12] |
| 76 | 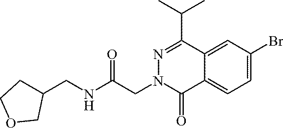 [SMILES: CC(C)c1nn(CC(=O)NCC2CCOC2)c(=O)c2ccc(Br)cc12] |
| 77 | 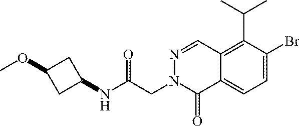 [SMILES: CO[C@H]1C[C@@H](NC(=O)Cn2ncc3c(C(C)C)c(Br)ccc3c2=O)C1] |
| 78 | 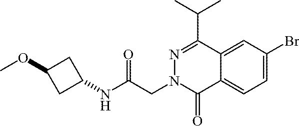 [SMILES: CO[C@H]1C[C@H](NC(=O)Cn2nc(C(C)C)c3cc(Br)ccc3c2=O)C1] |
| 79 | 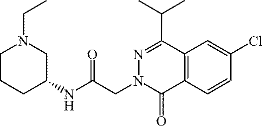 [SMILES: CCN1CCC[C@@H](NC(=O)Cn2nc(C(C)C)c3cc(Cl)ccc3c2=O)C1] |
| 80 | 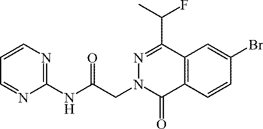 [SMILES: CC(F)c1nn(CC(=O)Nc2ncccn2)c(=O)c2ccc(Br)cc12] |
| 81 | 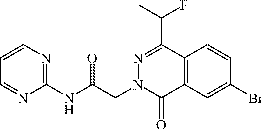 [SMILES: CC(F)c1nn(CC(=O)Nc2ncccn2)c(=O)c2cc(Br)ccc12] |
| 82 | 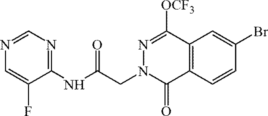 [SMILES: O=C(Cn1nc(OC(F)(F)F)c2cc(Br)ccc2c1=O)Nc1ncncc1F] |
| 83 | 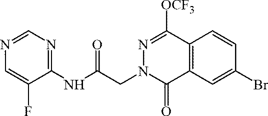 [SMILES: O=C(Cn1nc(OC(F)(F)F)c2ccc(Br)cc2c1=O)Nc1ncncc1F] |
| 84 | 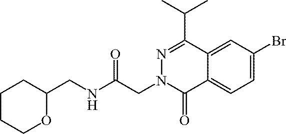 [SMILES: CC(C)c1nn(CC(=O)NCC2CCCCO2)c(=O)c2ccc(Br)cc12] |
| 85 | 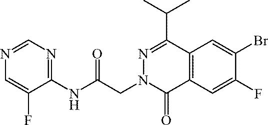 [SMILES: CC(C)c1nn(CC(=O)Nc2ncncc2F)c(=O)c2cc(F)c(Br)cc12] |
| 86 | 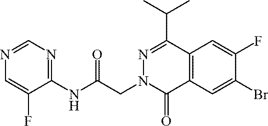 [SMILES: CC(C)c1nn(CC(=O)Nc2ncncc2F)c(=O)c2cc(Br)c(F)cc12] |
| 87 | 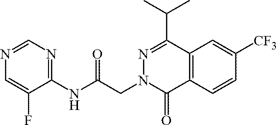 [SMILES: CC(C)c1nn(CC(=O)Nc2ncncc2F)c(=O)c2ccc(C(F)(F)F)cc12] |
| 88 | 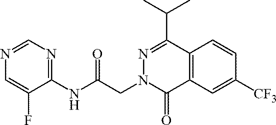 [SMILES: CC(C)c1nn(CC(=O)Nc2ncncc2F)c(=O)c2cc(C(F)(F)F)ccc12] |
| 89 | 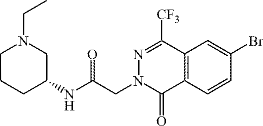 [SMILES: CCN1CCC[C@@H](NC(=O)Cn2nc(C(F)(F)F)c3cc(Br)ccc3c2=O)C1] |
| 90 | 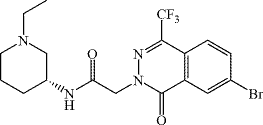 [SMILES: CCN1CCC[C@@H](NC(=O)Cn2nc(C(F)(F)F)c3ccc(Br)cc3c2=O)C1] |
| 91 | 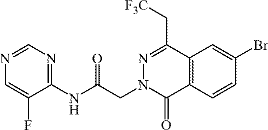 [SMILES: O=C(Cn1nc(CC(F)(F)F)c2cc(Br)ccc2c1=O)Nc1ncncc1F] |
| 92 | 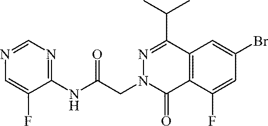 [SMILES: CC(C)c1nn(CC(=O)Nc2ncncc2F)c(=O)c2c(F)cc(Br)cc12] |
| 93 | 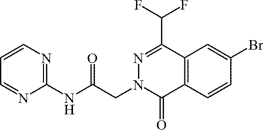 [SMILES: O=C(Cn1nc(C(F)F)c2cc(Br)ccc2c1=O)Nc1ncccn1] |
| 94 | 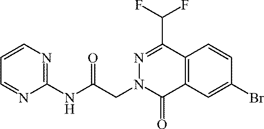 [SMILES: O=C(Cn1nc(C(F)F)c2ccc(Br)cc2c1=O)Nc1ncccn1] |
| 95 | 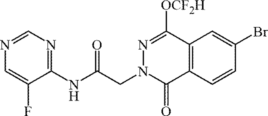 |
| 96 | 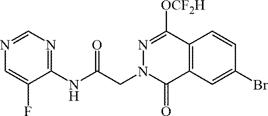 |
| 97 | 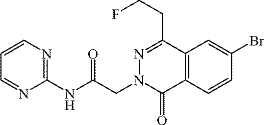 [SMILES: O=C(Cn1nc(CCF)c2cc(Br)ccc2c1=O)Nc1ncccn1] |
| 98 | 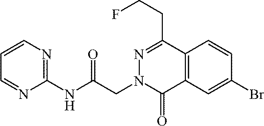 [SMILES: O=C(Cn1nc(CCF)c2ccc(Br)cc2c1=O)Nc1ncccn1] |
| 99 | 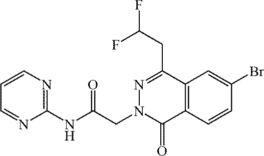 [SMILES: O=C(Cn1nc(CC(F)F)c2cc(Br)ccc2c1=O)Nc1ncccn1] |
| 100 | 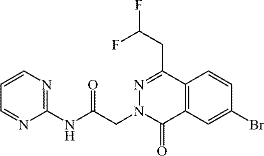 [SMILES: O=C(Cn1nc(CC(F)F)c2ccc(Br)cc2c1=O)Nc1ncccn1] |
| 101 | 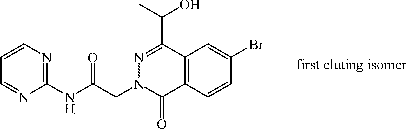 [SMILES: CC(O)c1nn(CC(=O)Nc2ncccn2)c(=O)c2ccc(Br)cc12] |
| 102 | 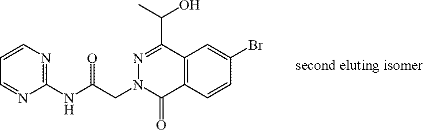 [SMILES: CC(O)c1nn(CC(=O)Nc2ncccn2)c(=O)c2ccc(Br)cc12] |
| 103 | 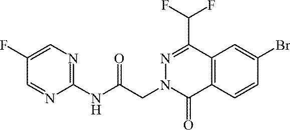 [SMILES: O=C(Cn1nc(C(F)F)c2cc(Br)ccc2c1=O)Nc1ncc(F)cn1] |
| 104 | 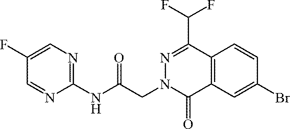 [SMILES: O=C(Cn1nc(C(F)F)c2ccc(Br)cc2c1=O)Nc1ncc(F)cn1] |
| 105 | 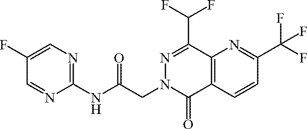 [SMILES: O=C(Cn1nc(C(F)F)c2nc(C(F)(F)F)ccc2c1=O)Nc1ncc(F)cn1] |
| 106 | 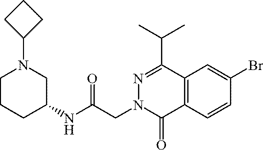 [SMILES: CC(C)c1nn(CC(=O)N[C@@H]2CCCN(C3CCC3)C2)c(=O)c2ccc(Br)cc12] |
| 107 | 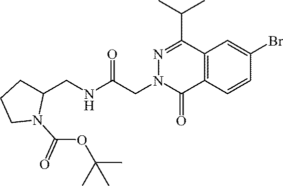 [SMILES: CC(C)c1nn(CC(=O)NCC2CCCN2C(=O)OC(C)(C)C)c(=O)c2ccc(Br)cc12] |
| 108 | 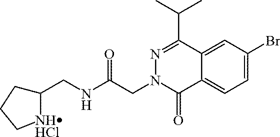 |
| 109 | 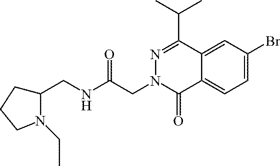 [SMILES: CCN1CCCC1CNC(=O)Cn1nc(C(C)C)c2cc(Br)ccc2c1=O] |
| 110 | 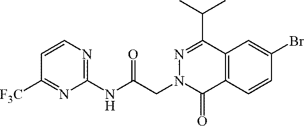 [SMILES: CC(C)c1nn(CC(=O)Nc2nccc(C(F)(F)F)n2)c(=O)c2ccc(Br)cc12] |
| 111 | 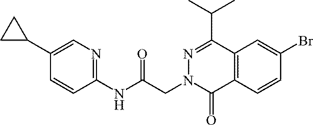 [SMILES: CC(C)c1nn(CC(=O)Nc2ccc(C3CC3)cn2)c(=O)c2ccc(Br)cc12] |
| 112 | 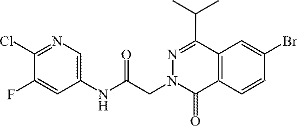 [SMILES: CC(C)c1nn(CC(=O)Nc2cnc(Cl)c(F)c2)c(=O)c2ccc(Br)cc12] |
| 113 | 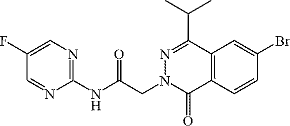 [SMILES: CC(C)c1nn(CC(=O)Nc2ncc(F)cn2)c(=O)c2ccc(Br)cc12] |
| 114 | 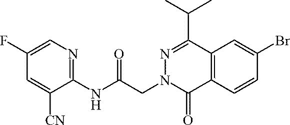 [SMILES: CC(C)c1nn(CC(=O)Nc2ncc(F)cc2C#N)c(=O)c2ccc(Br)cc12] |
| 115 | 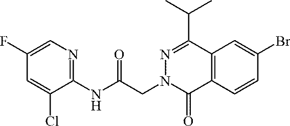 [SMILES: CC(C)c1nn(CC(=O)Nc2ncc(F)cc2Cl)c(=O)c2ccc(Br)cc12] |
| 116 | 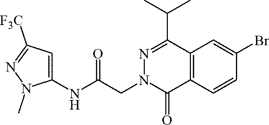 [SMILES: CC(C)c1nn(CC(=O)Nc2cc(C(F)(F)F)nn2C)c(=O)c2ccc(Br)cc12] |
| 117 | 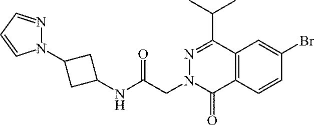 [SMILES: CC(C)c1nn(CC(=O)NC2CC(n3cccn3)C2)c(=O)c2ccc(Br)cc12] |
| 118 | 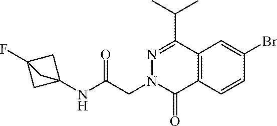 [SMILES: CC(C)c1nn(CC(=O)NC23CC(F)(C2)C3)c(=O)c2ccc(Br)cc12] |
| 119 | 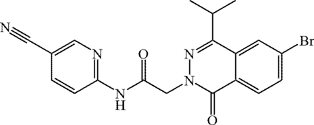 [SMILES: CC(C)c1nn(CC(=O)Nc2ccc(C#N)cn2)c(=O)c2ccc(Br)cc12] |
| 120 | 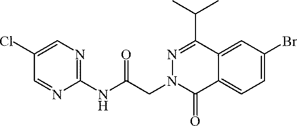 [SMILES: CC(C)c1nn(CC(=O)Nc2ncc(Cl)cn2)c(=O)c2ccc(Br)cc12] |
| 121 | 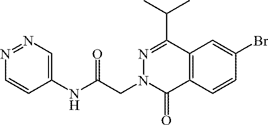 [SMILES: CC(C)c1nn(CC(=O)Nc2ccnnc2)c(=O)c2ccc(Br)cc12] |
| 122 | 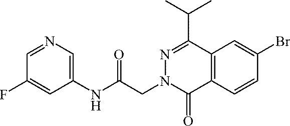 [SMILES: CC(C)c1nn(CC(=O)Nc2cncc(F)c2)c(=O)c2ccc(Br)cc12] |
| 123 | 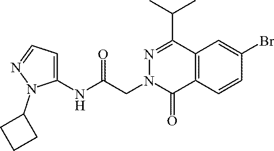 [SMILES: CC(C)c1nn(CC(=O)Nc2ccnn2C2CCC2)c(=O)c2ccc(Br)cc12] |
| 124 | 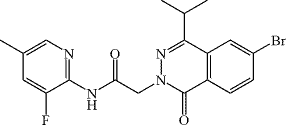 [SMILES: Cc1cnc(NC(=O)Cn2nc(C(C)C)c3cc(Br)ccc3c2=O)c(F)c1] |
| 125 | 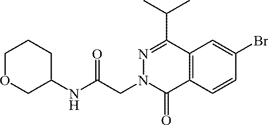 [SMILES: CC(C)c1nn(CC(=O)NC2CCCOC2)c(=O)c2ccc(Br)cc12] |
| 126 | 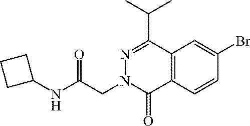 [SMILES: CC(C)c1nn(CC(=O)NC2CCC2)c(=O)c2ccc(Br)cc12] |
| 127 | 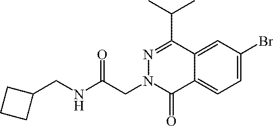 [SMILES: CC(C)c1nn(CC(=O)NCC2CCC2)c(=O)c2ccc(Br)cc12] |
| 128 | 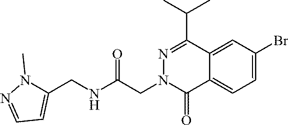 [SMILES: CC(C)c1nn(CC(=O)NCc2ccnn2C)c(=O)c2ccc(Br)cc12] |
| 129 | 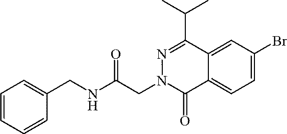 [SMILES: CC(C)c1nn(CC(=O)NCc2ccccc2)c(=O)c2ccc(Br)cc12] |
| 130 | 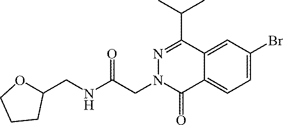 [SMILES: CC(C)c1nn(CC(=O)NCC2CCCO2)c(=O)c2ccc(Br)cc12] |
| 131 | 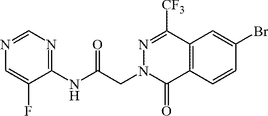 [SMILES: O=C(Cn1nc(C(F)(F)F)c2cc(Br)ccc2c1=O)Nc1ncncc1F] |
| 132 | 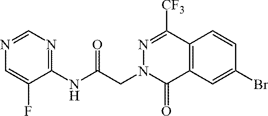 [SMILES: O=C(Cn1nc(C(F)(F)F)c2ccc(Br)cc2c1=O)Nc1ncncc1F] |
| 133 | 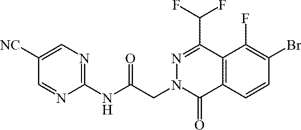 |
| 134 | 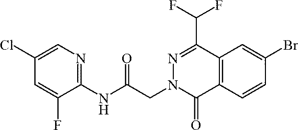 [SMILES: O=C(Cn1nc(C(F)F)c2cc(Br)ccc2c1=O)Nc1ncc(Cl)cc1F] |
| 135 | 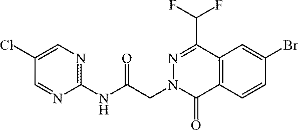 [SMILES: O=C(Cn1nc(C(F)F)c2cc(Br)ccc2c1=O)Nc1ncc(Cl)cn1] |
| 136 | 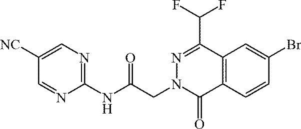 |
| 137 | 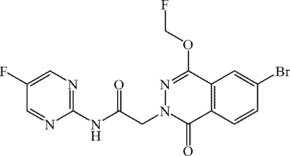 [SMILES: O=C(Cn1nc(OCF)c2cc(Br)ccc2c1=O)Nc1ncc(F)cn1] |
| 138 | 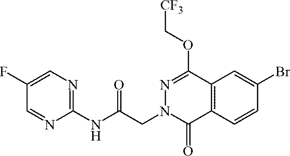 [SMILES: O=C(Cn1nc(OCC(F)(F)F)c2cc(Br)ccc2c1=O)Nc1ncc(F)cn1] |
| 139 | 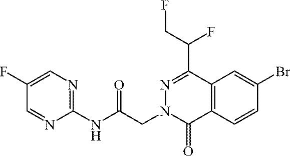 [SMILES: O=C(Cn1nc(C(F)CF)c2cc(Br)ccc2c1=O)Nc1ncc(F)cn1] |
| 140 | 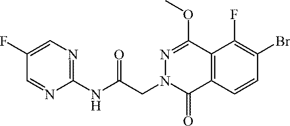 [SMILES: COc1nn(CC(=O)Nc2ncc(F)cn2)c(=O)c2ccc(Br)c(F)c12] |
| 141 | 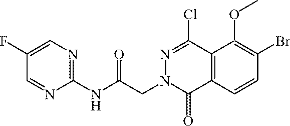 [SMILES: COc1c(Br)ccc2c(=O)n(CC(=O)Nc3ncc(F)cn3)nc(Cl)c12] |
| 142 | 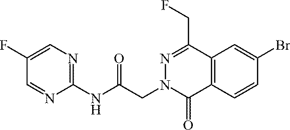 [SMILES: O=C(Cn1nc(CF)c2cc(Br)ccc2c1=O)Nc1ncc(F)cn1] |
| 143 | 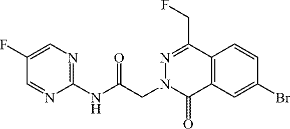 [SMILES: O=C(Cn1nc(CF)c2ccc(Br)cc2c1=O)Nc1ncc(F)cn1] |
| 144 | 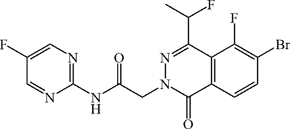 [SMILES: CC(F)c1nn(CC(=O)Nc2ncc(F)cn2)c(=O)c2ccc(Br)c(F)c12] |
| 145 | 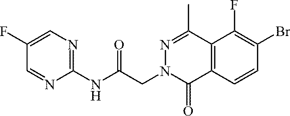 [SMILES: Cc1nn(CC(=O)Nc2ncc(F)cn2)c(=O)c2ccc(Br)c(F)c12] |
| 146 | 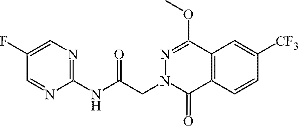 [SMILES: COc1nn(CC(=O)Nc2ncc(F)cn2)c(=O)c2ccc(C(F)(F)F)cc12] |
| 147 | 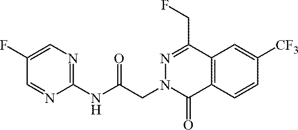 [SMILES: O=C(Cn1nc(CF)c2cc(C(F)(F)F)ccc2c1=O)Nc1ncc(F)cn1] |
| 148 | 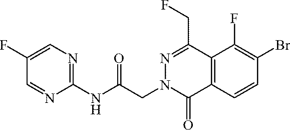 [SMILES: O=C(Cn1nc(CF)c2c(F)c(Br)ccc2c1=O)Nc1ncc(F)cn1] |
| 149 | 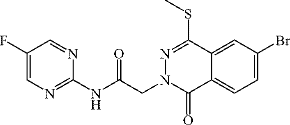 [SMILES: CSc1nn(CC(=O)Nc2ncc(F)cn2)c(=O)c2ccc(Br)cc12] |
| 150 | 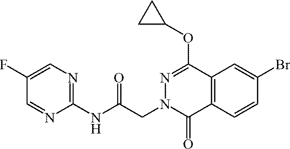 [SMILES: O=C(Cn1nc(OC2CC2)c2cc(Br)ccc2c1=O)Nc1ncc(F)cn1] |
| 151 | 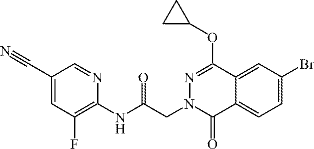 [SMILES: N#Cc1cnc(NC(=O)Cn2nc(OC3CC3)c3cc(Br)ccc3c2=O)c(F)c1] |
| 152 | 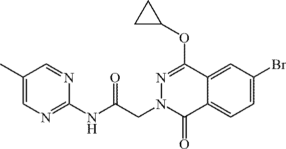 [SMILES: Cc1cnc(NC(=O)Cn2nc(OC3CC3)c3cc(Br)ccc3c2=O)nc1] |
| 153 | 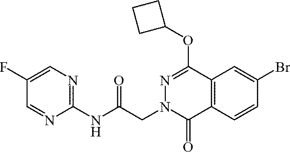 [SMILES: O=C(Cn1nc(OC2CCC2)c2cc(Br)ccc2c1=O)Nc1ncc(F)cn1] |
| 154 | 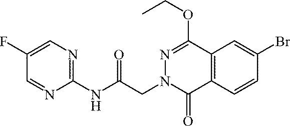 [SMILES: CCOc1nn(CC(=O)Nc2ncc(F)cn2)c(=O)c2ccc(Br)cc12] |
| 155 | 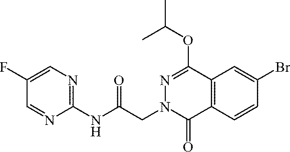 [SMILES: CC(C)Oc1nn(CC(=O)Nc2ncc(F)cn2)c(=O)c2ccc(Br)cc12] |
| 156 | 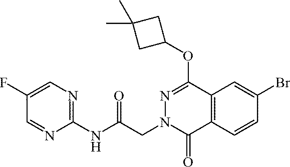 [SMILES: CC1(C)CC(Oc2nn(CC(=O)Nc3ncc(F)cn3)c(=O)c3ccc(Br)cc23)C1] |
| 157 | 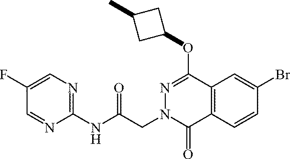 [SMILES: C[C@H]1C[C@@H](Oc2nn(CC(=O)Nc3ncc(F)cn3)c(=O)c3ccc(Br)cc23)C1] |
| 158 | 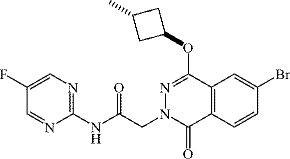 [SMILES: C[C@H]1C[C@H](Oc2nn(CC(=O)Nc3ncc(F)cn3)c(=O)c3ccc(Br)cc23)C1] |
| 159 | 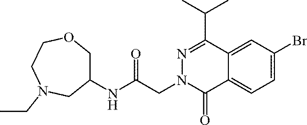 [SMILES: CCN1CCOCC(NC(=O)Cn2nc(C(C)C)c3cc(Br)ccc3c2=O)C1] |
| 160 | 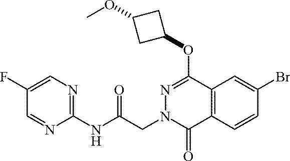 [SMILES: CO[C@H]1C[C@H](Oc2nn(CC(=O)Nc3ncc(F)cn3)c(=O)c3ccc(Br)cc23)C1] |
| 161 | 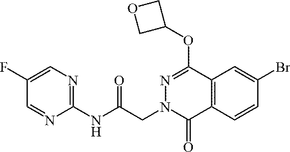 [SMILES: O=C(Cn1nc(OC2COC2)c2cc(Br)ccc2c1=O)Nc1ncc(F)cn1] |
| 162 | 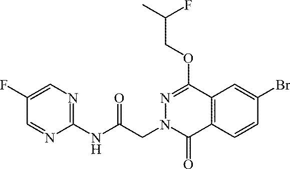 [SMILES: CC(F)COc1nn(CC(=O)Nc2ncc(F)cn2)c(=O)c2ccc(Br)cc12] |
| 163 | 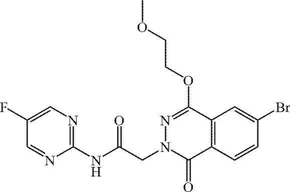 [SMILES: COCCOc1nn(CC(=O)Nc2ncc(F)cn2)c(=O)c2ccc(Br)cc12] |
| 164 | 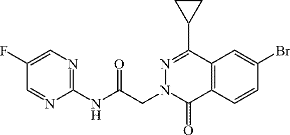 [SMILES: O=C(Cn1nc(C2CC2)c2cc(Br)ccc2c1=O)Nc1ncc(F)cn1] |
| 165 | 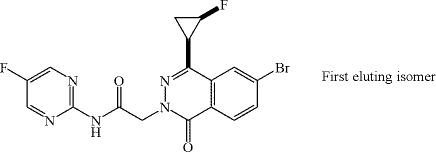 [SMILES: O=C(Cn1nc([C@H]2C[C@H]2F)c2cc(Br)ccc2c1=O)Nc1ncc(F)cn1] |
| 166 | 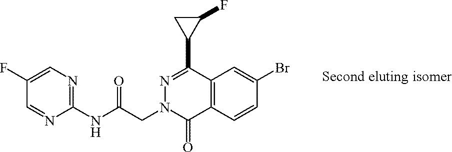 [SMILES: O=C(Cn1nc([C@H]2C[C@H]2F)c2cc(Br)ccc2c1=O)Nc1ncc(F)cn1] |
| 167 | 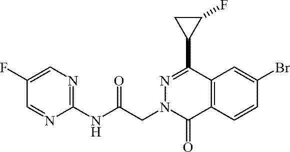 [SMILES: O=C(Cn1nc([C@H]2C[C@@H]2F)c2cc(Br)ccc2c1=O)Nc1ncc(F)cn1] |
| 168 | 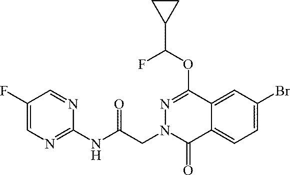 [SMILES: O=C(Cn1nc(OC(F)C2CC2)c2cc(Br)ccc2c1=O)Nc1ncc(F)cn1] |
| 169 | 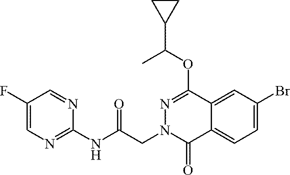 [SMILES: CC(Oc1nn(CC(=O)Nc2ncc(F)cn2)c(=O)c2ccc(Br)cc12)C1CC1] |
| 170 | 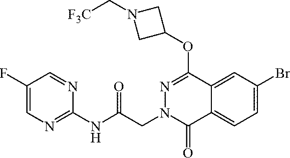 [SMILES: O=C(Cn1nc(OC2CN(CC(F)(F)F)C2)c2cc(Br)ccc2c1=O)Nc1ncc(F)cn1] |
| 171 | 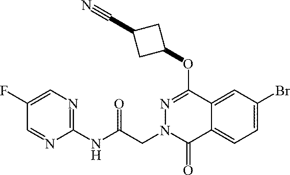 [SMILES: N#C[C@H]1C[C@@H](Oc2nn(CC(=O)Nc3ncc(F)cn3)c(=O)c3ccc(Br)cc23)C1] |
| 172 | 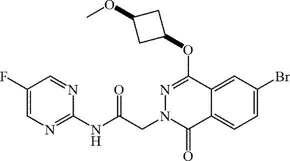 [SMILES: CO[C@H]1C[C@@H](Oc2nn(CC(=O)Nc3ncc(F)cn3)c(=O)c3ccc(Br)cc23)C1] |
| 173 | 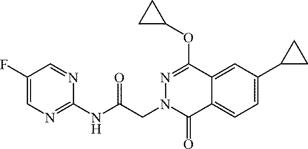 [SMILES: O=C(Cn1nc(OC2CC2)c2cc(C3CC3)ccc2c1=O)Nc1ncc(F)cn1] |
| 174 | 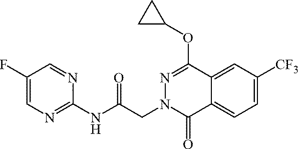 [SMILES: O=C(Cn1nc(OC2CC2)c2cc(C(F)(F)F)ccc2c1=O)Nc1ncc(F)cn1] |
| 175 | 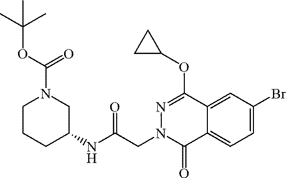 [SMILES: CC(C)(C)OC(=O)N1CCC[C@@H](NC(=O)Cn2nc(OC3CC3)c3cc(Br)ccc3c2=O)C1] |
| 176 | 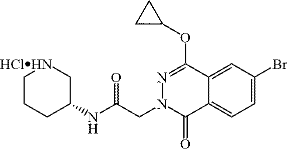 |
| 177 | 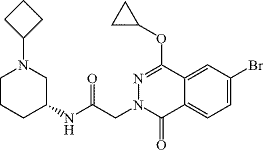 [SMILES: O=C(Cn1nc(OC2CC2)c2cc(Br)ccc2c1=O)N[C@@H]1CCCN(C2CCC2)C1] |
| 178 | 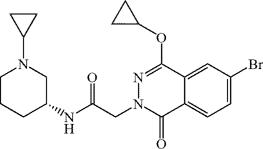 [SMILES: O=C(Cn1nc(OC2CC2)c2cc(Br)ccc2c1=O)N[C@@H]1CCCN(C2CC2)C1] |
| 179 | 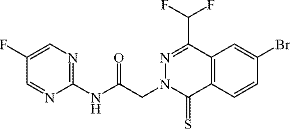 [SMILES: O=C(Cn1nc(C(F)F)c2cc(Br)ccc2c1=S)Nc1ncc(F)cn1] |
| 180 | 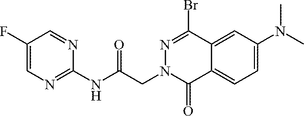 [SMILES: CN(C)c1ccc2c(=O)n(CC(=O)Nc3ncc(F)cn3)nc(Br)c2c1] |
| 181 | 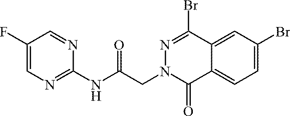 [SMILES: O=C(Cn1nc(Br)c2cc(Br)ccc2c1=O)Nc1ncc(F)cn1] |
| 182 | 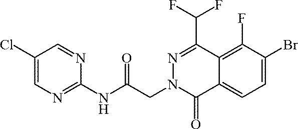 [SMILES: O=C(Cn1nc(C(F)F)c2c(F)c(Br)ccc2c1=O)Nc1ncc(Cl)cn1] |
| 183 | 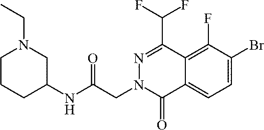 [SMILES: CCN1CCCC(NC(=O)Cn2nc(C(F)F)c3c(F)c(Br)ccc3c2=O)C1] |
| 184 | 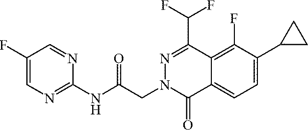 [SMILES: O=C(Cn1nc(C(F)F)c2c(F)c(C3CC3)ccc2c1=O)Nc1ncc(F)cn1] |
| 185 | 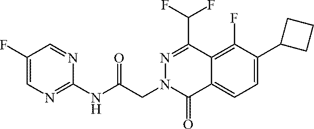 [SMILES: O=C(Cn1nc(C(F)F)c2c(F)c(C3CCC3)ccc2c1=O)Nc1ncc(F)cn1] |

In certain embodiments, provided is a compound selected from Table 2 or a pharmaceutically acceptable salt, isotopically enriched analog, prodrug, stereoisomer, or a mixture of stereoisomers thereof:

TABLE 2

Structure

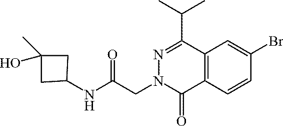 [SMILES: CC(C)c1nn(CC(=O)NC2CC(C)(O)C2)c(=O)c2ccc(Br)cc12]

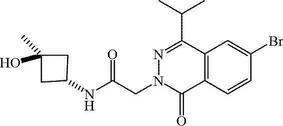 [SMILES: CC(C)c1nn(CC(=O)N[C@H]2C[C@](C)(O)C2)c(=O)c2ccc(Br)cc12]

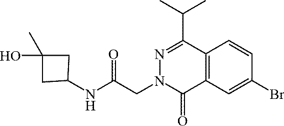 [SMILES: CC(C)c1nn(CC(=O)NC2CC(C)(O)C2)c(=O)c2cc(Br)ccc12]

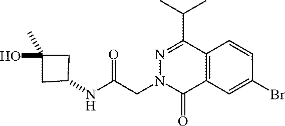 [SMILES: CC(C)c1nn(CC(=O)N[C@H]2C[C@](C)(O)C2)c(=O)c2cc(Br)ccc12]

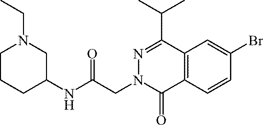 [SMILES: CCN1CCCC(NC(=O)Cn2nc(C(C)C)c3cc(Br)ccc3c2=O)C1]

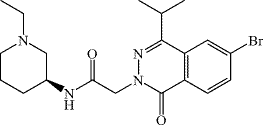 [SMILES: CCN1CCC[C@H](NC(=O)Cn2nc(C(C)C)c3cc(Br)ccc3c2=O)C1]

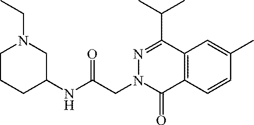 [SMILES: CCN1CCCC(NC(=O)Cn2nc(C(C)C)c3cc(C)ccc3c2=O)C1]

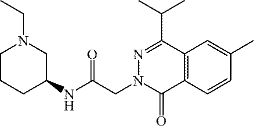 [SMILES: CCN1CCC[C@H](NC(=O)Cn2nc(C(C)C)c3cc(C)ccc3c2=O)C1]

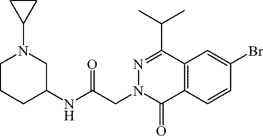 [SMILES: CC(C)c1nn(CC(=O)NC2CCCN(C3CC3)C2)c(=O)c2ccc(Br)cc12]

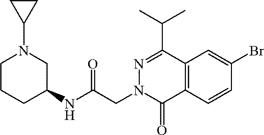 [SMILES: CC(C)c1nn(CC(=O)N[C@H]2CCCN(C3CC3)C2)c(=O)c2ccc(Br)cc12]

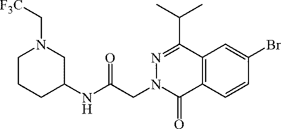 [SMILES: CC(C)c1nn(CC(=O)NC2CCCN(CC(F)(F)F)C2)c(=O)c2ccc(Br)cc12]

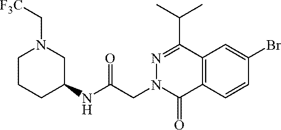 [SMILES: CC(C)c1nn(CC(=O)N[C@H]2CCCN(CC(F)(F)F)C2)c(=O)c2ccc(Br)cc12]

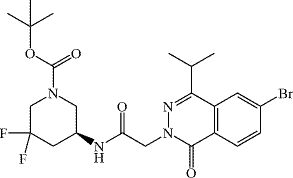 [SMILES: CC(C)c1nn(CC(=O)N[C@@H]2CN(C(=O)OC(C)(C)C)CC(F)(F)C2)c(=O)c2ccc(Br)cc12]

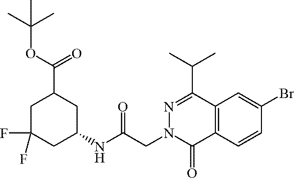 [SMILES: CC(C)c1nn(CC(=O)N[C@H]2CC(C(=O)OC(C)(C)C)CC(F)(F)C2)c(=O)c2ccc(Br)cc12]

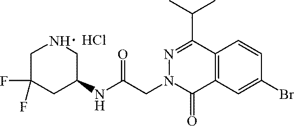

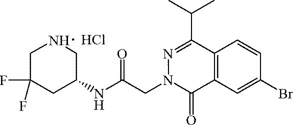

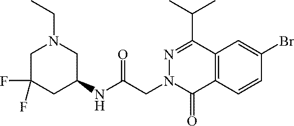 [SMILES: CCN1C[C@@H](NC(=O)Cn2nc(C(C)C)c3cc(Br)ccc3c2=O)CC(F)(F)C1]

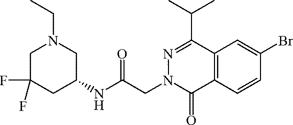 [SMILES: CCN1C[C@H](NC(=O)Cn2nc(C(C)C)c3cc(Br)ccc3c2=O)CC(F)(F)C1]

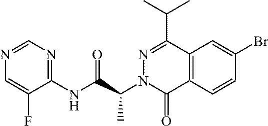

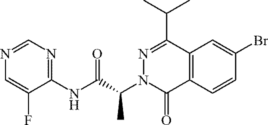

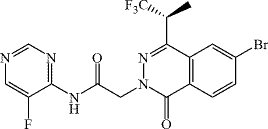

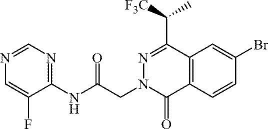

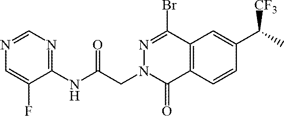

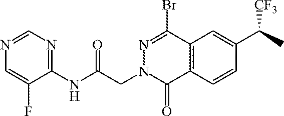

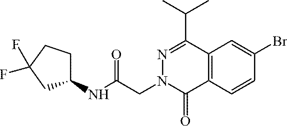

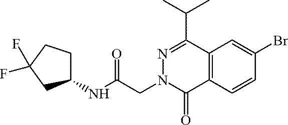

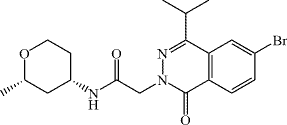 [SMILES: CC(C)c1nn(CC(=O)N[C@H]2CCO[C@@H](C)C2)c(=O)c2ccc(Br)cc12]

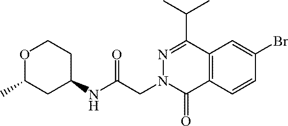 [SMILES: CC(C)c1nn(CC(=O)N[C@@H]2CCO[C@@H](C)C2)c(=O)c2ccc(Br)cc12]

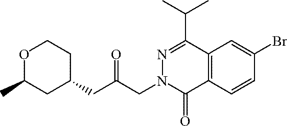 [SMILES: CC(C)c1nn(CC(=O)C[C@H]2CCO[C@H](C)C2)c(=O)c2ccc(Br)cc12]

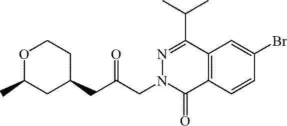 [SMILES: CC(C)c1nn(CC(=O)C[C@@H]2CCO[C@H](C)C2)c(=O)c2ccc(Br)cc12]

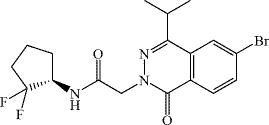

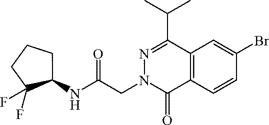 [SMILES: CC(C)c1nn(CC(=O)N[C@@H]2CCCC2(F)F)c(=O)c2ccc(Br)cc12]

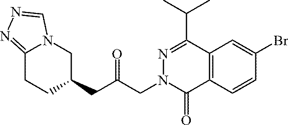

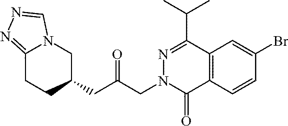

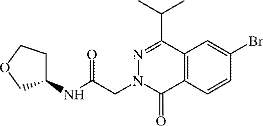

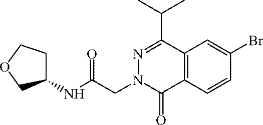

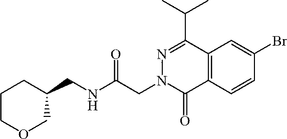

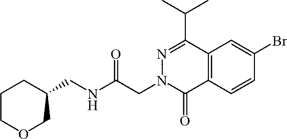

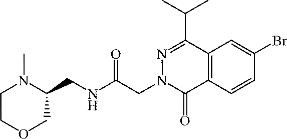

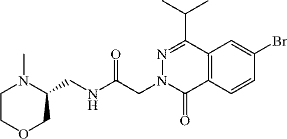

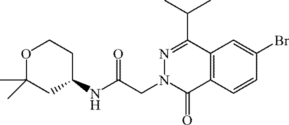

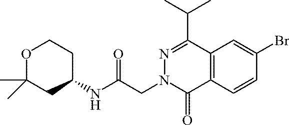

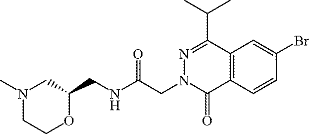

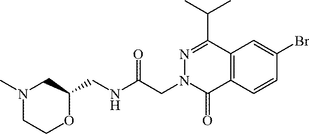

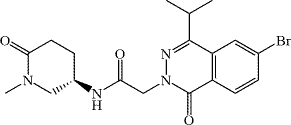

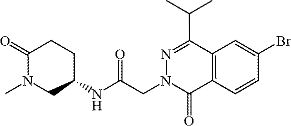

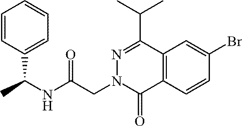

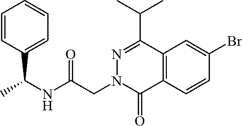

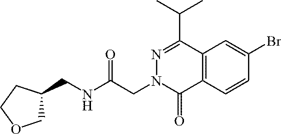

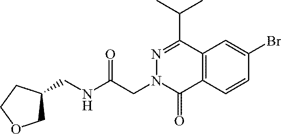

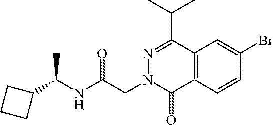

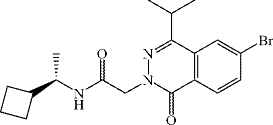

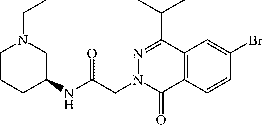 [SMILES: CCN1CCC[C@H](NC(=O)Cn2nc(C(C)C)c3cc(Br)ccc3c2=O)C1]

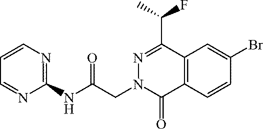

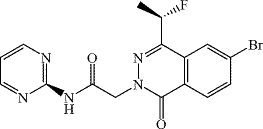

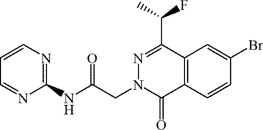

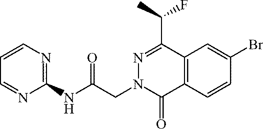

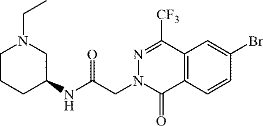 [SMILES: CCN1CCC[C@H](NC(=O)Cn2nc(C(F)(F)F)c3cc(Br)ccc3c2=O)C1]

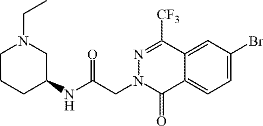 [SMILES: CCN1CCC[C@H](NC(=O)Cn2nc(C(F)(F)F)c3cc(Br)ccc3c2=O)C1]

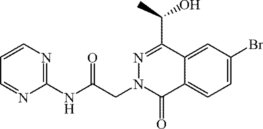

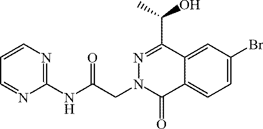

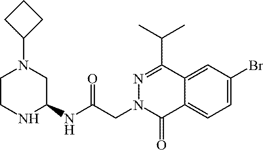 [SMILES: CC(C)c1nn(CC(=O)N[C@@H]2CN(C3CCC3)CCN2)c(=O)c2ccc(Br)cc12]

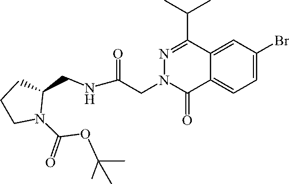

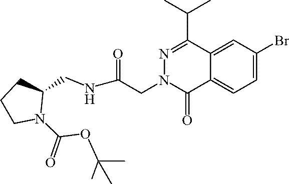

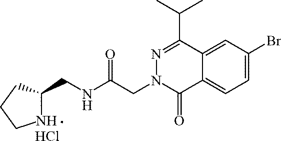

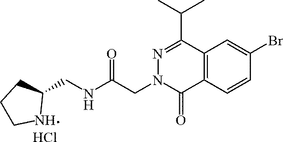

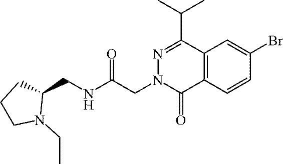

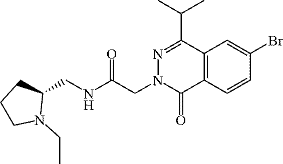

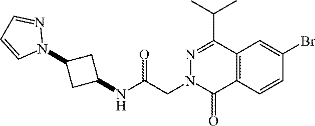 [SMILES: CC(C)c1nn(CC(=O)N[C@H]2C[C@@H](n3cccn3)C2)c(=O)c2ccc(Br)cc12]

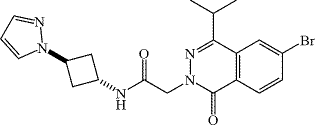 [SMILES: CC(C)c1nn(CC(=O)N[C@H]2C[C@H](n3cccn3)C2)c(=O)c2ccc(Br)cc12]

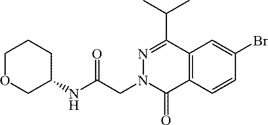 [SMILES: CC(C)c1nn(CC(=O)N[C@H]2CCCOC2)c(=O)c2ccc(Br)cc12]

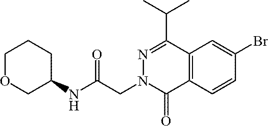 [SMILES: CC(C)c1nn(CC(=O)N[C@@H]2CCCOC2)c(=O)c2ccc(Br)cc12]

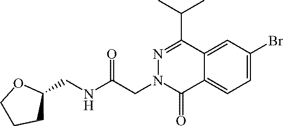

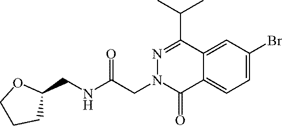

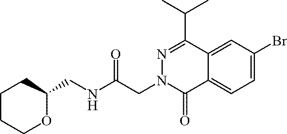

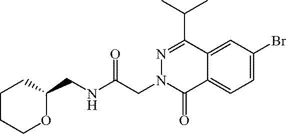

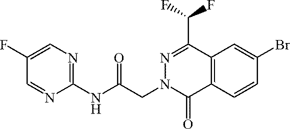

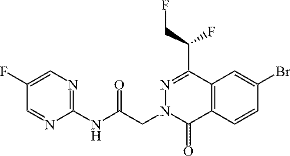

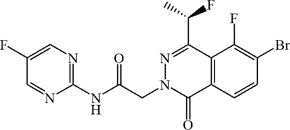

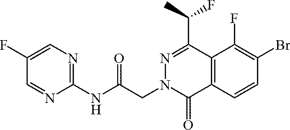

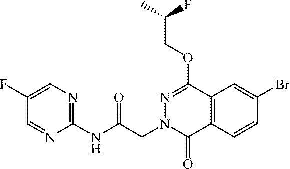

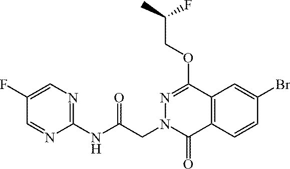

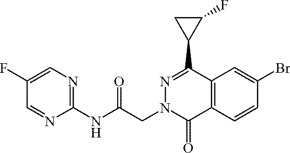 [SMILES: O=C(Cn1nc([C@H]2C[C@@H]2F)c2cc(Br)ccc2c1=O)Nc1ncc(F)cn1]

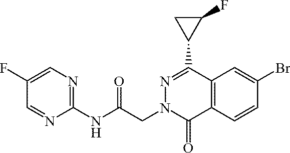 [SMILES: O=C(Cn1nc([C@@H]2C[C@H]2F)c2cc(Br)ccc2c1=O)Nc1ncc(F)cn1]

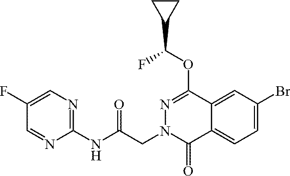

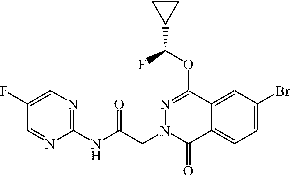

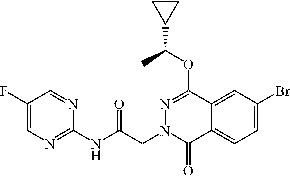

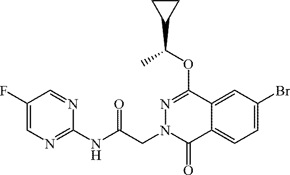

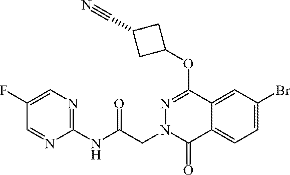

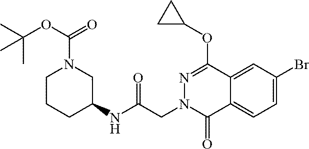 [SMILES: CC(C)(C)OC(=O)N1CCC[C@H](NC(=O)Cn2nc(OC3CC3)c3cc(Br)ccc3c2=O)C1]

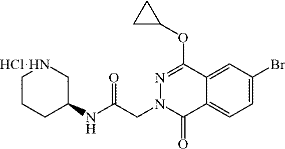

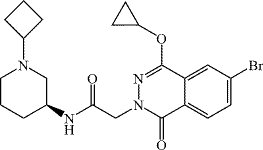 [SMILES: O=C(Cn1nc(OC2CC2)c2cc(Br)ccc2c1=O)N[C@H]1CCCN(C2CCC2)C1]

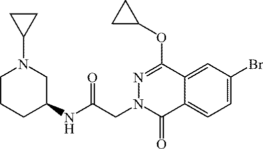 [SMILES: O=C(Cn1nc(OC2CC2)c2cc(Br)ccc2c1=O)N[C@H]1CCCN(C2CC2)C1]

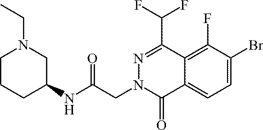 [SMILES: CCN1CCC[C@H](NC(=O)Cn2nc(C(F)F)c3c(F)c(Br)ccc3c2=O)C1]

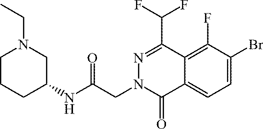 [SMILES: CCN1CCC[C@@H](NC(=O)Cn2nc(C(F)F)c3c(F)c(Br)ccc3c2=O)C1]

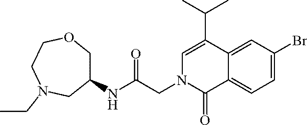 [SMILES: CCN1CCOC[C@@H](NC(=O)Cn2cc(C(C)C)c3cc(Br)ccc3c2=O)C1]

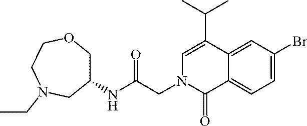 [SMILES: CCN1CCOC[C@H](NC(=O)Cn2cc(C(C)C)c3cc(Br)ccc3c2=O)C1]

### 3. Methods

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy, and/or veterinary applications. In some embodiments, the subject is a mammal. In certain embodiments, the subject is a human.

The term “therapeutically effective amount” or “effective amount” of a compound described herein or a pharmaceutically acceptable salt, isotopically enriched analog, stereoisomer, mixture of stereoisomers, or prodrug thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of as described herein. The therapeutically effective amount may vary depending on the subject, and disease or condition being treated, the weight and age of the subject, the severity of the disease or condition, and the manner of administering, which can readily be determined by one of ordinary skill in the art.

In certain embodiments, the compound for use in the methods described herein, is a compound of Formula I:

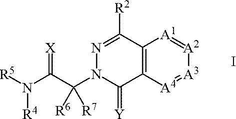

or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, -NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, -NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, C₃₋₁₀ cycloalkyl, —NO₂, —SF₅, -OR¹¹, -C(O)R¹¹, -C(O)OR¹¹, -SR¹¹, -NR¹¹S(O)₀₋₂-R¹¹, -NR¹¹S(O)₀₋₂N(R¹¹)₂, -NR¹¹C(O)N(R¹¹)₂, -NR¹¹C(O)OR¹¹, -OC(O)R¹¹, -OC(O)N(R¹¹)₂, halo, cyano, -NR¹¹C(O)R¹¹, -S(O)R¹¹, or -S(O)₂R¹¹; wherein the C₁₋₆ alkyl, C₂₋₆ alkenyl, C₂₋₆ alkynyl, C₁₋₆ haloalkyl, or C₃₋₁₀ cycloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹;
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{la};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(0)R¹¹, -C(O)OR^{II}, -S(0)₀₋₂R¹¹, -NR¹¹S(0)₀₋₂-R¹¹, -S(O)₀₋₂N(R¹¹)₂, -NR¹¹S(0)₀₋₂N(R¹¹)₂, -NR¹¹C(O)N(R¹¹)₂, -C(O)N(R¹¹)₂, -NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five _{Z}^{la};
each Z² is independently halo, cyano, -NO₂, —SF₅, -OR¹¹, -C(O)R¹¹, -C(O)OR¹¹, -NR¹¹S(O)₀₋₂-R¹¹, -NR¹¹S(O)₀₋₂N(R¹¹⁾₂, -NR¹¹C(O)N(R¹¹)₂, -NR¹¹C(O)R¹¹, -OC(O)N(R¹¹)₂, -NR¹¹C(O)OR¹¹, -N(R¹¹)₂, -C(O)N(R¹¹)₂, -S(O)₀₋₂R¹¹, or -S(O)₀₋₂N(R¹¹)₂;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
each Z^{la} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)ₒ₋₂R¹³, -NR¹³S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂ -NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, -N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, —C(O)—, —C(O)O—, —C(O)NH—, -C(O)N(C₁₋₆ alkyl)-, -C(O)N(C₂₋₆ alkenyl)-, -C(O)N(C₂₋₆ alkynyl)-, -C(O)N(C₁₋₆ haloalkyl)-, -C(O)N(C₃₋₁₀ cycloalkyl)-, -C(O)N(heterocyclyl)-, -C(O)N(aryl)-, -C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The compounds may be further characterized to examine the safety or tolerance dosage in human or non-human subjects. Such properties may be examined using commonly known methods to those skilled in the art.

In certain embodiments, provided are compounds, or a pharmaceutically acceptable salt, isotopically enriched analog, stereoisomer, mixture of stereoisomers, or prodrug thereof, that modulate the activity of NLR Family Pyrin Domain Containing 3 (NLRP3). In certain embodiments, the compounds provided herein, or a pharmaceutically acceptable salt, isotopically enriched analog, stereoisomer, mixture of stereoisomers, or prodrug thereof, inhibit the activation of NLRP3.

NLR proteins are involved in the immune system, helping to start and regulate the immune system’s response to injury, toxins, or invasion by microorganisms. NLRP3 (also known as cryopyrin, NALP3, LRR and PYD domains-containing protein 3), is a protein encoded by the NLRP3 gene (also known as CIAS1). Once activated, NLRP3 molecules assemble, along with other proteins, into inflammasomes. The activation of NLRP3 by cellular stress leads to inflammasome activation and downstream proteolytic events, including the formation of active proinflammatory cytokines such as interleukin (IL)-1β and IL-18 which are then secreted. Among other cytokines, IL-1β and IL-18 are known mediators of inflammation, e.g., artery wall inflammation, atherosclerosis and the aging process.

In certain embodiments, provided is a method of inhibiting inflammasome (e.g., the NLRP3 inflammasome) activity comprising contacting a cell with an effective amount of a compound disclosed herein, or a pharmaceutically acceptable salt, isotopically enriched analog, stereoisomer, mixture of stereoisomers, or prodrug thereof. The inhibiting can be in vitro or in vivo.

In certain embodiments, provided is a compound as disclosed herein, or a pharmaceutically acceptable salt, isotopically enriched analog, stereoisomer, mixture of stereoisomers, or prodrug thereof, for use in inhibiting inflammasome (e.g., the NLRP3 inflammasome) activity (e.g., in vitro or in vivo).

In certain embodiments, the present disclosure provides use of a compound as disclosed herein, or a pharmaceutically acceptable salt, isotopically enriched analog, stereoisomer, mixture of stereoisomers, or prodrug thereof, in the manufacture of a medicament for inhibiting inflammasome (e.g., the NLRP3 inflammasome) activity (e.g., in vitro or in vivo).

Chronic inflammation responses have been associated with various types of cancer. During malignant transformation or cancer therapy, inflammasomes may become activated in response to certain signals; and IL-Iβ expression is elevated in a variety of cancers (e.g., breast, prostate, colon, lung, head and neck cancers, melanomas, etc.), where patients with IL-Iβ producing tumors generally have a worse prognosis.

In certain embodiments, provided is a method for treating a disease or condition mediated, at least in part, by NLRP3, comprising administering an effective amount of a compound disclosed herein, or a pharmaceutically acceptable salt, isotopically enriched analog, stereoisomer, mixture of stereoisomers, or prodrug thereof, to a subject in need thereof.

In certain embodiments, provided is a method for treating a disease or condition selected from an autoinflammatory disorder, an autoimmune disorder, a neurodegenerative disease or cancer, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a compound as disclosed herein, or a pharmaceutically acceptable salt, isotopically enriched analog, stereoisomer, mixture of stereoisomers, or prodrug thereof, for use in treating an autoinflammatory disorder, an autoimmune disorder, a neurodegenerative disease or cancer in a subject in need thereof.

In certain embodiments, the present disclosure provides use of a compound as disclosed herein, or a pharmaceutically acceptable salt, isotopically enriched analog, stereoisomer, mixture of stereoisomers, or prodrug thereof in the manufacture of a medicament for treating or preventing an autoinflammatory disorder, an autoimmune disorder, a neurodegenerative disease or cancer in a subject in need thereof.

In certain embodiments, provided is a method for treating inflammation, an auto-immune disease, cancer, an infection, a central nervous system disease, a metabolic disease, a cardiovascular disease, a respiratory disease, a liver disease, a renal disease, an ocular disease, a skin disease, a lymphatic condition, a psychological disorder, graft versus host disease, allodynia, and any disease where an individual has been determined to carry a germline or somatic non-silent mutation in NLRP3, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, the disease or condition may be a disease or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen. It will be appreciated that these general embodiments defined according to broad categories of diseases, disorders and conditions are not mutually exclusive.

In certain embodiments, the disease or condition includes, inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity; auto-immune diseases such as acute disseminated encephalitis, Addison’s disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn’s disease, type 1 diabetes (T1D), Goodpasture’s syndrome, Graves’ disease, Guillain-Barré syndrome (GBS), Hashimoto’s disease, idiopathic thrombocytopenic purpura, Kawasaki’s disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord’s thyroiditis, pemphigus, pernicious anemia, polyarthritis, primary biliary cirrhosis, rheumatoid arthritis (RA), psoriatic arthritis, juvenile idiopathic arthritis or Still’s disease, refractory gouty arthritis, Reiter’s syndrome, Sjögren’s syndrome, systemic sclerosis a systemic connective tissue disorder, Takayasu’s arteritis, temporal arteritis, warm autoimmune hemolytic anemia, Wegener’s granulomatosis, alopecia universalis, Behçet’s disease, Chagas’ disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, vitiligo or vulvodynia; cancer including lung cancer, pancreatic cancer, gastric cancer, myelodysplastic syndrome, leukemia including acute lymphocytic leukemia (ALL) and acute myeloid leukemia (AML), adrenal cancer, anal cancer, basal and squamous cell skin cancer, bile duct cancer, bladder cancer, bone cancer, brain and spinal cord tumors, breast cancer, cervical cancer, chronic lymphocytic leukemia (CLL), chronic myeloid leukemia (CML), chronic myelomonocytic leukemia (CMML), colorectal cancer, endometrial cancer, oesophagus cancer, Ewing family of tumors, eye cancer, gallbladder cancer, gastrointestinal carcinoid tumors, gastrointestinal stromal tumor (GIST), gestational trophoblastic disease, glioma, Hodgkin lymphoma, Kaposi sarcoma, kidney cancer, laryngeal and hypopharyngeal cancer, liver cancer, lung carcinoid tumor, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumors, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumor; infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus, Helicobacter pylori, Bacillus anthracis, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes) and prion infections; central nervous system diseases such as Parkinson’s disease, Alzheimer’s disease, dementia, motor neuron disease, Huntington’s disease, cerebral malaria, brain injury from pneumococcal meningitis, intracranial aneurysms, traumatic brain injury, and amyotrophic lateral sclerosis; metabolic diseases such as type 2 diabetes (T2D), atherosclerosis, obesity, gout, and pseudo-gout; cardiovascular diseases such as hypertension, ischemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, and pericarditis including Dressler’s syndrome; respiratory diseases including chronic obstructive pulmonary disorder (COPD), asthma such as allergic asthma and steroid-resistant asthma, asbestosis, silicosis, nanoparticle induced inflammation, cystic fibrosis and idiopathic pulmonary fibrosis; liver diseases including non-alcoholic fatty liver disease (NAFLD) and non-alcoholic steatohepatitis (NASH) including advanced fibrosis stages F3 and F4; alcoholic fatty liver disease (AFLD), and alcoholic steatohepatitis (ASH); renal diseases including chronic kidney disease, oxalate nephropathy, nephrocalcinosis, glomerulonephritis, and diabetic nephropathy; ocular diseases including those of the ocular epithelium, age-related macular degeneration (AMD) (dry and wet), uveitis, corneal infection, diabetic retinopathy, optic nerve damage, dry eye, and glaucoma; skin diseases including dermatitis such as contact dermatitis and atopic dermatitis, contact hypersensitivity, sunburn, skin lesions, hidradenitis suppurativa (HS), other cyst-causing skin diseases, and acne conglobata; lymphatic conditions such as lymphangitis and Castleman’s disease; psychological disorders such as depression and psychological stress; graft versus host disease; allodynia including mechanical allodynia; and any disease where an individual has been determined to carry a germline or somatic non-silent mutation in NLRP3.

In certain embodiments, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), tumor Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still’s disease (AOSD), haploinsufficiency of A20 (HA20), pediatric granulomatous arthritis (PGA), PLCG2-associated antibody deficiency and immune dysregulation (PLAID), PLCG2- associated autoinflammatory, antibody deficiency and immune dysregulation (APLAID), or sideroblastic anemia with B-cell immunodeficiency, periodic fevers, and developmental delay (SIFD).

In certain embodiments, provided is a method for treating a disease or condition selected from an autoinflammatory disorder and/or an autoimmune disorder selected from cryopyrin-associated autoinflammatory syndrome (CAPS; e.g., familial cold autoinflammatory syndrome (FCAS)), Muckle-Wells syndrome (MWS), chronic infantile neurological cutaneous and articular (CINCA) syndrome, neonatal-onset multisystem inflammatory disease (NOMID), familial Mediterranean fever and nonalcoholic fatty liver disease (NAFLD), non-alcoholic steatohepatitis (NASH), gout, rheumatoid arthritis, osteoarthritis, Crohn’s disease, chronic obstructive pulmonary disease (COPD), chronic kidney disease (CKD), fibrosis, obesity, type 2 diabetes, and multiple sclerosis and neuroinflammation occurring in protein misfolding diseases (e.g., Prion diseases) comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a disease or condition selected from cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA); hyperimmunoglobulinemia D and periodic fever syndrome (HIDS), Tumor Necrosis Factor (TNF), Receptor-Associated Periodic Syndrome (TRAPS), systemic juvenile idiopathic arthritis, adult-onset Still’s disease (AOSD), relapsing polychondritis, Schnitzler’s syndrome, Sweet’s syndrome, Behcet’s disease, anti-synthetase syndrome, deficiency of interleukin 1 receptor antagonist (DIRA), and haploinsufficiency of A20 (HA20) comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a disease or condition selected from Alzheimer’s disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a disease or condition that is mediated, at least in part, by TNF-α. In certain embodiments, the disease or condition is resistant to treatment with an anti-TNF-α agent. In some embodiments, the disease is a gut disease or condition. In some embodiments the disease or condition is inflammatory bowel disease, Crohn’s disease, or ulcerative colitis. In some embodiments, a compound disclosed herein or a pharmaceutically acceptable salt, isotopically enriched analog, stereoisomer, mixture of stereoisomers, or prodrug thereof is administered in combination with an anti-TNF-α agent. In some embodiments, the anti-TNF-α agent is infliximab, etanercept, certolizumab pegol, golimumab, or adalimumab.

In certain embodiments, the disease or condition is an autoinflammatory disorder, an autoimmune disorder, a neurodegenerative disease, or cancer.

In certain embodiments, the disease or condition is an autoinflammatory disorder and/or an autoimmune disorder.

In certain embodiments, the disease or condition is a neurodegenerative disease.

In certain embodiments, the disease or condition is Parkinson’s disease or Alzheimer’s disease.

In certain embodiments, provided is a method for treating cancer, comprising administering an effective amount of a compound disclosed herein, or a pharmaceutically acceptable salt, isotopically enriched analog, stereoisomer, mixture of stereoisomers, or prodrug thereof, to a subject in need thereof.

In certain embodiments, the cancer is metastasizing cancer, gastrointestinal cancer, skin cancer, non-small-cell lung carcinoma, or colorectal adenocarcinoma.

In certain embodiments, provided is a compound as disclosed herein, or a pharmaceutically acceptable salt, isotopically enriched analog, stereoisomer, mixture of stereoisomers, or prodrug thereof for use in treating a neurodegenerative disease (e.g., Parkinson’s disease or Alzheimer’s disease) in a subject in need thereof.

In certain embodiments, provided is a compound as disclosed herein, or a pharmaceutically acceptable salt, isotopically enriched analog, stereoisomer, mixture of stereoisomers, or prodrug thereof, for use in treating cancer in a subject in need thereof.

In certain embodiments, a compound as disclosed herein, or a pharmaceutically acceptable salt, isotopically enriched analog, stereoisomer, mixture of stereoisomers, or prodrug thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e., by itself the adjuvant may only have minimal therapeutic benefit, but in combination with another therapeutic agent, the overall therapeutic benefit to the individual is enhanced). Alternatively, by way of example only, the benefit experienced by an individual may be increased by administering compound as disclosed herein, or a pharmaceutically acceptable salt, isotopically enriched analog, stereoisomer, mixture of stereoisomers, or prodrug thereof, with another therapeutic agent (which also includes a therapeutic regimen) that also has therapeutic benefit.

Other embodiments include use of the presently disclosed compounds in therapy.

### 4. Kits

Provided herein are also kits that include a compound of the disclosure, or a pharmaceutically acceptable salt, isotopically enriched analog, stereoisomer, mixture of stereoisomers, or prodrug thereof, and suitable packaging. In certain embodiments, a kit further includes instructions for use. In one aspect, a kit includes a compound of the disclosure, or a pharmaceutically acceptable salt, isotopically enriched analog, stereoisomer, mixture of stereoisomers, or prodrug thereof, and a label and/or instructions for use of the compounds in the treatment of the indications, including the diseases or conditions, described herein.

Provided herein are also articles of manufacture that include a compound described herein or a pharmaceutically acceptable salt, isotopically enriched analog, stereoisomer, mixture of stereoisomers, or prodrug thereof in a suitable container. The container may be a vial, jar, ampoule, preloaded syringe, or intravenous bag.

### 5. Pharmaceutical Compositions and Modes of Administration

Compounds provided herein are usually administered in the form of pharmaceutical compositions. Thus, provided herein are also pharmaceutical compositions that contain one or more of the compounds described herein, or a pharmaceutically acceptable salt, stereoisomer, mixture of stereoisomers, or prodrug thereof, and one or more pharmaceutically acceptable vehicles selected from carriers, adjuvants, and excipients. Suitable pharmaceutically acceptable vehicles may include, for example, inert solid diluents and fillers, diluents, including sterile aqueous solution and various organic solvents, permeation enhancers, solubilizers, and adjuvants. Such compositions are prepared in a manner well known in the pharmaceutical art. See, e.g., Remington’s Pharmaceutical Sciences, Mace Publishing Co., Philadelphia, Pa. 17th Ed. (1985); and Modern Pharmaceutics, Marcel Dekker, Inc. 3rd Ed. (G.S. Banker & C.T. Rhodes, Eds.).

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, isotopically enriched analog, stereoisomer, mixture of stereoisomers, or prodrug thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include, e.g., lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxybenzoates; sweetening agents; and flavoring agents.

The compositions that include at least one compound described herein or a pharmaceutically acceptable salt, isotopically enriched analog, stereoisomer, mixture of stereoisomers, or prodrug thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, isotopically enriched analog, stereoisomer, mixture of stereoisomers, or prodrug thereof. When referring to these preformulation compositions as homogeneous, the active ingredient may be dispersed evenly throughout the composition so that the composition may be readily subdivided into equally effective unit dosage forms such as tablets, pills, and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### 6. Dosing

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject’s body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. In some embodiments, a dosage of from about 0.0001 to about 100 mg per kg of body weight per day, from about 0.001 to about 50 mg of compound per kg of body weight, or from about 0.01 to about 10 mg of compound per kg of body weight may be appropriate. Normalizing according to the subject’s body weight is particularly useful when adjusting dosages between subjects of widely disparate size, such as occurs when using the drug in both children and adult humans or when converting an effective dosage in a non-human subject such as dog to a dosage suitable for a human subject.

### 7. Synthesis of the Compounds

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents and starting materials may be purchased commercially, e.g., from Sigma Aldrich or other chemical suppliers.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups (“PG”) may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene’s protective groups in organic synthesis. Hoboken, N.J., Wiley-Interscience, and references cited therein. For example, protecting groups for alcohols, such as hydroxy, include silyl ethers (including trimethylsilyl (TMS), tert-butyldimethylsilyl (TBDMS), tri-iso-propylsilyloxymethyl (TOM), and triisopropylsilyl (TIPS) ethers), which can be removed by acid or fluoride ion, such as NaF, TBAF (tetra-n-butylammonium fluoride), HF-Py, or HF-NEt₃. Other protecting groups for alcohols include acetyl, removed by acid or base, benzoyl, removed by acid or base, benzyl, removed by hydrogenation, methoxyethoxymethyl ether, removed by acid, dimethoxytrityl, removed by acid, methoxymethyl ether, removed by acid, tetrahydropyranyl or tetrahydrofuranyl, removed by acid, and trityl, removed by acid. Examples of protecting groups for amines include carbobenzyloxy, removed by hydrogenolysis p-methoxybenzyl carbonyl, removed by hydrogenolysis, tert-butyloxycarbonyl, removed by concentrated strong acid (such as HC1 or CF₃COOH), or by heating to greater than about 80° C., 9-fluorenylmethyloxycarbonyl, removed by base, such as piperidine, acetyl, removed by treatment with a base, benzoyl, removed by treatment with a base, benzyl, removed by hydrogenolysis, carbamate group, removed by acid and mild heating, p-methoxybenzyl, removed by hydrogenolysis, 3,4-dimethoxybenzyl, removed by hydrogenolysis, p-methoxyphenyl, removed by ammonium cerium(IV) nitrate, tosyl, removed by concentrated acid (such as HBr or H₂SO₄) and strong reducing agents (sodium in liquid ammonia or sodium naphthalenide), troc (trichloroethyl chloroformate), removed by Zn insertion in the presence of acetic acid, and sulfonamides (Nosyl & Nps), removed by samarium iodide or tributyltin hydride.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser’s Reagents for Organic Synthesis, Volumes 1-15 (John Wiley, and Sons, 1991), Rodd’s Chemistry of Carbon Compounds, Volumes 1-5, and Supplementals (Elsevier Science Publishers, 1989) organic Reactions, Volumes 1-40 (John Wiley, and Sons, 1991), March’s Advanced Organic Chemistry, (John Wiley, and Sons, 5th Edition, 2001), and Larock’s Comprehensive Organic Transformations (VCH Publishers Inc., 1989).

### General Synthesis

Scheme I illustrates a general methods which can be employed for the synthesis of compounds described herein, where each of X, Y, A¹-A⁴, R², R⁴, R⁵, R⁶, and R⁷ are independently as defined herein, each R^{Z} is independently H or C₁₋₆ alkyl, and each LG is a leaving group (e.g., halo). It should be understood that derivatization of any one or more of compounds I-1 and I-5, or any product obtained by the process outlined in Scheme I, can be performed to provide various compounds of Formula I.

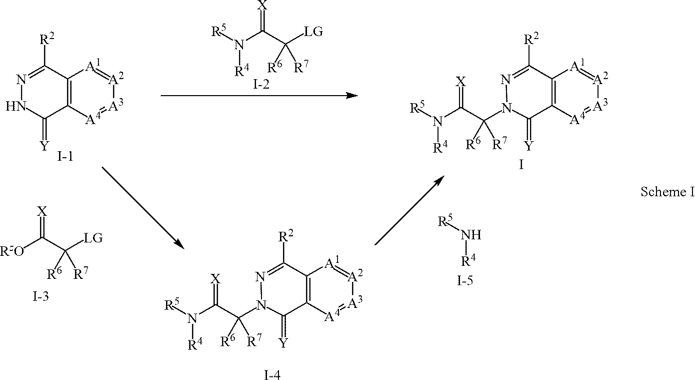

In Scheme I, compounds of Formula I can be prepared from compound I-1 by coupling with compound I-2. Alternatively, coupling of compound I-1 with compound 1-3 provides compound 1-4. An appropriately substituted amine 1-5 can be coupled directly with compound 1-4 under amide bond forming reaction conditions to yield compounds of Formula I. Alternatively, when R^{Z} is C₁₋₆ alkyl, the ester can be cleaved to yield the corresponding carboxylic acid derivative, which upon reaction with an appropriately substituted amine 1-5 under amide bond forming reaction conditions, yields compounds of Formula I.

Appropriate starting materials and reagents can be purchased or prepared by methods known to one of skill in the art. Upon each reaction completion, each of the intermediate or final compounds can be recovered, and optionally purified, by conventional techniques such as neutralization, extraction, precipitation, chromatography, filtration and the like.

In some embodiments, the various substituents of compounds I-1, I-2, I-3, I-4, and I-5 as used in Scheme I are as defined for Formula I. However, derivatization of compounds I-1, I-2, I-3, I-4, and I-5 provides various compounds of Formula I.

In certain embodiments, provided is a process for preparing a compound of Formula I, comprising:

contacting a compound of Formula I-1 with a compound of Formula I-2, under conditions suitable to provide a compound of Formula I.

In certain embodiments, provided is a process for preparing a compound of Formula I, comprising:

contacting a compound of Formula I-4 with a compound of Formula I-5, under conditions suitable to provide a compound of Formula I.

In certain embodiments, provided is a process for preparing a compound of Formula I, comprising:
contacting a compound of Formula I-1 with a compound of Formula I-3, under conditions suitable to provide a compound of Formula I-4; and
contacting a compound of Formula I-4 with a compound of Formula I-5, under conditions suitable to provide a compound of Formula I.

Scheme II illustrates a general methods which can be employed for the synthesis of compounds described herein, where each of A¹-A⁴, R², R⁴, R⁵, R⁶, and R⁷ are independently as defined herein, each R^{Z} is independently H or C₁₋₆ alkyl, and each LG is a leaving group (e.g., halo). It should be understood that derivatization of any one or more of compounds II-1 and I-5, or any product obtained by the process outlined in Scheme II, can be performed to provide various compounds of Formula II.

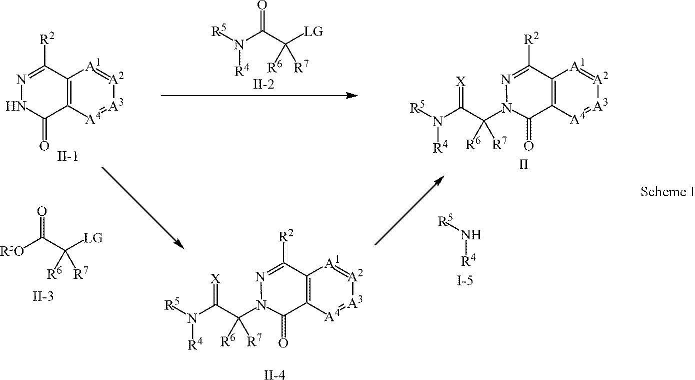

In Scheme II, compounds of Formula II can be prepared from compound II-1 by coupling with compound II-2. Alternatively, coupling of compound II-1 with compound II-3 provides compound II-4. An appropriately substituted amine I-5 can be coupled directly with compound II-4 under amide bond forming reaction conditions to yield compounds of Formula II. Alternatively, when R^{Z} is C₁₋₆ alkyl, the ester can be cleaved to yield the corresponding carboxylic acid derivative, which upon reaction with an appropriately substituted amine I-5 under amide bond forming reaction conditions, yields compounds of Formula II.

Appropriate starting materials and reagents can be purchased or prepared by methods known to one of skill in the art. Upon each reaction completion, each of the intermediate or final compounds can be recovered, and optionally purified, by conventional techniques such as neutralization, extraction, precipitation, chromatography, filtration and the like.

In some embodiments, the various substituents of compounds II-1, II-2, II-3, II-4, and I-5 as used in Scheme II are as defined for Formula II. However, derivatization of compounds II-1, II-2, II-3, II-4, and I-5 provides various compounds of Formula II.

In certain embodiments, provided is a process for preparing a compound of Formula II, comprising:

contacting a compound of Formula II-1 with a compound of Formula II-2, under conditions suitable to provide a compound of Formula II.

In certain embodiments, provided is a process for preparing a compound of Formula II, comprising:

contacting a compound of Formula II-4 with a compound of Formula I-5, under conditions suitable to provide a compound of Formula II.

In certain embodiments, provided is a process for preparing a compound of Formula II, comprising:
contacting a compound of Formula II-1 with a compound of Formula II-3, under conditions suitable to provide a compound of Formula II-4; and
contacting a compound of Formula II-4 with a compound of Formula I-5, under conditions suitable to provide a compound of Formula II.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### General Experimental Methods

All solvents used were commercially available and were used without further purification. Reactions were typically run using anhydrous solvents under an inert atmosphere of nitrogen.

NMR Spectroscopy: ¹H Nuclear magnetic resonance (NMR) spectroscopy was carried out using a Bruker Avance III equipped with a BBFO 300 MHz probe operating at 300 MHz or one of the following instruments: a Bruker Avance 400 instrument equipped with probe DUAL 400 MHz S1, a Bruker Avance 400 instrument equipped with probe 6 S1 400 MHz 5 mm ¹H-¹³C ID, a Bruker Avance III 400 instrument with nanobay equipped with probe Broadband BBFO 5 mm direct, a Bruker Mercury Plus 400 NMR spectrometer equipped with a Bruker 400 BBO probe operating at 400 MHz. All deuterated solvents contained typically 0.03% to 0.05% v/v tetramethylsilane, which was used as the reference signal (set at δ 0.00 for both ¹H and ¹³C). In certain cases, ¹H Nuclear magnetic resonance (NMR) spectroscopy was carried out using a Bruker Advance 400 instrument operating at 400 MHz using the stated solvent at around room temperature unless otherwise stated. In all cases, NMR data were consistent with the proposed structures. Characteristic chemical shifts (δ) are given in parts-per-million using conventional abbreviations for designation of major peaks: e.g. s, singlet; d, doublet; t, triplet; q, quartet; dd, doublet of doublets; dt, doublet of triplets; br, broad.

Thin Layer Chromatography: Where thin layer chromatography (TLC) has been used it refers to silica gel TLC using silica gel F254 (Merck) plates, Rf is the distance travelled by the compound divided by the distance travelled by the solvent on a TLC plate. Column chromatography was performed using an automatic flash chromatography system over silica gel cartridges or in the case of reverse phase chromatography over C18 cartridges. Alternatively, thin layer chromatography (TLC) was performed on Alugram® (Silica gel 60 F254) from Mancherey-Nagel and UV was typically used to visualize the spots. Additional visualization methods were also employed in some cases. In these cases the TLC plate was developed with iodine (generated by adding approximately 1 g of I₂ to 10 g silica gel and thoroughly mixing), ninhydrin (available commercially from Aldrich), or Magic Stain (generated by thoroughly mixing 25 g (NH₄)₆M₀₇O₂₄.4H₂O, 5 g (NH₄)₂Ce(IV)(NO₃)₆ in 450 mL water and 50 mL concentrated H₂SO₄) to visualize the compound.

Liquid Chromatography-Mass Spectrometry and HPLC Analysis: HPLC analysis was performed on Shimadzu 20AB HPLC system with a photodiode array detector and Luna-C18(2) 2.0×50 mm, 5 µm column at a flow rate of 1.2 mL/min with a gradient solvent Mobile phase A (MPA, H₂O+0.037% (v/v) TFA): Mobile phase B (MPB, ACN+0.018% (v/v) TFA) (0.01 min, 10% MPB; 4 min, 80% MPB; 4,9 min, 80% MPB; 4.92 min, 10% MPB; 5.5 min, 10% MPB). LCMS was detected under 220 and 254 nm or used evaporative light scattering (ELSD) detection as well as positive electrospray ionization (MS). Semi-preparative HPLC was performed by either acidic or neutral conditions. Acidic: Luna C18 100 × 30 mm, 5 µm; MPA: HCl/H₂O=0.04%, or formic acid/H₂O=0.2% (v/v); MPB: ACN. Neutral: Waters Xbridge 150 × 25, 5 µm; MPA: 10 mM NH₄HCO₃ in H₂O; MPB: ACN. Gradient for both conditions: 10% of MPB to 80% of MPB over 12 min at a flow rate of 20 mL/min, then 100% MPB over 2 min, 10% MPB over 2 min, UV detector. SFC analysis was performed on Thar analytical SFC system with a UV/Vis detector and series of chiral columns including AD, AS-H, OJ, OD, AY and IC, 4.6 × 100 mm, 3 µm column at a flow rate of 4 mL/min with a gradient solvent Mobile phase A (MPA, CO₂): Mobile phase B (MPB, MeOH+0.05% (v/v) IPAm) (0.01 min, 10% MPB; 3 min, 40% MPB; 3.5 min, 40% MPB; 3.56-5 min, 10% MPB). SFC preparative was performed on Thar 80 preparative SFC system with a UV/Vis detector and series of chiral preparative columns including AD-H, AS-H, OJ-H, OD-H, AY-H and IC-H, 30×250 mm, 5 µm column at a flow rate of 65 mL/min with a gradient solvent Mobile phase A (MPA, CO₂): Mobile phase B (MPB, MeOH+0.1% (v/v) NH₃H₂O) (0.01 min, 10% MPB; 5 min, 40% MPB; 6 min, 40% MPB; 6.1-10 min, 10% MPB). LC-MS data were also collected using an UPLC-MS Acquity® system equipped with PDA detector and coupled to a Waters single quadrupole mass spectrometer operating in alternated positive and negative electrospray ionization mode. The column used was a Cortecs UPLC C18, 1.6 µm, 2.1 × 50 mm. A linear gradient was applied, starting at 95% A (A: 0.1% formic acid in water) and ending at 95% B (B: 0.1% formic acid in MeCN) over 2.0 min with a total run time of 2.5 min. The column temperature was at 40° C. with the flow rate of 0.8 mL/min.

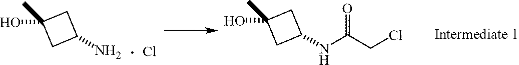

2-Chloro-N-(cis-3-hydroxy-3-methylcyclobutyl)acetamide: To a solution of cis-3-amino-1-methylcyclobutanol HC1 salt (1.1 g, 7.99 mmol) in DCM (15 mL) was added DMF (2 mL) and N-methylmorpholine (2.43 g, 24.0 mmol). To the reaction mixture was added a solution of 2-chloroacetyl chloride (903 mg, 7.99 mmol) in DCM (2 mL) dropwise at -78° C. The reaction mixture was stirred at 20° C. for 2 h. The reaction mixture was then concentrated under reduced pressure. The crude residue was purified by silica gel chromatography. ¹H NMR (400 MHz, CDC1₃): δ 6.81 (br s, 1H), 4.10-3.96 (m, 3H), 2.59-2.48 (m, 2H), 2.14-2.04 (m, 2H), 1.39 (s, 3H).

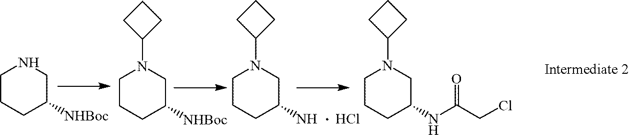

(R)-tert-butyl (1-cyclobutylpiperidin-3-yl)carbamate: To a solution of (R)-tert-butyl piperidin-3-ylcarbamate (10.0 g, 49.9 mmol) in methanol (100 mL) at 0° C. were added cyclobutanone (7.0 g, 100 mmol), acetic acid (6.0 g, 100 mmol) and sodium cyanoborohydride (5.33 g, 84.9 mmol). The reaction mixture was stirred at 20° C. for 16 h. The reaction mixture was filtered and the filtrate was concentrated under reduced pressure. The crude residue was diluted with water (100 mL) and extracted with EtOAc (3 × 40 mL). The combined organic layers were washed with brine (100 mL), dried over anhydrous Na₂SO₄, filtered, and concentrated under reduced pressure. The residue was purified by silica gel column chromatography. LCMS: m/z = 229.2 [M+H]⁺.

(R)-1-cyclobutylpiperidin-3-amine HC1 salt: (R)-tert-butyl (1-ethylpiperidin-3-yl)carbamate (6.5 g, 25.5 mmol) was dissolved in HC1 (50 mL, 4 N in dioxane). The reaction mixture was stirred at 20° C. for 1 h. The reaction mixture was concentrated under reduced pressure provide a residue that was used directly. LCMS: m/z = 191.1 [M+H]⁺.

(R)-2-chloro-N-(1-cyclobutylpiperidin-3-yl)acetamide: To a solution of (R)-1-ethylpiperidin-3-amine HC1 salt (5.7 g, 29.9 mmol) in DCM (50 mL) at 0° C. was added N-methylmorpholine (12.1 g, 120 mmol) followed by 2-chloroacetyl chloride (3.38 g, 29.9 mmol) dropwise. The reaction mixture was stirred at 20° C. for 1 h. The reaction mixture was diluted with ice-cold sat. aq. NaHCO₃ (10 mL) and extracted with DCM (3 × 50 mL). The combined organic layers were washed with brine (50 mL), dried over anhydrous Na₂SO₄, filtered, and concentrated under reduced pressure provide a residue that was used directly. LCMS: m/z = 231.1 [M+H]⁺.

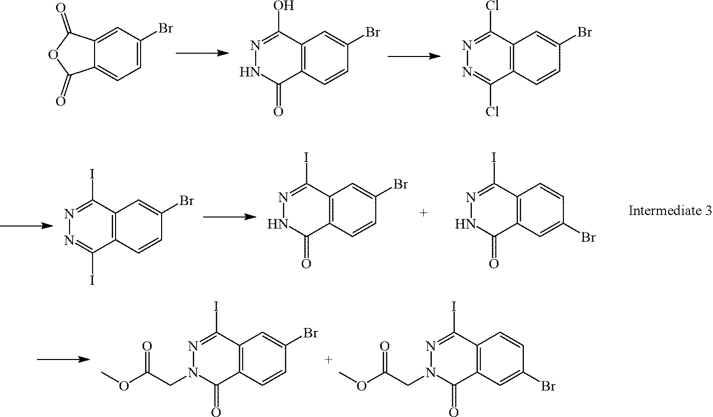

6-bromo-4-hydroxyphthalazin-1(2H)-one: A solution of 5-bromoisobenzofuran-1,3-dione (30.0 g, 132 mmol) in AcOH (800 mL) was stirred at 125° C. for 1 h. The mixture was cooled to 25° C. then hydrazine hydrate (7.10 g, 139 mmol) was added. The reaction mixture was stirred at 125° C. for 0.5 h. The reaction mixture cooled to 25° C., diluted with MeOH (400 mL), and concentrated under reduced pressure to provide a solid that was used directly. LCMS: m/z = 241.1, 243.1 [M+H]⁺.

6-bromo-1,4-dichlorophthalazine: A solution of 6-bromo-4-hydroxyphthalazin-1(2H)-one (90.0 g, 373 mmol) in POC1₃ (802 g, 5.23 mol) was stirred at 110° C. for 48 h. The reaction mixture was concentrated under reduced pressure. The residue was diluted with water (100 mL) and extracted with EtOAc (3 × 50 mL). The combined organic layers were washed with brine (50 mL), dried over anhydrous Na₂SO₄, filtered, and concentrated under reduced pressure to provide a residue that was used directly. LCMS: m/z = 277.0, 278.9 [M+H]⁺. 6-bromo-1,4-diiodophthalazine: To a solution of 6-bromo-1,4-dichlorophthalazine (50 g, 180 mmol) in acetone (600 mL) was added NaI (138 g, 918 mmol) and HI (2.0 g, 18.0 mmol). The reaction mixture was stirred at 50° C. for 16 h. The reaction mixture was diluted with water (1000 mL) and extracted with EtOAc (2 × 800 mL). The combined organic layers were washed with brine (500 mL), dried over anhydrous Na₂SO₄, filtered, and concentrated under reduced pressure. The residue was triturated with 1:2 mixture of MTBE/PE at 20° C. for 30 min to provide a residue that was used directly. LCMS: m/z = 460.8, 462.8 [M+H]⁺.

6-bromo-4-iodophthalazin-1-ol and 7-bromo-4-iodophthalazin-1-ol: To a solution of 6-bromo-1,4-diiodophthalazine (27.0 g, 58.6 mmol) in 1,4-dioxane (300 mL) was added NaOH (2 M, 293 mL). The reaction mixture was stirred at 50° C. for 16 h. The reaction mixture was concentrated under reduced pressure to remove the organic solvent. The resulting solution was adjusted to pH = 4-5 with 12 M HCl. The reaction mixture was filtered and the filter cake was dried under vacuum to provide a 1:1 mixture of 6-bromo-4-iodophthalazin-1-ol and 7-bromo-4-iodophthalazin-1-ol. LCMS: m/z = 350.9, 352.9 [M+H]⁺.

Methyl 2-(6-bromo-4-iodo-1-oxophthalazin-2(1H)-yl)acetate and methyl 2-(7-bromo-4-iodo-1-oxophthalazin-2(1H)-yl)acetate: To a solution of 7-bromo-4-iodo-phthalazin-1-ol and 6-bromo-4-iodo-phthalazin-1-ol (1:1 mixture, 17.0 g, 48.4 mmol) in DMF (200 mL) were added methyl 2-bromoacetate (15.0 g, 96.9 mmol) and Cs₂CO₃ (32.0 g, 96.9 mmol). The reaction mixture was stirred at 20° C. for 12 h. The mixture was diluted with water (500 mL) and extracted with EtOAc (2 × 200 mL). The combined organic layers were washed with brine (200 mL), dried over anhydrous Na₂SO₄, filtered, and concentrated under reduced pressure. The crude residue was triturated with a mixture of 1:2 MTBE/PE at 20° C. for 30 min then filtered to provide a 1:1 mixture of methyl 2-(6-bromo-4-iodo-1-oxophthalazin-2(1H)-yl)acetate and methyl 2-(7-bromo-4-iodo-1-oxophthalazin-2(1H)-yl)acetate. LCMS: m/z = 422.8, 424.8 [M+H]⁺.

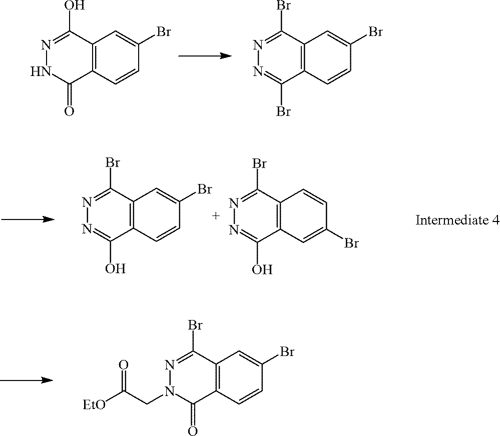

1,4,6-tribromophthalazine: To a solution of 6-bromo-4-hydroxyphthalazin-1(2H)-one (10.0 g, 41.5 mmol) in DCE (100 mL) was added PBr₅ (35.7 g, 82.9 mmol) at 25° C. The reaction mixture was stirred at 90° C. for 48 h. The reaction mixture was diluted with water (100 mL) and extracted with DCM (3 × 50 mL). The combined organic layers were washed with brine (100 mL), dried over anhydrous Na₂SO₄, filtered, and concentrated under reduced pressure to provide a residue which was used directly.¹H NMR (400 MHz, DMSO-d₆): δ 8.41-8.34 (m, 2H), 8.16 (d, J= 8.8 Hz, 1H).

4,6-dibromophthalazin-1-ol and 4,7-dibromophthalazin-1-ol: A solution of 1,4,6-tribromophthalazine (11.0 g, 30.0 mmol) in AcOH (110 mL) was stirred at 60° C. for 16 h. The reaction mixture was concentrated under reduced pressure. The residue was diluted with water (100 mL). The resulting mixture was filtered and the filter cake was dried under reduced pressure to provide a residue that was used directly as a 1:1 mixture of 4,6-dibromophthalazin-1-ol and 4,7-dibromophthalazin-1-ol. LCMS: m/z = 304.9, 306.9, 302.9 [M+H]⁺.

Ethyl 2-(4,6-dibromo-1-oxophthalazin-2(1H)-yl)acetate: To a mixture of 4,6-dibromophthalazin-1-ol and 4,7-dibromophthalazin-1-ol (1:1 mixture, 2.10 g, 6.91 mmol) in DMF (21 mL) at 0° C. was added NaH (277 mg, 6.91 mmol, 60% purity) followed by ethyl 2-bromoacetate (1.15 g, 6.91 mmol). The reaction mixture was stirred at 20° C. for 16 h. The reaction mixture was diluted with water (40 mL) and extracted with EtOAc (3 × 15 mL). The combined organic layers were washed with brine (40 mL), dried over anhydrous Na₂SO₄, filtered, and concentrated under reduced pressure. The crude residue was purified by preparative SFC. ¹H NMR (400 MHz, DMSO-d₆): δ 8.20-8.16 (m, 2H), 8.08 (s, 1H), 5.03-4.75 (s, 2H), 4.19-4.15 (m, 2H), 1.28-1.07 (m, 3H).

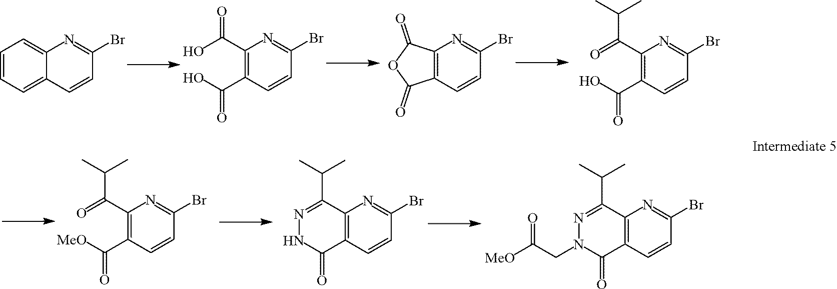

6-bromopyridine-2,3-dicarboxylic acid: To a solution of periodic acid (49.3 g, 216 mmol) and CC1₄ (180 mL) in water (360 mL) were added RuCl₃ (1.79 g, 8.65 mmol) and 2-bromoquinoline (9.00 g, 43.3 mmol). The reaction mixture was stirred at 20° C. for 16 h and then at 50° C. for 80 h. The reaction mixture was extracted with EtOAc (3 × 200 mL). The combined organic layers were dried over anhydrous Na₂SO₄, filtered, and concentrated under reduced pressure to provide a residue which was used directly. LCMS: m/z = 245.9, 247.9 [M+H]⁺.

2-bromofuro[3,4-b]pyridine-5,7-dione: A solution of 6-bromopyridine-2,3-dicarboxylic acid (6.00 g, 24.4 mmol) in acetic anhydride (10 mL) was stirred at 120° C. for 12 h. The reaction mixture was concentrated under reduced pressure. The residue was recrystallized from 1:5 mixture of MTBE and PE (10 mL) to provide a solid that was used directly.

6-bromo-2-isobutyrylnicotinic acid: To a mixture of 2-bromofuro[3,4-b]pyridine-5,7-dione (2.00 g, 8.77 mmol) and CuBr (126 mg, 0.87 mmol) in THF (20 mL) at -78° C. was added isopropyl magnesium chloride (4.39 mL, 2 M in THF). The reaction mixture was stirred at -78° C. for 1 h. The reaction mixture was quenched by the addition of sat. aq. NH₄C1 (50 mL) and extracted with EtOAc (3 × 15 mL). The combined organic layers were washed with brine (20 mL), dried over anhydrous Na₂SO₄, filtered, and concentrated under reduced pressure. The residue was purified by reverse-phase preparative HPLC. LCMS: m/z = 270.0, 272.0 [M-H]⁻.

Methyl 6-bromo-2-isobutyrylnicotinate: To a solution of 6-bromo-2-isobutyrylnicotinic acid (530 mg, 1.95 mmol) in THF (5.0 mL) at 0° C. was added TMSCHN₂ (1.46 mL, 2 M in n-hexane). The reaction mixture was stirred at 20° C. for 12 h and then concentrated under reduced pressure. The residue was purified by silica gel column chromatography. LCMS: m/z = 286.1, 288.1 [M+H]⁺.

2-bromo-8-isopropylpyrido[2,3-d]pyridazin-5(6H)-one: To a solution of methyl 6-bromo-2-isobutyrylnicotinate (350 mg, 1.22 mmol) in MeOH (5.0 mL) was added hydrazine monohydrate (44 mg, 1.35 mmol). The reaction mixture was stirred at 25° C. for 3 h. The reaction mixture was concentrated under reduced pressure to provide a residue which was used directly. ¹H NMR (400 MHz, CDC1₃): δ = 10.28 (br s, 1H), 8.51 (d, J= 8.4 Hz, 1H), 7.82 (d, J= 8.4 Hz, 1H), 3.90-3.86 (m, 1H), 1.35 (d, J= 6.8 Hz, 6H).

Methyl 2-(2-bromo-8-isopropyl-5-oxopyrido[2,3-d]pyridazin-6(5H)-yl)acetate: To a solution of 2-bromo-8-isopropylpyrido[2,3-d]pyridazin-5(6H)-one (246 mg, 0.91 mmol) and methyl 2-bromoacetate (155 mg, 1.01 mmol) in DMF (5 mL) was added Cs₂CO₃ (598 mg, 1.84 mmol). The reaction mixture was stirred at 25° C. for 12 h. The reaction mixture was poured into ice water (30 mL) and extracted with EtOAc (3 × 10 mL). The combined organic layers were washed with brine (3 × 10 mL), dried over anhydrous Na₂SO₄, filtered, and concentrated under reduced pressure. LCMS: m/z = 340.1, 342.1 [M+H]⁺.

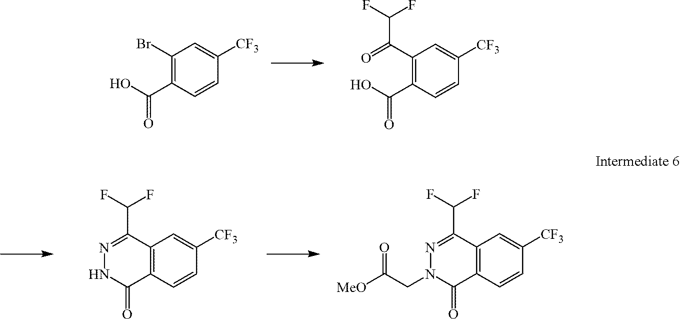 [SMILES: O=C(O)c1ccc(C(F)(F)F)cc1Br ; O=C(O)c1ccc(C(F)(F)F)cc1C(=O)C(F)F ; O=c1[nH]nc(C(F)F)c2cc(C(F)(F)F)ccc12 ; COC(=O)Cn1nc(C(F)F)c2cc(C(F)(F)F)ccc2c1=O]

2-(2,2-difluoroacetyl)-4-(trifluoromethyl)benzoic acid: To a solution of 2-bromo-4-(trifluoromethyl)benzoic acid (1.0 g, 3.72 mmol) in THF (20 mL) at -78 C was added n-BuLi (2.5 M in THF, 3.0 mL). The reaction mixture was stirred at -78° C. for 1 h followed by the dropwise addition of 2,2-difluoro-N-methoxy-N-methylacetamide (517 mg, 3.72 mmol) as a solution in THF (3.0 mL) at -78° C. The reaction mixture was then stirred at 20° C. for a further 12 h. The reaction mixture was poured into ice-cold water (20 mL) and adjusted to pH = 10 by the addition of sat. aq. Na₂HCO₃. The mixture was extracted with MTBE (3 × 10 mL) and the organics were discarded. The aqueous phase was then adjusted to pH = 3 with aq. HC1 (3 N) and extracted with EtOAc (4 10 mL). The combined organic layers were washed with brine (2 10 mL), dried over anhydrous Na₂SO₄, filtered, and concentrated under reduced pressure to provide a residue that was used directly. LCMS: m/z = 266.8 [M-H]⁻.

4-(difluoromethyl)-6-(trifluoromethyl)phthalazin-1(2H)-one: To a solution of 2-(2,2-difluoroacetyl)-4-(trifluoromethyl)benzoic acid (600 mg, 2.24 mmol) in EtOH (10 mL) was added hydrazine monohydrate (220 mg, 4.78 mmol). The reaction mixture was stirred at 90° C. for 12 h. Toluene (10 mL) was then added and the reaction mixture was stirred at 110° C. for 2 h and then at 125° C. for an additional 12 h. The reaction mixture was cooled to ambient temperature and concentrated under reduced pressure to provide residue that was used directly. LCMS: m/z = 265.0 [M+H]⁺.

Methyl 2-(4-(difluoromethyl)-1-oxo-6-(trifluoromethyl)phthalazin-2(1H)-yl)acetate: To a solution of 4-(difluoromethyl)-6-(trifluoromethyl)phthalazin-1(2H)-one (360 mg, 1.36 mmol) in DMF (10 mL) was added Cs₂CO₃ (444 mg, 1.36 mmol). The reaction mixture was stirred at 20° C. for 30 min and cooled to 0° C. To the reaction mixture at 0° C. was added methyl 2-bromoacetate (208 mg, 1.36 mmol) dropwise. The reaction mixture was stirred at 20° C. for 2 h. The mixture was diluted with water (50 mL) and extracted with EtOAc (3×10 mL). The combined organic layers were washed with brin (20 mL), dried over anhydrous Na₂SO₄, filtered, and concentrated under reduced pressure. The residue was purified by silica gel column chromatography. LCMS: m/z = 337.0 [M+H]⁺. ¹H NMR (400 MHz, CDC1₃): δ 8.62 (d, J= 8.4 Hz, 1H), 8.44 (s, 1H), 8.10-8.03 (m, 1H), 6.64 (t, J= 53.2 Hz, 1H), 4.99 (s, 2H), 3.82 (s, 3H).

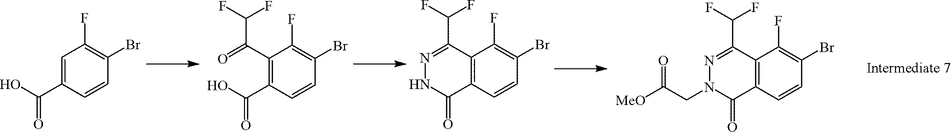 [SMILES: O=C(O)c1ccc(Br)c(F)c1 ; O=C(O)c1ccc(Br)c(F)c1C(=O)C(F)F ; O=c1[nH]nc(C(F)F)c2c(F)c(Br)ccc12 ; COC(=O)Cn1nc(C(F)F)c2c(F)c(Br)ccc2c1=O]

4-bromo-2-(2,2-difluoroacetyl)-3-fluorobenzoic acid: To a solution of n-BuLi (2.5 M in hexanes, 19.2 mL) in THF (50 mL) at -78° C. was added TMP (6.45 g, 45.7 mmol). The reaction mixture was stirred at -78° C. for 0.5 h. To the reaction mixture was added 4-bromo-3-fluorobenzoic acid (5.00 g, 22.8 mmol) as a solution in THF (10 mL). The reaction mixture was stirred at -78° C. for 2 h and then warmed to -60° C. To the reaction mixture was added 2,2-difluoro-N-methoxy-N-methylacetamide (3.49 g, 25.1 mmol). The mixture was stirred at -25° C. for 4 h. The reaction mixture was warmed to 0° C., quenched by addition of sat. aq. citric acid (30 mL), and filtered through a thin pad of celite. The filtrate was extracted with EtOAc (3 10 mL). The combined organic layers were washed with brine (10 mL), dried over anhydrous Na₂SO₄, filtered, and concentrated under reduced pressure to provide a residue that was used directly. LCMS: m/z = 294.8, 296.8 [M-H]⁻.

6-bromo-4-(difluoromethyl)-5-fluorophthalazin-1(2H)-one: To a solution of 4-bromo-2-(2,2-difluoroacetyl)-3-fluorobenzoic acid (1.0 g, 3.37 mmol) in EtOH (10 mL) and toluene (4 mL) was added hydrazine monohydrate (207 mg, 4.04 mmol). The reaction mixture was stirred at 120° C. for 16 h. The reaction mixture was concentrated under reduced pressure to provide a residue that was used directly. ¹H NMR (400 MHz, DMSO-d₆): δ 10.35 (br s, 1H), 8.26-8.22 (m, 1H), 8.07 (d, J= 8.4 Hz, 1H), 7.14 (t, J= 53.6, 1H).

Methyl 2-(6-bromo-4-(difluoromethyl)-5-fluoro-1-oxophthalazin-2(1H)-yl)acetate: To a solution of 6-bromo-4-(difluoromethyl)-5-fluorophthalazin-1(2H)-one (180 mg, 0.62 mmol) in DMF (2.0 mL) at 0° C. were added Cs₂CO₃ (200 mg, 0.62 mmol) and methyl 2-bromoacetate (104 mg, 0.66 mmol). The reaction mixture was stirred at 20° C. for 1 h. The reaction mixture was diluted with water (5 mL) and extracted with EtOAc (3 5 mL). The combined organic layers were washed with brine (5 mL), dried over anhydrous Na₂SO₄, filtered, and concentrated under reduced pressure. The residue was purified by silica gel column chromatography. ¹H NMR (400 MHz, CDC1₃): δ 8.20 (dd, J = 4.0, 7.6 Hz, 1H), 8.07-8.00 (m, 1H), 6.76 (t, J = 53.6 Hz, 1H), 4.99 (s, 2H), 3.81 (s, 3H).

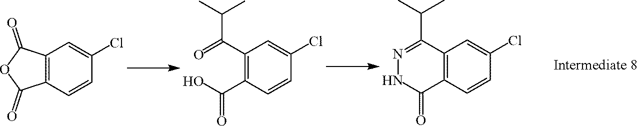

4-chloro-2-isobutyrylbenzoic acid: To a solution of 5-chloroisobenzofuran-1,3-dione (10.0 g, 54.8 mmol) in THF (100 mL) at 0° C. was added i-PrMgCl (30.1 mL, 2 M in THF). The reaction mixture was stirred at 0° C. for 4 h. The reaction mixture was quenched with sat. aq. NH₄Cl (50 mL) and extracted with EtOAc (3 × 20 mL). The combined organic layers were dried over anhydrous Na₂SO₄, filtered, and concentrated under reduced pressure. The residue was purified by reverse-phase preparative HPLC. ¹H NMR (400 MHz, CDC1₃): δ 7.77 (d, J= 8.4 Hz, 1H), 7.39 (dd, J= 8.4, 2.4 Hz, 1H), 7.02 (d, J= 2.0 Hz, 1H), 3.02-2.94 (m, 1H), 1.01 (d, J = 6.8 Hz, 6H).

6-chloro-4-isopropylphthalazin-1(2H)-one: To a solution of 4-chloro-2-isobutyrylbenzoic acid (730 mg, 3.22 mmol) in EtOH (10 mL) was added hydrazine monohydrate (200 mg, 3.92 mmol). The reaction mixture was stirred at 90° C. for 2 h. The reaction mixture was concentrated under reduced pressure. The residue was triturated with 1:1MTBE/PE (20 mL), filtered, and the collected solid from the filter cake was used directly. ¹H NMR (400 MHz, DMSO-d₆): δ 8.20 (d, J = 2.4 Hz, 1H), 8.10 (s, 1H), 7.96 (dd, J = 8.8, 2.4 Hz, 1H), 3.49-3.62 (m, 1H), 1.22-1.27 (m, 6H).

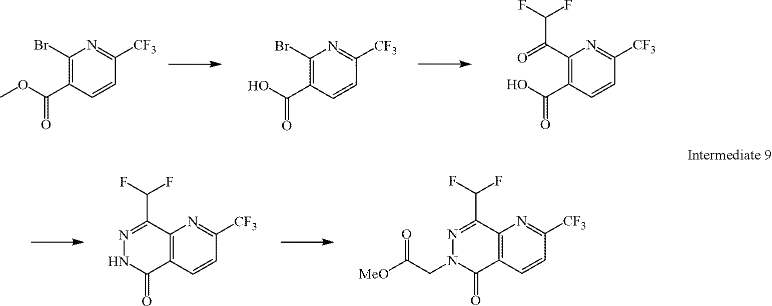 [SMILES: COC(=O)c1ccc(C(F)(F)F)nc1Br ; O=C(O)c1ccc(C(F)(F)F)nc1Br ; O=C(O)c1ccc(C(F)(F)F)nc1C(=O)C(F)F ; O=c1[nH]nc(C(F)F)c2nc(C(F)(F)F)ccc12 ; COC(=O)Cn1nc(C(F)F)c2nc(C(F)(F)F)ccc2c1=O]

2-bromo-6-(trifluoromethyl)nicotinic acid: To a solution of methyl 2-bromo-6-(trifluoromethyl)nicotinate (2.50 g, 8.80 mmol) in THF (20 mL) and H₂O (5 mL) was added LiOH•H₂O (1.10 g, 26.4 mmol). The reaction mixture was stirred at 20° C. for 2 h. The reaction mixture was adjusted to pH = 4 with aq. HC1 (1 N) and extracted with EtOAc (15 × 2 mL). The combined organic layers were washed with brine (15 mL), dried over anhydrous Na₂SO₄, filtered, and concentrated under reduced pressure to provide a residue that was used directly. ¹H NMR (400 MHz, CDC1₃): δ 10.99-10.51 (m, 1H), 8.38 (d, J= 7.8 Hz, 1H), 7.76 (d, J= 7.8 Hz, 1H).

2-(2,2-difluoroacetyl)-6-(trifluoromethyl)nicotinic acid: To a solution of 2-bromo-6-(trifluoromethyl)nicotinic acid (2.20 g, 8.15 mmol) in THF (20 mL) at -78° C. were added n-BuLi (2.5 M in hexanes, 7.2 mL) and 2,2-difluoro-N-methoxy-N-methylacetamide (1.50 g, 10.6 mmol). The reaction mixture was warmed to 20° C. and stirred for 16 h. The reaction mixture was diluted with water (10 mL) and extracted with EtOAc (3 10 mL). The combined organic layers were washed with brine (20 mL), dried over anhydrous Na₂SO₄, filtered, and concentrated under reduced pressure. The residue was purified by reverse-phase preparative HPLC. ¹H NMR (400 MHz, CDC1₃): δ 8.63-8.35 (m, 1H), 8.04 (d, J= 8.0 Hz, 1H), 6.27 (t, J = 54.0 Hz, 1H).

8-(difluoromethyl)-2-(trifluoromethyl)pyrido[2,3-d]pyridazin-5(6H)-one: To a solution of 2-(2,2-difluoroacetyl)-6-(trifluoromethyl)nicotinic acid (130 mg, 0.48 mmol) in EtOH (2 mL) was added hydrazine hydrate (27 mg, 0.51 mmol). The reaction mixture was stirred at 80° C. for 16 h. The reaction mixture was diluted with water (2 mL) and extracted with EtOAc (2 3 mL). The combined organic layers were washed with brine (10 mL), dried over anhydrous Na₂SO₄, filtered, and concentrated under reduced pressure to provide a residue that was used directly. ¹H NMR (400 MHz, CDC1₃): δ 9.01-8.90 (m, 1H), 8.12 (d, J = 8.4 Hz, 1H), 7.27 (t, J = 52.8 Hz, 1H).

Methyl 2-(8-(difluoromethyl)-5-oxo-2-(trifluoromethyl)pyrido[2,3-d]pyridazin-6(5H)-yl)acetate: To a solution of 8-(difluoromethyl)-2-(trifluoromethyl)pyrido[2,3-d]pyridazin-5(6H)-one (120 mg, 0.45 mmol) in DMF (2.0 mL) were added Cs₂CO₃ (295 mg, 0.90 mmol) and methyl 2-bromoacetate (55 mg, 0.36 mmol). The reaction mixture was stirred at 20° C. for 12 h. The reaction mixture was diluted with water (10 mL) and extracted with EtOAc (10 3 mL). The combined organic layers were washed with brine (10 mL), dried over anhydrous Na₂SO₄, filtered, and concentrated under reduced pressure. The residue was purified by silica gel column chromatography.¹H NMR (400 MHz, CDC1₃): δ 8.94 (d, J= 8.4 Hz, 1H), 8.17-8.01 (m, 1H), 7.25 (t, J = 53.2 Hz, 1H), 5.06 (s, 2H), 3.89-3.74 (m, 3H).

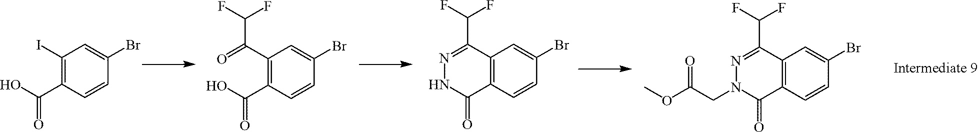 [SMILES: O=C(O)c1ccc(Br)cc1I ; O=C(O)c1ccc(Br)cc1C(=O)C(F)F ; O=c1[nH]nc(C(F)F)c2cc(Br)ccc12 ; COC(=O)Cn1nc(C(F)F)c2cc(Br)ccc2c1=O]

4-bromo-2-(2,2-difluoroacetyl)benzoic acid: To a solution of 4-bromo-2-iodobenzoic acid (20.0 g, 61.2 mmol) in THF (200 mL) at -78° C. was added n-BuLi (2.5 M in THF, 49 mL). The reaction mixture was stirred at -78° C. for 0.5 h followed by the dropwise addition of a solution of 2,2-difluoro-N-methoxy-N-methylacetamide (9.36 g, 67.3 mmol) in THF (20 mL) at -78° C. The reaction mixture was then stirred at 20° C. for a further 12 h. The reaction mixture was diluted with aq. sat. NH₄Cl (200 mL), extracted with MTBE (3 × 50 mL), and the organics were discarded. The aqueous phase was cooled to 0° C., adjusted to pH=3 using aq. HCl (3 N), and extracted with EtOAc (3 × 100 mL). These combined organic layers were washed with brine (100 mL), dried over anhydrous Na₂SO₄, filtered, and concentrated under reduced pressure to provide a residue that was used directly. LCMS: m/z = 276.9, 278.9 [M-H]⁻.

6-bromo-4-(difluoromethyl)phthalazin-1(2H)-one: To a solution of 4-bromo-2-(2,2-difluoroacetyl)benzoic acid (2.0 g, 7.2 mmol) in toluene (30 mL) was added NH₂NH₂•H₂O (703 mg, 13.8 mmol). The reaction mixture was stirred at 95° C. for 12 h. The reaction mixture was concentrated under reduced pressure to provide a residue that was used directly. LCMS: m/z = 272.9, 274.9 [M-H]⁻.

methyl 2-(6-bromo-4-(difluoromethyl)-1-oxophthalazin-2(1H)-yl)acetate: To a solution of 6-bromo-4-(difluoromethyl)phthalazin-1(2H)-one (1.5 g, 5.45 mmol) in DMF (20 mL) at 0° C. was added Cs₂CO₃ (1.95 g, 6.00 mmol). The reaction mixture was stirred at 0° C. for 30 min. followed by the addition of methyl 2-bromoacetate (834 mg, 5.45 mmol). The reaction mixture was then stirred at 20° C. for a further 2 h. The reaction mixture was diluted with water (50 mL) and extracted with EtOAc (3 × 20 mL). The combined organic layers were washed with brine (20 mL), dried over anhydrous Na₂SO₄, filtered, and concentrated under reduced pressure. The crude residue was purified silica gel column chromatography. LCMS: m/z = 347.0, 349.0 [M+H]⁺.

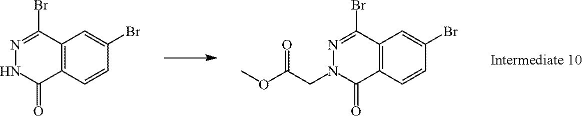 [SMILES: O=c1[nH]nc(Br)c2cc(Br)ccc12 ; COC(=O)Cn1nc(Br)c2cc(Br)ccc2c1=O]

methyl 2-(4,6-dibromo-1-oxophthalazin-2(1H)-yl)acetate: To a mixture of 4,6-dibromo-2H-phthalazin-1-one (13.0 g, 43.0 mmol) and methyl 2-bromoacetate (13.0 g, 86.0 mmol) in DMF (130 mL) was added Cs₂CO₃ (28.0 g, 85.5 mmol). The reaction mixture was stirred at 20° C. for 12 h. The reaction mixture was diluted with water (100 mL) and filtered. The solid was collected and dried under reduced pressure to provide a residue that was used directly. LCMS: m/z = 374.8, 376.8, 378.8 [M+H]⁺.

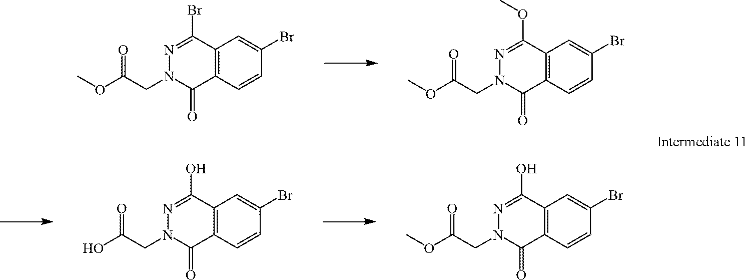 [SMILES: COC(=O)Cn1nc(Br)c2cc(Br)ccc2c1=O ; COC(=O)Cn1nc(OC)c2cc(Br)ccc2c1=O ; O=C(O)Cn1nc(O)c2cc(Br)ccc2c1=O ; COC(=O)Cn1nc(O)c2cc(Br)ccc2c1=O]

methyl 2-(6-bromo-4-methoxy-1-oxophthalazin-2(1H)-yl)acetate: To a mixture of sodium metal (3.0 g, 133 mmol) in MeOH (70 mL) was stirred at 20° C. for 30 min and then concentrated under reduced pressure. The residue was then added portion-wise to a mixture of methyl 2-(4,6-dibromo-1-oxophthalazin-2(1H)-yl)acetate (5.0 g, 13.0 mmol) in MeOH (70 mL). The reaction mixture was stirred at 60° C. for 5 h. The reaction mixture was diluted with water (100 mL) and extracted with EtOAc (3 × 15 mL). The combined organic layers were washed with brine (15 mL), dried over anhydrous Na₂SO₄, filtered, and concentrated under reduced pressure to provide a residue that was used directly. LCMS: m/z = 326.9, 329.0 [M+H]⁺.

2-(6-bromo-4-hydroxy-1-oxophthalazin-2(1H)-yl)acetic acid: To a mixture of methyl 2-(6-bromo-4-methoxy-1-oxo-phthalazin-2-yl)acetate (1.8 g, 5.50 mmol) in 1,4-dioxane (5 mL) was added HBr (15 mL, 47% purity in water). The reaction mixture was stirred at 100° C. for 12 h. The reaction mixture was concentrated under reduced pressure to provide a residue that was used directly. LCMS: m/z = 299.0, 300.9 [M+H]⁺.

methyl 2-(6-bromo-4-hydroxy-1-oxophthalazin-2(1H)-yl)acetate: To a solution of 2-(6-bromo-4-hydroxy-1-oxophthalazin-2(1H)-yl)acetic acid (10.0 g, 33.4 mmol) in MeOH (5 mL) at 0° C. was added SOCl₂ (11.9 g, 100 mmol). The reaction mixture was stirred at 25° C. for 2 h. The reaction mixture was concentrated under reduced pressure to provide a residue that was used directly. LCMS: m/z = 312.9, 314.9 [M+H]⁺.

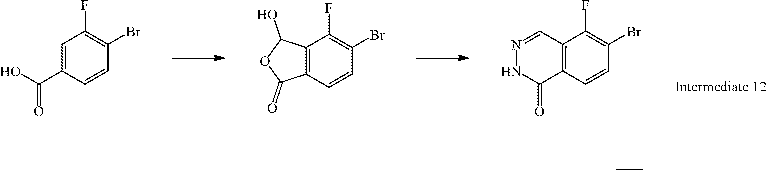

5-bromo-4-fluoro-3-hydroxyisobenzofuran-1(3H)-one: To a solution of TMP (80.6 g, 571 mmol) in THF (500 mL) at 0° C. was added n-BuLi (2.5 M in hexane, 219 mL) dropwise. The reaction mixture was stirred at 0° C. for 0.5 h and then cooled to -45° C. To the reaction mixture was added a solution of 4-bromo-3-fluorobenzoic acid (50.0 g, 228 mmol) in THF (200 mL) dropwise. The reaction mixture was stirred at -45° C. for a further 5 h followed by the addition of DMF (25.0 g, 343 mmol). The reaction mixture was then stirred at 20° C. for a further 14.5 h. The reaction mixture was diluted with aq. HCl (3 M, 500 mL) and extracted with DCM (3 × 200 mL). The combined organics were washed with brine (400 mL), dried over anhydrous Na₂SO₄, filtered, and concentrated under reduced pressure. The crude residue was purified by silica gel column chromatography. ¹H NMR(400 MHz, CD₃CN): δ 7.89 (dd, J = 6.0, 8.0 Hz, 1H), 7.55 (d, J = 8.0 Hz, 1H), 6.72 (s, 1H), 5.99 (br s, 1H).

6-bromo-5-fluorophthalazin-1(2H)-one: To a solution of 5-bromo-4-fluoro-3-hydroxyisobenzofuran-1(3H)-one (2.0 g, 8.10 mmol) in THF (40 mL) was added NH₂NH₂•H₂O (405 mg, 8.10 mmol). The reaction mixture was stirred at 20° C. for 3 h. The reaction mixture was diluted with water (50 mL), filtered, and the filter cake was dried under reduced pressure to provide a residue that was used directly. LCMS: m/z = 242.9, 244.9 [M+H]⁺.

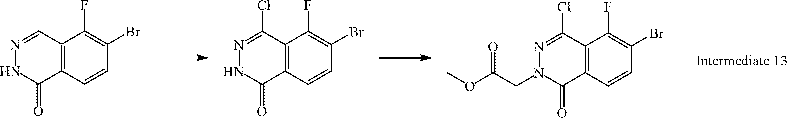 [SMILES: O=c1[nH]ncc2c(F)c(Br)ccc12 ; O=c1[nH]nc(Cl)c2c(F)c(Br)ccc12 ; COC(=O)Cn1nc(Cl)c2c(F)c(Br)ccc2c1=O]

6-bromo-4-chloro-5-fluorophthalazin-1(2H)-one: To a solution of 6-bromo-5-fluorophthalazin-1(2H)-one (5.0 g, 20.6 mmol) in DMF (50 mL) was added 1,3,5-trichloro-1,3,5-triazinane-2,4,6-trione (12.0 g, 51.4 mmol). The reaction mixture was stirred at 50° C. for 4 h. The reaction mixture was diluted with water (60 mL) and extracted with EtOAc (3 × 20 mL). The combined organics were washed with brine (40 mL), dried over anhydrous Na₂SO₄, filtered, and concentrated under reduced pressure. The crude residue was purified by silica gel column chromatography and further purified by reverse-phase preparative HPLC. LCMS: m/z = 276.8, 278.8, 280.8 [M+H]⁺.

methyl 2-(6-bromo-4-chloro-5-fluoro-1-oxophthalazin-2(1H)-yl)acetate: To a solution of 6-bromo-4-chloro-5-fluorophthalazin-1(2H)-one (200 mg, 0.72 mmol) in DMF (4.0 mL) were added Cs₂CO₃ (470 mg, 1.44 mmol) and methyl 2-bromoacetate (132 mg, 0.86 mmol). The reaction mixture was stirred at 20° C. for 2 h. The reaction mixture was diluted with water (8 mL) and extracted with EtOAc (3 × 3 mL). The combined organics were washed with brine (5 mL), dried over anhydrous Na₂SO₄, filtered, and concentrated under reduced pressure. The crude residue was purified by silica gel column chromatography. LCMS: m/z = 348.8, 350.8, 352.8 [M+H]⁺.

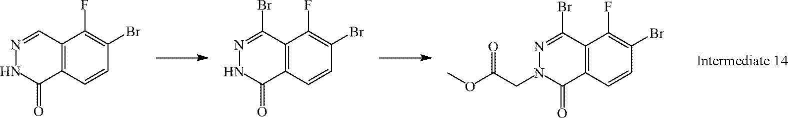 [SMILES: O=c1[nH]ncc2c(F)c(Br)ccc12 ; O=c1[nH]nc(Br)c2c(F)c(Br)ccc12 ; COC(=O)Cn1nc(Br)c2c(F)c(Br)ccc2c1=O]

4,6-dibromo-5-fluorophthalazin-1(2H)-one: To a solution of 6-bromo-5-fluorophthalazin-1(2H)-one (15.0 g, 61.7 mmol) in DMF (500 mL) at 0° C. were added K₂CO₃ (17.1 g, 123 mmol) and benzyltrimethylammonium tribromide (48.1 g, 123 mmol). The reaction mixture was stirred at 40° C. for 5 h. The reaction mixture was diluted with water and filtered. The filter cake was washed with water (3 × 500 mL) and dried under reduced pressure to provide a residue that was used directly. LCMS: m/z = 321.0, 323.0, 324.9 [M+H]⁺.

methyl 2-(4,6-dibromo-5-fluoro-1-oxophthalazin-2(1H)-yl)acetate: To a solution of 4,6-dibromo-5-fluorophthalazin-1(2H)-one (20.0 g, 62.1 mmol) in DMF (500 mL) at 0° C. was added Cs₂CO₃ (22.3 g, 68.3 mmol). The reaction mixture was stirred at 0° C. for 0.5 h followed by the dropwise addition of methyl 2-bromoacetate (9.50 g, 62.1 mmol). The reaction mixture was then stirred at 20° C. for a further 2 h. The reaction mixture was cooled to 0° C., diluted with water (1000 mL), and extracted with EtOAc (3 × 500 mL). The combined organic layers were washed with brine (500 mL), dried over anhydrous Na₂SO₄, filtered, and concentrated under reduced pressure. The crude residue was purified by silica gel column chromatography. LCMS: m/z = 393.0, 395.0, 397.0 [M+H]⁺.

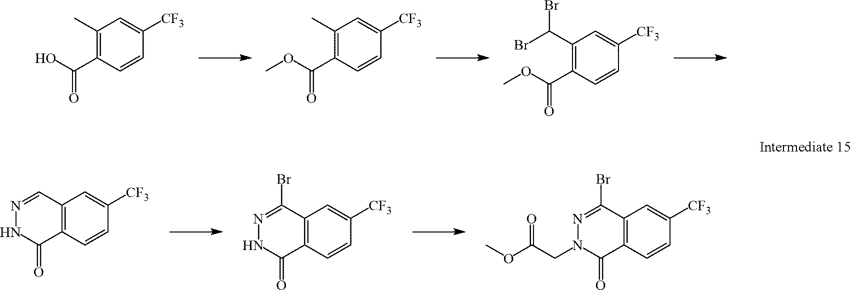 [SMILES: Cc1cc(C(F)(F)F)ccc1C(=O)O ; COC(=O)c1ccc(C(F)(F)F)cc1C ; COC(=O)c1ccc(C(F)(F)F)cc1C(Br)Br ; O=c1[nH]ncc2cc(C(F)(F)F)ccc12 ; O=c1[nH]nc(Br)c2cc(C(F)(F)F)ccc12 ; COC(=O)Cn1nc(Br)c2cc(C(F)(F)F)ccc2c1=O]

methyl 2-methyl-4-(trifluoromethyl)benzoate: To a solution of 2-methyl-4-(trifluoromethyl)benzoic acid (5.0 g, 24.5 mmol) in DMF (50 mL) at 0° C. were added K₂CO₃ (5.08 g, 36.7 mmol) and CH₃I (3.82 g, 26.9 mmol). The reaction mixture was stirred at 20° C. for 3 h. The reaction mixture was diluted with water (150 mL) and extracted with EtOAc (3 × 40 mL). The combined organic layers were washed with brine (2 × 20 mL), dried over anhydrous Na₂SO₄, filtered, and concentrated under reduced pressure. The crude residue was purified by silica gel column chromatography. ¹H NMR (400 MHz, CDCl₃): δ 8.00 (d, J = 8.0 Hz, 1H), 7.55-7.47 (m, 2H), 3.93 (s, 3H), 2.66 (s, 3H).

methyl 2-(dibromomethyl)-4-(trifluoromethyl)benzoate: To a solution of NBS (15.9 g, 89.4 mmol) in CCl₄ (50 mL) was added benzoyl peroxide (866 mg, 3.58 mmol) and methyl 2-methyl-4-(trifluoromethyl)benzoate (3.9 g, 17.9 mmol). The reaction mixture was stirred at 85° C. for 12 h. The reaction mixture was cooled to 20° C., filtered, and the filtrate was concentrated under reduced pressure. The crude residue was purified by silica gel column chromatography. ¹H NMR (400 MHz, CDCl₃): δ 8.42 (s, 1H), 8.05-7.98 (m, 2H), 7.63 (dd, J= 1.2, 8.4 Hz, 1H), 4.00 (s, 3H).

6-(trifluoromethyl)phthalazin-1-ol: To a solution of methyl 2-(dibromomethyl)-4-(trifluoromethyl)benzoate (6.4 g, 17.0 mmol) in MeOH (100 mL) was added NH₂NH₂•H₂O (3.5 g, 68.1 mmol). The reaction mixture was stirred at 80° C. for 12 h. The reaction mixture was concentrated under reduced pressure. The residue was triturated with MeOH (15 mL) and filtered. The filter cake was then dried under reduced pressure, triturated with water (20 mL), and filtered. The filter cake was then dried under reduced pressure to provide a residue that was used directly. LCMS: m/z = 215.2 [M+H]⁺.

4-bromo-6-(trifluoromethyl)phthalazin-1-ol: To a solution of 6-(trifluoromethyl)phthalazin-1-ol (1.77 g, 8.27 mmol) in DMF (50 mL) at 0° C. were added K₂CO₃ (2.28 g, 16.5 mmol) and benzyltrimethylammonium tribromide (6.45 g, 16.5 mmol). The reaction mixture was stirred at 40° C. for 5 h. The reaction mixture was diluted with water (100 mL) and filtered. The collected solid was washed with water (3 × 20 mL) and dried under reduced pressure to provide a residue that was used directly. LCMS: m/z = 293.1, 295.1 [M+H]⁺.

methyl 2-(4-bromo-1-oxo-6-(trifluoromethyl)phthalazin-2(1H)-yl)acetate: To a solution of 4-bromo-6-(trifluoromethyl)phthalazin-1-ol (500 mg, 1.71 mmol) in DMF (10 mL) at 0° C. was added Cs₂CO₃ (556 mg, 1.71 mmol). The reaction mixture was stirred at 0° C. for 30 min followed by the addition of methyl 2-bromoacetate (261 mg, 1.71 mmol). The reaction mixture was stirred at 20° C. for a further 1 h. The reaction mixture was diluted with water (40 mL) and extracted with EtOAc (3 × 10 mL). The combined organic layers were washed with brine (20 mL), dried over anhydrous Na₂SO₄, filtered and concentrated under reduced pressure. The crude residue was triturated with PE:MTBE (10:1, 11 mL) and dried under reduced pressure to provide a residue that was used directly. LCMS: m/z = 365.1, 367.1 [M+H]⁺.

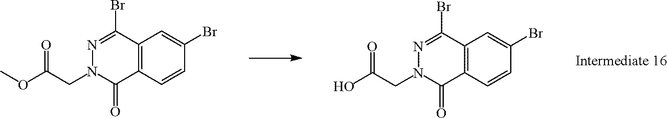 [SMILES: COC(=O)Cn1nc(Br)c2cc(Br)ccc2c1=O ; O=C(O)Cn1nc(Br)c2cc(Br)ccc2c1=O]

2-(4,6-dibromo-1-oxo-phthalazin-2-yl)acetic acid: To a solution of methyl 2-(4,6-dibromo-1-oxo-phthalazin-2-yl)acetate (1.5 g, 4.0 mmol) in THF (18 mL) was added aq. LiOH (8.0 mL, 1 M) at 25° C. The mixture was stirred at 40° C. for 2h. Aq. HCl (10.0 mL, 1 M) was added at 25° C. and the mixture was diluted and extracted with EtOAc (2 × 50 mL). The combined organic layers were washed with brine (20 mL), dried over anhydrous Na₂SO₄, filtered and concentrated under reduced pressure to provide a residue that was used directly. LCMS: m/z = 361.0, 363.0, 365.0 [M+H]⁺.

### Example 1 & 2

### 2-Bromo-4-Isopropyl-1-Oxophthalazin-2(1H)-yl)-N-(Cis-3-Hydroxy-3-Methylcyclobutyl)Acetamide and 2-(7-Bromo-4-Isopropyl-1-Oxophthalazin-2(1H)-yl)-N- (Cis-3-Hydroxy-3-Methylcyclobutyl)Acetamide

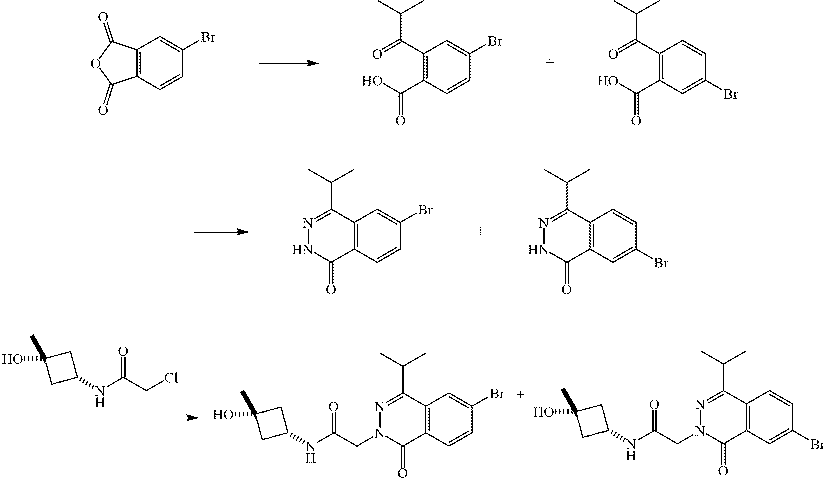

4-Bromo-2-isobutyrylbenzoic acid and 5-bromo-2-isobutyrylbenzoic acid: To a solution of 5-bromoisobenzofuran-1,3-dione (1.0 g, 4.41 mmol) in THF (10 mL) was added dropwise isopropyl magnesium chloride (2 M in THF, 2.43 mL, 4.86 mmol) at -10° C. The reaction mixture was stirred at 0° C. for 3 h. The reaction mixture was poured into sat. aq. NH₄Cl (30 mL) and extracted with EtOAc (3 × 20 mL). The combined organic layers were dried over anhydrous Na₂SO₄, filtered, and concentrated under reduced pressure to provide a residue as a mixture of isomers (1:1 molar ratio) that was used directly. LCMS: m/z = 268.9, 270.8 [M-H]⁻.

6-Bromo-4-isopropylphthalazin-1(2H)-one and 7-bromo-4-isopropylphthalazin-1(2H)-one: To a mixture of 4-bromo-2-isobutyrylbenzoic acid and 5-bromo-2-isobutyrylbenzoic acid (300 mg, 1.11 mmol, 1:1 molar ratio) in EtOH (5 mL) was added NH₂NH₂•H₂O (169 mg, 3.32 mmol, 98% purity). The reaction mixture was stirred at 90° C. for 2 h. The reaction mixture was concentrated under reduced pressure to provide a residue as a mixture of isomers (1:1 molar ratio) that was used directly. LCMS: m/z = 267.0, 269.0 [M+H]⁺.

2-(6-Bromo-4-isopropyl-1-oxophthalazin-2(1H)-yl)-N- (cis-3-hydroxy-3-methylcyclobutyl)acetamide and 2-(7-bromo-4-isopropyl-1-oxophthalazin-2(1H)-yl)-N-(cis-3-hydroxy-3-methylcyclobutyl)acetamide: To a mixture of 6-bromo-4-isopropylphthalazin-1(2H)-one and 7-bromo-4-isopropylphthalazin-1(2H)-one) (100 mg, 0.37 mmol, 1:1 molar ratio) and 2-chloro-N-(cis-3-hydroxy-3-methylcyclobutyl)acetamide (66 mg, 0.37 mmol) in DMF (1.5 mL) was added Cs₂CO₃ (146 mg, 0.45 mmol). The reaction mixture was stirred at 90° C. for 1 h. The reaction mixture was poured into water (10 mL) and extracted with EtOAc (4 × 5 mL). The combined organic layers were dried over anhydrous Na₂SO₄, filtered, and concentrated under reduced pressure. The crude residue was purified by reverse-phase HPLC to provide:

2-(6-Bromo-4-isopropyl-1-oxophthalazin-2(1H)-yl)-N- (cis-3-hydroxy-3-methylcyclobutyl)acetamide: LCMS: m/z = 408.0, 410.0 [M+H]⁺. ¹H NMR (400 MHz, CDCl₃): δ 8.32 (br d, J = 8.8 Hz, 1H), 8.02 (s, 1H), 7.87 (d, J= 8.8 Hz, 1H), 6.54 (br s, 1H), 4.83 (s, 2H), 4.01 (m, 1H), 3.43 (m, 1H), 2.50 (brt, J= 10.0 Hz, 2H), 2.29 (br s, 1H), 2.01 (br t,J = 10.0 Hz, 2H), 1.39-1.33 (m, 9H).

2-(7-Bromo-4-isopropyl-1-oxophthalazin-2(1H)-yl)-N- (cis-3-hydroxy-3-methylcyclobutyl)acetamide: LCMS: m/z = 408.0, 410.0 [M+H]⁺. ¹H NMR (400 MHz, CDCl₃): δ 8.62 (d, J = 2.0 Hz, 1H), 7.94 (dd, J = 2.0, 8.8 Hz, 1H), 7.76 (d, J= 8.8 Hz, 1H), 6.50 (br d, J= 6.8 Hz, 1H), 4.84 (s, 2H), 4.06-3.96 (m, 1H), 3.45 (m, 1H), 2.55-2.46 (m, 2H), 2.06-1.97 (m, 2H), 1.37-1.34 (m, 9H).

### Example 3

### (R)(6-Bromo-4-Isopropyl-1-Oxophthalazin-2(1H)-yl)-N-(1-Ethylpiperidin-3-yl)Acetamide

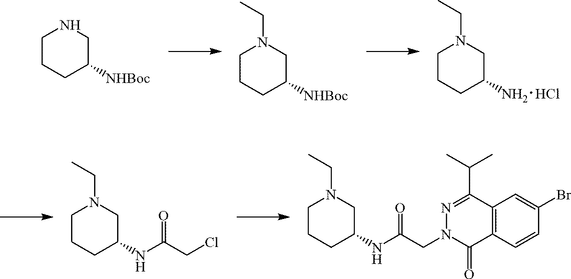

(R)-tert-Butyl (1-ethylpiperidin-3-yl)carbamate: To a solution of (R)-tert-butyl piperidin-3-ylcarbamate (10.0 g, 49.9 mmol) in MeCN (100 mL) at 0° C. was added K₂CO₃ (10.4 g, 74.9 mmol) followed by a solution of iodoethane (8.57 g, 54.9 mmol) in MeCN (10 mL) dropwise. The reaction mixture was stirred at 20° C. for 16 h. The reaction mixture was filtered and the filtrate was concentrated under reduced pressure. The crude residue was diluted with water (100 mL) and extracted with EtOAc (3 × 40 mL). The combined organic layers were washed with brine (100 mL), dried over anhydrous Na₂SO₄, filtered, and concentrated under reduced pressure provide a residue that was used directly. LCMS: m/z = 229.2 [M+H]⁺.

(R)-1-Ethylpiperidin-3-amine HCl salt: (R)-tert-Butyl (1-ethylpiperidin-3-yl)carbamate (5.0 g, 21.9 mmol) was dissolved in HCl (50 mL, 4 N in EtOAc). The reaction mixture was stirred at 20° C. for 1 h. The reaction mixture was concentrated under reduced pressure to provide a residue that was used directly. LCMS: m/z = 129.2 [M+H]⁺.

(R)-2-Chloro-N-(1-ethylpiperidin-3-yl)acetamide: To a solution of (R)-1-ethylpiperidin-3-amine HCl salt (1.0 g, 4.97 mmol) in DCM (10 mL) at 0° C. was added Et₃N (3.0 g, 29.8 mmol) followed by 2-chloroacetyl chloride (618 mg, 5.50 mmol) dropwise. The reaction mixture was stirred at 20° C. for 1 h. The reaction mixture was diluted with ice-cold sat. aq. NaHCO₃ (10 mL) and extracted with DCM (3 × 10 mL). The combined organic layers were washed with brine (10 mL), dried over anhydrous Na₂SO₄, filtered, and concentrated under reduced pressure to provide a residue that was used directly. LCMS: m/z = 205.1 [M+H]⁺.

(R)-2-(6-Bromo-4-isopropyl-1-oxophthalazin-2(1H)-yl)-N-(1-ethylpiperidin-3-yl)acetamide: To a solution of (R)-2-chloro-N-(1-ethylpiperidin-3-yl)acetamide (153 mg, 0.75 mmol) and 6-bromo-4-isopropylphthalazin-1(2H)-one (200 mg, 0.75 mmol) in DMF (3 mL) was added Cs₂CO₃ (488 mg, 1.50 mmol). The reaction mixture was stirred at 90° C. for 1 h. The reaction mixture was diluted with ice-cold water (15 mL) and extracted with EtOAc (3 × 10 mL). The combined organic layers were washed with brine (10 mL), dried over anhydrous Na₂SO₄, filtered, and concentrated under reduced pressure. The crude residue was purified by reverse-phase HPLC. LCMS: m/z = 435.0, 437.0 [M+H]⁺. ¹H NMR (400 MHz, CDCl₃): δ 8.36 (d, J = 8.4 Hz, 1H), 8.02 (d, J= 1.6 Hz, 1H), 7.87 (dd, J= 1.6, 2.0 Hz, 1H), 6.55 (br s, 1H), 4.96-4.76 (m, 2H), 4.17-4.02 (m, 1H), 3.43 (m, 1H), 2.48 (br s, 1H), 2.42-2.31 (m, 2H), 2.31-2.20 (m, 2H), 2.13 (br s, 1H), 1.62-1.46 (m, 4H), 1.37 (d, J = m, 6H), 0.87 (t, J = 7.2 Hz, 3H).

### Example 4

### (R)-N-Ethylpiperidin-3-yl)-2-(4-Isopropyl-6-Methyl-1-Oxophthalazin-2(1H)-yl)Acetamide

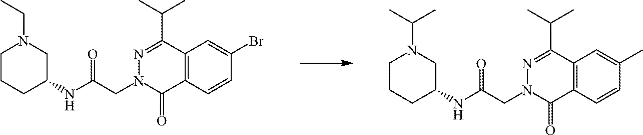 [SMILES: CCN1CCC[C@@H](NC(=O)Cn2nc(C(C)C)c3cc(Br)ccc3c2=O)C1 ; Cc1ccc2c(=O)n(CC(=O)N[C@@H]3CCCN(C(C)C)C3)nc(C(C)C)c2c1]

To a solution of (R)-2-(6-bromo-4-isopropyl-1-oxophthalazin-2(1H)-yl)-N-(1-ethylpiperidin-3-yl)acetamide (30 mg, 0.07 mmol) and 2,4,6-trimethyl-1,3,5,2,4,6-trioxatriborinane (26 mg, 0.21 mmol) in 1,4-dioxane (0.5 mL) and water (0.1 mL) were added Pd(dppf)Cl₂ (5 mg, 0.007 mmol) and Cs₂CO₃ (45 mg, 0.14 mmol). The reaction mixture was stirred at 100° C. for 2 h. The reaction mixture was diluted with ice-cold water (5 mL) and extracted with EtOAc (3 × 5 mL). The combined organic layers were washed with brine (5 mL), dried over anhydrous Na₂SO₄, filtered, and concentrated under reduced pressure. The crude residue was purified by reverse-phase HPLC. LCMS: m/z = 371.1 [M+H]⁺. ¹H NMR (400 MHz, CDCl₃): δ 8.39 (d, J = 8.4 Hz, 1H), 7.65 (s, 1H), 7.59 (d, J= 8.0 Hz, 1H), 6.59 (br s, 1H), 4.97-4.78 (m, 2H), 4.09 (br s, 1H), 3.45-3.55 (m, 1H), 2.58 (s, 3H), 2.43 (br s, 1H), 2.35 (br s, 2H), 2.28-2.22 (m, 2H), 2.14 (br s, 1H), 1.53 (m, 4H), 1.36 (d, J= 6.8 Hz, 6H), 0.83 (t, J = 7.2 Hz, 3H).

### Example 5

### (R)(6-Bromo-4-Isopropyl-1-Oxophthalazin-2(1H)-yl)-N-(1-Cyclopropylpiperidin-3-yl)Acetamide

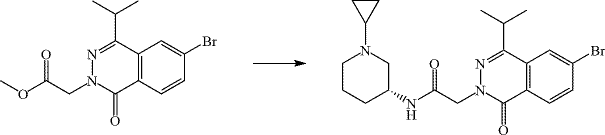 [SMILES: COC(=O)Cn1nc(C(C)C)c2cc(Br)ccc2c1=O ; CC(C)c1nn(CC(=O)N[C@@H]2CCCN(C3CC3)C2)c(=O)c2ccc(Br)cc12]

To a mixture of methyl 2-(6-bromo-4-isopropyl-1-oxophthalazin-2(1H)-yl)acetate (80 mg, 0.24 mmol) and (R)-1-cyclopropylpiperidin-3-amine HCl salt (125 mg, 0.71 mmol) in toluene (2 mL) and THF (2 mL) was added AlMe₃ (0.35 mL, 2 M in toluene). The reaction mixture was stirred at 110° C. for 3 h. The reaction mixture was poured into ice-cold water (10 mL) and extracted with EtOAc (2 × 5 mL). The organic layers were combined and washed with brine (10 mL), dried over anhydrous Na₂SO₄, filtered, and concentrated under reduced pressure. The residue was purified by reverse-phase HPLC. LCMS: m/z = 447.1, 449.1 [M+H]⁺. ¹H NMR (400 MHz, CDCl₃): δ 8.36 (d, J= 8.4 Hz, 1H), 8.03 (d, J = 1.8 Hz, 1H), 7.89 (dd, J = 8.4, 1.8 Hz, 1H), 6.42 (br s, 1H), 4.92-4.77 (m, 2H), 4.04 (br s, 1H), 3.50-3.37 (m, 1H), 2.63 (br s, 1H), 2.51 (br s, 1H), 2.27 (br s, 1H), 1.74-1.63 (m, 2H), 1.55-1.45 (m, 4H), 1.36 (m, 6H), 0.33-0.23 (m, 2H), -0.06 (m, 2H).

### Example 6

### 2-Bromo-4-Isopropyl-1-Oxophthalazin-2(1H)-yl)-N-(5-Fluoropyrimidin-4-yl)Acetamide

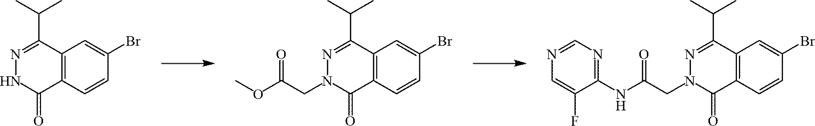 [SMILES: CC(C)c1n[nH]c(=O)c2ccc(Br)cc12 ; COC(=O)Cn1nc(C(C)C)c2cc(Br)ccc2c1=O ; CC(C)c1nn(CC(=O)Nc2ncncc2F)c(=O)c2ccc(Br)cc12]

Methyl 2-(6-bromo-4-isopropyl-1-oxophthalazin-2(1H)-yl)acetate: To a solution of 6-bromo-4-isopropylphthalazin-1(2H)-one (0.50 g, 1.87 mmol) in DMF (5 mL) were added Cs₂CO₃ (1.22 g, 3.74 mmol) and methyl 2-bromoacetate (315 mg, 2.06 mmol). The reaction mixture was stirred at 90° C. for 1 h. The reaction mixture was diluted with EtOAc (10 mL), washed with brine (2 × 5 mL), dried over anhydrous Na₂SO₄, filtered, and concentrated under reduced pressure to provide a residue that was used directly. LCMS: m/z = 339.1, 341.0 [M+H]⁺. ¹H NMR (400 MHz, CDCl₃): δ 8.34 (d, J = 8.8 Hz, 1H), 8.01 (d, J= 1.6 Hz, 1H), 7.85 (m, 1H), 4.94 (s, 2H), 3.79 (s, 3H), 3.41 (m, 1H), 1.35 (d, J = 6.8 Hz, 6H).

2-(6-Bromo-4-isopropyl-1-oxophthalazin-2(1H)-yl)-N-(5-fluoropyrimidin-4-yl)acetamide: To a solution of 5-fluoropyrimidin-4-amine (100 mg, 0.88 mmol) and methyl 2-(6-bromo-4-isopropyl-1-oxophthalazin-2(1H)-yl)acetate (100 mg, 0.29 mmol) in toluene (1 mL) and THF (1 mL) was added AlMe₃ (0.44 mL, 2 M in toluene). The reaction mixture was stirred at 110° C. for 3 h. The reaction mixture was quenched by the addition of water (0.5 mL) and filtered. The filtrate was extracted with EtOAc (1 × 5 mL) and the organic phase was dried over anhydrous Na₂SO₄, filtered, and concentrated under reduced pressure. The residue was purified by reverse-phase HPLC. LCMS: m/z = 419.9, 421.9 [M+H]⁺. ¹H NMR (400 MHz, CDCl₃): δ 8.77 (d, J= 2.0 Hz, 1H), 8.50 (d, J= 2.8 Hz, 1H), 8.37 (d, J = 8.4 Hz, 1H), 8.04 (d, J= 1.6 Hz, 1H), 7.89 (m, 1H), 5.42 (s, 2H), 4.77 (s, 1H), 3.51-3.39 (m, 1H), 1.37 (d, J = 6.8 Hz, 6H).

### Example 7

### (R)(6-Bromo-4-Isopropyl-1-Oxophthalazin-2(1H)-yl)-N-(1-(2,2,2-Trifluoroethyl)Piperidin-3-yl)Acetamide

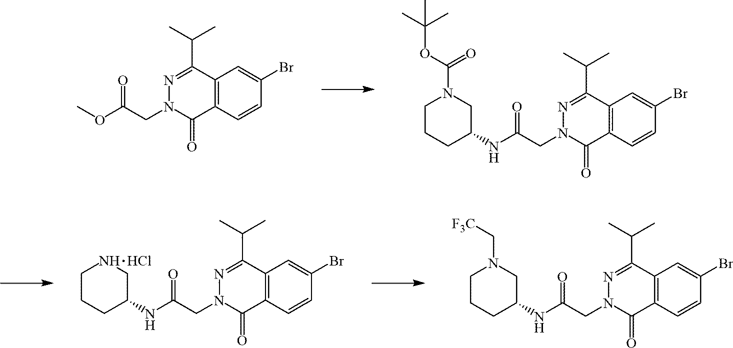

(R)-tert-Butyl-3-(2-(6-bromo-4-isopropyl-1-oxophthalazin-2(1H)-yl)acetamido)piperidine-1-carboxylate: To a mixture of methyl 2-(6-bromo-4-isopropyl-1-oxophthalazin-2(1H)-yl)acetate (150 mg, 0.44 mmol) and (R)-tert-butyl 3-aminopiperidine-1-carboxylate (89 mg, 0.44 mmol) in THF (2 mL) was added AlMe₃ (0.66 mL, 2 M in toluene). The reaction mixture was stirred at 110° C. for 3 h. The reaction mixture was poured into ice-cold water (10 mL) and extracted with EtOAc (2 × 5 mL). The organic layers were combined and washed with brine (10 mL), dried over anhydrous Na₂SO₄, filtered, and concentrated under reduced pressure. The residue was purified by column chromatograph. LCMS: m/z = 407.1, 409.1 [M-99]⁺.

(R)-2-(6-Bromo-4-isopropyl-1-oxophthalazin-2(1H)-yl)-N-(piperidin-3-yl)acetamide HCl salt: (R)-tert-Butyl-3-(2-(6-bromo-4-isopropyl-1-oxophthalazin-2(1H)-yl)acetamido)piperidine-1-carboxylate (70 mg, 0.14 mmol) was dissolved in HCl (10 mL, 4 N in EtOAc). The reaction mixture was stirred at 20° C. for 1 h. The reaction mixture was concentrated under reduced pressure to provide a residue that was used directly. LCMS: m/z = 407.0, 409.0 [M+H]⁺.

(R)-2-(6-Bromo-4-isopropyl-1-oxophthalazin-2(1H)-yl)-N-(1-(2,2,2-trifluoroethyl)piperidin-3-yl)acetamide: To a solution of (R)-2-(6-bromo-4-isopropyl-1-oxophthalazin-2(1H)-yl)-N-(piperidin-3-yl)acetamide HCl salt (70 mg, 0.16 mmol) and 2,2,2-trifluoroethyl trifluoromethanesulfonate (55 mg, 0.24 mmol) in DMF (2 mL) was added DIPEA (61 mg, 0.47 mmol). The reaction mixture was stirred at 20° C. for 2 h. The reaction mixture was poured into ice-cold water (10 mL) and extracted with EtOAc (2 × 5 mL). The organic layers were combined and washed with brine (10 mL), dried over anhydrous Na₂SO₄, filtered, and concentrated under reduced pressure. The residue was purified by reverse-phase HPLC. LCMS: m/z = 489.1, 491.1 [M+H]⁺. ¹H NMR (400 MHz, CDCl₃): δ 8.35 (d, J = 8.6 Hz, 1H), 8.02 (d, J = 1.6 Hz, 1H), 7.88 (dd, J = 8.6, 1.8 Hz, 1H), 6.51 (br d, J= 7.6 Hz, 1H), 4.97-4.78 (m, 2H), 4.15-4.06 (m, 1H), 3.49-3.38 (m, 1H), 2.82 (quind, J = 9.6, 5.6 Hz, 2H), 2.75-2.63 (m, 2H), 2.62-2.55 (m, 1H), 2.43 (br t, J = 10.4 Hz, 1H), 1.79-1.63 (m, 2H), 1.55-1.45 (m, 2H), 1.36 (dd, J = 6.8, 2.0 Hz, 6H).

### Example 8

### 2-Bromo-4-Isopropyl-1-Oxophthalazin-2(1H)-yl)-N-(3-Fluoropyridin-4-yl)Acetamide

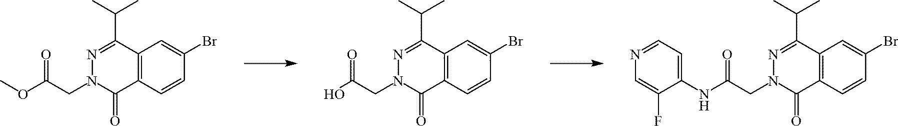 [SMILES: COC(=O)Cn1nc(C(C)C)c2cc(Br)ccc2c1=O ; CC(C)c1nn(CC(=O)O)c(=O)c2ccc(Br)cc12 ; CC(C)c1nn(CC(=O)Nc2ccncc2F)c(=O)c2ccc(Br)cc12]

2-(6-Bromo-4-isopropyl-1-oxophthalazin-2(1H)-yl)acetic acid: To a solution of methyl 2-(6-bromo-4-isopropyl-1-oxophthalazin-2(1H)-yl)acetate (0.50 g, 1.47 mmol) in THF (10 mL) and water (10 mL) was added LiOH•H₂O (124 mg, 2.95 mmol). The reaction mixture was stirred at 25° C. for 2 h. The reaction mixture was poured into water (50 mL) and extracted with MTBE (2 × 20 mL). The aqueous layer was then adjusted to pH = 3-4 by the addition of aq. HCl (3 M) and then extracted with EtOAc (3 × 20 mL). The combined organic layers were dried over anhydrous Na₂SO₄, filtered, and concentrated under reduced pressure to provide a residue that was used directly. ¹H NMR (400 MHz, DMSO-d6): δ 8.29 (d, J= 1.6 Hz, 1H), 8.21 (d, J= 8.4 Hz, 1H), 8.05 (dd, J= 2.0, 8.4 Hz, 1H), 4.78 (s, 2H), 3.69-3.55 (m, 1H), 1.25 (d, J= 6.8 Hz, 6H).

2-(6-Bromo-4-isopropyl-1-oxophthalazin-2(1H)-yl)-N-(3-fluoropyridin-4-yl)acetamide: To a solution of 2-(6-bromo-4-isopropyl-1-oxophthalazin-2(1H)-yl)acetic acid (50 mg, 0.14 mmol) in DMF (1 mL) were added 3-fluoropyridin-4-amine (19 mg, 0.17 mmol), DIPEA (80 mg, 0.63 mmol), and HATU (117 mg, 0.31 mmol). The reaction mixture was stirred at 20° C. for 3 h. The reaction mixture was poured into ice-cold water (10 mL) and extracted with EtOAc (2 × 5 mL). The organic layers were combined and washed with brine (10 mL), dried over anhydrous Na₂SO₄, filtered, and concentrated under reduced pressure. The residue was purified by reverse-phase HPLC. LCMS: m/z = 418.9, 420.9 [M+H]⁺. ¹H NMR (400 MHz, CDCl₃): δ 9.09 (br s, 1H), 8.39 (m, 2H), 8.35-8.28 (m, 2H), 8.06 (d, J= 1.2 Hz, 1H), 7.92 (dd, J = 1.6, 8.4 Hz, 1H), 5.05 (s, 2H), 3.46 (m, 1H), 1.39 (d, J = 6.8 Hz, 6H).

### Example 9

### 2-Bromo-5-Fluoro-4-Isopropyl-1-Oxophthalazin-2(1H)-yl)-N-(5-Fluoropyrimidin-4-yl)Acetamide

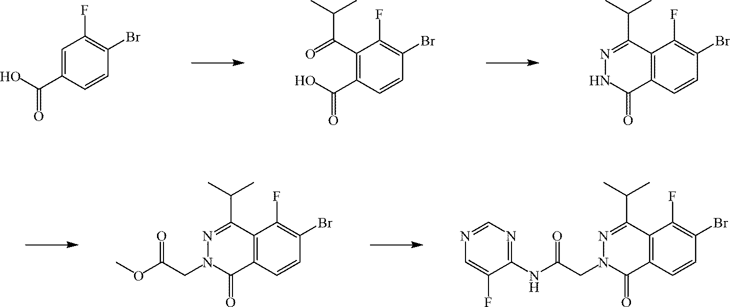 [SMILES: O=C(O)c1ccc(Br)c(F)c1 ; CC(C)C(=O)c1c(C(=O)O)ccc(Br)c1F ; CC(C)c1n[nH]c(=O)c2ccc(Br)c(F)c12 ; COC(=O)Cn1nc(C(C)C)c2c(F)c(Br)ccc2c1=O ; CC(C)c1nn(CC(=O)Nc2ncncc2F)c(=O)c2ccc(Br)c(F)c12]

4-Bromo-3-fluoro-2-isobutyrylbenzoic acid: To a solution of n-BuLi (18.0 mL, 2.5 M in THF) in THF (50 mL) at -78° C. was added 2,2,6,6-tetramethylpiperidine (6.77 g, 47.9 mmol). The reaction mixture was stirred at -78° C. for 30 minutes and then a solution of 4-bromo-3-fluorobenzoic acid (5.0 g, 22.8 mmol) in THF (10 mL) was added dropwise. The reaction mixture was stirred at -78° C. for a further 2 h. After this time, the reaction mixture was adjusted to -60° C. and N-methoxy-N-methylisobutyramide (3.29 g, 25.1 mmol) was added. The reaction mixture was stirred at -25° C. for a further 4 h. The reaction mixture was allowed to warm to 0° C., quenched by the addition of sat. aq. citric acid (30 mL), and extracted with EtOAc (3 × 10 mL). The combined organic layers were washed with brine (10 mL), dried over anhydrous Na₂SO₄, filtered, and concentrated under reduced pressure to provide a residue that was used directly. LCMS: m/z = 286.9, 288.9 [M-H]⁻. ¹H NMR (400 MHz, DMSO-d6): δ 13.21 (br s, 1H), 7.95 (br s, 1H), 7.72-7.59 (m, 1H), 2.93-2.85 (m, 1H), 1.00 (d, J= 6.8 Hz, 6H).

6-Bromo-5-fluoro-4-isopropylphthalazin-1(2H)-one: To a solution of 4-bromo-3-fluoro-2-isobutyrylbenzoic acid (2.5 g, 8.65 mmol) in EtOH (20 mL) was added NH₂NH₂•H₂O (530 mg, 10.4 mmol). The reaction mixture was stirred at 90° C. for 12 h. The reaction mixture was concentrated under reduced pressure. The residue was triturated with EtOH (10 mL) to provide a residue that was used directly. LCMS: m/z = 285.1, 287.0 [M+H]⁺. ¹H NMR (400 MHz, DMSO-d6): δ 12.43 (br s, 1H), 8.18-8.08 (m, 1H), 8.03 (m, 1H), 3.60-3.47 (m, 1H), 1.23 (br d, J = 6.0 Hz, 6H).

Methyl 2-(6-bromo-5-fluoro-4-isopropyl-1-oxophthalazin-2(1H)-yl)acetate: To a solution of 6-bromo-5-fluoro-4-isopropylphthalazin-1(2H)-one (450 mg, 1.58 mmol) in DMF (10 mL) at 0° C. was added Cs₂CO₃ (514 mg, 1.58 mmol). The reaction mixture was stirred at 0° C. for 30 min followed by the dropwise addition of a solution of methyl 2-bromoacetate (241 mg, 1.58 mmol) in DMF (2 mL). The resultant mixture was stirred at 20° C. for 2.5 h. The reaction mixture was diluted with water (50 mL) and extracted with EtOAc (3 × 10 mL). The combined organic layers were washed with brine (20 mL), dried over anhydrous Na₂SO₄, filtered, and concentrated under reduced pressure. The residue was purified by silica gel column chromatography. LCMS: m/z = 357.1, 359.1 [M+H]⁺. ¹H NMR (400 MHz, CDCl₃): δ 8.18 (dd, J= 0.8, 8.4 Hz, 1H), 7.91 (dd, J = 6.4, 8.4 Hz, 1H), 4.93 (s, 2H), 3.79 (s, 3H), 3.65-3.61 (m, 1H), 1.31 (dd, J= 1.2, 6.8 Hz, 6H).

2-(6-Bromo-5-fluoro-4-isopropyl-1-oxophthalazin-2(1H)-yl)-N-(5-fluoropyrimidin-4-yl)acetamide: To a mixture of 5-fluoropyrimidin-4-amine (142 mg, 1.26 mmol) and methyl 2-(6-bromo-5-fluoro-4-isopropyl-1-oxophthalazin-2(1H)-yl)acetate (150 mg, 0.42 mmol) in toluene (3 mL) and THF (3 mL) was added dropwise AlMe₃ (0.6 mL, 2 M in toluene). The reaction mixture was stirred for 3 h at 110° C. The reaction mixture was poured into ice-cold water (10 mL) and extracted with EtOAc (4 × 5 mL). The combined organic layers were washed with brine (2 × 10 mL), dried over anhydrous Na₂SO₄, filtered, and concentrated under reduced pressure. The residue was triturated with MTBE (10 mL) to provide the desired product. LCMS: m/z = 438.0, 440.0 [M+H]⁺. ¹H NMR (400 MHz, CDCl₃): δ 8.77 (d, J = 2.0 Hz, 1H), 8.63 (br s, 1H), 8.50 (d, J = 2.4 Hz, 1H), 8.22 (dd, J= 0.8, 8.4 Hz, 1H), 7.95 (dd, J = 6.4, 8.4 Hz, 1H), 5.46 (s, 2H), 3.73-3.59 (m, 1H), 1.33 (dd, J= 1.2, 6.8 Hz, 6H).

### Example 10

### Tert-Butyl-5-[[2-(6-Bromo-4-Isopropyl-1-Oxo-Phthalazin-2-yl)Acetyl]Amino]-3,3-Difluoro-Piperidine-1-Carboxylate

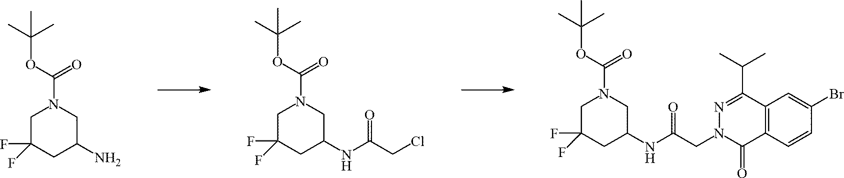 [SMILES: CC(C)(C)OC(=O)N1CC(N)CC(F)(F)C1 ; CC(C)(C)OC(=O)N1CC(NC(=O)CCl)CC(F)(F)C1 ; CC(C)c1nn(CC(=O)NC2CN(C(=O)OC(C)(C)C)CC(F)(F)C2)c(=O)c2ccc(Br)cc12]

tert-Butyl 5-[(2-chloroacetyl)amino]-3,3-difluoro-piperidine-1-carboxylate: To a mixture of tert-butyl 5-amino-3,3-difluoro-piperidine-1-carboxylate (295 mg, 1.25 mmol) and N-methylmorpholine (379 mg, 3.74 mmol) in DMF (0.44 mL) and DCM (2.2 mL) at -78° C. was added a solution of 2-chloroacetyl chloride (141 mg, 1.25 mmol) in DCM (2 mL). The reaction mixture was stirred at 23° C. for 3 h. The reaction mixture was poured into water (10 mL) and extracted with EtOAc (4 × 5 mL). The combined organic layers were washed with brine (2 × 10 mL), dried over anhydrous Na₂SO₄, filtered, and concentrated under reduced pressure to provide a residue that was used directly. ¹H NMR (400 MHz, DMSO-d6): δ 8.30-8.26 (m, 1H), 4.16-4.05 (m, 2H), 4.04-3.80 (m, 4H), 2.97-2.72 (m, 1H), 2.35-2.29 (m, 1H), 2.05-1.99 (m, 1H), 1.45-1.29 (m, 9H).

tert-Butyl-5-[[2-(6-bromo-4-isopropyl-1-oxo-phthalazin-2-yl)acetyl]amino]-3,3-difluoro-piperidine-1-carboxylate: To a mixture of 6-bromo-4-isopropyl-2H-phthalazin-1-one (150 mg, 0.56 mmol) and tert-butyl 5-[(2-chloroacetyl)amino]-3,3-difluoro-piperidine-1-carboxylate (193 mg, 0.62 mmol) in MeCN (7.7 mL) was added Cs₂CO₃ (276 mg, 0.84 mmol). The reaction mixture was stirred at 60° C. for 18 h. The reaction mixture was poured into ice water (30 mL) and extracted with EtOAc (30 mL). The organic layer was washed with water (2 × 25 mL) and brine (25 mL), dried over anhydrous Na₂SO₄, filtered, and concentrated under reduced pressure. The residue was purified by silica gel column chromatography. LCMS: m/z = 543.1, 545.1 [M+H]⁺. ¹H NMR (400 MHz, DMSO-d6): δ 8.29 (d, J= 1.8 Hz, 1H), 8.24-8.20 (m, 2H), 8.05 (dd, J = 8.5, 1.8 Hz, 1H), 4.71 (s, 2H), 4.09-4.00 (m, 2H), 3.87-3.79 (m, 2H), 3.65-3.58 (m, 1H), 3.44-3.40 (m, 2H), 2.35-2.30 (m, 1H), 1.40 (s, 9H), 1.25 (d, J= 6.7 Hz, 6H).

### Example 11

### 2-Bromo-4-Isopropyl-1-Oxophthalazin-2(1H)-yl)-N-(5,5-Difluoropiperidin-3-yl)Acetamide HCl Salt

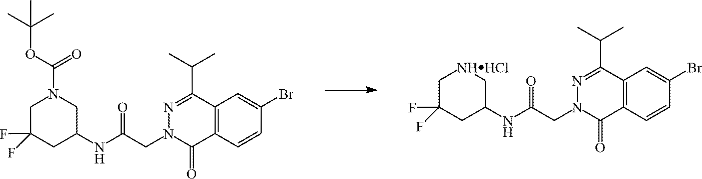

tert-Butyl-5-[[2-(6-bromo-4-isopropyl-1-oxo-phthalazin-2-yl)acetyl]amino]-3,3-difluoro-piperidine-1-carboxylate (314 mg, 0.58 mmol) was dissolved in HCl (10 mL, 4 N in 1,4-dioxane). The reaction mixture was stirred at 23° C. for 3 h. The reaction mixture was concentrated under reduced pressure to provide a residue that was used directly. LCMS: m/z = 443.1, 445.1 [M+H]⁺. ¹H NMR (400 MHz, DMSO-d6): δ 8.61 (d, J = 7.5 Hz, 1H), 8.29 (d, J = 1.8 Hz, 1H), 8.20 (d, J = 8.5 Hz, 1H), 8.05 (m, 1H), 4.73 (s, 2H), 4.19-4.13 (m, 1H), 3.71-3.62 (m, 3H), 3.27-3.23 (m, 2H), 2.88 (m, 1H), 2.45-2.39 (m, 1H), 2.24-2.11 (m, 1H), 1.27-1.15 (m, 6H).

### Example 12

### 2-Bromo-4-Isopropyl-1-Oxophthalazin-2(1H)-yl)-N-(1-Ethyl-5,5-Difluoropiperidin-3-yl)Acetamide

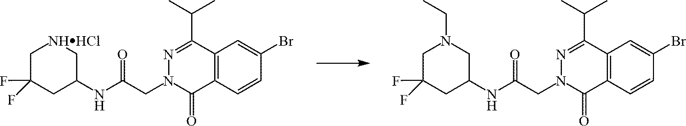

To a mixture of 2-(6-bromo-4-isopropyl-1-oxophthalazin-2(lH)-yl)-N-(5,5-difluoropiperidin-3-yl)acetamide HCl salt (140 mg, 0.29 mmol) in MeCN (10 mL) were added iodoethane (55 mg, 0.35 mmol) and K₂CO₃ (121 mg, 0.88 mmol). The reaction mixture was stirred at 60° C. for 18 h. The reaction mixture was poured into ice water (50 mL) and extracted with EtOAc (50 mL). The organic layer was washed with water (2 × 30 mL) and brine (30 mL), dried over anhydrous Na₂SO₄, filtered, and concentrated under reduced pressure. The residue was purified by silica gel column chromatography. LCMS: m/z = 471.1, 473.1 [M+H]⁺. ¹H NMR (400 MHz, DMSO-d6): δ 8.29 (m, 1H), 8.21 (d, J= 8.5 Hz, 1H), 8.10 (t, J= 0.4 Hz, 1H), 8.05 (m, 1H), 4.70 (s, 2H), 4.02-3.85 (m, 2H), 3.65-3.58 (m, 2H), 2.98-2.90 (m, 1H), 2.84-2.79 (m, 1H), 2.35-2.17 (m, 3H), 2.01-1.95 (m, 1H), 1.26-1.21 (m, 6H), 0.98 (t, J = 7.2 Hz, 3H).

### Example 13

### 2-Bromo-4-(1,1-Difluoroethyl)-1-Oxophthalazin-2-yl]-N-(5-Fluoropyrimidin-4-yl)Acetamide

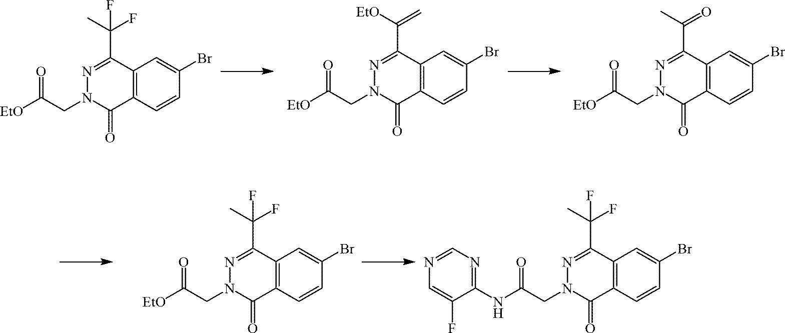 [SMILES: CCOC(=O)Cn1nc(C(C)(F)F)c2cc(Br)ccc2c1=O ; C=C(OCC)c1nn(CC(=O)OCC)c(=O)c2ccc(Br)cc12 ; CCOC(=O)Cn1nc(C(C)=O)c2cc(Br)ccc2c1=O ; CCOC(=O)Cn1nc(C(C)(F)F)c2cc(Br)ccc2c1=O ; CC(F)(F)c1nn(CC(=O)Nc2ncncc2F)c(=O)c2ccc(Br)cc12]

Ethyl 2-(6-bromo-4-(1-ethoxyvinyl)-1-oxophthalazin-2(1H)-yl)acetate: To a solution of ethyl 2-(4,6-dibromo-1-oxo-phthalazin-2-yl)acetate (0.50 g, 1.28 mmol) and tributyl(1-ethoxyvinyl)stannane (463 mg, 1.28 mmol) in DMF (8 mL) was added Pd(PPh₃)₄ (148 mg, 1.28 mmol). The reaction mixture was stirred at 80° C. for 3 h. The reaction mixture was quenched by addition of sat. aq. KF (10 mL) and then diluted with sat. aq. NaHCO₃ (10 mL). The reaction mixture was extracted with DCM (3 × 10 mL). The combined organic layers were washed with brine (10 mL), dried over anhydrous Na₂SO₄, filtered, and concentrated under reduced pressure. The crude residue was purified by silica gel column chromatography. LCMS: m/z = 381.0, 383.0 [M+H]⁺.

Ethyl 2-(4-acetyl-6-bromo-1-oxophthalazin-2(1H)-yl)acetate: To a solution of ethyl 2-(6-bromo-4-(1-ethoxyvinyl)-1-oxophthalazin-2(1H)-yl)acetate (300 mg, 0.78 mmol) in 1,4-dioxane (8 mL) and water (1.5 mL) was added aq. HCl (3 M, 0.78 mL). The reaction mixture was stirred at 50° C. for 0.5 h. The reaction mixture was poured into water (10 mL) and adjusted to pH = 7 with sat. aq. NaHCO₃. The mixture was extracted with EtOAc (3 × 10 mL). The combined organic layers were washed with brine (10 mL), dried over anhydrous Na₂SO₄, filtered, and concentrated under reduced pressure. LCMS: m/z = 353.0, 355.1 [M+H]⁺.

Ethyl 2-(6-bromo-4-(1,1-difluoroethyl)-1-oxophthalazin-2(1H)-yl)acetate: A solution of ethyl 2-(4-acetyl-6-bromo-1-oxo-phthalazin-2-yl)acetate (50 mg, 0.14 mmol) in BAST (2.51 g, 11.33 mmol) was stirred at 80° C. for 5 h. The reaction mixture was poured into sat. aq. NaHCO₃ (30 mL) and extracted with EtOAc (3 × 10 mL). The combined organic layers were washed with brine (10 mL), dried over anhydrous Na₂SO₄, filtered, and concentrated under reduced pressure. The residue was purified by silica gel column chromatography. LCMS: m/z = 375.1, 377.2 [M+H]⁺.

2-[6-bromo-4-(1,1-difluoroethyl)-1-oxophthalazin-2-yl]-N-(5-fluoropyrimidin-4-yl)acetamide: To solution of ethyl 2-(6-bromo-4-(1,1-difluoroethyl)-1-oxophthalazin-2(1H)-yl)acetate (70 mg, 0.18 mmol) and 5-fluoropyrimidin-4-amine (25 mg, 0.22 mmol) in toluene (3.0 mL) was added AlMe₃ (0.12 mL, 2 M in toluene). The reaction mixture was stirred at 80° C. for 4 h. The reaction mixture was poured into water (15 mL) and filtered. The filtrate was extracted with EtOAc (3 × 5 mL). The combined organic layers were washed with brine (5 mL), dried over anhydrous Na₂SO₄, filtered, and concentrated under reduced pressure. The residue was purified by reverse-phase preparative HPLC. LCMS: m/z = 442.0, 444.0 [M+H]⁺. ¹H NMR (400 MHz, CDCl₃) δ 8.77 (d, J= 2.0 Hz, 1H), 8.52 (d, J = 2.4 Hz, 1H), 8.42 (s, 1H), 8.35 (d, J= 8.4 Hz, 2H), 8.00-7.90 (m, 1H), 5.56 (s, 2H), 2.10 (t, J = 19.2 Hz, 3H).

### Example 14

### 2-Bromo-1-Oxo-4-Propan-2-Ylphthalazin-2-yl)-N-(5-Fluoropyrimidin-4-yl)Propanamide

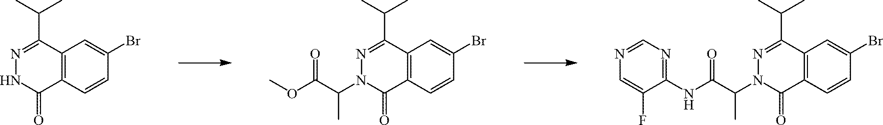 [SMILES: CC(C)c1n[nH]c(=O)c2ccc(Br)cc12 ; COC(=O)C(C)n1nc(C(C)C)c2cc(Br)ccc2c1=O ; CC(C)c1nn(C(C)C(=O)Nc2ncncc2F)c(=O)c2ccc(Br)cc12]

Methyl 2-(6-bromo-4-isopropyl-1-oxophthalazin-2(1H)-yl)propanoate: To a solution of 6-bromo-4-isopropylphthalazin-1(2H)-one (100 mg, 0.37 mmol) and methyl 2-bromopropanoate (66 mg, 0.39 mmol) in DMF (3.0 mL) was added Cs₂CO₃ (244 mg, 0.75 mmol). The reaction mixture was stirred at 25° C. for 2 h. The reaction mixture was poured into water (15 mL) and extracted with EtOAc (3 × 6 mL). The combined organic layers were washed with brine (6 mL), dried over anhydrous Na₂SO₄, filtered, and concentrated under reduced pressure. LCMS: m/z = 353.0, 355.0 [M+H]⁺.

2-(6-bromo-1-oxo-4-propan-2-ylphthalazin-2-yl)-N-(5-fluoropyrimidin-4-yl)propanamide: To a solution of methyl 2-(6-bromo-4-isopropyl-1-oxophthalazin-2(1H)-yl)propanoate (120 mg, 0.34 mmol) and 5-fluoropyrimidin-4-amine (50 mg, 0.44 mmol) in toluene (3.0 mL) was added AlMe₃ (0.51 mL, 2 M in toluene). The reaction mixture was stirred at 80° C. for 4 h. The reaction mixture was quenched by addition of water (12 mL) and extracted with EtOAc (3 × 4 mL). The combined organic layers were washed with brine (4 mL), dried over anhydrous Na₂SO₄, filtered, and concentrated under reduced pressure. The residue was purified by reverse-phase preparative HPLC. LCMS: m/z = 434.0, 436.1 [M+H]⁺. ¹H NMR (400 MHz, CDCl₃) δ 9.23 (br s, 1H), 8.76 (d, J= 2.4 Hz, 1H), 8.47 (d, J= 2.4 Hz, 1H), 8.38 (d, J= 8.4 Hz, 1H), 8.04 (s, 1H), 7.90 (d, J= 8.4 Hz, 1H), 6.01-5.89 (m, 1H), 3.48-3.44 (m, 1H), 1.80 (d, J = 7.2 Hz, 3H), 1.40 (d, J = 6.8 Hz, 3H), 1.36 (d, J = 6.8 Hz, 3H).

### Example 15

### N-Fluoropyrimidin-4-yl)-2-[1-Oxo-4-Propan-2-yl-6-(Trifluoromethoxy)Phthalazin-2-yl]Acetamide

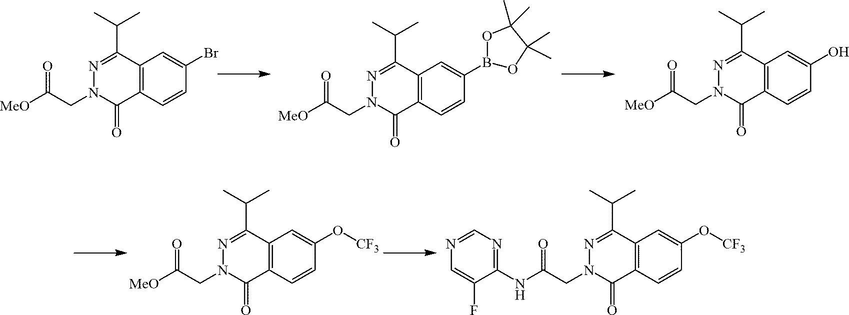 [SMILES: COC(=O)Cn1nc(C(C)C)c2cc(Br)ccc2c1=O ; COC(=O)Cn1nc(C(C)C)c2cc(B3OC(C)(C)C(C)(C)O3)ccc2c1=O ; COC(=O)Cn1nc(C(C)C)c2cc(O)ccc2c1=O ; COC(=O)Cn1nc(C(C)C)c2cc(OC(F)(F)F)ccc2c1=O ; CC(C)c1nn(CC(=O)Nc2ncncc2F)c(=O)c2ccc(OC(F)(F)F)cc12]

Methyl 2-(4-isopropyl-1-oxo-6-(4,4,5,5-tetramethyl-1,3,2-dioxaborolan-2-yl)phthalazin-2(1H)-yl)acetate: To a solution of methyl 2-(6-bromo-4-isopropyl-1-oxophthalazin-2(1H)-yl)acetate (500 mg, 1.47 mmol) in 1,4-dioxane (10 mL) were added 4,4,4′,4′,5,5,5′,5′-octamethyl-2,2′-bi(1,3,2-dioxaborolane) (562 mg, 2.21 mmol), KOAc (434 mg, 4.42 mmol) and Pd(dppf)Cl₂ (11 mg, 0.01 mmol). The reaction mixture was stirred at 80° C. for 5 h. The reaction mixture was filtered and the filtrate was concentrated under reduced pressure. The residue was purified by silica gel column chromatography. ¹H NMR (400 MHz, CDCl₃): δ 8.44 (d, J = 8.0 Hz, 1H), 8.30 (s, 1H), 8.14 (d, J = 8.0 Hz, 1H), 4.96 (s, 2H), 3.78 (s, 3H), 3.61 (m, 1H), 1.35 (d, J = 6.0 Hz, 6H), 1.26 (s, 12H).

Methyl 2-(6-hydroxy-4-isopropyl-1-oxophthalazin-2(1H)-yl)acetate: To a solution of methyl 2-(4-isopropyl-1-oxo-6-(4,4,5,5-tetramethyl-1,3,2-dioxaborolan-2-yl)phthalazin-2(1H)-yl)acetate (730 mg, 1.89 mmol) in 1,4-dioxane (3.0 mL) at 0° C. was added a solution of Oxone (1.28 g, 2.08 mmol) in water (3 mL). The reaction mixture was stirred at 20° C. for 4 h. The reaction mixture was poured into sat. aq. Na₂S₂O₃ (10 mL) and extracted with EtOAc (3 × 10 mL). The combined organic layers were washed with brine (10 mL), dried over anhydrous Na₂SO₄, filtered, and concentrated under reduced pressure. The residue was triturated with MTBE to provide a residue that was used directly. ¹H NMR (400 MHz, DMSO-d₆): δ 8.14 (d, J = 8.8 Hz, 1H), 7.24-7.31 (m, 2H), 4.84 (s, 2H), 3.68 (s, 3H), 3.39 (m, 1H), 1.25 (d, J = 6.8 Hz, 6H).

Methyl 2-(4-isopropyl-1-oxo-6-(trifluoromethoxy)phthalazin-2(1H)-yl)acetate: A roundbottomed flask containing CsF (330 mg, 2.17 mmol) was heated to 170° C. under vacuum for 0.5 h and then vessel was backfilled with nitrogen and cooled to ambient temperature before addition of AgOTf (465 mg, 1.81 mmol), Selectfluor (256 mg, 0.72 mmol), 2,4-ditert-butylphenol (149 mg, 0.72 mmol), and N-(benzenesulfonyl)-N-fluoro-benzenesulfonamide (228 mg, 0.72 mmol). To the mixture of solids was then added a solution of methyl 2-(6-hydroxy-4-isopropyl-1-oxophthalazin-2(1H)-yl)acetate (100 mg, 0.36 mmol) in toluene (7 mL), followed by 2-fluoropyridine (176 mg, 1.81 mmol) and trimethyl(trifluoromethyl)silane (257 mg, 1.81 mmol). The reaction mixture was stirred at 20° C. for 16 h. The reaction mixture was diluted with EtOAc (10 mL), filtered through a thin pad of celite, which was washed with EtOAc (20 mL). The filtrate was concentrated under reduced pressure. The residue was purified by silica gel column chromatography. ¹H NMR (400 MHz, CDCl₃): δ 8.54 (d, J = 8.8 Hz, 1H), 8.02 (br d, J = 7.2 Hz, 1H), 7.73-7.81 (m, 1H), 4.96 (s, 2H), 3.80 (s, 3H), 3.41 (m, 1H), 1.36 (d, J = 6.8 Hz, 6H).

N-(5-fluoropyrimidin-4-yl)-2-[1-oxo-4-propan-2-yl-6-(trifluoromethoxy)phthalazin-2-yl]acetamide: To a solution of 5-fluoropyrimidin-4-amine (30 mg, 0.26 mmol), methyl 2-(4-isopropyl-1-oxo-6-(trifluoromethoxy)phthalazin-2(1H)-yl)acetate (30 mg, 0.09 mmol) in toluene (2.0 mL) and THF (1.0 mL) was added AlMe₃ (0.13 mL, 2 M in toluene). The reaction mixture was stirred at 100° C. for 6 h. The reaction mixture was quenched by the addition of water (1 mL) and concentrated under reduced pressure. The residue was purified by reverse-phase preparative HPLC. LCMS: m/z = 426.0 [M+H]⁺. ¹H NMR (400 MHz, CDCl₃): δ 8.77 (s, 1H), 8.66 (br s, 1H), 8.58 (d, J = 8.4 Hz, 1H), 8.50 (s, 1H), 7.68 (s, 1H), 7.62 (d, J = 8.4 Hz, 1H), 5.46 (s, 2H), 3.44 (m, 1H), 1.38 (d, J = 6.8 Hz, 6H).

### Example 16

### 2-(Difluoromethoxy)-1-Oxo-4-Propan-2-Ylphthalazin-2-yl]-N-(5-Fluoropyrimidin-4-yl)Acetamide

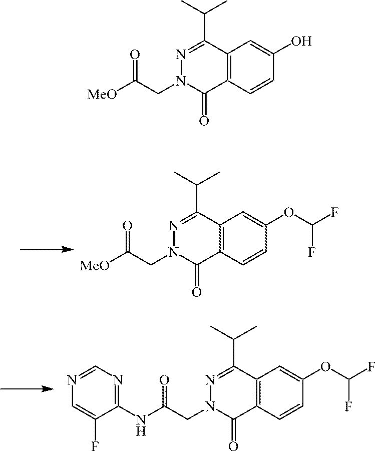

Methyl 2-(6-(difluoromethoxy)-4-isopropyl-1-oxophthalazin-2(1H)-yl)acetate: To a solution of methyl 2-(6-hydroxy-4-isopropyl-1-oxophthalazin-2(1H)-yl)acetate (100 mg, 0.36 mmol) and sodium 2-chloro-2,2-difluoroacetate (127 mg, 0.83 mmol) in DMF (3 mL) was added K₂CO₃ (125 mg, 0.90 mmol). The reaction mixture was stirred at 110° C. for 16 h. The reaction mixture was diluted with EtOAc (10 mL) and washed with H₂O (3 × 5 mL). The organics were then dried over anhydrous Na₂SO₄, filtered, and concentrated under reduced pressure. The residue was purified by silica gel column chromatography. ¹H NMR (400 MHz, CDCl₃): δ 8.51 (d, J = 8.8 Hz, 1H), 7.56-7.47 (m, 2H), 6.67 (t, J = 72 Hz, 1H), 4.96 (s, 2H), 3.79 (s, 3H), 3.44-3.38 (m, 1H), 1.35 (d, J = 6.8 Hz, 6H).

2-[6-(difluoromethoxy)-1-oxo-4-propan-2-ylphthalazin-2-yl]-N-(5-fluoropyrimidin-4-yl)acetamide: To a solution of methyl 2-(6-(difluoromethoxy)-4-isopropyl-1-oxophthalazin-2(1H)-yl)acetate (86 mg, 0.26 mmol), 5-fluoropyrimidin-4-amine (89 mg, 0.79 mmol) in toluene (1.0 mL) and THF (1.0 mL) was added AlMe₃ (0.4 mL, 2 M in toluene). The reaction mixture was stirred at 110° C. for 3 h. The mixture was quenched with water (0.5 mL), filtered, and extracted with EtOAc (5 mL). The organics were then dried over anhydrous Na₂SO₄, filtered, and concentrated under reduced pressure. The residue was purified by reverse-phase preparative HPLC. LCMS: m/z = 408.0 [M+H]⁺. ¹H NMR (400 MHz, CDCl₃): δ 8.81 (br s, 1H), 8.77 (d, J = 2.0 Hz, 1H), 8.59-8.46 (m, 2H), 7.59-7.48 (m, 2H), 6.69 (t, J = 72.0 Hz, 1H), 5.42 (s, 2H), 3.47-3.41 (m, 1H), 1.37 (d, J = 6.8 Hz, 6H).

### Example 17

### 2-Bromo-1-Oxo-4-(1,1,1-Trifluoropropan-2-yl)Phthalazin-2-yl]-N-(5-Fluoropyrimidin-4-yl)Acetamide

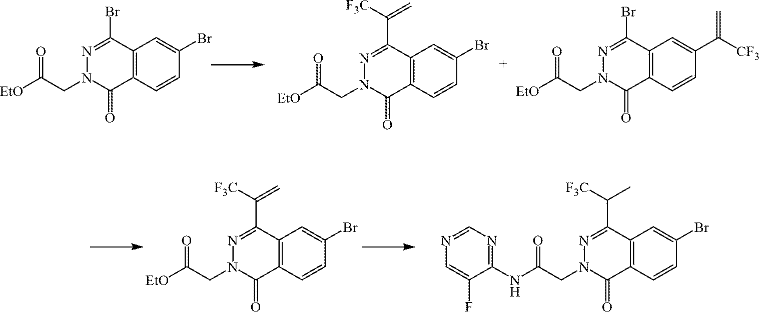

Ethyl 2-(6-bromo-1-oxo-4-(3,3,3-trifluoroprop-1-en-2-yl)phthalazin-2(1H)-yl)acetate and ethyl 2-(4-bromo-1-oxo-6-(3,3,3-trifluoroprop-1-en-2-yl)phthalazin-2(1H)-yl)acetate: To a solution of ethyl 2-(4,6-dibromo-1-oxophthalazin-2(1H)-yl)acetate (300 mg, 0.77 mmol) in 1,4-dioxane (3.0 mL) and water (1.5 mL) were added 4,4,6-trimethyl-2-(3,3,3-trifluoroprop-1-en-2-yl)-1,3,2-dioxaborinane (171 mg, 0.77 mmol), CsF (234 mg, 1.54 mmol), and Pd(dppf)Cl₂ (56 mg, 0.08 mmol). The reaction mixture was stirred at 100° C. for 16 h. The reaction mixture was diluted with water (2 mL) and extracted with EtOAc (3 × 10 mL). The combined organic layers were washed with brine (2 mL), dried over anhydrous Na₂SO₄, filtered, and concentrated under reduced pressure. The residue was purified by reverse-phase preparative HPLC to provide:

Ethyl 2-(6-bromo-1-oxo-4-(3,3,3-trifluoroprop-1-en-2-yl)phthalazin-2(1H)-yl)acetate: ¹H NMR (400 MHz, CDCl₃): δ 8.35 (d, J = 8.4 Hz, 1H), 7.92 (d, J = 8.4 Hz, 1H), 7.84 (s, 1H), 6.49 (s, 1H), 5.94 (s, 1H), 4.97 (s, 2H), 4.28-4.24 (m, 2H), 1.31-1.27 (m, 3H).

Ethyl 2-(4-bromo-1-oxo-6-(3,3,3-trifluoroprop-1-en-2-yl)phthalazin-2(1H)-yl)acetate:

¹H NMR (400 MHz, CDCl₃): δ 8.46 (d, J = 8.8 Hz, 1H), 8.04 (s, 1H), 7.91 (d, J = 8.4 Hz, 1H), 6.23 (s, 1H), 6.01 (s, 1H), 4.95 (s, 2H), 4.30-4.22 (m, 2H), 1.31-1.27 (m, 3H).

Ethyl 2-(6-bromo-1-oxo-4-(1,1,1-trifluoropropan-2-yl)phthalazin-2(1H)-yl)acetate: To a solution of ethyl 2-(6-bromo-1-oxo-4-(3,3,3-trifluoroprop-1-en-2-yl)phthalazin-2(1H)-yl)acetate (80 mg, 0.20 mmol) in THF (1.0 mL) and water (0.5 mL) were added TosN₂H₃ (221 mg, 1.18 mmol) and AcONa (97 mg, 1.18 mmol). The reaction mixture was stirred at 70° C. for 16 h. The reaction mixture was cooled to 20° C., diluted with water (3 mL), and extracted with EtOAc (3 × 2 mL). The combined organic were washed with brine (3 mL), dried over anhydrous Na₂SO₄, filtered, and concentrated under reduced pressure. The residue was purified by silica gel column chromatography. LCMS: m/z = 406.9, 408.9 [M+H]⁺.

2-[6-bromo-1-oxo-4-(1,1,1-trifluoropropan-2-yl)phthalazin-2-yl]-N-(5-fluoropyrimidin-4-yl)acetamide: To a solution of ethyl 2-(6-bromo-1-oxo-4-(1,1,1-trifluoropropan-2-yl)phthalazin-2(1H)-yl)acetate (30 mg, 0.07 mmol) in toluene (1.0 mL) and THF (1.0 mL) were added 5-fluoropyrimidin-4-amine (25 mg, 0.22 mmol) and AlMe₃ (0.11 mL, 2 M in toluene). The reaction mixture was stirred at 90° C. for 3 h. The reaction mixture was diluted with water (3 mL) and extracted with EtOAc (3 × 2 mL). The combined organic layers were washed with brine (2 mL), dried over anhydrous Na₂SO₄, filtered, and concentrated under reduced pressure. The residue was purified by reverse-phase preparative HPLC. LCMS: m/z = 473.9, 475.9 [M+H]⁺. ¹H NMR (400 MHz, CDCl₃): δ 8.77 (d, J = 2.0 Hz, 1H), 8.51 (d, J = 2.4 Hz, 1H), 8.42-8.30 (m, 2H), 7.99 (s, 1H), 7.93 (dd, J = 1.6, 8.4 Hz, 1H), 5.67 (d, J = 16.8 Hz, 1H), 5.45 (d, J = 16.8 Hz, 1H), 4.12-4.04 (m, 1H), 1.63 (d, J = 7.2 Hz, 3H).

### Example 18

### 2-Bromo-1-Oxo-6-(1,1,1-Trifluoropropan-2-yl)Phthalazin-2-yl]-N-(5-Fluoropyrimidin-4-yl)Acetamide

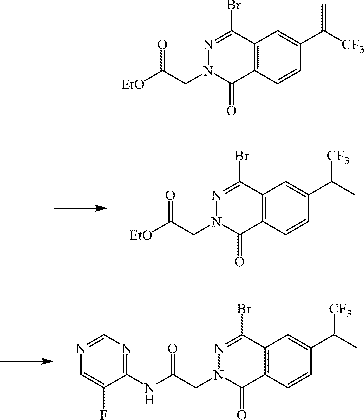 [SMILES: C=C(c1ccc2c(=O)n(CC(=O)OCC)nc(Br)c2c1)C(F)(F)F ; CCOC(=O)Cn1nc(Br)c2cc(C(C)C(F)(F)F)ccc2c1=O ; CC(c1ccc2c(=O)n(CC(=O)Nc3ncncc3F)nc(Br)c2c1)C(F)(F)F]

Ethyl 2-(4-bromo-1-oxo-6-(1,1,1-trifluoropropan-2-yl)phthalazin-2(1H)-yl)acetate: To a solution of ethyl 2-(4-bromo-1-oxo-6-(3,3,3-trifluoroprop-1-en-2-yl)phthalazin-2(1H)-yl)acetate (70 mg, 0.17 mmol) in THF (1.0 mL) and water (0.5 mL) were added TosN₂H₃ (193 mg, 1.04 mmol) and AcONa (85 mg, 1.04 mmol). The reaction mixture was stirred at 70° C. for 16 h. The reaction mixture was diluted with water (3.0 mL) and extracted with EtOAc (3 × 2 mL). The combined organics were washed with brine (3 mL), dried over anhydrous Na₂SO₄, filtered, and concentrated under reduced pressure. The residue was purified by silica gel column chromatography. LCMS: m/z = 406.9, 408.9 [M+H]⁺.

2-[4-bromo-1-oxo-6-(1,1,1-trifluoropropan-2-yl)phthalazin-2-yl]-N-(5-fluoropyrimidin-4-yl)acetamide: To a solution of ethyl 2-(4-bromo-1-oxo-6-(1,1,1-trifluoropropan-2-yl)phthalazin-2(1H)-yl)acetate (30 mg, 0.07 mmol) in toluene (1.0 mL) and THF (1.0 mL) were added 5-fluoropyrimidin-4-amine (25 mg, 0.22 mmol) and AlMe₃ (0.11 mL, 2 M in toluene). The reaction mixture was stirred at 90° C. for 3 h. The reaction mixture was diluted with water (3 mL) and extracted with EtOAc (3 × 1.5 mL). The combined organic layers were washed with brine (2 mL), dried over anhydrous Na₂SO₄, filtered, and concentrated under reduced pressure. The residue was purified by reverse-phase preparative HPLC. LCMS: m/z = 474.0, 476.0 [M+H]⁺. ¹H NMR (400 MHz, CDCl₃): δ 8.75 (d, J = 2.0 Hz, 1H), 8.51 (d, J = 2.0 Hz, 1H), 8.46 (d, J = 8.4 Hz, 1H), 8.32 (br s, 1H), 7.93 (s, 1H), 7.83 (d, J = 7.6 Hz, 1H), 5.58 (s, 2H), 3.76-3.67 (m, 1H), 1.64 (d, J = 7.2 Hz, 3H).

### Example 19

### 2-Cyclopropyl-1-Oxo-4-Propan-2-Ylphthalazin-2-yl)-N-(5-Fluoropyrimidin-4-yl)Acetamide

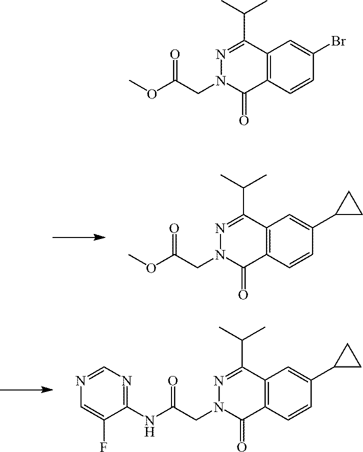 [SMILES: COC(=O)Cn1nc(C(C)C)c2cc(Br)ccc2c1=O ; COC(=O)Cn1nc(C(C)C)c2cc(C3CC3)ccc2c1=O ; CC(C)c1nn(CC(=O)Nc2ncncc2F)c(=O)c2ccc(C3CC3)cc12]

Methyl 2-(6-cyclopropyl-4-isopropyl-1-oxophthalazin-2(1H)-yl)acetate: To a solution of methyl 2-(6-bromo-4-isopropyl-1-oxo-phthalazin-2-yl)acetate (0.1 g, 0.29 mmol) in water (1.0 mL) and THF (2.0 mL) were added 2-cyclopropyl-4,4,5,5-tetramethyl-1,3,2-dioxaborolane (248 mg, 1.47 mmol), CsF (134 mg, 0.88 mmol), and Pd(dppf)Cl₂ (21.6 mg, 0.03 mmol). The reaction mixture was stirred at 100° C. for 16 h. The reaction mixture was quenched by addition of water (10 mL) and extracted with EtOAc (3 × 10 mL). The combined organic layers were washed with brine (10 mL), dried over anhydrous Na₂SO₄, filtered, and concentrated under reduced pressure. The residue was purified by silica gel column chromatography. LCMS: m/z = 301.1 [M+H]⁺.

2-(6-cyclopropyl-1-oxo-4-propan-2-ylphthalazin-2-yl)-N-(5-fluoropyrimidin-4-yl)acetamide: To a solution of the methyl 2-(6-cyclopropyl-4-isopropyl-1-oxophthalazin-2(1H)-yl)acetate (69 mg, 0.23 mmol) and 5-fluoropyrimidin-4-amine (29 mg, 0.25 mmol) in toluene (3.0 mL) was added AlMe₃ (0.15 mL, 2 M in toluene). The reaction mixture was stirred at 80° C. for 3 h. The reaction mixture was quenched by addition of water (10 mL) and filtered. The filtrate was extracted with EtOAc (3 × 5 mL). The combined organic layers were washed with brine (5 mL), dried over anhydrous Na₂SO₄, filtered, and concentrated under reduced pressure. The residue was purified by reverse-phase preparative HPLC. LCMS: m/z = 382.2 [M+H]⁺. ¹H NMR (400 MHz, CDCl₃) δ 9.10 (br s, 1H), 8.78 (d, J = 2.0 Hz, 1H), 8.48 (d, J = 2.4 Hz, 1H), 8.39 (d, J = 8.4 Hz, 1H), 7.57 (s, 1H), 7.42 (d, 8.4 Hz, 1H), 5.33 (s, 2H), 3.54-3.50 (m, 1H), 2.17-2.06 (m, 1H), 1.37 (d, J = 6.8 Hz, 6H), 1.23-1.12 (m, 2H), 0.93-0.79 (m, 2H).

### Example 20

### N-Fluoropyrimidin-4-yl)-2-(6-Iodo-1-Oxo-4-Propan-2-Ylphthalazin-2-yl)Acetamide

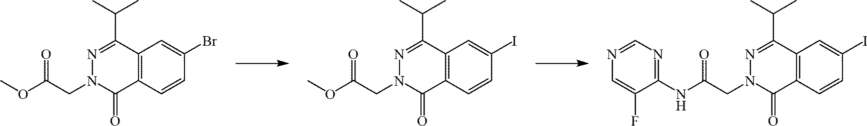 [SMILES: COC(=O)Cn1nc(C(C)C)c2cc(Br)ccc2c1=O ; COC(=O)Cn1nc(C(C)C)c2cc(I)ccc2c1=O ; CC(C)c1nn(CC(=O)Nc2ncncc2F)c(=O)c2ccc(I)cc12]

Methyl 2-(6-iodo-4-isopropyl-1-oxophthalazin-2(1H)-yl)acetate: To a solution of methyl 2-(6-bromo-4-isopropyl-1-oxophthalazin-2(1H)-yl)acetate (50 mg, 0.17 mmol) in 1,4-dioxane (5.0 mL) were added CuI (1.4 mg, 0.07 mmol), NaI (44 mg, 0.30 mmol), and (1R,2R)-N1,N2-dimethylcyclohexane-1,2-diamine (2.1 mg, 0.14 mmol). The reaction mixture was degassed and purged with N₂ three times then stirred at 110° C. for 15 h. The reaction mixture was diluted with water (10 mL) and extracted with EtOAc (3 × 10 mL). The combined organic layers were washed with brine (20 mL), dried over anhydrous Na₂SO₄, filtered, and concentrated under reduced pressure. The residue was purified by preparatory TLC. LCMS: m/z = 386.8 [M+H]⁺.

N-(5-fluoropyrimidin-4-yl)-2-(6-iodo-1-oxo-4-propan-2-ylphthalazin-2-yl)acetamide: To a solution of methyl 2-(6-iodo-4-isopropyl-1-oxophthalazin-2(1H)-yl)acetate (50 mg, 0.13 mmol) in toluene (1.0 mL) and THF (1.0 mL) were added 5-fluoropyrimidin-4-amine (29 mg, 0.26 mmol) and AlMe₃ (0.06 mL, 2 M in toluene). The reaction mixture was stirred at 90° C. for 2 h. The reaction mixture was quenched by addition of water (2 mL) and extracted with EtOAc (3 × 10 mL). The combined organic layers were washed with brine (20 mL), dried over anhydrous Na₂SO₄, filtered, and concentrated under reduced pressure. The residue was purified by reverse-phase preparative HPLC. LCMS: m/z = 468.1 [M+H]⁺.¹H NMR (400 MHz, CDCl₃): δ 8.77 (d, J = 2.0 Hz, 1H), 8.71 (br s, 1H), 8.50 (d, J = 2.4 Hz, 1H), 8.26 (s, 1H), 8.21 (d, J = 8.4 Hz, 1H), 8.11 (d, J = 8.4 Hz, 1H), 5.41 (s, 2H), 3.48-3.41 (m, 1H), 1.36 (d, J = 6.8 Hz, 6H).

### Example 21

### 2-(Difluoromethyl)-1-Oxo-4-Propan-2-Ylphthalazin-2-yl]-N-(5-Fluoropyrimidin-4-yl)Acetamide

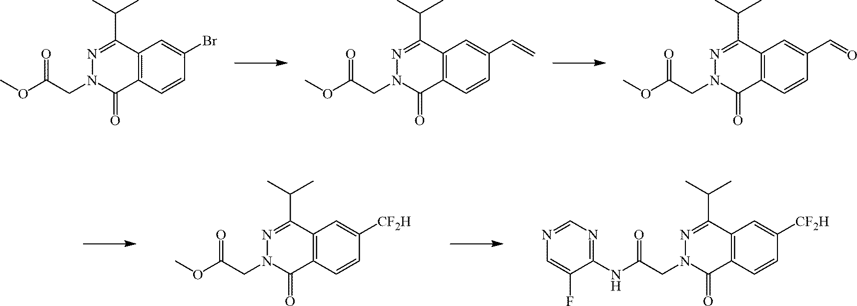 [SMILES: COC(=O)Cn1nc(C(C)C)c2cc(Br)ccc2c1=O ; C=Cc1ccc2c(=O)n(CC(=O)OC)nc(C(C)C)c2c1 ; COC(=O)Cn1nc(C(C)C)c2cc(C=O)ccc2c1=O ; COC(=O)Cn1nc(C(C)C)c2cc(C(F)F)ccc2c1=O ; CC(C)c1nn(CC(=O)Nc2ncncc2F)c(=O)c2ccc(C(F)F)cc12]

Methyl 2-(4-isopropyl-1-oxo-6-vinylphthalazin-2(1H)-yl)acetate: To a solution of methyl 2-(6-bromo-4-isopropyl-1-oxophthalazin-2(1H)-yl)acetate (400 mg, 1.18 mmol) and potassium trifluoro(vinyl)borate (474 mg, 3.54 mmol) in DMSO (8.0 mL) were added K₂CO₃ (326 mg, 2.36 mmol) and Pd(dppf)Cl₂ (86 mg, 0.12 mmol). The reaction mixture was stirred at 100° C. for 3 h. The reaction mixture was poured into water (30 mL) and extracted with EtOAc (3 × 10 mL). The combined organic layers were washed with brine (15 mL), dried over anhydrous Na₂SO₄, filtered, and concentrated under reduced pressure. The residue was purified by silica gel column chromatography. LCMS: m/z = 287.2 [M+H]⁺.

Methyl 2-(6-formyl-4-isopropyl-1-oxophthalazin-2(1H)-yl)acetate: A solution of methyl 2-(4-isopropyl-1-oxo-6-vinyl-phthalazin-2-yl)acetate (440 mg, 1.54 mmol) in DCM (40 mL) was stirred at -78° C. under ozone for 0.5 h at 15 psi. The reaction was quenched by the addition of Me₂S (1.8 g, 29.0 mmol) and stirred at 20° C. for a further 16 h. The reaction mixture was diluted with water (150 mL) and extracted with DCM (3 × 50 mL). The combined organic layers were washed with brine (50 mL), dried over anhydrous Na₂SO₄, filtered, and concentrated under reduced pressure to provide a residue that was used directly. LCMS: m/z = 289.2 [M+H]⁺.

Methyl 2-(6-(difluoromethyl)-4-isopropyl-1-oxophthalazin-2(1H)-yl)acetate: A solution of methyl 2-(6-formyl-4-isopropyl-1-oxo-phthalazin-2-yl)acetate (220 mg, 0.76 mmol) in BAST (5.05 g, 22.8 mmol) was stirred at 20° C. for 16 h. The reaction mixture was diluted with sat. aq. NaHCO₃ (15 mL) and extracted with EtOAc (3 × 5 mL). The combined organic layers were washed with brine (5 mL), dried over anhydrous Na₂SO₄, filtered, and concentrated under reduced pressure. LCMS: m/z = 311.2 [M+H]⁺.

2-[6-(difluoromethyl)-1-oxo-4-propan-2-ylphthalazin-2-yl]-N-(5-fluoropyrimidin-4-yl)acetamide: To a solution of 5-fluoropyrimidin-4-amine (27 mg, 0.24 mmol) and methyl 2-(6-(difluoromethyl)-4-isopropyl-1-oxophthalazin-2(1H)-yl)acetate (50 mg, 0.16 mmol) in toluene (1.0 mL) and THF (1.0 mL) was added AlMe₃ (0.24 mL, 2 M in toluene). The reaction mixture was stirred at 90° C. for 16 h. The reaction mixture was poured into water (10 mL) and extracted with EtOAc (3 × 5 mL). The combined organic layers were washed with brine (5 mL), dried over anhydrous Na₂SO₄, filtered, and concentrated under reduced pressure. The residue was purified by reverse-phase preparative HPLC. LCMS: m/z = 392.1 [M+H]⁺. ¹H NMR (400 MHz, CDCl₃): δ 8.78 (d, J = 1.2 Hz, 1H), 8.68 (br s, 1H), 8.62 (d, J = 8.4 Hz, 1H), 8.50 (d, J = 2.4 Hz, 1H), 8.04 (s, 1H), 7.91 (d, J = 8.0 Hz, 1H), 6.83 (t, J = 56 Hz, 1H), 5.46 (s, 2H), 3.59-3.51 (m, 1H), 1.39 (d, J = 6.8 Hz, 6H).

### Example 22

### 2-Bromo-5-Oxo-8-Propan-2-Ylpyrido[2,3-d]Pyridazin-6-yl)-N-(5-Fluoropyrimidin-4-yl)Acetamide

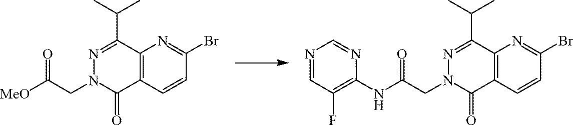 [SMILES: COC(=O)Cn1nc(C(C)C)c2nc(Br)ccc2c1=O ; CC(C)c1nn(CC(=O)Nc2ncncc2F)c(=O)c2ccc(Br)nc12]

2-(2-bromo-5-oxo-8-propan-2-ylpyrido[2,3-d]pyridazin-6-yl)-N-(5-fluoropyrimidin-4-yl)acetamide: To a solution of methyl 2-(2-bromo-8-isopropyl-5-oxopyrido[2,3-d]pyridazin-6(5H)-yl)acetate (30 mg, 0.09 mmol) and 5-fluoropyrimidin-4-amine (11 mg, 0.10 mmol) in toluene (2.0 mL) was added AlMe₃ (0.06 mL, 2 M in toluene). The reaction mixture was stirred at 80° C. for 2 h. The reaction mixture was cooled to ambient temperature, poured into sat. aq. NH₄Cl (10 mL), and extracted with EtOAc (3 × 5 mL). The combined organic layers were washed with brine (3 × 5 mL), dried over anhydrous Na₂SO₄, filtered, and concentrated under reduced pressure. The residue was purified by reverse-phase preparative HPLC. LCMS: m/z = 421.0, 423.0 [M+H]⁺. ¹H NMR (400 MHz, CDCl₃) δ 8.77 (d, J =2.0 Hz, 1H), 8.57-8.49 (m, 2H), 8.40 (br s, 1H), 7.82 (d, J= 8.6 Hz, 1H), 5.52 (s, 2H), 3.95-3.85 (m, 1H), 1.35 (d, J = 6.8 Hz, 6H).

### Example 23

### N-Fluoropyrimidin-4-yl)-2-(2-Methylsulfanyl-5-Oxo-8-Propan-2-Ylpyrido[2,3-d]Pyridazin-6-yl)Acetamide

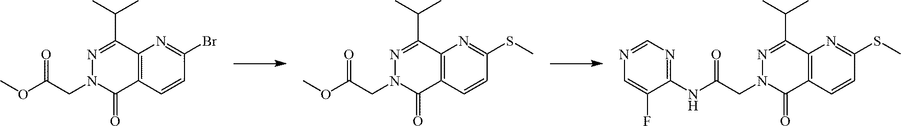

Methyl 2-(8-isopropyl-2-(methylthio)-5-oxopyrido[2,3-d]pyridazin-6(5H)-yl)acetate: To a solution of methyl 2-(2-bromo-8-isopropyl-5-oxopyrido[2,3-d]pyridazin-6(5H)-yl)acetate (100 mg, 0.29 mmol) in DMF (2.0 mL) was added sodium thiomethoxide (25 mg, 0.35 mmol). The reaction mixture was stirred at 25° C. for 12 h. The reaction mixture was poured into ice water (15 mL) and extracted with EtOAc (3 × 5 mL). The combined organic layers were washed with brine (10 mL), dried over anhydrous Na₂SO₄, filtered, and concentrated under reduced pressure to provide a residue which was used directly. LCMS: m/z = 308.1 [M+H]⁺.

N-(5-fluoropyrimidin-4-yl)-2-(2-methylsulfanyl-5-oxo-8-propan-2-ylpyrido[2,3-d]pyridazin-6-yl)acetamide: To a solution of methyl 2-(8-isopropyl-2-(methylthio)-5-oxopyrido[2,3-d]pyridazin-6(5H)-yl)acetate (62 mg, 0.20 mmol) and 5-fluoropyrimidin-4-amine (25 mg, 0.22 mmol) in toluene (3.0 mL) was added AlMe₃ (0.13 mL, 2 M in toluene). The reaction mixture was stirred at 80° C. for 12 h. The reaction mixture was poured into sat. aq. NH₄Cl (15 mL) and extracted with EtOAc (3 × 5 mL). The combined organic layers were washed with brine (10 mL), dried over anhydrous Na₂SO₄, filtered, and concentrated under reduced pressure. The residue was purified by reverse phase HPLC. LCMS: m/z = 389.1 [M+H]⁺. ¹H NMR (400 MHz, CDCl₃): δ 8.77 (s, 2H), 8.50 (d, J = 2.4 Hz, 1H), 8.41 (d, J = 8.6 Hz, 1H), 7.50 (d, J = 8.6 Hz, 1H), 5.42 (s, 2H), 3.92 (quin, J = 6.8 Hz, 1H), 2.69 (s, 3H), 1.44-1.28 (m, 6H).

### Example 24

### N-Fluoropyrimidin-4-yl)-2-[5-Oxo-8-Propan-2-yl-2-(Trifluoromethyl)Pyrido[2,3-d]Pyridazin-6-yl] Acetamide

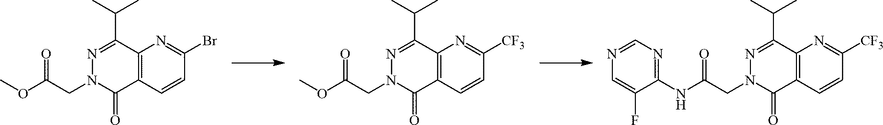 [SMILES: COC(=O)Cn1nc(C(C)C)c2nc(Br)ccc2c1=O ; COC(=O)Cn1nc(C(C)C)c2nc(C(F)(F)F)ccc2c1=O ; CC(C)c1nn(CC(=O)Nc2ncncc2F)c(=O)c2ccc(C(F)(F)F)nc12]

Methyl 2-(8-isopropyl-5-oxo-2-(trifluoromethyl)pyrido[2,3-d]pyridazin-6(5H)-yl)acetate: To a solution of methyl 2-(2-bromo-8-isopropyl-5-oxopyrido[2,3-d]pyridazin-6(5H)-yl)acetate (100 mg, 0.29 mmol) and methyl 2,2-difluoro-2-(fluorosulfonyl)acetate (282 mg, 1.47 mmol) in DMF (1.0 mL) was added CuI (56 mg, 0.29 mmol). The reaction mixture was stirred at 100° C. for 12 h. The reaction mixture was diluted with water (5 mL) and extracted with EtOAc (2 × 3 mL). The combined organic layers were washed with brine (2 mL), dried over anhydrous Na₂SO₄, filtered, and concentrated under reduced pressure. The residue was purified by silica gel column chromatography. LCMS: m/z = 330.1 [M+H]⁺.

N-(5-fluoropyrimidin-4-yl)-2-[5-oxo-8-propan-2-yl-2-(trifluoromethyl)pyrido[2,3-d]pyridazin-6-yl]acetamide: To a solution of methyl 2-(8-isopropyl-5-oxo-2-(trifluoromethyl)pyrido[2,3-d]pyridazin-6(5H)-yl)acetate (50 mg, 0.15 mmol) and 5-fluoropyrimidin-4-amine (26 mg, 0.23 mmol) in THF (0.5 mL) and toluene (1.0 mL) was added AlMe₃ (0.23 mL, 2 M in toluene). The reaction mixture was stirred at 90° C. for 3 h. The reaction mixture was diluted with water (10 mL) and extracted with EtOAc (3 × 10 mL). The combined organic layers were washed with brine (20 mL), dried over anhydrous Na₂SO₄, filtered, and concentrated under reduced pressure. The residue was purified by reverse-phase preparative HPLC. LCMS: m/z = 411.0 [M+H]⁺. ¹H NMR (400 MHz, CDCl₃): δ 8.95 (d, J = 8.4 Hz, 1H), 8.80-8.75 (m, 1H), 8.49 (s, 1H), 8.38-8.26 (m, 1H), 8.03 (d, J = 8.4 Hz, 1H), 5.56 (s, 2H), 4.04-4.03 (m, 1H), 1.38 (d, J = 6.8 Hz, 6H).

### Example 25

### 2-(Difluoromethyl)-1-Oxo-6-(Trifluoromethyl)Phthalazin-2-yl]-N-(5-Fluoropyrimidin-2-yl)Acetamide

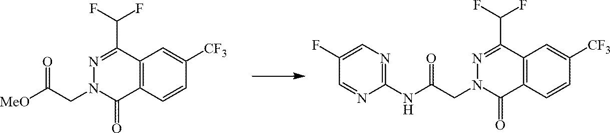 [SMILES: COC(=O)Cn1nc(C(F)F)c2cc(C(F)(F)F)ccc2c1=O ; O=C(Cn1nc(C(F)F)c2cc(C(F)(F)F)ccc2c1=O)Nc1ncc(F)cn1]

2-[4-(difluoromethyl)-1-oxo-6-(trifluoromethyl)phthalazin-2-yl]-N-(5-fluoropyrimidin-2-yl)acetamide: To a mixture of methyl 2-(4-(difluoromethyl)-1-oxo-6-(trifluoromethyl)phthalazin-2(1H)-yl)acetate (100 mg, 0.30 mmol), 5-fluoropyrimidin-2-amine (67 mg, 0.60 mmol) in toluene (2.0 mL) and THF (2.0 mL) was added AlMe₃ (0.45 mL, 2 M in toluene). The reaction mixture was stirred for 3 h at 90° C. The reaction mixture was poured into ice-cold water (10 mL) and extracted with EtOAc (4 × 5 mL). The combined organic layers were washed with brine (2 × 10 mL), dried over anhydrous Na₂SO₄, filtered, and concentrated under reduced pressure. The residue was purified by reverse-phase preparative HPLC. LCMS: m/z = 418.0 [M+H]⁺. ¹H NMR (400 MHz, DMSO-d₆): δ 11.24 (br s, 1H), 8.79 (s, 2H), 8.57 (d, J = 8.4 Hz, 1H), 8.38-8.30 (m, 2H), 7.32 (t, J = 52.8 Hz, 1H), 5.23 (s, 2H).

### Example 26

### 2-Bromo-4-(Difluoromethyl)-5-Fluoro-1-Oxophthalazin-2-yl]-N-(5-Fluoropyrimidin-2-yl)Acetamide

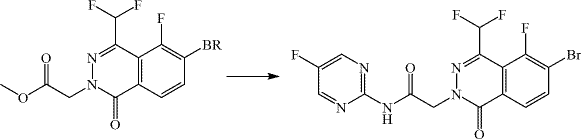

2-[6-bromo-4-(difluoromethyl)-5-fluoro-1-oxophthalazin-2-yl]-N-(5-fluoropyrimidin-2-yl)acetamide: To a solution of methyl 2-(6-bromo-4-(difluoromethyl)-5-fluoro-1-oxophthalazin-2(1H)-yl)acetate (75 mg, 0.21 mmol) in toluene (1.0 mL) and THF (1.0 mL) were added 5-fluoropyrimidin-2-amine (70 mg, 0.62 mmol) and AlMe₃ (0.31 mL, 2 M in toluene). The reaction mixture was stirred at 90° C. for 3 h. The reaction mixture was diluted with water (5 mL) and extracted with EtOAc (3 × 2 mL). The combined organics were washed with brine (5 mL), dried over anhydrous Na₂SO₄, filtered, and concentrated under reduced pressure. The residue was purified by reverse-phase preparative HPLC. LCMS: m/z = 446.0, 448.0 [M+H]⁺. ¹H NMR (400 MHz, CDCl₃): δ 8.75 (br s, 1H), 8.50 (s, 2H), 8.23-8.19 (m, 1H), 8.06-8.00 (m, 1H), 6.80-6.76 (m, 1H), 5.57 (s, 2H).

### Example 27

### 2-Bromo-4-(Difluoromethyl)-5-Fluoro-1-Oxophthalazin-2-yl]-N-Pyrimidin-2-Ylacetamide

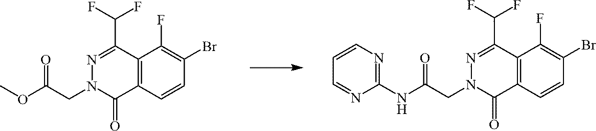 [SMILES: COC(=O)Cn1nc(C(F)F)c2c(F)c(Br)ccc2c1=O ; O=C(Cn1nc(C(F)F)c2c(F)c(Br)ccc2c1=O)Nc1ncccn1]

2-[6-bromo-4-(difluoromethyl)-5-fluoro-1-oxophthalazin-2-yl]-N-pyrimidin-2-ylacetamide: To a solution of methyl 2-(6-bromo-4-(difluoromethyl)-5-fluoro-1-oxophthalazin-2(1H)-yl)acetate (300 mg, 0.82 mmol) in toluene (3.0 mL) and THF (1.0 mL) were added pyrimidin-2-amine (117 mg, 1.23 mmol) and AlMe₃ (1.23 mL, 2 M in toluene). The reaction mixture was stirred at 90° C. for 4 h. The reaction mixture was diluted with water (2 mL) and extracted with EtOAc (3 × 2 mL). The combined organic layers were washed with brine (2 mL), dried over anhydrous Na₂SO₄, filtered, and concentrated under reduced pressure. The residue was purified by reverse-phase preparative HPLC and further purified by preparatory TLC. LCMS: m/z = 427.9, 429.9 [M+H]⁺. ¹H NMR (400 MHz, CDCl₃): δ 8.97 (s, 1H), 8.64 (d, J = 4.8 Hz, 2H), 8.22 (d, J = 8.4 Hz, 1H), 8.02 (dd, J = 6.0, 8.4 Hz, 1H), 7.06 (t, J = 4.8 Hz, 1H), 6.78 (t, J = 53.6 Hz, 1H), 5.67 (s, 2H).

### Example 28

### 2-Bromo-4-(Difluoromethyl)-5-Fluoro-1-Oxophthalazin-2-yl]-N-(5-Cyano-3-Fluoropyridin-2-yl)Acetamide

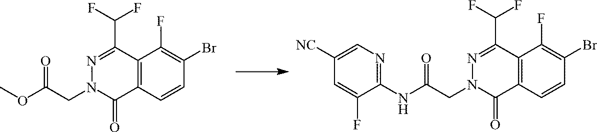

2-[6-bromo-4-(difluoromethyl)-5-fluoro-1-oxophthalazin-2-yl]-N-(5-cyano-3-fluoropyridin-2-yl)acetamide: To a solution of methyl 2-(6-bromo-4-(difluoromethyl)-5-fluoro-1-oxophthalazin-2(1H)-yl)acetate (100 mg, 0.27 mmol) and 6-amino-5-fluoronicotinonitrile (75 mg, 0.55 mmol) in toluene (2.0 mL) was added DABAL-Me₃ (70 mg, 0.27 mmol). The reaction mixture was stirred at 60° C. for 1 h. The reaction mixture was quenched by addition of water (10 mL) and extracted with EtOAc (3 × 10 mL). The combined organic layers were washed with brine (20 mL), dried over with anhydrous Na₂SO₄, filtered, and concentrated under reduced pressure. The residue was purified by reverse-phase preparative HPLC. LCMS: m/z = 469.9, 471.9 [M+H]⁺. ¹H NMR (400 MHz, CDCl₃): δ 8.49 (d, J = 1.6 Hz, 1H), 8.24 (s, 1H), 8.21 (d, J = 8.4 Hz, 1H), 8.06-8.02 (m, 1H), 7.73 (dd, J = 9.2 Hz, 1.6 Hz, 1H), 6.79 (t, J = 52.8 Hz, 1H), 5.58 (s, 2H).

The following compounds were, or can be, made via similar procedures as those described above.

| Ex. | Structure | Name | NMR | LCMS |
|---|---|---|---|---|
| 29 | 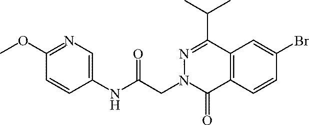 [SMILES: COc1ccc(NC(=O)Cn2nc(C(C)C)c3cc(Br)ccc3c2=O)cn1] | 2-(6-bromo-1-oxo-4-propan-2-ylphthalazin-2-yl)-N-(6-methoxypyridin-3-yl)acetamide | ¹H NMR (400 MHz, DMSO-d₆): δ 10.30 (s, 1H), 8.36-8.31 (m, 2H), 8.22 (d, J = 8.5 Hz, 1H), 8.06 (dd, J = 8.5, 1.8 Hz, 1H), 7.88 (dd, J = 8.9, 2.7 Hz, 1H), 6.81 (d, J = 8.9, 1H), 4.92 (s, 2H), 3.82 (s, 3H), 3.66-3.62 (m, 1H), 1.26 (d, J = 6.7 Hz, 6H). | m/z = 431.3, 433.3 [M+H]⁺ |
| 30 | 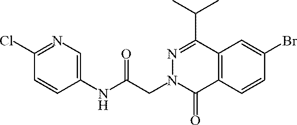 [SMILES: CC(C)c1nn(CC(=O)Nc2ccc(Cl)nc2)c(=O)c2ccc(Br)cc12] | 2-(6-bromo-1-oxo-4-propan-2-ylphthalazin-2-yl)-N-(6-chloropyridin-3-yl)acetamide | ¹H NMR (400 MHz, DMSO-d₆): δ 10.71 (s, 1H), 8.61 (d, J = 2.8, 1H), 8.32 (d, J = 1.8 Hz, 1H), 8.22 (d, J = 8.5 Hz, 1H), 8.09-8.04 (m, 2H), 7.49 (d, J = 8.7, 1H), 4.96 (s, 2H), 3.68-3.61 (m, 1H), 1.26 (d, J = 6.7 Hz, 6H) | m/z = 435.3, 437.3 [M+H]⁺ |
| 31 | 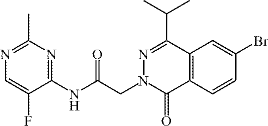 [SMILES: Cc1ncc(F)c(NC(=O)Cn2nc(C(C)C)c3cc(Br)ccc3c2=O)n1] | 2-(6-bromo-1-oxo-4-propan-2-ylphthalazin-2-yl)-N-(5-fluoro-2-methylpyrimidin-4-yl)acetamide | ¹H NMR (400 MHz, DMSO-d₆): δ 11.10 (s, 1H), 8.66 (d, J = 3.1 Hz, 1H), 8.31 (d, J = 1.8 Hz, 1H), 8.22 (d, J = 8.5 Hz, 1H), 8.06 (dd, J = 8.5, 1.8 Hz, 1H), 5.09 (s, 2H), 3.66-3.61 (m, 6.7 Hz, 1H), 2.56 (d, J = 1.0 Hz, 3H), 1.26 (d, J = 6.7 Hz, 6H) | m/z = 434.3, 436.3 [M+H]⁺ |

### Example 32

### 2-Bromo-4-Methoxy-1-Oxophthalazin-2-yl)-N-(5-Fluoropyrimidin-2-yl)Acetamide (32)

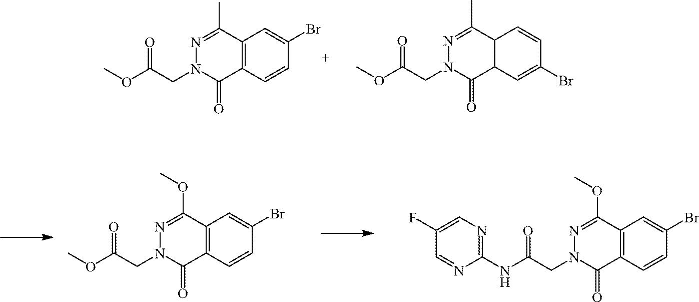

Methyl 2-(6-bromo-4-methoxy-1-oxophthalazin-2(1H)-yl)acetate: To a solution of methyl 2-(6-bromo-4-iodo-1-oxophthalazin-2(1H)-yl)acetate and methyl 2-(7-bromo-4-iodo-1-oxophthalazin-2(1H)-yl)acetate (1:1 mixture, 500 mg, 1.18 mmol) in MeOH (10 mL) at 0° C. was added MeONa (426 mg, 2.36 mmol, 5.4 M in MeOH). The reaction mixture was stirred at 80° C. for 16 h. The reaction mixture was poured into sat. aq. NH₄Cl (15 mL) and extracted with EtOAc (3 × 5 mL). The combined organic layers were washed with brine (10 mL), dried over anhydrous Na₂SO₄, filtered, and concentrated under reduced pressure. The residue was purified by silica gel chromatography. LCMS: m/z = 327.0, 329.0 [M+H]⁺.

2-(6-bromo-4-methoxy-1-oxophthalazin-2-yl)-N-(5-fluoropyrimidin-2-yl)acetamide: To a solution of methyl 2-(6-bromo-4-methoxy-1-oxophthalazin-2(1H)-yl)acetate (100 mg, 0.31 mmol) in toluene (2.0 mL) and THF (1.0 mL) were added 5-fluoropyrimidin-2-amine (52 mg, 0.46 mmol) and AlMe₃ (0.46 mL, 2 M in toluene). The reaction mixture was stirred at 90° C. for 6 h. The reaction mixture was diluted with water (5 mL) and extracted with EtOAc (3 × 2 mL). The combined organic layers were washed with brine (5 mL), dried over anhydrous Na₂SO₄, filtered, and concentrated under reduced pressure. The residue was purified by reverse-phase preparative HPLC. LCMS: m/z = 408.0, 410.0 [M+H]⁺. ¹H NMR (400 MHz, CDCl₃): δ 8.84 (br s, 1H), 8.48 (s, 2H), 8.30 (d, J = 8.4 Hz, 1H), 8.16 (s, 1H), 7.91 (d, J = 9.2 Hz, 1H), 5.24 (s, 2H), 4.00 (s, 3H).

The following compounds were, or can be, made via similar procedures as those described above.

| Ex. | Structure | Name | NMR | LCMS |
|---|---|---|---|---|
| 33 | 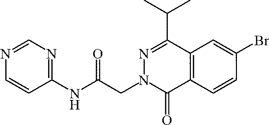 [SMILES: CC(C)c1nn(CC(=O)Nc2ccncn2)c(=O)c2ccc(Br)cc12] | 2-(6-bromo-1-oxo-4-propan-2-ylphthalazin-2-yl)-N-pyrimidin-4-ylacetamide | ¹H NMR (400 MHz, CDCl₃) δ 8.99 (s, 1H), 8.86 (s, 1H), 8.63 (d, J = 5.6 Hz, 1H), 8.39 (d, J = 8.4 Hz, 1H), 8.15 (d, J = 5.6 Hz, 1H), 8.05 (s, 1H), 7.91 (d, J= 8.4 Hz, 1H), 5.07 (s, 2H), 3.46 (m, 1H), 1.39 (d, J = 6.8 Hz, 6H) | m/z = 402.1, 404.0 [M+H]⁺ |
| 34 | 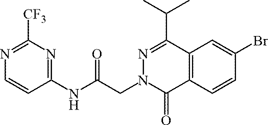 [SMILES: CC(C)c1nn(CC(=O)Nc2ccnc(C(F)(F)F)n2)c(=O)c2ccc(Br)cc12] | 2-(6-bromo-1-oxo-4-propan-2-ylphthalazin-2-yl)-N-[2-(trifluoromethyl)pyrimid in-4-yl]acetamide | ¹H NMR (400 MHz, CDCl₃) δ 9.17 (s, 1H), 8.76 (d, J = 6.0 Hz, 1H), 8.39 (d, J = 8.4 Hz, 1H), 8.32 (d, J = 5.6 Hz, 1H), 8.07 (s, 1H), 7.96 -7.88 (m, 1H), 5.09 (s, 2H), 3.55-3.35 (m, 1H), 1.41-1.35 (m, 6H) | m/z ⁼ 470.0, 472.0 [M+H]⁺ |
| 35 | 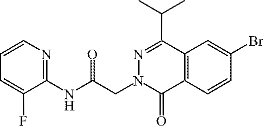 [SMILES: CC(C)c1nn(CC(=O)Nc2ncccc2F)c(=O)c2ccc(Br)cc12] | 2-(6-bromo-1-oxo-4-propan-2-ylphthalazin-2-yl)-N-(3-fluoropyridin-2-yl)acetamide | ¹H NMR (400 MHz, CDCl₃) δ 8.38 (d, J = 8.4 Hz, 1H), 8.20 (d, J = 4.8 Hz, 1H), 8.03 (d, J = 1.6 Hz, 1H), 7.88 (dd, J = 2.0, 8.8 Hz, 1H), 7.46-7.42 (m, 1H), 7.13-7.07 (m, 1H), 5.34 (s, 2H), 3.48-3.39 (m, 1H), 1.38 (d, J = 6.8 Hz, 6H) | m/z = 418.9, 421.0 [M+H]⁺ |
| 36 | 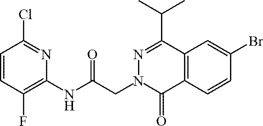 [SMILES: CC(C)c1nn(CC(=O)Nc2nc(Cl)ccc2F)c(=O)c2ccc(Br)cc12] | 2-(6-bromo-1-oxo-4-propan-2-ylphthalazin-2-yl)-N-(6-chloro-3-fluoropyridin-2-yl)acetamide | ¹H NMR (400 MHz, DMSO-d₆) δ 10.84 (s, 1H), 8.30 (d, J = 1.6 Hz, 1H), 8.21 (d, J = 8.4 Hz, 1H), 8.05 (dd, J = 1.6, 8.4 Hz, 1H), 7.91-7.87 (m, 1H), 7.48-7.42 (m, 1H), 4.99 (s, 2H), 3.64 - 3.59 (m, 1H), 1.25 (d, J = 6.8 Hz, 6H) | m/z = 452.9, 454.9 [M+H]⁺ |
| 37 | 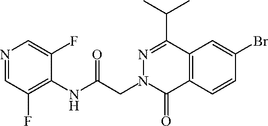 [SMILES: CC(C)c1nn(CC(=O)Nc2c(F)cncc2F)c(=O)c2ccc(Br)cc12] | 2-(6-bromo-1-oxo-4-propan-2-ylphthalazin-2-yl)-N-(3,5-difluoropyridin-4-yl)acetamide | ¹H NMR (400 MHz, CDCl₃) δ 8.70 (br s, 1H), 8.41-8.31 (m, 3H), 8.06 (d, J = 1.2 Hz, 1H), 7.95-7.90 (m, 1H), 5.13 (s, 2H), 3.52-3.41(m, 1H), 1.39 (d, J = 6.8 Hz, 6H) | m/z = 437.0, 439.0 [M+H]⁺ |
| 38 | 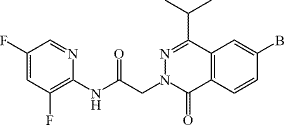 | 2-(6-bromo-1-oxo-4-propan-2-ylphthalazin-2-yl)-N-(3,5-difluoropyridin-2-yl)acetamide | ¹H NMR (400 MHz, CDCl₃): δ 8.55 (br s, 1H), 8.39 (d, J= 8.8 Hz, 1H), 8.15 (d, J = 2.4 Hz, 1H), 8.05 (d, J = 2.0 Hz, 1H), 7.92-7.88 (m, 1H), 7.34-7.29 (m, 1H), 5.24 (s, 2H), 3.51-3.32 (m, 1H), 1.39 (d, J = 6.8 Hz, 6H) | m/z = 436.9, 438.9 [M+H]⁺ |
| 39 | 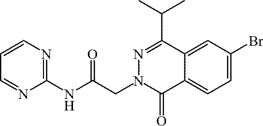 [SMILES: CC(C)c1nn(CC(=O)Nc2ncccn2)c(=O)c2ccc(Br)cc12] | 2-(6-bromo-1-oxo-4-propan-2-ylphthalazin-2-yl)-N-pyrimidin-2-ylacetamide | ¹H-NMR (400 MHz, CDCl₃): δ 9.71 (s, 1H), 8.67 (d, J = 4.9 Hz, 2H), 8.39 (d, J = 8.5 Hz, 1H), 8.03 (d, J= 1.8 Hz, 1H), 7.87 (dd, J = 8.5, 1.8 Hz, 1H), 7.03 (t, J = 4.9 Hz, 1H), 5.53 (s, 2H), 3.44 (m, 1H), 1.37 (d, J = 6.8 Hz, 6H) | m/z = 402.4, 404.3 [M+H]⁺ |
| 40 | 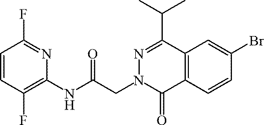 [SMILES: CC(C)c1nn(CC(=O)Nc2nc(F)ccc2F)c(=O)c2ccc(Br)cc12] | 2-(6-bromo-1-oxo-4-propan-2-ylphthalazin-2-yl)-N-(3,6-difluoropyridin-2-yl)acetamide | ¹H NMR (400 MHz, CDCl₃): δ 8.50-8.39 (m, 1H), 8.37 (d, J= 8.6 Hz, 1H), 8.03 (s, 1H), 7.90-7.85 (m, 1H), 7.58-7.53 (m, 1H), 6.72-6.65 (m, 1H), 5.33 (s, 2H), 3.46-3.42 (m, 1H), 1.37 (d, J = 6.8 Hz, 6H) | m/z = 437.0, 439.0 [M+H]⁺ |

### Example 41

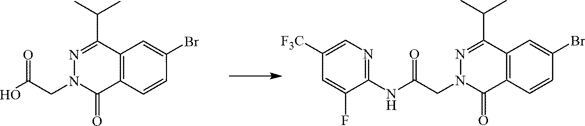 [SMILES: CC(C)c1nn(CC(=O)O)c(=O)c2ccc(Br)cc12 ; CC(C)c1nn(CC(=O)Nc2ncc(C(F)(F)F)cc2F)c(=O)c2ccc(Br)cc12]

2-(6-bromo-1-oxo-4-propan-2-ylphthalazin-2-yl)-N-[3-fluoro-5-(trifluoromethyl)pyridin-2-yl]acetamide: To a solution of 2-(6-bromo-4-isopropyl-1-oxo-phthalazin-2-yl)acetic acid (31 mg, 0.10 mmol) in DCM (2.5 mL) were added 3-fluoro-5-(trifluoromethyl)pyridin-2-amine (52 mg, 0.29 mmol) and DMAP (14 mg, 0.12 mmol). The reaction mixture was stirred at 23° C. for 15 min. To the reaction mixture was added EDC (46 mg, 0.24 mmol). The reaction mixture was stirred at 23° C. for 24 h. The reaction mixture diluted with sat. aq. NH₄Cl solution (10 mL) and extracted with EtOAc (3 × 10 mL). The combined organic layers were dried over anhydrous Na₂SO₄, filtered, and concentrated under reduced pressure. The crude residue was purified by reverse-phase HPLC. LCMS: m/z = 487.5, 489.5 [M+H]⁺. ¹H-NMR (400 MHz, CDCl₃): δ 8.75 (s, 1H), 8.47 (d, J= 0.4 Hz, 1H), 8.36 (d, J= 8.5 Hz, 1H), 8.03 (d, J = 1.8 Hz, 1H), 7.88 (dd, J= 8.5, 1.8 Hz, 1H), 7.66 (dd, J= 9.5, 1.8 Hz, 1H), 5.30 (s, 2H), 3.47-3.38 (m, 1H), 1.36 (d, J = 6.8 Hz, 6H).

The following compound was, or can be, made via similar procedures as those described above (coupling reagents employed include T3P, DIC, EDC, and HATU).

| Ex. | Structure | Name | NMR | LCMS |
|---|---|---|---|---|
| 42 | 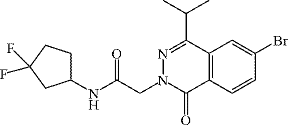 [SMILES: CC(C)c1nn(CC(=O)NC2CCC(F)(F)C2)c(=O)c2ccc(Br)cc12] | 2-(6-bromo-1-oxo-4-propan-2-ylphthalazin-2-yl)-N-(3,3-difluorocyclopentyl)acet amide | ¹H NMR (400 MHz, DMSO-d₆): δ 8.35 (d, J = 7.2 Hz, 1H), 8.29 (d, J = 1.8 Hz, 1H), 8.21 (dd, J = 8.5, 0.2 Hz, 1H), 8.05 (dd, J = 8.5, 1.8 Hz, 1H), 4.69 (d, J = 0.6 Hz, 2H), 4.26-4.20 (m, 1H), 3.65-3.58 (m, 1H), 2.47-2.38 (m, 1H), 2.25-2.18 (m, 1H), 2.12-1.95 (m, 3H), 1.70-1.65 (m, 1H), 1.24 (d, J = 6.7 Hz, 6H) | m/z = 428.3, 430.3 [M+H]⁺ |
| 43 | 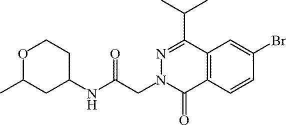 [SMILES: CC1CC(NC(=O)Cn2nc(C(C)C)c3cc(Br)ccc3c2=O)CCO1] | 2-(6-bromo-1-oxo-4-propan-2-ylphthalazin-2-yl)-N-(2-methyloxan-4-yl)acetamide | ¹H NMR (400 MHz, DMSO-d₆): δ 8.28 (d, J = 1.9 Hz, 2H), 8.20 (d, J = 8.5, 1H), 8.04 (dd, J = 8.5, 1.9 Hz, 1H), 4.75 (s, 2H), 4.05-4.00 (m, 1H), 3.77-3.72 (m, 1H), 3.69-3.66 (m, 2H), 3.65-3.57 (m, 1H), 1.70-1.59 (m, 2H), 1.52-1.37 (m, 2H), 1.24 (d, J = 6.7 Hz, 6H), 1.07 (d, J = 6.2 Hz, 3H) | m/z = 422.3, 424.3 [M+H]⁺ |
| 44 | 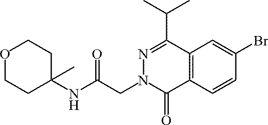 [SMILES: CC(C)c1nn(CC(=O)NC2(C)CCOCC2)c(=O)c2ccc(Br)cc12] | 2-(6-bromo-1-oxo-4-propan-2-ylphthalazin-2-yl)-N-(4-methyloxan-4-yl)acetamide | ¹H NMR (400 MHz, DMSO-d₆): δ 8.28 (d, J = 1.9 Hz, 1H), 8.21 (d, J = 8.5, 1H), 8.04 (dd, J = 8.5, 1.9 Hz, 1H), 7.76 (s, 1H), 4.71 (s, 2H), 3.63-3.54 (m, 5H), 2.04-2.00 (m, 2H), 1.51-1.43 (m, 2H), 1.30 (s, 3H), 1.25 (d, J = 6.7 Hz, 6H) | m/z = 422.3, 424.3 [M+H]⁺ |
| 45 | 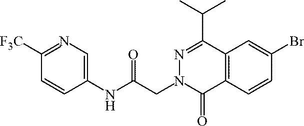 [SMILES: CC(C)c1nn(CC(=O)Nc2ccc(C(F)(F)F)nc2)c(=O)c2ccc(Br)cc12] | 2-(6-bromo-1-oxo-4-propan-2-ylphthalazin-2-yl)-N-[6-(trifluoromethyl)pyridin-3-yl]acetamide | ¹H NMR (400 MHz, DMSO-d₆): δ 10.97 (s, 1H), 8.90 (d, J = 2.4 Hz, 1H), 8.33 (d, J = 1.8 Hz, 1H), 8.31-8.28 (m, 1H), 8.22 (d, J = 8.5 Hz, 1H), 8.07 (dd, J = 8.5, 1.8 Hz, 1H), 7.89 (d, J = 8.7 Hz, 1H), 5.01 (s, 2H), 3.68-3.62 (m, 1H), 1.26 (d, J = 6.7 Hz, 6H) | m/z = 469.3, 471.3 [M+H]⁺ |
| 46 | 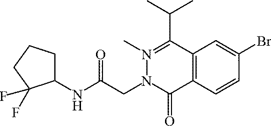 | 2-(6-bromo-1-oxo-4-propan-2-ylphthalazin-2-yl)-N-(2,2-difluorocyclopentyl)acet amide | ¹H NMR (400 MHz, DMSO-d₆): δ 8.36 (d, J = 8.7 Hz, 1H), 8.29 (d, J = 1.8 Hz, 1H), 8.20 (d, J = 8.5 Hz, 1H), 8.05 (dd, J = 8.5, 1.8 Hz, 1H), 4.81-4.72 (m, 2H), 4.42-4.33 (m, 1H), 3.64-3.58 (m, 1H), 2.20-1.99 (m, 3H), 1.78-1.57 (m, 3H), 1.24 (d, J = 6.8 Hz, 6H) | m/z = 428.3, 430.3 [M+H]⁺ |
| 47 | 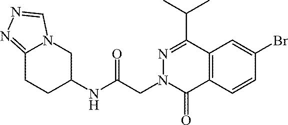 [SMILES: CC(C)c1nn(CC(=O)NC2CCc3nncn3C2)c(=O)c2ccc(Br)cc12] | 2-(6-bromo-1-oxo-4-propan-2-ylphthalazin-2-yl)-N-(5,6,7,8-tetrahydro-[1,2,4]triazolo[4,3-a]pyridin-6-yl)acetamide | ¹H NMR (400 MHz, CDCl₃): δ 8.24 (d, J = 8.5 Hz, 1H), 8.00 (d, J = 1.8 Hz, 1H), 7.87 (d, J = 1.8 Hz, 1H), 7.81 (s, 1H), 7.35 (d, J = 7.4 Hz, 1H), 4.88 (d, J = 3.6 Hz, 2H), 4.64-4.62 (m, 1H), 4.39 (dd, J = 13.0, 4.9 Hz, 1H), 4.10 (dd, J = 12.7, 5.2 Hz, 1H), 3.41 (q, J = 6.9 Hz, 1H), 3.06-2.98 (m, 2H), 2.18-2.12 (m, 2H), 1.34 (d, J = 6.8 Hz, 6H) | m/z = 445.3, 447.3 [M+H]⁺ |
| 48 | 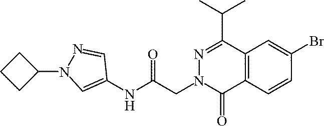 [SMILES: CC(C)c1nn(CC(=O)Nc2cnn(C3CCC3)c2)c(=O)c2ccc(Br)cc12] | 2-(6-bromo-1-oxo-4-propan-2-ylphthalazin-2-yl)-N-(1-cyclobutylpyrazol-4-yl)acetamide | ¹H NMR (400 MHz, CDCl₃): δ 8.62 (s, 1H), 8.35 (d, J = 8.5 Hz, 1H), 8.02 (s, 1H), 7.93 (s, 1H) 7.88 (d, J = 8.4 Hz, 1H), 7.42 (s, 1H), 5.00 (s, 2H), 4.75-4.66 (m, 1H), 3.47-3.39 (m, 1H), 2.51-2.40 (m, 4H), 1.87-1.76 (m, 2H), 1.36 (d, J = 6.8 Hz, 6H) | m/z = 444.7, 446.7 [M+H]⁺ |
| 49 | 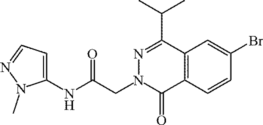 [SMILES: CC(C)c1nn(CC(=O)Nc2ccnn2C)c(=O)c2ccc(Br)cc12] | 2-(6-bromo-1-oxo-4-propan-2-ylphthalazin-2-yl)-N-(2-methylpyrazol-3-yl)acetamide | ¹H NMR (400 MHz, CDCl₃): δ 9.57 (s, 1H), 8.34 (d, J = 8.5 Hz, 1H), 8.05 (s, 1H), 7.91 (dd, J = 8.5, 1.5 Hz, 1H), 7.44 (d, J = 2.0 Hz, 1H), 6.45-6.44 (m, 1H), 5.09 (s, 2H), 3.83 (s, 3H), 3.48-3.41 (m, 1H), 1.36 (d, J = 6.8 Hz, 6H) | m/z = 404.5, 406.6 [M+H]⁺ |
| 50 | 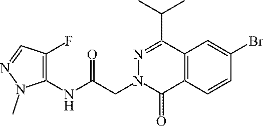 [SMILES: CC(C)c1nn(CC(=O)Nc2c(F)cnn2C)c(=O)c2ccc(Br)cc12] | 2-(6-bromo-1-oxo-4-propan-2-ylphthalazin-2-yl)-N-(4-fluoro-2-methylpyrazol-3-yl)acetamide | ¹H NMR (400 MHz, CDCl₃): δ 8.33 (d, J = 8.5 Hz, 1H), 8.23 (s, 1H), 8.04 (s, 1H), 7.91-7.88 (m, 1H), 7.32 (d, J = 4.3 Hz, 1H), 5.06 (s, 2H), 3.68 (s, 3H), 3.48-3.41 (m, 1H), 1.37 (d, J = 6.8 Hz, 6H) | m/z = 422.5, 424.4 [M+H]⁺ |
| 51 | 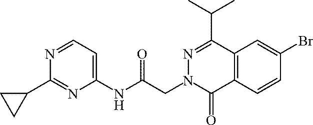 [SMILES: CC(C)c1nn(CC(=O)Nc2ccnc(C3CC3)n2)c(=O)c2ccc(Br)cc12] | 2-(6-bromo-1-oxo-4-propan-2-ylphthalazin-2-yl)-N-(2-cyclopropylpyrimidin-4-yl)acetamide | ¹H NMR (400 MHz, CDCl₃): δ 9.21-9.17 (m, 1H), 8.45 (d, J = 6.1 Hz, 1H), 8.37 (d, J = 8.5 Hz, 1H), 8.05 (s, 1H), 7.99-7.97 (m, 1H), 7.91 (dd, J= 8.5, 1.8 Hz, 1H), 5.07 (s, 2H), 3.50-3.43 (m, 1H), 2.26-2.22 (m, 1H), 1.39 (d, J = 6.8 Hz, 6H), 1.14-1.13 (m, 4H) | m/z = 442.5, 444.4 [M+H]⁺ |
| 52 | 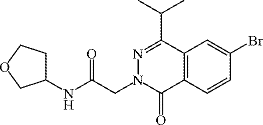 [SMILES: CC(C)c1nn(CC(=O)NC2CCOC2)c(=O)c2ccc(Br)cc12] | 2-(6-bromo-1-oxo-4-propan-2-ylphthalazin-2-yl)-N-(oxolan-3-yl)acetamide | ¹H NMR (400 MHz, CDCl₃): δ 8.33 (d, J = 8.5 Hz, 1H), 8.02 (s, 1H), 7.88 (dd, J = 8.5, 1.8 Hz, 1H), 6.46-6.43 (m, 1H), 4.84 (s, 2H), 4.57-4.52 (m, 1H), 3.91-3.85 (m, 1H), 3.82-3.73 (m, 2H), 3.64 (dd, J = 9.6, 2.8 Hz, 1H), 3.48-3.38 (m, 1H), 2.29-2.20 (m, 1H), 1.83-1.81 (m, 1H), 1.35 (d, J = 6.8 Hz, 6H) | m/z = 394.3, 396.6 [M+H]⁺ |
| 53 | 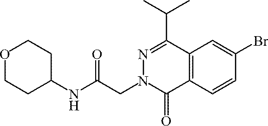 [SMILES: CC(C)c1nn(CC(=O)NC2CCOCC2)c(=O)c2ccc(Br)cc12] | 2-(6-bromo-1-oxo-4-propan-2-ylphthalazin-2-yl)-N-(oxan-4-yl)acetamide | ¹H NMR (400 MHz, CDCl₃): δ 8.33 (dd, J = 8.5, 0.3 Hz, 1H), 8.02 (s, 1H), 7.87 (dd, J = 8.5, 1.8 Hz, 1H), 6.18-6.16 (m, 1H), 4.84 (s, 2H), 4.06-3.97 (m, 1H), 3.93-3.88 (m, 2H), 3.48-3.43 (m, 2H), 3.43-3.40 (m, 1H), 1.91-1.86 (m, 2H), 1.49-1.39 (m, 2H), 1.35 (d, J = 6.8 Hz, 6H) | m/z = 408.5, 410.5 [M+H]⁺ |
| 54 | 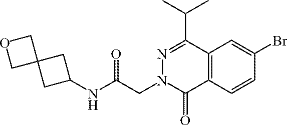 [SMILES: CC(C)c1nn(CC(=O)NC2CC3(COC3)C2)c(=O)c2ccc(Br)cc12] | 2-(6-bromo-1-oxo-4-propan-2-ylphthalazin-2-yl)-N-(2-oxaspiro[3.3]heptan-6-yl)acetamide | ¹H NMR (400 MHz, CDCl₃): δ 8.31 (dd, J = 8.5, 0.4 Hz, 1H), 8.01 (s, 1H), 7.87 (dd, J = 8.5, 1.8 Hz, 1H), 6.50-6.48 (m, 1H), 4.80 (s, 2H), 4.69 (s, 2H), 4.56 (s, 2H), 4.22-4.12 (m, 1H), 3.45-3.37 (m, 1H), 2.69-2.63 (m, 2H), 2.07-2.00 (m, 2H), 1.34 (d, J = 6.8 Hz, 6H) | m/z = 420.3, 422.2 [M+H]⁺ |
| 55 | 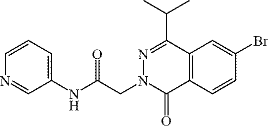 [SMILES: CC(C)c1nn(CC(=O)Nc2cccnc2)c(=O)c2ccc(Br)cc12] | 2-(6-bromo-1-oxo-4-propan-2-ylphthalazin-2-yl)-N-pyridin-3-ylacetamide | ¹H NMR (400 MHz, CDCl₃): δ 9.57 (s, 1H), 8.74 (d, J = 0.2 Hz, 1H), 8.34 (d, J = 8.5 Hz, 1H), 8.31-8.29 (m, 2H), 8.02 (d, J = 1.8 Hz, 1H), 7.88 (dd, J = 8.5, 1.8 Hz, 1H), 7.36-7.32 (m, 1H), 5.10 (s, 2H), 3.47-3.40 (m, 1H), 1.37 (d, J = 6.8 Hz, 6H) | m/z = 401.4, 403.3 [M+H]⁺ |
| 56 | 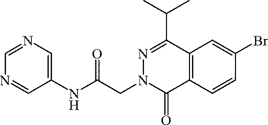 [SMILES: CC(C)c1nn(CC(=O)Nc2cncnc2)c(=O)c2ccc(Br)cc12] | 2-(6-bromo-1-oxo-4-propan-2-ylphthalazin-2-yl)-N-pyrimidin-5-ylacetamide | ¹H NMR (400 MHz, CDCl₃): δ 9.62 (s, 1H), 9.04 (s, 2H), 8.94 (s, 1H), 8.34 (d, J = 8.5 Hz, 1H), 8.04 (d, J = 1.7 Hz, 1H), 7.89 (dd, J = 8.5, 1.7 Hz, 1H), 5.11 (s, 2H), 3.50-3.39 (m, 1H), 1.37 (d, J = 6.8 Hz, 6H) | m/z = 402.2, 404.2 [M+H]⁺ |
| 57 | 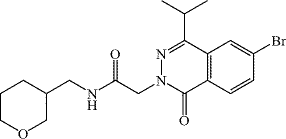 [SMILES: CC(C)c1nn(CC(=O)NCC2CCCOC2)c(=O)c2ccc(Br)cc12] | 2-(6-bromo-1-oxo-4-propan-2-ylphthalazin-2-yl)-N-(oxan-3-ylmethyl)acetamide | ¹H NMR (400 MHz, CDCl₃): δ 8.33 (d, J = 8.5 Hz, 1H), 8.02 (d, J = 1.8 Hz, 1H), 7.87 (dd, J = 8.5, 1.8 Hz, 1H), 6.34-6.30 (m, 1H), 4.85 (s, 2H), 3.80-3.76 (m, 2H), 3.45-3.39 (m, 1H), 3.39-3.32 (m, 1H), 3.21-3.11 (m, 3H), 1.82-1.73 (m, 2H), 1.63-1.52 (m, 3H), 1.35 (d, J = 6.8 Hz, 6H) | m/z = 422.6, 424.5 [M+H]⁺ |
| 58 | 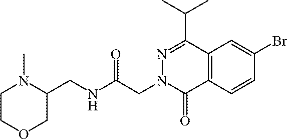 [SMILES: CC(C)c1nn(CC(=O)NCC2COCCN2C)c(=O)c2ccc(Br)cc12] | 2-(6-bromo-1-oxo-4-propan-2-ylphthalazin-2-yl)-N-[(4-methylmorpholin-3-yl)methyl]acetamide | ¹H NMR (400 MHz, CDCl₃): δ 8.32 (d, J = 8.5 Hz, 1H), 8.00 (d, J = 1.8 Hz, 1H), 7.85 (dd, J = 8.5, 1.8 Hz, 1H), 7.29-7.29 (m, 1H), 4.87 (s, 2H), 3.83-3.78 (m, 2H), 3.69-3.63 (m, 2H), 3.53 (dd, J = 12.2, 10.1 Hz, 1H), 3.45-3.38 (m, 2H), 2.89 (dt, J= 12.0, 2.1 Hz, 1H), 2.66-2.62 (m, 1H), 2.60-2.53 (m, 1H), 2.48 (s, 3H), 1.34 (d, J = 6.8 Hz, 6H) | m/z = 437.4, 439.4 [M+H]⁺ |
| 59 | 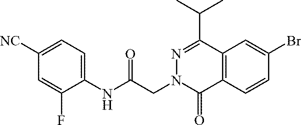 | 2-(6-bromo-1-oxo-4-propan-2-ylphthalazin-2-yl)-N-(4-cyano-2-fluorophenyl)acetamide | ¹H NMR (400 MHz, DMSO-d₆): δ 10.56 (s, 1H), 8.31 (d, J = 1.8 Hz, 1H), 8.25 (t, J = 8.3 Hz, 1H), 8.22-8.20 (m, 1H), 8.06 (dd, J = 8.5, 1.8 Hz, 1H), 7.94 (dd, J = 11.1, 1.8 Hz, 1H), 7.67-7.64 (m, 1H), 5.05 (s, 2H), 3.67-3.60 (m, 1H), 1.25 (d, J = 6.8 Hz, 6H) | m/z = 443.2, 445.2 [M+H]⁺ |
| 60 | 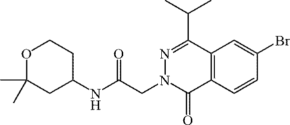 [SMILES: CC(C)c1nn(CC(=O)NC2CCOC(C)(C)C2)c(=O)c2ccc(Br)cc12] | 2-(6-bromo-1-oxo-4-propan-2-ylphthalazin-2-yl)-N-(2,2-dimethyloxan-4-yl)acetamide | ¹H NMR (400 MHz, CDCl₃): δ 8.33 (d, J = 8.5 Hz, 1H), 8.01 (d, J = 1.8 Hz, 1H), 7.87 (dd, J = 8.5, 1.8 Hz, 1H), 6.04-6.02 (m, 1H), 4.83 (s, 2H), 4.21-4.11 (m, 1H), 3.76-3.64 (m, 2H), 3.46-3.39 (m, 1H), 1.90-1.85 (m, 1H), 1.83-1.79 (m, 1H), 1.35 (d, J = 6.8 Hz, 6H), 1.29 (dd, J = 12.3, 5.6 Hz, 1H), 1.24 (dd, J = 5.0, 3.5 Hz, 1H), 1.22 (s, 3H), 1.19 (s, 3H) | m/z = 436.4, 438.3 [M+H]⁺ |
| 61 | 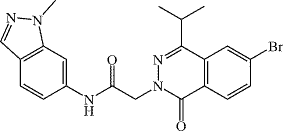 [SMILES: CC(C)c1nn(CC(=O)Nc2ccc3cnn(C)c3c2)c(=O)c2ccc(Br)cc12] | 2-(6-bromo-1-oxo-4-propan-2-ylphthalazin-2-yl)-N-(1-methylindazol-6-yl)acetamide | ¹H NMR (400 MHz, CDCl₃): δ 8.94 (s, 1H), 8.38 (d, J = 8.5 Hz, 1H), 8.09 (s, 1H), 8.04 (d, J = 1.8 Hz, 1H), 7.89 (dd, J= 8.5, 1.8 Hz, 1H), 7.85 (d, J = 0.8 Hz, 1H), 7.55 (d, J= 8.6 Hz, 1H), 6.84 (dd, J = 8.6, 1.6 Hz, 1H), 5.07 (s, 2H), 3.97 (s, 3H), 3.48-3.41 (m, 1H), 1.37 (d, J = 6.8 Hz, 6H) | m/z = 454.4, 456.3 [M+H]⁺ |
| 62 | 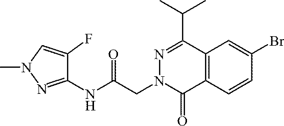 [SMILES: CC(C)c1nn(CC(=O)Nc2nn(C)cc2F)c(=O)c2ccc(Br)cc12] | 2-(6-bromo-1-oxo-4-propan-2-ylphthalazin-2-yl)-N-(4-fluoro-1-methylpyrazol-3-yl)acetamide | ¹H NMR (400 MHz, CDCl₃): δ 8.35 (d, J = 8.6 Hz, 1H), 8.34-8.30 (m, 1H), 8.04-7.98 (m, 2H), 7.90-7.86 (m, 1H), 5.05 (s, 2H), 3.76 (s, 3H), 3.44-3.40 (m, 1H), 1.36 (d, J = 6.8 Hz, 6H) | m/z = 422.3, 424.3 [M+H]⁺ |
| 63 | 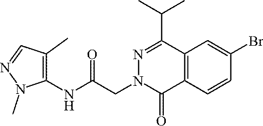 [SMILES: Cc1cnn(C)c1NC(=O)Cn1nc(C(C)C)c2cc(Br)ccc2c1=O] | 2-(6-bromo-1-oxo-4-propan-2-ylphthalazin-2-yl)-N-(2,4-dimethylpyrazol-3-yl)acetamide | ¹H NMR (400 MHz, CDCl₃): δ 8.33 (d, J = 8.5 Hz, 1H), 8.26 (s, 1H), 8.04 (d, J = 1.6 Hz, 1H), 7.89 (dd, J = 8.5, 1.7 Hz, 1H), 7.28 (s, 1H), 5.06 (s, 2H), 3.69 (s, 3H), 3.48-3.41 (m, 1H), 1.90 (s, 3H), 1.36 (d, J = 6.8 Hz, 6H) | m/z = 418.5, 420.5 [M+H]⁺ |
| 64 | 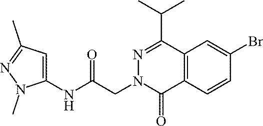 [SMILES: Cc1cc(NC(=O)Cn2nc(C(C)C)c3cc(Br)ccc3c2=O)n(C)n1] | 2-(6-bromo-1-oxo-4-propan-2-ylphthalazin-2-yl)-N-(2,5-dimethylpyrazol-3-yl)acetamide | ¹H NMR (400 MHz, CDCl₃): δ 9.27 (s, 1H), 8.34 (d, J = 8.5 Hz, 1H), 8.05 (d, J = 1.1 Hz, 1H), 7.92-7.89 (m, 1H), 6.17 (s, 1H), 5.05 (s, 2H), 3.68 (s, 3H), 3.47-3.39 (m, 1H), 2.20 (s, 3H), 1.36 (d, J = 6.8 Hz, 6H) | m/z = 418.4, 420.3 [M+H]⁺ |
| 65 | 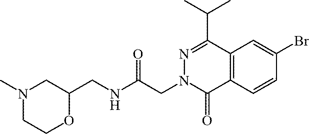 [SMILES: CC(C)c1nn(CC(=O)NCC2CN(C)CCO2)c(=O)c2ccc(Br)cc12] | 2-(6-bromo-1-oxo-4-propan-2-ylphthalazin-2-yl)-N-[(4-methylmorpholin-2-yl)methyl]acetamide | ¹H NMR (400 MHz, CDCl₃): δ 8.27 (d, J = 8.5 Hz, 1H), 8.21 (s, 1H), 8.00 (d, J = 1.8 Hz, 1H), 7.84 (dd, J = 8.5, 1.8 Hz, 1H), 6.80-6.77 (m, 1H), 4.84 (s, 2H), 3.98-3.92 (m, 1H), 3.91-3.89 (m, 2H), 3.49-3.43 (m, 2H), 3.43-3.37 (m, 1H), 3.28-3.21 (m, 2H), 2.63 (s, 3H), 2.51 (t, J = 11.4 Hz, 1H), 1.34 (d, J = 6.8 Hz, 6H) | m/z = 437.4, 439.6 [M+H]⁺ |
| 66 | 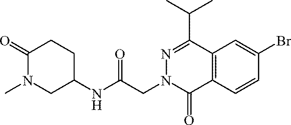 [SMILES: CC(C)c1nn(CC(=O)NC2CCC(=O)N(C)C2)c(=O)c2ccc(Br)cc12] | 2-(6-bromo-1-oxo-4-propan-2-ylphthalazin-2-yl)-N-(1-methyl-6-oxopiperidin-3-yl)acetamide | ¹H NMR (400 MHz, CDCl₃): δ 8.30 (d, J = 8.5 Hz, 1H), 8.00 (d, J = 1.8 Hz, 1H), 7.86 (dd, J = 8.5, 1.8 Hz, 1H), 6.86 (d, J = 7.4 Hz, 1H), 4.85 (d, J = 2.4 Hz, 2H), 4.35-4.27 (m, 1H), 3.57-3.53 (m, 1H), 3.46-3.36 (m, 1H), 3.18-3.13 (m, 1H), 2.88 (s, 3H), 2.49-2.38 (m, 2H), 1.98-1.82 (m, 2H), 1.34 (d, J = 6.8 Hz, 6H) | m/z = 435.6, 437.5 [M+H]⁺ |
| 67 | 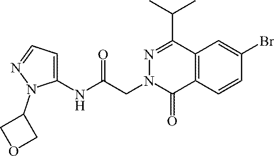 [SMILES: CC(C)c1nn(CC(=O)Nc2ccnn2C2COC2)c(=O)c2ccc(Br)cc12] | 2-(6-bromo-1-oxo-4-propan-2-ylphthalazin-2-yl)-N-[2-(oxetan-3-yl)pyrazol-3-yl]acetamide | ¹H NMR (400 MHz, CDCl₃): δ 9.09 (s, 1H), 8.34 (d, J = 8.5 Hz, 1H), 8.05 (s, 1H), 7.91 (d, J = 8.5 Hz, 1H), 7.51 (s, 1H), 6.36-6.32 (m, 1H), 5.36-5.28 (m, 1H), 5.08-4.96 (m, 4H), 4.97-4.89 (m, 2H), 3.48-3.39 (m, 1H), 1.36 (d, J = 6.8 Hz, 6H) | m/z = 446.4, 448.4 [M+H]⁺ |
| 68 | 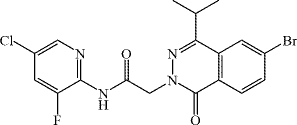 [SMILES: CC(C)c1nn(CC(=O)Nc2ncc(Cl)cc2F)c(=O)c2ccc(Br)cc12] | 2-(6-bromo-1-oxo-4-propan-2-ylphthalazin-2-yl)-N-(5-chloro-3-fluoropyridin-2-yl)acetamide | ¹H NMR (400 MHz, CDCl₃): δ 8.59-8.56 (m, 1H), 8.36 (d, J = 8.5 Hz, 1H), 8.18 (d, J = 2.1 Hz, 1H), 8.02 (d, J = 1.8 Hz, 1H), 7.87 (dd, J = 8.5, 1.8 Hz, 1H), 7.49 (dd, J = 9.3, 2.1 Hz, 1H), 5.25-5.24 (m, 2H), 3.43 (t, J = 6.8 Hz, 1H), 1.36 (d, J = 6.8 Hz, 6H) | m/z = 453.3, 455.3 [M+H]⁺ |
| 69 | 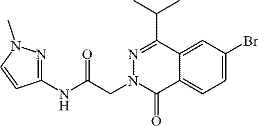 [SMILES: CC(C)c1nn(CC(=O)Nc2ccn(C)n2)c(=O)c2ccc(Br)cc12] | 2-(6-bromo-1-oxo-4-propan-2-ylphthalazin-2-yl)-N-(1-methylpyrazol-3-yl)acetamide | ¹H NMR (400 MHz, DMSO-d₆): δ 10.72 (s, 1H), 8.30 (d, J = 1.8 Hz, 1H), 8.21 (d, J = 8.5 Hz, 1H), 8.05 (dd, J = 8.5, 1.8 Hz, 1H), 7.55 (d, J = 2.2 Hz, 1H), 6.37 (d, J = 2.2 Hz, 1H), 4.87 (s, 2H), 3.74 (s, 3H), 3.62 (dt, J = 13.6, 6.8 Hz, 1H), 1.25 (d, J = 6.7 Hz, 6H) | m/z = 404.4, 406.4 [M+H]⁺ |
| 70 | 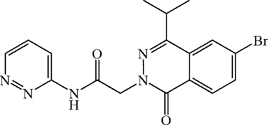 [SMILES: CC(C)c1nn(CC(=O)Nc2cccnn2)c(=O)c2ccc(Br)cc12] | 2-(6-bromo-1-oxo-4-propan-2-ylphthalazin-2-yl)-N-pyridazin-3-ylacetamide | ¹H NMR (400 MHz, DMSO-d₆): δ 11.51 (t, J = 0.4 Hz, 1H), 9.00-8.98 (m, 1H), 8.32 (t, J = 1.6 Hz, 1H), 8.26-8.23 (m, 1H), 8.23-8.20 (m, 1H), 8.07 (dd, J = 8.5, 1.7 Hz, 1H), 7.69 (dd, J = 9.0, 4.7 Hz, 1H), 5.06 (s, 2H), 3.67-3.60 (m, 1H), 1.26 (d, J = 6.7 Hz, 6H) | m/z = 402.6, 404.5 [M+H]⁺ |
| 71 | 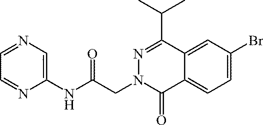 [SMILES: CC(C)c1nn(CC(=O)Nc2cnccn2)c(=O)c2ccc(Br)cc12] | 2-(6-bromo-1-oxo-4-propan-2-ylphthalazin-2-yl)-N-pyrazin-2-ylacetamide | ¹H NMR (400 MHz, CDCl₃): δ 9.59-9.48 (m, 1H), 9.05-9.00 (m, 1H), 8.42-8.26 (m, 3H), 8.06 (d, J = 1.6 Hz, 1H), 7.91 (dd, J = 8.5, 1.6 Hz, 1H), 5.12 (s, 2H), 3.47 (m, 1H), 1.40 (d, J = 6.8 Hz, 6H) | m/z = 402.5, 404.4 [M+H]⁺ |
| 72 | 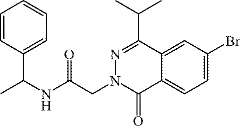 [SMILES: CC(C)c1nn(CC(=O)NC(C)c2ccccc2)c(=O)c2ccc(Br)cc12] | 2-(6-bromo-1-oxo-4-propan-2-ylphthalazin-2-yl)-N-(1-phenylethyl)acetamide | ¹H NMR (400 MHz, CDCl₃): δ 8.36 (dd, J = 8.5, 0.3 Hz, 1H), 8.02 (d, J = 1.8 Hz, 1H), 7.89 (dd, J = 8.5, 1.8 Hz, 1H), 7.34-7.25 (m, 5H), 6.45-6.43 (m, 1H), 5.17 (quintet, J = 7.3 Hz, 1H), 4.90 (q, J = 16.2 Hz, 2H), 3.43 (dt, J = 13.6, 6.8 Hz, 1H), 1.50 (d, J = 6.9 Hz, 3H), 1.34 (dd, J = 7.9, 6.8 Hz, 6H) | m/z = 428.4, 430.3 [M+H]⁺ |
| 73 | 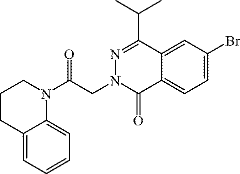 [SMILES: CC(C)c1nn(CC(=O)N2CCCc3ccccc32)c(=O)c2ccc(Br)cc12] | 6-bromo-2-[2-(3,4-dihydro-2H-quinolin-1-yl)-2-oxoethyl]-4-propan-2-ylphthalazin-1-one | ¹H NMR (400 MHz, CDCl₃): δ 8.33 (dd, J = 8.5, 0.2 Hz, 1H), 8.00 (d, J = 1.8 Hz, 1H), 7.84 (dd, J = 8.5, 1.8 Hz, 1H), 7.47 (s, 1H), 7.27-7.15 (m, 3H), 5.13 (s, 2H), 3.88 (t, J = 6.6 Hz, 2H), 3.42 (7, J = 6.8 Hz, 1H), 2.82 (t, J = 6.6 Hz, 2H), 2.05 (quintet, J = 6.6 Hz, 2H), 1.36 (d, J = 6.8 Hz, 6H) | m/z = 440.5, 442.4 [M+H]⁺ |
| 74 | 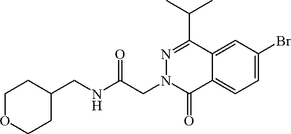 [SMILES: CC(C)c1nn(CC(=O)NCC2CCOCC2)c(=O)c2ccc(Br)cc12] | 2-(6-bromo-1-oxo-4-propan-2-ylphthalazin-2-yl)-N-(oxan-4-ylmethyl)acetamide | ¹H NMR (400 MHz, CDCl₃): δ 8.36 (dd, J = 8.5, 0.4 Hz, 1H), 8.04 (d, J = 1.8 Hz, 1H), 7.90 (dd, J = 8.5, 1.8 Hz, 1H), 6.39-6.34 (m, 1H), 4.90-4.86 (m, 2H), 3.98-3.93 (m, 2H), 3.45 (t, J = 6.8 Hz, 1H), 3.35 (td, J = 11.8, 2.1 Hz, 2H), 3.19 (t, J = 6.5 Hz, 2H), 1.80-1.73 (m, 1H), 1.60-1.55 (m, 2H), 1.38-1.35 (m, 6H), 1.34-1.24 (m, 2H) | m/z = 422.7, 424.6 [M+H]⁺ |
| 75 | 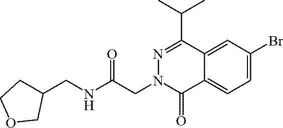 [SMILES: CC(C)c1nn(CC(=O)NCC2CCOC2)c(=O)c2ccc(Br)cc12] | 2-(6-bromo-1-oxo-4-propan-2-ylphthalazin-2-yl)-N-(oxolan-3-ylmethyl)acetamide | ¹H NMR (400 MHz, CDCl₃): δ 8.36 (dd, J = 8.5, 0.3 Hz, 1H), 8.04 (d, J = 1.8 Hz, 1H), 7.90 (dd, J = 8.5, 1.8 Hz, 1H), 6.44 (s, 1H), 4.92-4.84 (m, 2H), 3.84-3.66 (m, 3H), 3.50-3.42 (m, 2H), 3.37-3.25 (m, 2H), 2.52-2.45 (m, 1H), 2.06-1.97 (m, 1H), 1.59 (dddd, J = 12.6, 8.0, 7.0, 5.6 Hz, 1H), 1.38 (d, J = 6.8 Hz, 6H) | m/z = 408.4, 410.3 [M+H]⁺ |
| 76 | 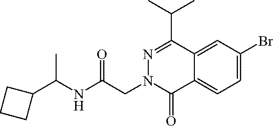 [SMILES: CC(C)c1nn(CC(=O)NC(C)C2CCC2)c(=O)c2ccc(Br)cc12] | 2-(6-bromo-1-oxo-4-propan-2-ylphthalazin-2-yl)-N-(1-cyclobutylethyl)acetami de | ¹H NMR (400 MHz, CDCl₃): δ 8.38-8.36 (m, 1H), 8.04 (d, J = 1.8 Hz, 1H), 7.90 (dd, J = 8.5, 1.8 Hz, 1H), 5.90-5.87 (m, 1H), 4.94-4.79 (m, 2H), 4.04-3.95 (m, 1H), 3.48-3.41 (m, 1H), 2.27-2.16 (m, 1H), 1.98-1.93 (m, 1H), 1.89-1.82 (m, 1H), 1.77-1.68 (m, 3H), 1.37 (dd, J = 6.8, 2.2 Hz, 6H), 1.03 (d, J = 6.6 Hz, 3H) | m/z = 406.6, 408.5 [M+H]⁺ |
| 77 | 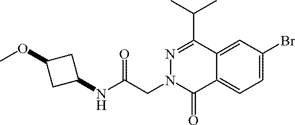 [SMILES: CO[C@H]1C[C@@H](NC(=O)Cn2nc(C(C)C)c3cc(Br)ccc3c2=O)C1] | 2-(6-bromo-1-oxo-4-propan-2-ylphthalazin-2-yl)-N-(3-cis-methoxycyclobutyl)acet amide | ¹H NMR (400 MHz, CDCl₃): δ 8.36 (d, J = 8.5 Hz, 1H), 8.04 (d, J = 1.8 Hz, 1H), 7.90 (dd, J = 8.5, 1.8 Hz, 1H), 6.38-6.35 (m, 1H), 4.85 (s, 2H), 4.13-4.03 (m, 1H), 3.66-3.59 (m, 1H), 3.49-3.40 (m, 1H), 3.23 (s, 3H), 2.78-2.72 (m, 2H), 1.80-1.72 (m, 2H), 1.38 (d, J = 6.8 Hz, 6H) | m/z = 408.4, 410.3 [M+H]⁺ |
| 78 | 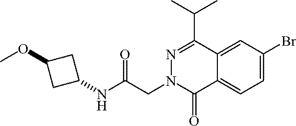 [SMILES: CO[C@H]1C[C@H](NC(=O)Cn2nc(C(C)C)c3cc(Br)ccc3c2=O)C1] | 2-(6-bromo-1-oxo-4-propan-2-ylphthalazin-2-yl)-N-(3-trans-methoxycyclobutyl)acet amide | ¹H NMR (400 MHz, CDCl₃): δ 8.40 (d, J = 7.2 Hz, 1H), 8.28 (d, J = 1.8 Hz, 1H), 8.19 (d, J = 8.5 Hz, 1H), 8.04 (dd, J = 8.5, 1.8 Hz, 1H), 4.66 (s, 2H), 4.26-4.17 (m, 1H), 3.97-3.91 (m, 1H), 3.64-3.57 (m, 1H), 3.12 (s, 3H), 2.22-2.08 (m, 4H), 1.24 (d, J = 6.7 Hz, 6H) | m/z = 408.3, 410.3 [M+H]⁺ |

### Example 79

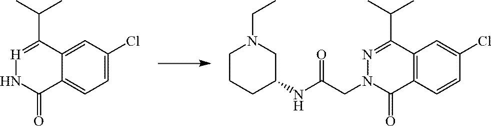

2-(6-chloro-1-oxo-4-propan-2-ylphthalazin-2-yl)-N-[(3R)-1-ethylpiperidin-3-yl]acetamide: To a solution of 6-chloro-4-isopropylphthalazin-1(2H)-one (150 mg, 0.67 mmol) and (R)-2-chloro-N-(1-ethylpiperidin-3-yl)acetamide (207 mg, 1.01 mmol) in DMF (3.0 mL) was added Cs₂CO₃ (439 mg, 1.35 mmol). The reaction mixture was stirred at 90° C. for 1 h. The reaction mixture was filtered and the filtrate was concentrated under reduced pressure. The residue was purified by reverse-phase preparative HPLC. LCMS: m/z = 391.2 [M+H]⁺. ¹H NMR (400 MHz, CDC1₃): δ 8.44 (d, J = 8.4 Hz, 1H), 7.84 (s, 1H), 7.71 (d, J = 8.4 Hz, 1H), 6.57 (s, 1H), 4.92-4.80 (m, 2H), 4.10 (s, 1H), 3.53-3.33 (m, 1H), 2.50 (s, 1H), 2.43-2.22 (m, 4H), 2.20-2.10 (m, 1H), 1.90-1.70 (m, 1H), 1.68-1.60 (m, 2H), 1.60-1.50 (m, 1H), 1.37 (d, J = 6.8 Hz, 6H), 0.94-0.73 (m, 3H).

### Examples 80 and 81

### 2-Bromo-4-(1-Fluoroethyl)-1-Oxo-Phthalazin-2-yl]-N-Pyrimidin-2-yl-Acetamide (80) and 2-[7-Bromo-4-(1-Fluoroethyl)-1-Oxo-Phthalazin-2-yl]-N-Pyrimidin-2-yl-Acetamide (81)

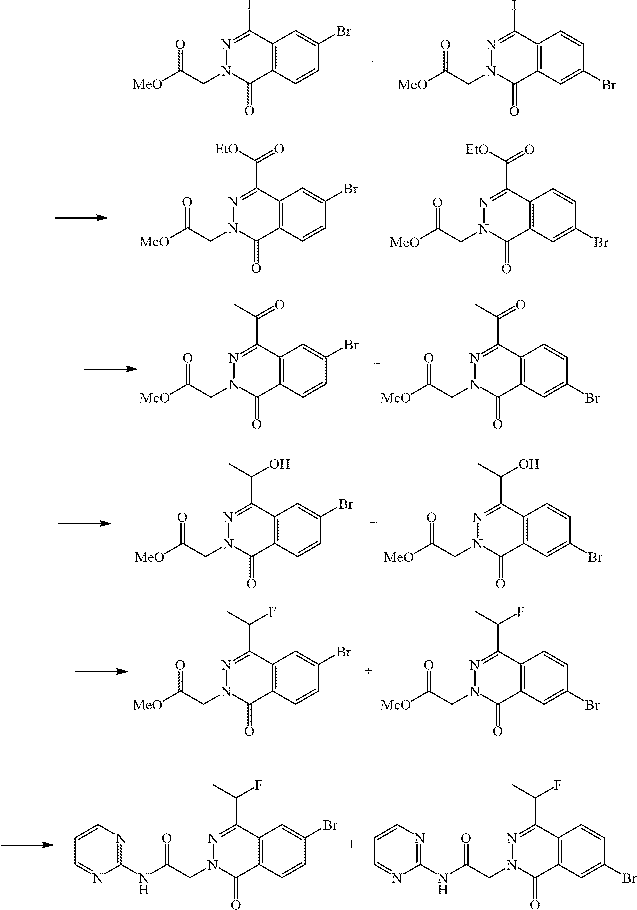

2-(6-bromo-4-(1-ethoxyvinyl)-1-oxophthalazin-2(1H)-yl)acetate and methyl 2-(7-bromo-4-(1-ethoxyvinyl)-1-oxophthalazin-2(1H)-yl)acetate: To a solution of methyl 2-(6-bromo-4-iodo-1-oxo-phthalazin-2-yl)acetate and methyl 2-(7-bromo-4-iodo-1-oxophthalazin-2(1H)-yl)acetate (1:1 mixture, 1.0 g, 2.36 mmol) in 1,4-dioxane (30 mL) were added tributyl(1-ethoxyvinyl)stannane (854 mg, 2.36 mmol), Pd(PPh₃)₄ (137 mg, 0.12 mmol), LiC1 (301 mg, 7.09 mmol) and CuI (1.35 g, 7.09 mmol). The reaction mixture was stirred at 50° C. for 16 h. The reaction mixture was poured into brine (50 mL) and extracted with EtOAc (3 × 30 mL). The combined organic layers were washed with brine (50 mL), dried over anhydrous Na₂SO₄, filtered, and concentrated under reduced pressure. The residue was purified by silica gel column chromatography to provide a 1:1 mixture of 2-(6-bromo-4-(1-ethoxyvinyl)-1-oxophthalazin-2(1H)-yl)acetate and methyl 2-(7-bromo-4-(1-ethoxyvinyl)-1-oxophthalazin-2(1H)-yl)acetate. LCMS: m/z = 336.9, 338.9 [M+H]⁺.

Methyl 2-(4-acetyl-6-bromo-1-oxophthalazin-2(1H)-yl)acetate and methyl 2-(4-acetyl-7-bromo-1-oxophthalazin-2(1H)-yl)acetate: To a solution of methyl 2-(6-bromo-4-(1-ethoxyvinyl)-1-oxophthalazin-2(1H)-yl)acetate and methyl 2-(7-bromo-4-(1-ethoxyvinyl)-1-oxophthalazin-2(1H)-yl)acetate (1:1 mixture, 600 mg, 1.63 mmol) in 1,4-dioxane (5 mL) was added aq. HC1 (12 M, 2.72 mL). The reaction mixture was stirred at 23° C. for 5 h. The reaction mixture was poured into saturated sat. aq. NaHCO₃ (30 mL) and extracted with EtOAc (3 × 10 mL). The combined organic layers were washed with brine (10 mL), dried over anhydrous Na₂SO₄, filtered, and concentrated to provide a 1:1 mixture of methyl 2-(4-acetyl-6-bromo-1-oxophthalazin-2(1H)-yl)acetate and methyl 2-(4-acetyl-7-bromo-1-oxophthalazin-2(1H)-yl)acetate. LCMS: m/z = 339.1, 341.0 [M+H]⁺.

Methyl 2-(6-bromo-4-(1-hydroxyethyl)-1-oxophthalazin-2(1H)-yl)acetate and methyl 2-(7-bromo-4-(1-hydroxyethyl)-1-oxophthalazin-2(1H)-yl)acetate: To a solution of methyl 2-(4-acetyl-6-bromo-1-oxophthalazin-2(1H)-yl)acetate and methyl 2-(4-acetyl-7-bromo-1-oxophthalazin-2(1H)-yl)acetate (1:1 mixture, 500 mg, 1.47 mmol) in THF (10 mL) at 0° C. was added NaBH₄ (56 mg, 1.47 mmol). The reaction mixture was stirred at 0° C. for 5 h. The reaction mixture was poured into brine (10 mL) and extracted with EtOAc (3 × 10 mL). The combined organic layers were washed with brine (10 mL), dried over anhydrous Na₂SO₄, filtered, and concentrated. The residue was purified by silica gel column chromatography to provide a 1:1 mixture of methyl 2-(6-bromo-4-(1-hydroxyethyl)-1-oxophthalazin-2(1H)-yl)acetate and methyl 2-(7-bromo-4-(1-hydroxyethyl)-1-oxophthalazin-2(1H)-yl)acetate. LCMS: m/z = 341.0, 343.0 [M+H]⁺.

Methyl 2-(6-bromo-4-(1-fluoroethyl)-1-oxophthalazin-2(1H)-yl)acetate and methyl 2-(7-bromo-4-(1-fluoroethyl)-1-oxophthalazin-2(1H)-yl)acetate: To a 1:1 mixture of methyl 2-(6-bromo-4-(1-hydroxyethyl)-1-oxophthalazin-2(1H)-yl)acetate and methyl 2-(7-bromo-4-(1-hydroxyethyl)-1-oxophthalazin-2(1H)-yl)acetate (250 mg, 0.73 mmol) was added BAST (1.20 g, 5.43 mmol). The reaction mixture was stirred at 20° C. for 16 h. The reaction mixture was poured into sat. aq. NaHCO₃ (10 mL) and extracted with EtOAc (3 × 5 mL). The combined organic layers were washed with brine (5 mL), dried over anhydrous Na₂SO₄, filtered, and concentrated under reduced pressure. The residue was purified by silica gel column chromatography to provide a 3:7 mixture of methyl 2-(6-bromo-4-(1-fluoroethyl)-1-oxophthalazin-2(1H)-yl)acetate and methyl 2-(7-bromo-4-(1-fluoroethyl)-1-oxophthalazin-2(1H)-yl)acetate. LCMS: m/z = 342.9, 344.9 [M+H]⁺.

2-[6-bromo-4-(1-fluoroethyl)-1-oxo-phthalazin-2-yl]-N-pyrimidin-2-yl-acetamide and 2-[7-bromo-4-(1-fluoroethyl)-1-oxo-phthalazin-2-yl]-N-pyrimidin-2-yl-acetamide: To a solution of methyl 2-(6-bromo-4-(1-fluoroethyl)-1-oxophthalazin-2(1H)-yl)acetate and methyl 2-(7-bromo-4-(1-fluoroethyl)-1-oxophthalazin-2(1H)-yl)acetate (3:7 ratio, 150 mg, =0.44 mmol) ) in toluene (5.0 mL) and THF (1.0 mL) was added pyrimidin-2-amine (83 mg, 0.87 mmol) and AlMe₃ (0.65 mL, 2 M in toluene). The reaction mixture was stirred at 95° C. for 5 h. The reaction mixture was diluted with water (5 mL) and extracted with EtOAc (3 × 2 mL). The combined organic layers were washed with brine (5 mL), dried over anhydrous Na₂SO₄, filtered, and concentrated under reduced pressure. The residue was purified by reverse-phase preparative HPLC followed by preparative SFC to provide:

2-[6-bromo-4-(1-fluoroethyl)-1-oxo-phthalazin-2-yl]-N-pyrimidin-2-yl-acetamide (80): LCMS: m/z = 406.0, 408.0 [M+H] ⁺. ¹H NMR (400 MHz, CDC1₃): δ 9.10 (s, 1H), 8.64 (d, J= 4.0 Hz, 2H), 8.36 (d, J= 8.4 Hz, 1H), 8.26 (s, 1H), 7.90 (d, J = 8.4 Hz, 1H), 7.04 (s, 1H), 6.05-5.80 (m, 1H), 5.70-5.58 (m, 1H), 5.54-5.42 (m, 1H), 1.88-1.77 (m, 3H).

2-[7-bromo-4-(1-fluoroethyl)-1-oxo-phthalazin-2-yl]-N-pyrimidin-2-yl-acetamide (81): LCMS: m/z = 406.0, 408.0 [M+H] ⁺. ¹H NMR (400 MHz, CDC1₃): δ 8.65 (d, J= 1.6 Hz, 1H), 8.61 (d, J = 4.0 Hz, 2H), 8.53 (s, 1H), 8.02-7.97 (m, 2H), 7.07-7.00 (m, 1H), 6.07-5.83 (m, 1H), 5.54-5.41 (m, 2H), 1.87-1.76 (m, 3H).

### Examples 82 and 83

### 2-Bromo-1-Oxo-4-(Trifluoromethoxy)Phthalazin-2-yl]-N-(5-Fluoropyrimidin-4-yl)Acetamide (82) and 2-[7-Bromo-1-Oxo-4-(Trifluoromethoxy)Phthalazin-2-yl]-N-(5-Fluoropyrimidin-4-yl)Acetamide (83)

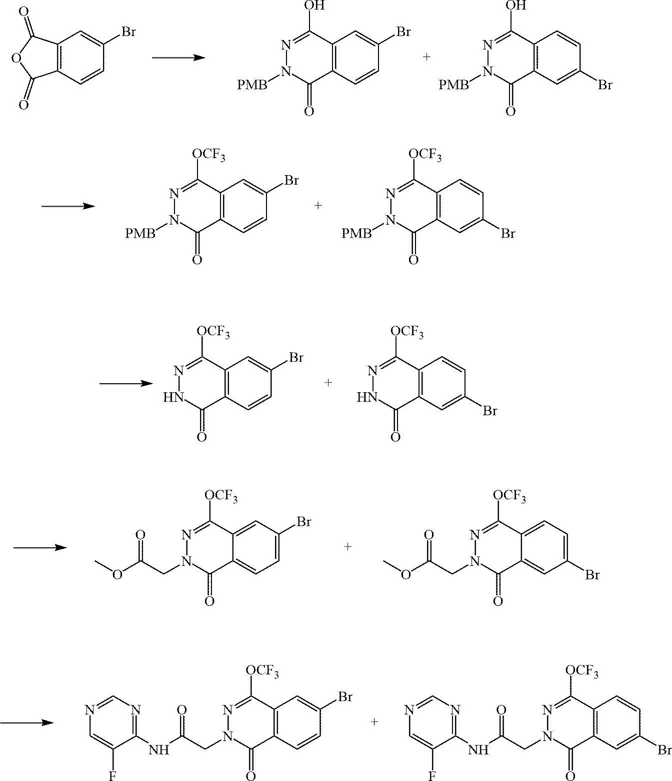

6-bromo-4-hydroxy-2-(4-methoxybenzyl)phthalazin-1(2H)-one and 7-bromo-4-hydroxy-2-(4-methoxybenzyl)phthalazin-1(2H)-one: To a solution of (4-methoxyphenyl)methylhydrazine (3.7 g, 24.3 mmol) in AcOH (10 mL) was added 5-bromoisobenzofuran-1,3-dione (5.5 g, 24.3 mmol). The reaction mixture was stirred at 140° C. for 16 h. The reaction mixture was cooled to ambient temperature, poured into sat. aq. NH₄Cl (10 mL), and extracted with EtOAc (3 × 10 mL). The combined organic layers were washed with brine (10 mL), dried over anhydrous Na₂SO₄, filtered, and concentrated. The residue was triturated in MTBE (50 mL) to provide 1:1 mixture of 6-bromo-4-hydroxy-2-(4-methoxybenzyl)phthalazin-1(2H)-one and 7-bromo-4-hydroxy-2-(4-methoxybenzyl)phthalazin-1(2H)-one. LCMS: m/z = 361.0, 363.0 [M+H] ⁺.

6-bromo-2-(4-methoxybenzyl)-4-(trifluoromethoxy)phthalazin-1(2H)-one and 7-bromo-2-(4-methoxybenzyl)-4-(trifluoromethoxy)phthalazin-1(2H)-one: A round-bottomed flask containing CsF (1.26 g, 8.3 mmol) was heated to 170° C. under vacuum for 0.5 h. Then flask was backfilled with N₂ and cooled to room temperature before the addition of AgOTf (1.77 g, 6.9 mmol), Selectfluor (980 mg, 2.76 mmol), 2,4-ditert-butylphenol (570 mg, 2.76 mmol), and N-(benzenesulfonyl)-N-fluoro-benzenesulfonamide (870 mg, 2.76 mmol). To the solid mixture was added a solution of 6-bromo-4-hydroxy-2-(4-methoxybenzyl)phthalazin-1(2H)-one and 7-bromo-4-hydroxy-2-(4-methoxybenzyl)phthalazin-1(2H)-one (1:1 mixture, 500 mg, 1.38 mmol) in toluene (30 mL), followed by 2-fluoropyridine (670 mg, 6.90 mmol) and trimethyl(trifluoromethyl)silane (981 mg, 6.90 mmol). The reaction mixture was stirred at 20° C. for 32 h. The reaction mixture was filtered to remove the solid and the filtrate was concentrated under reduced pressure. The residue was purified by silica gel column chromatography to provide a 1:1 mixture of 6-bromo-2-(4-methoxybenzyl)-4-(trifluoromethoxy)phthalazin-1(2H)-one and 7-bromo-2-(4-methoxybenzyl)-4-(trifluoromethoxy)phthalazin-1(2H)-one. LCMS: m/z = 429.0, 430.9 [M+H] ⁺.

6-bromo-4-(trifluoromethoxy)phthalazin-1(2H)-one and 7-bromo-4-(trifluoromethoxy)phthalazin-1(2H)-one: To a solution of 6-bromo-2-(4-methoxybenzyl)-4-(trifluoromethoxy)phthalazin-1(2H)-one and 7-bromo-2-(4-methoxybenzyl)-4-(trifluoromethoxy)phthalazin-1(2H)-one (1:1 mixture, 4.4 g, 10.3 mmol) in MeCN (50 mL) and water (10 mL) at 0° C. was added CAN (11.2 g, 20.5 mmol). The reaction mixture was stirred at 20° C. for 16 h. The reaction mixture was poured into sat. aq. NaHCO₃ (50 mL) and extracted with EtOAc (3 × 50 mL). The combined organic layers were washed with brine (50 mL), dried over anhydrous Na₂SO₄, filtered, and concentrated to under reduced pressure. The residue was purified by reverse-phase preparative HPLC to provide a 1:1 mixture of 6-bromo-4-(trifluoromethoxy)phthalazin-1(2H)-one and 7-bromo-4-(trifluoromethoxy)phthalazin-1(2H)-one. LCMS: m/z = 308.0, 310.9 [M+H] ⁺.

Methyl 2-(6-bromo-1-oxo-4-(trifluoromethoxy)phthalazin-2(1H)-yl)acetate and methyl 2-(7-bromo-1-oxo-4-(trifluoromethoxy)phthalazin-2(1H)-yl)acetate: To a solution of 6-bromo-4-(trifluoromethoxy)phthalazin-1(2H)-one and 7-bromo-4-(trifluoromethoxy)phthalazin-1(2H)-one (1:1 mixture, 135 mg, 0.44 mmol) in DMF (5 mL) were added methyl 2-bromoacetate (100 mg, 0.66 mmol) and Cs₂CO₃ (427 mg, 1.31 mmol). The reaction mixture was stirred at 50° C. for 5 h. The reaction mixture was poured into brine (10 mL) and extracted with EtOAc (3 × 5 mL). The combined organic layers were washed with brine (5 mL), dried over anhydrous Na₂SO₄, filtered, and concentrated under reduced pressure. The residue was purified by silica gel column to provide a 1:1 mixture of methyl 2-(6-bromo-1-oxo-4-(trifluoromethoxy)phthalazin-2(1H)-yl)acetate and methyl 2-(7-bromo-1-oxo-4-(trifluoromethoxy)phthalazin-2(1H)-yl)acetate. LCMS: m/z = 380.9, 382.9 [M+H] ⁺.

2-(6-bromo-1-oxo-4-(trifluoromethoxy)phthalazin-2(1H)-yl)-N-(5-fluoropyrimidin-4-yl)acetamide and 2-(7-bromo-1-oxo-4-(trifluoromethoxy)phthalazin-2(1H)-yl)-N-(5-fluoropyrimidin-4-yl)acetamide: To a solution of methyl 2-(6-bromo-1-oxo-4-(trifluoromethoxy)phthalazin-2(1H)-yl)acetate and 2-(7-bromo-1-oxo-4-(trifluoromethoxy)phthalazin-2(1H)-yl)acetate (1:1 mixture, 110 mg, 0.29 mmol) in toluene (5.0 mL) were added 5-fluoropyrimidin-4-amine (65 mg, 0.58 mmol) and AlMe₃ (0.43 mL, 2 M in toluene). The reaction mixture was stirred at 110° C. for 3 h. The reaction mixture was poured into sat. aq. NH₄Cl (10 mL) and extracted with EtOAc (3 × 5 mL). The combined organic layers were washed with brine (5 mL), dried over anhydrous Na₂SO₄, filtered, and concentrated under reduced pressure. The residue was purified by reverse-phase preparative HPLC to provide:

2-[6-bromo-1-oxo-4-(trifluoromethoxy)phthalazin-2-yl]-N-(5-fluoropyrimidin-4-yl)acetamide: LCMS: m/z = 461.9, 463.9 [M+H] ⁺. ¹H NMR (400 MHz, CDC1₃): δ 8.77 (s, 1H), 8.63 (d, J= 2.0 Hz, 1H), 8.52 (s, 1H), 8.19 (s, 1H), 8.03 (dd, J=2.0, 8.4 Hz, 1H), 7.81 (d, J = 8.8 Hz, 1H), 5.52 (s, 2H).

2-[7-bromo-1-oxo-4-(trifluoromethoxy)phthalazin-2-yl]-N-(5-fluoropyrimidin-4-yl)acetamide: LCMS: m/z = 461.9, 463.9 [M+H]⁺. ¹H NMR (400 MHz, CDC1₃): δ 8.76 (s, 1H), 8.52 (s, 1H), 8.33 (d, J = 8.4 Hz, 1H), 8.19 (s, 1H), 8.08 (s, 1H), 8.00 (d, J = 8.4 Hz, 1H), 5.51 (s, 2H).

The following compound was, or can be, made via similar procedures as those described above.

| Ex. | Structure | Name | NMR | LCMS |
|---|---|---|---|---|
| 84 | 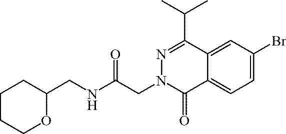 [SMILES: CC(C)c1nn(CC(=O)NCC2CCCCO2)c(=O)c2ccc(Br)cc12] | 2-(6-bromo-1-oxo-4-propan-2-ylphthalazin-2-yl)-N-(oxan-2-ylmethyl)acetamide | ¹H NMR (400 MHz, CDCl₃): δ 8.35 (dd, J = 8.5, 0.4 Hz, 1H), 8.02-8.01 (m, 1H), 7.87 (dd, J = 8.5, 1.8 Hz, 1H), 6.43-6.42 (m, 1H), 4.92-4.84 (m, 2H), 3.90-3.87 (m, 1H), 3.58 (ddd, J = 13.7, 7.0, 3.2 Hz, 1H), 3.48-3.34 (m, 3H), 3.07 (ddd, J = 13.7, 8.0, 4.3 Hz, 1H), 1.86-1.80 (m, 2H), 1.58-1.53 (m, 1H), 1.51-1.45 (m, 2H), 1.37 (d, J = 6.8 Hz, 6H), 1.33-1.21 (m, 1H) | m/z = 422.6, 424.5 [M+H]⁺ |

### Examples 85 and 86

### 2-Bromo-7-Fluoro-1-Oxo-4-Propan-2-Ylphthalazin-2-yl)-N-(5-Fluoropyrimidin-4-yl)Acetamide (85) and 2-(7-Bromo-6-Fluoro-1-Oxo-4-Propan-2-Ylphthalazin-2-yl)-N-(5-Fluoropyrimidin-4-yl)Acetamide (86)

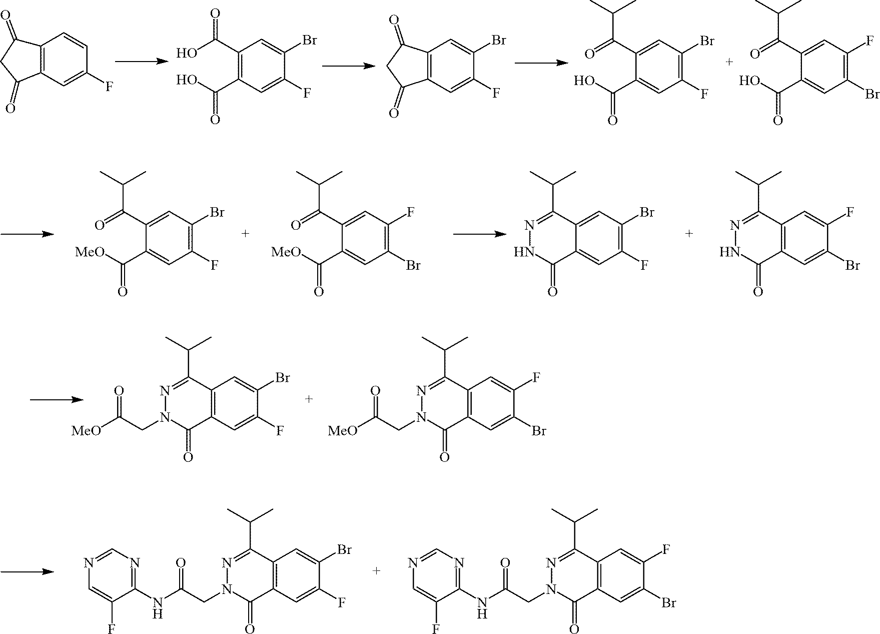

4-bromo-5-fluorophthalic acid: To a mixture of 5-fluoroisobenzofuran-1,3-dione (2.00 g, 12.0 mmol) in conc. H₂SO₄ (20 mL) was added NBS (4.29 g, 24.1 mmol). The reaction mixture was stirred at 50° C. for 12 h. The reaction mixture was poured into ice water (100 mL). The aqueous phase was extracted with EtOAc (3 × 30 mL). The combined organic layers were washed with brine (30 mL), dried over anhydrous Na₂SO₄, filtered, and concentrated under reduced pressure. The residue was purified by reverse-phase preparative HPLC. ¹H NMR (400 MHz, CDC1₃): δ 8.03 (d, J = 6.6 Hz, 1H), 7.67 (d, J= 8.8 Hz, 1H).

5-bromo-6-fluoroisobenzofuran-1,3-dione: A mixture of 4-bromo-5-fluorophthalic acid (1.20 g, 4.56 mmol) in SOC1₂ (8.20 g, 68.9 mmol) was stirred at 90° C. for 2 h. The reaction mixture was concentrated under reduced pressure to provide a residue that was used directly. ¹H NMR (400 MHz, CDC1₃): δ 8.27 (d, J = 5.6 Hz, 1H), 7.73 (d, J = 6.0 Hz, 1H).

4-bromo-5-fluoro-2-isobutyrylbenzoic acid and 5-bromo-4-fluoro-2-isobutyrylbenzoic acid: To a solution of 5-bromo-6-fluoroisobenzofuran-1,3-dione (1.12 g, 4.57 mmol) in THF (20 mL) at -10° C. was added isopropyl magnesium chloride (2.40 mL, 2 M in THF). The reaction mixture was stirred at -10° C. for 0.5 h. The reaction mixture was poured into sat. aq. NH₄Cl (50 mL) and extracted with EtOAc (3 × 25 mL). The combined organic layers were dried with anhydrous Na₂SO₄, filtered, and concentrated under reduced pressure to provide a 1:1 mixture of 4-bromo-5-fluoro-2-(2-methylpropanoyl)benzoic acid and 5-bromo-4-fluoro-2-(2-methylpropanoyl)benzoic acid. LCMS: m/z = 289.0, 291.0 [M+H]⁺.

Methyl 4-bromo-5-fluoro-2-isobutyrylbenzoate and methyl 5-bromo-4-fluoro-2-isobutyrylbenzoate: To a solution of 4-bromo-5-fluoro-2-isobutyrylbenzoic acid and 5-bromo-4-fluoro-2-isobutyrylbenzoic acid (1:1 mixture, 1.16 g, 4.01 mmol) in THF (10 mL) at 0° C. was added TMSCHN₂ (4.01 mL, 2 M in n-hexane). The reaction mixture was stirred at 25° C. for 12 h. The reaction mixture was concentrated under reduced pressure. The residue was purified by silica column gel column chromatography to provide a 1:1 mixture of methyl 4-bromo-5-fluoro-2-(2-methylpropanoyl)benzoate and methyl 5-bromo-4-fluoro-2-(2-methylpropanoyl)benzoate. LCMS: m/z = 303.0, 305.0 [M+H]⁺.

6-bromo-7-fluoro-4-isopropylphthalazin-1(2H)-one and 7-bromo-6-fluoro-4-isopropylphthalazin-1(2H)-one: To a solution of methyl 4-bromo-5-fluoro-2-isobutyrylbenzoate and methyl 5-bromo-4-fluoro-2-isobutyrylbenzoate (1:1 mixture, 465 mg, 1.53 mmol) in MeOH (10 mL) was added hydrazine monohydrate (96.8 mg, 1.84 mmol). The reaction mixture was stirred at 25° C. for 3 h. The reaction mixture was concentrated under reduced pressure provide a residue that was used directly as a 1:1 mixture of 6-bromo-7-fluoro-4-isopropylphthalazin-1(2H)-one and 7-bromo-6-fluoro-4-isopropylphthalazin-1(2H)-one. LCMS: m/z = 285.0, 287.0 [M+H]⁺.

Methyl 2-(6-bromo-7-fluoro-4-isopropyl-1-oxophthalazin-2(1H)-yl)acetate and methyl 2-(7-bromo-6-fluoro-4-isopropyl-1-oxophthalazin-2(1H)-yl)acetate: To a solution of 6-bromo-7-fluoro-4-isopropylphthalazin-1(2H)-one and 7-bromo-6-fluoro-4-isopropylphthalazin-1(2H)-one (1:1 mixture, 456 mg, 1.60 mmol) in DMF (5.0 mL) were added Cs₂CO₃ (1.04 g, 3.20 mmol) and methyl 2-bromoacetate (294 mg, 1.92 mmol). The reaction mixture was stirred at 25° C. for 12 h. The reaction mixture was poured into ice water (15 mL) and extracted with EtOAc (3 × 5 mL). The combined organic layers were washed with brine (3 × 10 mL), dried over anhydrous Na₂SO₄, filtered, and concentrated under reduced pressure. The residue was purified by silica gel column chromatography to provide a 1:1 mixture of methyl 2-(6-bromo-7-fluoro-4-isopropyl-1-oxophthalazin-2(1H)-yl) acetate and methyl 2-(7-bromo-6-fluoro-4-isopropyl-1-oxophthalazin-2(1H)-yl) acetate. LCMS: m/z = 357.0, 359.0 [M+H]⁺.

2-(6-bromo-7-fluoro-1-oxo-4-propan-2-ylphthalazin-2-yl)-N-(5-fluoropyrimidin-4-yl)acetamide and 2-(7-bromo-6-fluoro-1-oxo-4-propan-2-ylphthalazin-2-yl)-N-(5-fluoropyrimidin-4-yl)acetamide: To a solution of methyl 2-(6-bromo-7-fluoro-4-isopropyl-1-oxophthalazin-2(1H)-yl)acetate and methyl 2-(7-bromo-6-fluoro-4-isopropyl-1-oxophthalazin-2(1H)-yl)acetate (1:1 mixture, 175 mg, 0.49 mmol) in toluene (5.0 mL) were added 5-fluoropyrimidin-4-amine (83 mg, 0.73 mmol) and AlMe₃ (0.37 mL, 2 M in toluene). The reaction mixture was stirred at 80° C. for 12 h. The reaction mixture was poured into ice water (15 mL) and extracted with EtOAc (3 × 5 mL). The combined organic layers were washed with brine (10 mL), dried over anhydrous Na₂SO₄, filtered, and concentrated under reduced pressure. The residue was purified by reverse-phase preparative HPLC to provide:

2-(6-bromo-7-fluoro-1-oxo-4-propan-2-ylphthalazin-2-yl)-N-(5-fluoropyrimidin-4-yl)acetamide: LCMS: m/z = 438.0, 440.0 [M+H]⁺. ¹H NMR (400 MHz, CDC1₃): δ 8.77 (d, J= 1.6 Hz, 1H), 8.63 (br s, 1H), 8.50 (d, J = 2.2 Hz, 1H), 8.18 (d, J = 8.2 Hz, 1H), 8.14 (d, J= 6.0 Hz, 1H), 5.45 (s, 2H), 3.46-3.36 (m, 1H), 1.37 (d, J = 6.8 Hz, 6H).

2-(7-bromo-6-fluoro-1-oxo-4-propan-2-ylphthalazin-2-yl)-N-(5-fluoropyrimidin-4-yl)acetamide: LCMS: m/z = 438.0, 440.0 [M+H]⁺. ¹H NMR (400 MHz, CDC1₃): δ 8.83-8.72 (m, 1H), 8.72-8.70 (m, 1H), 8.68 (br s, 1H), 8.50 (d, J= 2.0 Hz, 1H), 7.58-7.54 (m, 1H), 5.45 (s, 2H), 3.45-3.26 (m, 1H), 1.36 (d, J=6.8 Hz, 6H).

### Examples 87 and 88

### N-fluoropyrimidin-4-yl)-2-[1-oxo-4-propan-2-yl-6-(trifluoromethyl)phthalazin-2-yl]acetamide (87) and N-fluoropyrimidin-4-yl)-2-[1-oxo-4-propan-2-yl-7-(trifluoromethyl)phthalazin-2-yl]acetamide (88)

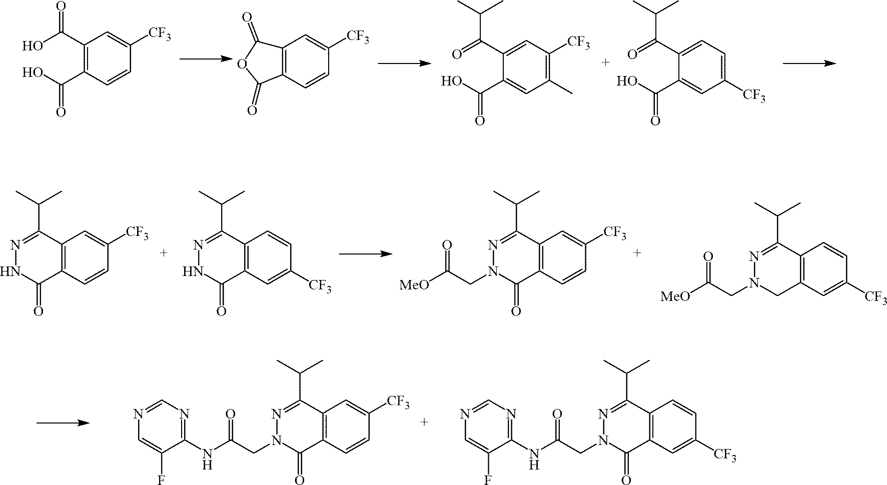

5-(trifluoromethyl)isobenzofuran-1,3-dione: A solution of 4-(trifluoromethyl)phthalic acid (1.00 g, 4.27 mmol) in SOC1₂ (15.2 g, 128 mmol) was stirred at 50° C. for 2 h. The reaction mixture was concentrated under reduced pressure. The residue was used directly.

2-isobutyryl-4-(trifluoromethyl)benzoic acid and 2-isobutyryl-5-(trifluoromethyl)benzoic acid: To a solution of 5-(trifluoromethyl)isobenzofuran-1,3-dione (100 mg, 0.46 mmol) in THF (2 mL) at -10° C. was added isopropyl magnesium chloride (0.23 mL, 2 M in THF). The reaction mixture was stirred at -10° C. for 2 h. The reaction mixture was diluted with water (2 mL) and extracted with EtOAc (2 × 3 mL). The combined organic layers were washed with brine (2 mL), dried over anhydrous Na₂SO₄, filtered, and concentrated under reduced pressure to provide a residue that was used directly as a 1:1 mixture of 2-isobutyryl-4-(trifluoromethyl)benzoic acid and 2-isobutyryl-5-(trifluoromethyl)benzoic acid. LCMS: m/z = 258.9 [M-H]⁻.

4-isopropyl-6-(trifluoromethyl)phthalazin-1(2H)-one and 4-isopropyl-7-(trifluoromethyl)phthalazin-1(2H)-one: To a solution of 2-isobutyryl-4-(trifluoromethyl)benzoic acid and 2-isobutyryl-5-(trifluoromethyl)benzoic acid (1:1 mixture, 1.40 g, 5.38 mmol) in EtOH (2 mL) was added hydrazine hydrate (302 mg, 5.92 mmol). The reaction mixture was stirred at 90° C. for 12 h. The reaction mixture was diluted with water (100 mL) and extracted with EtOAc (3 × 50 mL). The combined organic layers were washed with brine (50 mL), dried over anhydrous Na₂SO₄, filtered, and concentrated under reduced pressure to provide a residue that was used directly as a 1:1 mixture of 4-isopropyl-6-(trifluoromethyl)phthalazin-1(2H)-one and 4-isopropyl-7-(trifluoromethyl)phthalazin-1(2H)-one. LCMS: m/z = 257.1 [M+H]⁺.

Methyl 2-(4-isopropyl-1-oxo-6-(trifluoromethyl)phthalazin-2(1H)-yl)acetate and methyl 2-(4-isopropyl-1-oxo-7-(trifluoromethyl)phthalazin-2(1H)-yl)acetate: To a solution of 4-isopropyl-6-(trifluoromethyl)phthalazin-1(2H)-one and 4-isopropyl-7-(trifluoromethyl)phthalazin-1(2H)-one (1:1 mixture, 1.00 g, 3.90 mmol) in DMF (10 mL) were added methyl 2-chloroacetate (847 mg, 7.81 mmol) and Cs₂CO₃ (3.81 g, 11.71 mmol). The reaction mixture was stirred at 20° C. for 2 h. The reaction mixture was diluted with water (50 mL) and extracted with EtOAc (3 × 50 mL). The combined organic layers were washed with brine (50 mL), dried over anhydrous Na₂SO₄, filtered, and concentrated under reduced pressure. The residue was purified by silica gel column chromatography to provide a 1:1 mixture of methyl 2-(4-isopropyl-1-oxo-6-(trifluoromethyl)phthalazin-2(1H)-yl)acetate and methyl 2-(4-isopropyl-1-oxo-7-(trifluoromethyl)phthalazin-2(1H)-yl)acetate. LCMS: m/z = 329.1 [M+H]⁺.

N-(5-fluoropyrimidin-4-yl)-2-(4-isopropyl-1-oxo-6-(trifluoromethyl)phthalazin-2(1H)-yl)acetamide and N-(5-fluoropyrimidin-4-yl)-2-(4-isopropyl-1-oxo-7-(trifluoromethyl)phthalazin-2(1H)-yl)acetamide: To a solution of methyl 2-(4-isopropyl-1-oxo-6-(trifluoromethyl)phthalazin-2(1H)-yl)acetate and methyl 2-(4-isopropyl-1-oxo-7-(trifluoromethyl)phthalazin-2(1H)-yl)acetate (1:1 mixture, 140 mg, 0.43 mmol) in THF (2.0 mL) and toluene (2.0 mL) were added 5-fluoropyrimidin-4-amine (96 mg, 0.85 mmol) and AlMe₃ (0.64 mL, 2 M in toluene). The reaction mixture was stirred at 90° C. for 2 h. The reaction mixture was diluted with water (20 mL) and extracted with EtOAc (3 × 20 mL). The combined organic layers were washed with brine (20 mL), dried over anhydrous Na₂SO₄, filtered, and concentrated under reduced pressure. The residue was purified by reverse-phase preparative HPLC to provide:

N-(5-fluoropyrimidin-4-yl)-2-[1-oxo-4-propan-2-yl-6-(trifluoromethyl)phthalazin-2-yl]acetamide: LCMS: m/z = 410.2 [M+H]⁺. ¹H NMR (400 MHz, CDC1₃): δ 8.77 (d, J = 2.0 Hz, 1H), 8.65 (d, J= 8.4 Hz, 2H), 8.50 (d, J = 2.4 Hz, 1H), 8.16 (s, 1H), 8.00 (d, J= 8.4 Hz, 1H), 5.50 (s, 2H), 3.63-3.44 (m, 1H), 1.39 (d, J = 6.8 Hz, 6H).

N-(5-fluoropyrimidin-4-yl)-2-[1-oxo-4-propan-2-yl-7-(trifluoromethyl)phthalazin-2-yl]acetamide: LCMS: m/z = 410.2 [M+H]⁺. ¹H NMR (400 MHz, CDC1₃): δ 8.79 (d, J = 12 Hz, 2H), 8.63 (br s, 1H), 8.51 (d, J= 2.4 Hz, 1H), 8.12-7.99 (m, 2H), 5.50 (s, 2H), 3.58-3.49 (m, 1H), 1.39 (d, J= 6.8 Hz, 6H).

### Examples 89 and 90

### 2-Bromo-1-Oxo-4-(Trifluoromethyl)Phthalazin-2-yl]-N-[(3R)-1-Ethylpiperidin-3-yl]Acetamide (89) and 2-[7-Bromo-1-Oxo-4-(Trifluoromethyl)Phthalazin-2-yl]-N-[(3R)-1-Ethylpiperidin-3-yl]Acetamide (90)

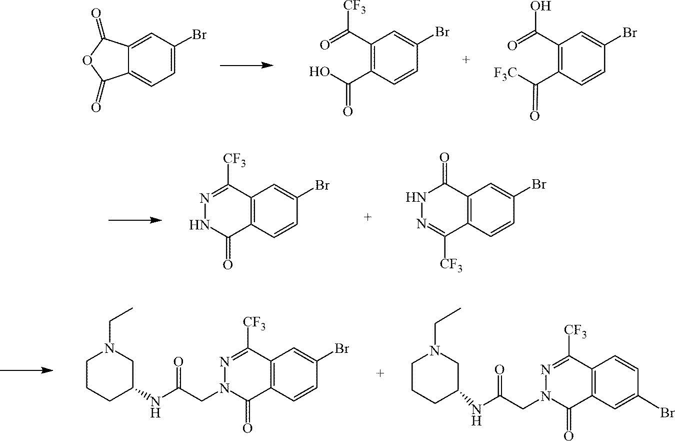

4-bromo-2-(2,2,2-trifluoroacetyl)benzoic acid and 5-bromo-2-(2,2,2-trifluoroacetyl)benzoic acid: To a solution of 5-bromoisobenzofuran-1,3-dione (3.00 g, 13.2 mmol) in MeCN (30 mL) at 0° C. were added CsF (2.00 g, 13.2 mmol) and TMSCF₃ (1.88 g, 13.2 mmol). The reaction mixture was stirred at 20° C. for 16 h. The reaction mixture was then adjusted pH = 11 with aq. NaOH (2 N), extracted with EtOAc (3 × 10 mL), and the organics were discarded. The aqueous layer was adjusted to pH = 3 with aq. HC1 (3 N) and extracted with EtOAc (3 × 10 mL). These combined organic layers were washed with brine, dried over anhydrous Na₂SO₄, filtered, and concentrated under reduced pressure to provide a residue that was used directly as a 1:1 mixture of 4-bromo-2-(2,2,2-trifluoroacetyl)benzoic acid and 5-bromo-2-(2,2,2-trifluoroacetyl)benzoic acid. LCMS: m/z = 294.8, 296.8 [M-H]⁻.

6-bromo-4-(trifluoromethyl)phthalazin-l(2H)-one and 7-bromo-4-(trifluoromethyl)phthalazin-1(2H)-one: To a solution of 4-bromo-2-(2,2,2-trifluoroacetyl)benzoic acid and 5-bromo-2-(2,2,2-trifluoroacetyl)benzoic acid (1:1 mixture, 2.30 g, 7.74 mmol) in EtOH (15 mL) was added hydrazine monohydrate (465 mg, 9.29 mmol). The reaction mixture was stirred at 90° C. for 16 h. To the reaction mixture was added toluene (10 ml). The reaction mixture was stirred at 110° C. for a further 12 h. The reaction mixture was concentrated under reduced pressure. The residue was triturated with MTBE to provide a residue that was used directly as a 1:1 mixture of 6-bromo-4-(trifluoromethyl)phthalazin-1(2H)-one and 7-bromo-4-(trifluoromethyl)phthalazin-1(2H)-one. LCMS: m/z = 293.0, 295.0 [M+H]⁺.

2-[6-bromo-1-oxo-4-(trifluoromethyl)phthalazin-2-yl]-N-[(3R)-1-ethylpiperidin-3-yl]acetamide and 2-[7-bromo-1-oxo-4-(trifluoromethyl)phthalazin-2-yl]-N-[(3R)-1-ethylpiperidin-3-yl]acetamide: To a solution of 6-bromo-4-(trifluoromethyl)phthalazin-1(2H)-one and 7-bromo-4-(trifluoromethyl)phthalazin-1(2H)-one (1:1 mixture, 200 mg, 0.69 mmol) in DMF (3 mL) were added (R)-2-chloro-N-(1-ethylpiperidin-3-yl)acetamide (154 mg, 0.75 mmol) and Cs₂CO₃ (222 mg, 0.68 mmol). The reaction mixture was stirred at 90° C. for 2 h. The reaction mixture was diluted with water (20 mL) and extracted with EtOAc (3 × 5 mL). The combined organic layers were concentrated under reduced pressure. The residue was purified by reverse-phase preparative HPLC to provide:

2-[6-bromo-1-oxo-4-(trifluoromethyl)phthalazin-2-yl]-N-[(3R)-1-ethylpiperidin-3-yl]acetamide: LCMS: m/z = 461.1, 463.1 [M+H]⁺. ¹H NMR (400 MHz, CDC1₃): δ 8.34 (d, J= 8.4 Hz, 1H), 8.07 (s, 1H), 7.95 (dd, J = 8.6, 1.6 Hz, 1H), 6.76 (s, 1H), 4.90 (s, 2H), 4.14 (d, J = 3.6 Hz, 1H), 2.74-2.49 (m, 3H), 2.43-2.31 (m, 3H), 2.16-2.10 (m, 1H), 1.77-1.63 (m, 2H), 1.34-1.17 (m, 1H), 0.99 (t, J= 7.2 Hz, 3H).

2-[7-bromo-1-oxo-4-(trifluoromethyl)phthalazin-2-yl]-N-[(3R)-1-ethylpiperidin-3-yl]acetamide: LCMS: m/z = 461.1, 463.1 [M+H]⁺. ¹H NMR (400 MHz, CDC1₃): δ 8.65 (d, J= 2.0 Hz, 1H), 8.00 (dd, J = 8.8, 2.0 Hz, 1H), 7.83 (dd, J = 8.8, 1.5 Hz, 1H), 6.72 (br s, 1H), 4.93 (s, 2H), 4.18 (br s, 1H), 2.53-2.86 (m, 1H), 2.43 (br s, 3H), 2.18 (br s, 1H), 1.75 (br s, 1H), 1.42-1.59 (m, 3H), 1.34-1.17 (m, 1H), 0.99 (t, J = 7.2 Hz, 3H).

### Example 91

### 2-Bromo-1-Oxo-4-(2,2,2-Trifluoroethyl)Phthalazin-2-yl]-N-(5-Fluoropyrimidin-4-yl)Acetamide

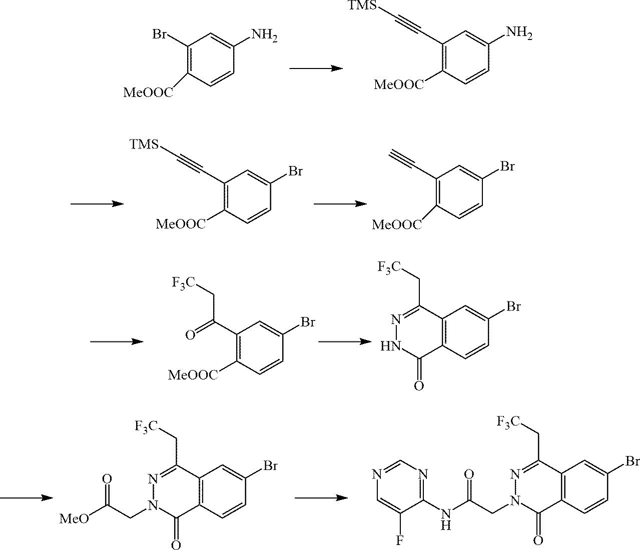

Methyl 4-bromo-2-((trimethylsilyl)ethynyl)benzoate: To a solution of methyl 4-amino-2-bromobenzoate (500 mg, 2.17 mmol) and ethynyltrimethylsilane (640 mg, 6.52 mmol) in DMF (10 mL) were added Pd(PPh₃)₂Cl₂ (153 mg, 0.22 mmol), CuI (83 mg, 0.43 mmol) and Et₃N (440 mg, 4.35 mmol). The reaction mixture was stirred at 110° C. for 2 h. The reaction mixture was poured into water (100 mL) and extracted with EtOAc (3 × 30 mL). The combined organic layers were washed with brine (2 × 30 mL), dried over anhydrous Na₂SO₄, filtered, and concentrated under reduced pressure. The residue was purified by silica gel column chromatography. ¹H NMR (400 MHz, CDC1₃): δ 7.81 (d, J = 8.4 Hz, 1H), 6.83 (d, J= 2.4 Hz, 1H), 6.60 (dd, J = 2.4, 8.4 Hz, 1H), 4.01 (s, 2H), 3.87 (s, 3H), 0.27 (s, 9H).

Methyl 4-bromo-2-((trimethylsilyl)ethynyl)benzoate: To a solution of methyl 4-bromo-2-((trimethylsilyl)ethynyl)benzoate (1.70 g, 6.87 mmol) and t-BuONO (2.13 g, 20.6 mmol) in MeCN (35 mL) at 0° C. was added CuBr₂ (1.53 g, 6.87 mmol). The reaction mixture was stirred at 25° C. for 1 h. The reaction mixture was poured into water (30 mL) and extracted with EtOAc (3 × 15 mL). The combined organic layers were washed with brine (15 mL), dried over anhydrous Na₂SO₄, filtered, and concentrated under reduced pressure. The residue was purified by silica gel column chromatography.

Methyl 4-bromo-2-ethynylbenzoate: To a solution of methyl 4-bromo-2-((trimethylsilyl)ethynyl)benzoate (1.28 g, 4.11 mmol) in MeOH (20 mL) was added K₂CO₃ (1.14 g, 8.23 mmol). The reaction mixture was stirred at 25° C. for 30 min. The reaction mixture was adjusted to pH = 7 with aq. HC1 (1 N) and extracted with EtOAc (3 × 5 mL). The combined organic layers were washed with brine (10 mL), dried over anhydrous Na₂SO₄, filtered, and concentrated under reduced pressure. The residue was purified by silica gel column chromatography. ¹H NMR (400 MHz, CDC1₃): δ 7.85-7.75 (m, 2H), 7.56-7.53 (m, 1H), 3.93 (d, J = 3.6 Hz, 3H), 3.46 (s, 1H).

Methyl 4-bromo-2-(3,3,3-trifluoropropanoyl)benzoate: To a solution of methyl 4-bromo-2-ethynylbenzoate (735 mg, 3.07 mmol) and sodium trifluoromethanesulfinate (576 mg, 3.69 mmol) in NMP (14 mL) was added AgNO₃ (104 mg, 0.61 mmol). The reaction mixture was stirred at 70° C. under O₂ (15 psi) for 16 h. The reaction mixture was diluted with EtOAc (10 mL) and filtered through a thin celite pad. The filtrate was diluted with water (20 mL) and extracted with EtOAc (3 × 5 mL). The combined organic layers were washed with brine (3 × 5 mL), dried over anhydrous Na₂SO₄, filtered, and concentrated under reduced pressure. The residue was purified by silica gel column chromatography. LCMS: m/z = 322.9, 324.9 [M-H]⁻.

6-bromo-4-(2,2,2-trifluoroethyl)phthalazin-1(2H)-one: To a solution of methyl 4-bromo-2-(3,3,3-trifluoropropanoyl)benzoate (170 mg, 0.52 mmol) in EtOH (5 mL) was added hydrazine hydrate (26 mg, 0.52 mmol). The reaction mixture was stirred at 80° C. for 16 h. The reaction mixture was concentrated under reduced pressure to provide a residue that was used directly. LCMS: m/z = 307.0, 309.0 [M+H]⁺.

Methyl 2-(6-bromo-1-oxo-4-(2,2,2-trifluoroethyl)phthalazin-2(1H)-yl)acetate: To a solution of 6-bromo-4-(2,2,2-trifluoroethyl)phthalazin-1(2H)-one (100 mg, 0.33 mmol) in DMF (1.0 mL) were added Cs₂CO₃ (318 mg, 0.98 mmol) and methyl 2-bromoacetate (75 mg, 0.49 mmol). The reaction mixture was stirred at 20° C. for 3 h. The reaction mixture was diluted with water (3 mL) and extracted with EtOAc (3 × 2 mL). The combined organic layers were washed with brine, dried over anhydrous Na₂SO₄, filtered, and concentrated under reduced pressure. The residue was purified by silica gel chromatography. LCMS: m/z = 379.1, 381.1 [M+H]⁺.

2-[6-bromo-1-oxo-4-(2,2,2-trifluoroethyl)phthalazin-2-yl]-N-(5-fluoropyrimidin-4-yl)acetamide: To a solution of methyl 2-(6-bromo-1-oxo-4-(2,2,2-trifluoroethyl)phthalazin-2(1H)-yl)acetate (100 mg, 0.26 mmol) in toluene (1.0 mL) and THF (1.0 mL) was added 5-fluoropyrimidin-4-amine (89 mg, 0.80 mmol) and AlMe₃ (0.40 mL, 2 M in toluene). The reaction mixture was stirred at 100° C. for 3 h. The reaction mixture was diluted with water (2 mL) and extracted with EtOAc (3 × 1 mL). The combined organic layers were washed with brine (2 mL), dried over anhydrous Na₂SO₄, filtered, and concentrated under reduced pressure. The residue was purified by reverse-phase preparative. HPLC. LCMS: m/z = 460.0, 462.0 [M+H]⁺. ¹H NMR (400 MHz, CDC1₃): δ 8.76 (d, J= 2.0 Hz, 1H), 8.51 (d, J = 2.4 Hz, 1H), 8.37 (d, J = 9.2 Hz, 1H), 7.97-7.91 (m, 2H), 5.57 (s, 2H), 3.76 (q, J = 10.0 Hz, 2H).

### Example 92

### 2-Bromo-8-Fluoro-1-Oxo-4-Propan-2-Ylphthalazin-2-yl)-N-(5-Fluoropyrimidin-4-yl)Acetamide (92)

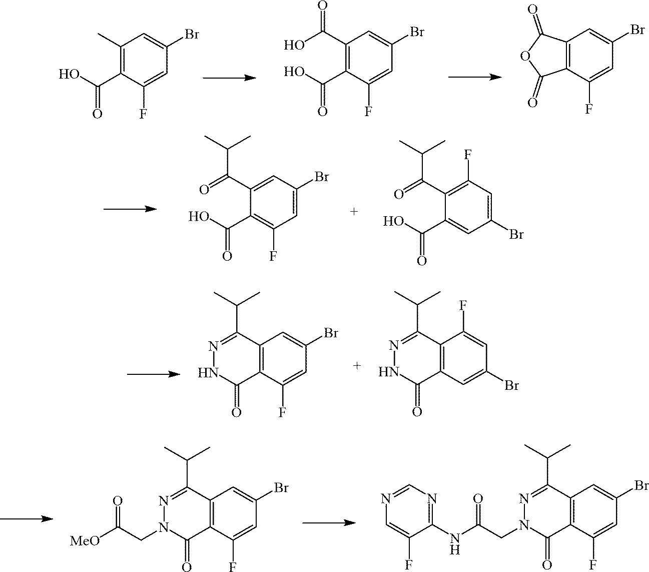

5-bromo-3-fluorophthalic acid: To a solution of 4-bromo-2-fluoro-6-methyl-benzoic acid (8.00 g, 34.3 mmol) in water (80 mL) were added KMnO₄ (16.28 g, 103 mmol) and NaOH (5.49 g, 137 mmol). The reaction mixture was stirred at 100° C. for 16 h. The reaction mixture was filtered through a thin layer of celite. The filtrate was adjusted to pH = 3 with aq. HC1 (2 N) and extracted with DCM (3 × 150 mL). The combined organic layers were washed with brine (150 mL), dried over anhydrous Na₂SO₄, filtered, and concentrated under reduced pressure to provide a residue that was used directly. ¹H NMR (400 MHz, DMSO-d6): δ 13.78 (br s, 2H), 7.95 (d, J= 1.6 Hz, 1H), 7.85 (s, 1H).

6-bromo-4-fluoroisobenzofuran-1,3-dione: A solution of 5-bromo-3-fluorophthalic acid (6.40 g, 24.3 mmol) in SOC1₂ (164 g, 1.38 mol) was stirred at 90° C. for 2 h. The reaction mixture was concentrated under reduced pressure to provide a residue that was used directly. ¹H NMR (400 MHz, CDC1₃): δ 8.00 (s, 1H), 7.76 (dd, J = 0.8, 7.6 Hz, 1H).

5-bromo-3-fluoro-2-isobutyrylbenzoic acid and 4-bromo-2-fluoro-6-isobutyrylbenzoic acid: To a solution of 6-bromo-4-fluoroisobenzofuran-1,3-dione (1.00 g, 4.08 mmol) in THF (20 mL) at -10° C. was added isopropyl magnesium chloride (2.04 mL, 2 M in THF). The reaction mixture was stirred at -10° C. for 3 h. The reaction mixture was quenched with NH₄Cl (20 mL), adjusted to pH = 10 with aq. Na₂CO₃ (2 N), and extracted with MTBE (20 mL). These organics were discarded. The aqueous layer was then adjusted to pH = 3 with aq. HC1 (2 N) and extracted with EtOAc (3 × 20 mL). These combined organic layers were washed with brine (20 mL), dried over anhydrous Na₂SO₄, filtered, and concentrated under reduced pressure to provide a residue that was used directly as a 1:1 mixture of 5-bromo-3-fluoro-2-isobutyrylbenzoic acid and 4-bromo-2-fluoro-6-isobutyrylbenzoic acid.

6-bromo-8-fluoro-4-isopropylphthalazin-1(2H)-one and 7-bromo-5-fluoro-4-isopropylphthalazin-1(2H)-one: To a solution of 5-bromo-3-fluoro-2-isobutyrylbenzoic acid and 4-bromo-2-fluoro-6-isobutyrylbenzoic acid (1:1 mixture, 970 mg, 3.36 mmol) in EtOH (10 mL) was added hydrazine hydrate (137 mg, 2.68 mmol). The reaction mixture was stirred at 90° C. for 12 h. The reaction mixture was concentrated under reduced pressure to provide a residue that was used directly as a 1:1 mixture of 6-bromo-8-fluoro-4-isopropylphthalazin-1(2H)-one and 7-bromo-5-fluoro-4-isopropylphthalazin-1(2H)-one. LCMS: m/z = 285.1, 287.1 [M+H]⁺.

Methyl 2-(6-bromo-8-fluoro-4-isopropyl-1-oxophthalazin-2(1H)-yl)acetate: To a solution of 6-bromo-8-fluoro-4-isopropylphthalazin-1(2H)-one and 7-bromo-5-fluoro-4-isopropylphthalazin-1(2H)-one (1:1 mixture, 890 mg, 3.12 mmol) in DMF (10 mL) were added methyl 2-bromoacetate (955 mg, 6.24 mmol) and Cs₂CO₃ (2.03 g, 6.24 mmol). The reaction mixture was stirred at 50° C. for 3 h. The reaction mixture was diluted with water (50 mL) and extracted with EtOAc (3 × 30 mL). The combined organic layers were washed with brine (30 mL), dried over anhydrous Na₂SO₄, filtered, and concentrated under reduced pressure. The residue was purified by silica gel column chromatography.

2-(6-bromo-8-fluoro-1-oxo-4-propan-2-ylphthalazin-2-yl)-N-(5-fluoropyrimidin-4-yl)acetamide: To a solution of methyl 2-(6-bromo-8-fluoro-4-isopropyl-1-oxophthalazin-2(1H)-yl)acetate (216 mg, 0.60 mmol) in toluene (2.0 mL) and THF (2.0 mL) were added 5-fluoropyrimidin-4-amine (82 mg, 0.72 mmol) and AlMe₃ (0.9 mL, 2 M in toluene). The reaction mixture was stirred at 90° C. for 3 h. The reaction mixture was diluted with water (15 mL) and extracted with EtOAc (3 × 10 mL). The combined organic layers were washed with brine (10 mL), dried over anhydrous Na₂SO₄, filtered, and concentrated under reduced pressure. The residue was purified by reverse-phase preparative HPLC. LCMS: m/z = 438.0, 440.0 [M+H]⁺. ¹H NMR (400 MHz, CDCl₃): δ 8.76 (s, 1H), 8.59 (br s, 1H), 8.50 (d, J = 2.0 Hz, 1H), 7.83 (s, 1H), 7.60-7.55 (m, 1H), 5.43 (s, 2H), 3.46-3.32 (m, 1H), 1.36 (d, J = 6.8 Hz, 6H).

### Examples 93 and 94

### 2-Bromo-4-(Difluoromethyl)-1-Oxo-Phthalazin-2-yl]-N-Pyrimidin-2-yl-Acetamide (93) and 2-[7-Bromo-4-(Difluoromethyl)-1-Oxo-Phthalazin-2-yl]-N-Pyrimidin-2-yl-Acetamide (94)

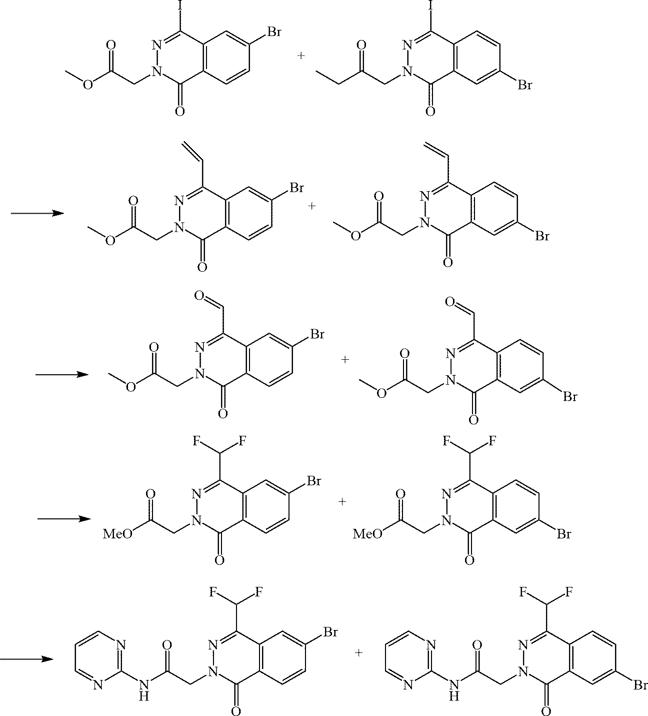

Methyl 2-(6-bromo-1-oxo-4-vinylphthalazin-2(1H)-yl)acetate and methyl 2-(7-bromo-1-oxo-4-vinylphthalazin-2(1H)-yl)acetate mixture: To a solution of methyl 2-(6-bromo-4-iodo-1-oxophthalazin-2(1H)-yl)acetate and methyl 2-(7-bromo-4-iodo-1-oxophthalazin-2(1H)-yl)acetate (1:1 mixture, 2.00 g, 4.73 mmol) in 1,4-dioxane (30 mL) were added potassium trifluoro(vinyl)borate (697 mg, 5.20 mmol), CsF (2.15 g, 14.2 mmol), and Pd(dppf)Cl₂ (346 mg, 0.47 mmol). The reaction mixture was stirred at 90° C. for 12 h. The reaction mixture was concentrated under reduced pressure. The residue was purified by silica gel column chromatography to provide a 1:1 mixture of methyl 2-(6-bromo-1-oxo-4-vinylphthalazin-2(1H)-yl)acetate and methyl 2-(7-bromo-1-oxo-4-vinylphthalazin-2(1H)-yl)acetate. LCMS: m/z = 323.1, 325.1 [M+H]⁺.

Methyl 2-(6-bromo-4-formyl-1-oxophthalazin-2(1H)-yl)acetate and methyl 2-(7-bromo-4-formyl-1-oxophthalazin-2(1H)-yl)acetate mixture: A solution of methyl 2-(6-bromo-1-oxo-4-vinylphthalazin-2(1H)-yl)acetate and methyl 2-(7-bromo-1-oxo-4-vinylphthalazin-2(1H)-yl)acetate (1:1 mixture, 1.50 g, 4.64 mmol) in DCM (10 mL) and EtOAc (15 mL) was stirred at -78° C. under an ozone atmosphere for 0.5 h at 15 psi. To the reaction mixture was then added Me₂S (2.88 g, 46.4 mmol) and the reaction was stirred at 20° C. for a further 16 h. The reaction mixture was diluted with water (20 mL) and extracted with DCM (3 × 10 mL). The combined organic layers were washed with brine (20 mL), dried over anhydrous Na₂SO₄, filtered, and concentrated under reduced pressure to provide a residue that was used directly as a 1:1 mixture of methyl 2-(6-bromo-4-formyl-1-oxophthalazin-2(1H)-yl)acetate and methyl 2-(7-bromo-4-formyl-1-oxophthalazin-2(1H)-yl)acetate. LCMS: m/z = 325.0, 327.0 [M+H]⁺.

Methyl 2-(6-bromo-4-(difluoromethyl)-1-oxophthalazin-2(1H)-yl)acetate and methyl 2-(7-bromo-4-(difluoromethyl)-1-oxophthalazin-2(1H)-yl)acetate: A solution of methyl 2-(6-bromo-4-formyl-1-oxophthalazin-2(1H)-yl)acetate and methyl 2-(7-bromo-4-formyl-1-oxophthalazin-2(1H)-yl)acetate mixture (1:1 mixture, 300 mg, 0.92 mmol) in BAST (3.03 g, 13.7 mmol) was stirred at 20° C. for 6 h. The reaction mixture was poured into aq. Na₂CO₃ (2 N, 5 mL) and extracted with EtOAc (3 × 2 mL). The combined organic layers were washed with brine (5 mL), dried over anhydrous Na₂SO₄, filtered, and concentrated under reduced pressure. The residue was purified by silica gel column chromatography to provide a 1:1 mixture of methyl 2-(6-bromo-4-(difluoromethyl)-1-oxophthalazin-2(1H)-yl)acetate & methyl 2-(7-bromo-4-(difluoromethyl)-1-oxophthalazin-2(1H)-yl)acetate. LCMS: m/z = 347.0, 349.0 [M+H]⁺.

2-(6-bromo-4-(difluoromethyl)-1-oxophthalazin-2(1H)-yl)-N-(pyrimidin-2-yl)acetamide and 2-(7-bromo-4-(difluoromethyl)-1-oxophthalazin-2(1H)-yl)-N-(pyrimidin-2-yl)acetamide: To a solution of methyl 2-(6-bromo-4-(difluoromethyl)-1-oxophthalazin-2(1H)-yl)acetate and methyl 2-(7-bromo-4-(difluoromethyl)-1-oxophthalazin-2(1H)-yl)acetate mixture (1:1 mixture, 180 mg, 0.52 mmol) in toluene (1.0 mL) and THF (1.0 mL) were added pyrimidin-2-amine (99 mg, 1.04 mmol) and AlMe₃ (0.78 mL, 2 M in toluene). The reaction mixture was stirred at 100° C. for 3 h. The reaction mixture was diluted with water (5 mL) and extracted with EtOAc (3 × 2 mL). The combined organic layers were washed with brine (5 mL), dried over anhydrous Na₂SO₄, filtered, and concentrated under reduced pressure. The residue was purified by reverse-phase preparative HPLC to provide:

2-[6-bromo-4-(difluoromethyl)-1-oxo-phthalazin-2-yl]-N-pyrimidin-2-yl-acetamide: LCMS: m/z = 410.1, 412.1 [M+H]⁺. ¹H NMR (400 MHz, CDCl₃): δ 8.78 (br s, 1H), 8.63 (d, J = 4.8 Hz, 2H), 8.38-8.31 (m, 2H), 7.97-7.91 (m, 1H), 7.08-7.04 (m, 1H), 6.61 (t, J = 53.6 Hz, 1H), 5.61 (s, 2H).

2-[7-bromo-4-(difluoromethyl)-1-oxo-phthalazin-2-yl]-N-pyrimidin-2-yl-acetamide: LCMS: m/z = 410.1, 412.0 [M+H]⁺. ¹H NMR (400 MHz, CDCl₃): δ 9.06 (s, 1H), 8.67-8.62 (m, 3H), 8.09-8.03 (m, 1H), 8.01-7.96 (m, 1H), 7.08-7.03 (m, 1H), 6.61 (t, J= 53.6 Hz, 1H), 5.62 (s, 2H).

### Examples 95 and 96

### 2-Bromo-4-(Difluoromethoxy)-1-Oxophthalazin-2-yl]-N-(5-Fluoropyrimidin-4-yl)Acetamide (95) and 2-[7-Bromo-4-(Difluoromethoxy)-1-Oxophthalazin-2-yl]-N-(5-Fluoropyrimidin-4-yl)Acetamide (96)

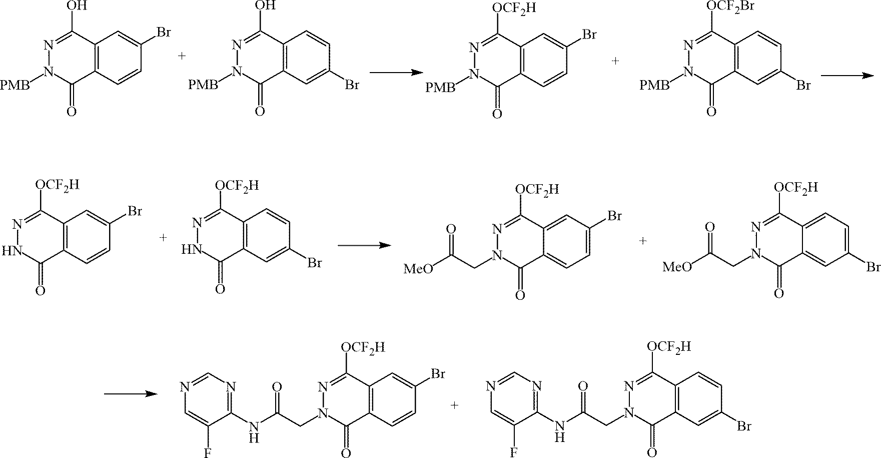

6-bromo-4-(difluoromethoxy)-2-(4-methoxybenzyl)phthalazin-1(2H)-one and 7-bromo-4-(difluoromethoxy)-2-(4-methoxybenzyl)phthalazin-1(2H)-one: To a solution of 6-bromo-4-hydroxy-2-(4-methoxybenzyl)phthalazin-1(2H)-one and 7-bromo-4-hydroxy-2-(4-methoxybenzyl)phthalazin-1(2H)-one (1:1 mixture, 500 mg, 1.38 mmol) in DMF (10 mL) at 0° C. were added TBAB (22 mg, 0.07 mmol) and NaH (66 mg, 1.66 mmol, 60% purity). The reaction mixture was stirred at 0° C. for 1 h. To the reaction mixture was then added dibromo(difluoro)methane (1.20 g, 5.52 mmol). The reaction mixture was stirred at 20° C. for 16 h. The reaction mixture was poured into sat. aq. NH₄Cl (30 mL) and extracted with EtOAc (3 × 10 mL). The combined organic layers were washed with brine (10 mL), dried over anhydrous Na₂SO₄, filtered, and concentrated under reduced pressure. The residue was purified by reverse-phase preparative HPLC to provide a 1:1 mixture of 6-bromo-4-(difluoromethoxy)-2-(4-methoxybenzyl)phthalazin-1(2H)-one and 7-bromo-4-(difluoromethoxy)-2-(4-methoxybenzyl)phthalazin-1(2H)-one. LCMS: m/z = 361.0, 363.0 [M+H]⁺.

6-bromo-4-(difluoromethoxy)phthalazin-1(2H)-one and 7-bromo-4-(difluoromethoxy)phthalazin-1(2H)-one): To a solution of 6-bromo-4-(difluoromethoxy)-2-(4-methoxybenzyl)phthalazin-1(2H)-one and 7-bromo-4-(difluoromethoxy)-2-(4-methoxybenzyl)phthalazin-1(2H)-one (1:1 mixture, 50 mg, 0.12 mmol) in MeCN (5.0 mL) and water (1.0 mL) was added CAN (200 mg, 0.36 mmol). The reaction mixture was stirred at 20° C. for 16 h. The reaction mixture was poured into saturated sat. aq. NaHCO₃ (10 mL) and extracted with EtOAc (3 × 5 mL). The combined organic layers were washed with brine (5 mL), dried over anhydrous Na₂SO₄, filtered, and concentrated under educed pressure to afford a 1.4:1 mixture of 6-bromo-4-(difluoromethoxy)phthalazin-1(2H)-one and 7-bromo-4-(difluoromethoxy)phthalazin-1(2H)-one. LCMS: m/z = 291.0, 293.0 [M+H]⁺.

Methyl 2-(6-bromo-4-(difluoromethoxy)-1-oxophthalazin-2(1H)-yl)acetate and methyl 2-(7-bromo-4-(difluoromethoxy)-1-oxophthalazin-2(1H)-yl)acetate: To a solution of 6-bromo-4-(difluoromethoxy)phthalazin-1(2H)-one and 7-bromo-4-(difluoromethoxy)phthalazin-1(2H)-one (1.4:1 mixture; 70 mg, 0.24 mmol) in DMF (2.0 mL) were added methyl 2-bromoacetate (55 mg, 0.36 mmol) and Cs₂CO₃ (235 mg, 0.72 mmol). The reaction mixture was stirred at 50° C. for 12 h. The reaction mixture was concentrated under reduced pressure. The residue was purified by silica gel column chromatography to afford a mixture (1.3:1) of 2-(6-bromo-4-(difluoromethoxy)-1-oxophthalazin-2(1H)-yl)acetate and methyl 2-(7-bromo-4-(difluoromethoxy)-1-oxophthalazin-2(1H)-yl)acetate. LCMS: m/z = 362.9, 364.9 [M+H]⁺.

2-[6-bromo-4-(difluoromethoxy)-1-oxophthalazin-2-yl]-N-(5-fluoropyrimidin-4-yl)acetamide and 2-(7-bromo-4-(difluoromethoxy)-1-oxophthalazin-2(1H)-yl)-N-(5-fluoropyrimidin-4-yl)acetamide: To a solution of methyl 2-(6-bromo-4-(difluoromethoxy)-1-oxophthalazin-2(1H)-yl)acetate and methyl 2-(7-bromo-4-(difluoromethoxy)-1-oxophthalazin-2(1H)-yl)acetate (1.3:1 mixture, 15 mg, 0.04 mmol) in toluene (5.0 mL) were added AlMe₃ (0.06 mL, 2 M in toluene) and 5-fluoropyrimidin-4-amine (9 mg, 0.08 mmol). The reaction mixture was stirred at 90° C. for 5 h. The reaction mixture was poured into sat. aq. NH₄Cl (10 mL) and extracted with EtOAc (3 × 5 mL). The combined organic layers were washed with brine (5 mL), dried over anhydrous Na₂SO₄, filtered, and concentrated under reduced pressure. The residue was purified by reverse-phase preparative HPLC to provide a 1.7:1 mixture of 2-[6-bromo-4-(difluoromethoxy)-1-oxophthalazin-2-yl]-N-(5-fluoropyrimidin-4-yl)acetamide and 2-[7-bromo-4-(difluoromethoxy)-1-oxo-phthalazin-2-yl]-N-(5-fluoropyrimidin-4-yl)acetamide. LCMS: m/z = 444.0, 446.0 [M+H]⁺.

### Examples 97 and 98

### 2-Bromo-4-(2-Fluoroethyl)-1-Oxo-Phthalazin-2-yl]-N-Pyrimidin-2-yl-Acetamide (97) and 2-[7-Bromo-4-(2-Fluoroethyl)-1-Oxo-Phthalazin-2-yl]-N-Pyrimidin-2-yl-Acetamide (98)

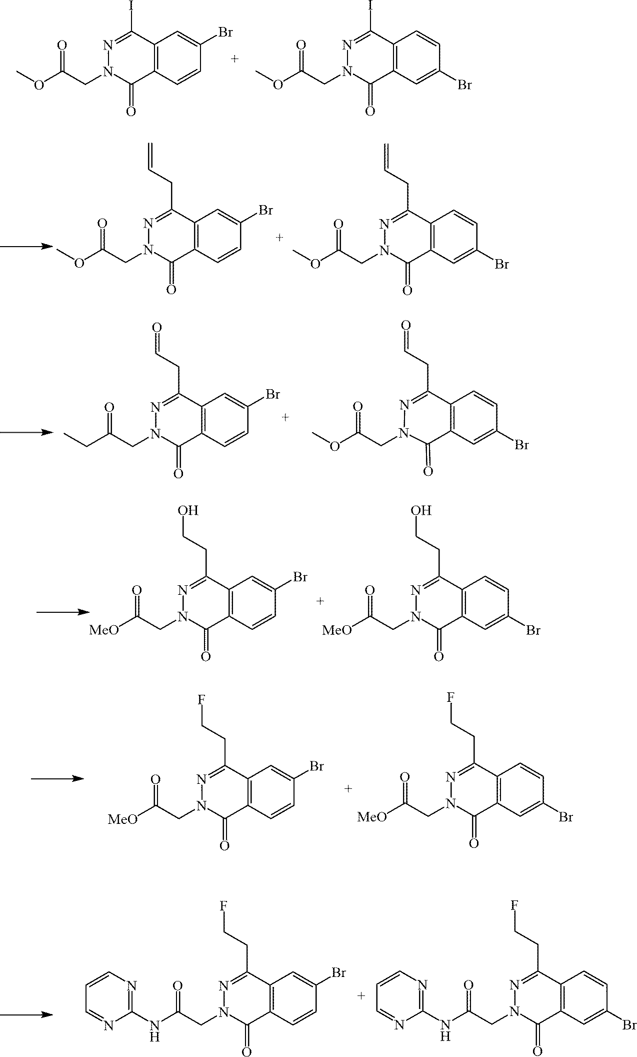

Methyl 2-(4-allyl-6-bromo-1-oxophthalazin-2(1H)-yl)acetate and methyl 2-(4-allyl-7-bromo-1-oxophthalazin-2(1H)-yl)acetate: To a solution of methyl 2-(6-bromo-4-iodo-1-oxophthalazin-2(1H)-yl)acetate and methyl 2-(7-bromo-4-iodo-1-oxophthalazin-2(1H)-yl)acetate mixture (1:1 mixture, 1.50 g, 3.55 mmol) in 1,4-dioxane (20 mL) were added 2-allyl-4,4,5,5-tetramethyl-X1,3,2-dioxaborolane (656 mg, 3.90 mmol), K₂CO₃ (1.47 g, 10.6 mmol), and Pd(PPh₃)₄ (410 mg, 0.36 mmol). The reaction mixture was stirred at 120° C. for 3 h. The reaction mixture was diluted with water (20 mL) and extracted EtOAc (3 × 8 mL). The combined organic layers were washed with brine (20 mL), dried over anhydrous Na₂SO₄, filtered, and concentrated under reduced pressure. The residue was purified by silica gel column chromatography to provide a 1:1 mixture methyl 2-(4-allyl-6-bromo-1-oxophthalazin-2(1H)-yl)acetate and methyl 2-(4-allyl-7-bromo-1-oxophthalazin-2(1H)-yl)acetate. LCMS: m/z = 337.1, 339.1 [M+H]⁺.

Methyl 2-(6-bromo-1-oxo-4-(2-oxoethyl)phthalazin-2(1H)-yl)acetate and methyl 2-(7-bromo-1-oxo-4-(2-oxoethyl)phthalazin-2(1H)-yl)acetate: A solution of methyl 2-(4-allyl-6-bromo-1-oxophthalazin-2(1H)-yl)acetate and methyl 2-(4-allyl-7-bromo-1-oxophthalazin-2(1H)-yl)acetate (1:1 mixture, 1.00 g, 2.97 mmol) in DCM (20 mL) was stirred at -78° C. for 12 min under an atmosphere of ozone at 15 psi. To the reaction mixture was then added Me₂S (2.95 g, 47.5 mmol). The reaction mixture was stirred at 20° C. for 16 h. The reaction mixture was diluted with water (20 mL) and extracted with DCM (3 × 10 mL). The combined organic layers were washed with brine (20 mL), dried over anhydrous Na₂SO₄, filtered, and concentrated under reduced pressure. The residue was purified by silica gel column chromatography to provide 1:1 mixture of methyl 2-(6-bromo-1-oxo-4-(2-oxoethyl)phthalazin-2(1H)-yl)acetate and methyl 2-(7-bromo-1-oxo-4-(2-oxoethyl)phthalazin-2(1H)-yl)acetate mixture. LCMS: m/z = 339.0, 341.0 [M+H]⁺.

Methyl 2-(6-bromo-4-(2-hydroxyethyl)-1-oxophthalazin-2(1H)-yl)acetate and methyl 2-(7-bromo-4-(2-hydroxyethyl)-1-oxophthalazin-2(1H)-yl)acetate: To a solution of methyl 2-(6-bromo-1-oxo-4-(2-oxoethyl)phthalazin-2(1H)-yl)acetate and methyl 2-(7-bromo-1-oxo-4-(2-oxoethyl)phthalazin-2(1H)-yl)acetate (1:1 mixture, 1.00 g, 2.95 mmol) in THF (10 mL) at 0° C. was added NaBH₄ (112 mg, 2.95 mmol). The reaction mixture was stirred at 0° C. for 1 h. The reaction mixture was diluted with brine (10 mL) and extracted with EtOAc (3 × 4 mL). The combined organic layers were washed brine (10 mL), dried over anhydrous Na₂SO₄, filtered, and concentrated under reduced pressure. The residue was purified by silica gel column chromatography to provide 1:1 mixture of methyl 2-(6-bromo-4-(2-hydroxyethyl)-1-oxophthalazin-2(1H)-yl)acetate & methyl 2-(7-bromo-4-(2-hydroxyethyl)-1-oxophthalazin-2(1H)-yl)acetate. LCMS: m/z = 341.0, 343.0 [M+H]⁺.

Methyl 2-(6-bromo-4-(2-fluoroethyl)-1-oxophthalazin-2(1H)-yl)acetate and methyl 2-(7-bromo-4-(2-fluoroethyl)-1-oxophthalazin-2(1H)-yl)acetate: A solution of methyl 2-(6-bromo-4-(2-hydroxyethyl)-1-oxophthalazin-2(1H)-yl)acetate and methyl 2-(7-bromo-4-(2-hydroxyethyl)-1-oxophthalazin-2(1H)-yl)acetate (1:1 mixture, 200 mg, 0.59 mmol) in BAST (130 mg, 0.59 mmol) was stirred at 20° C. for 16 h. The reaction mixture was diluted with sat. aq. NaHCO₃ (2 mL) and extracted with EtOAc (3 × 1 mL). The organics were washed with brine (2 mL), dried over anhydrous Na₂SO₄, filtered, and concentrated under reduced pressure. The residue was purified by silica gel column chromatography to provide a 1:1 mixture of methyl 2-(6-bromo-4-(2-fluoroethyl)-1-oxophthalazin-2(1H)-yl)acetate and methyl 2-(7-bromo-4-(2-fluoroethyl)-1-oxophthalazin-2(1H)-yl)acetate. LCMS: m/z = 343.0, 345.0 [M+H]⁺.

2-[6-bromo-4-(2-fluoroethyl)-1-oxo-phthalazin-2-yl]-N-pyrimidin-2-yl-acetamide and 2-[7-bromo-4-(2-fluoroethyl)-1-oxo-phthalazin-2-yl]-N-pyrimidin-2-yl-acetamide: To a solution of methyl 2-(6-bromo-4-(2-fluoroethyl)-1-oxophthalazin-2(1H)-yl)acetate and methyl 2-(7-bromo-4-(2-fluoroethyl)-1-oxophthalazin-2(1H)-yl)acetate (1:1 mixture, 70 mg, 0.21 mmol) in toluene (1.0 mL) and THF (1.0 mL) were added pyrimidin-2-amine (39 mg, 0.41 mmol) and DABAL-Me₃ (18 mg, 0.25 mmol). The reaction mixture was stirred at 100° C. for 3 h. The reaction mixture was diluted with water (5 mL) and extracted with EtOAc (3 × 2 mL). The combined organic layers were washed with brine (5 mL), dried over anhydrous Na₂SO₄, filtered, and concentrated under reduced pressure. The residue was purified by reverse-phase HPLC to afford a 2:3 mixture of 2-[6-bromo-4-(2-fluoroethyl)-1-oxo-phthalazin-2-yl]-N-pyrimidin-2-yl-acetamide and 2-[7-bromo-4-(2-fluoroethyl)-1-oxo-phthalazin-2-yl]-N-pyrimidin-2-yl-acetamide. LCMS: m/z = 406.0, 408.0 [M+H]⁺.

### Examples 99 and 100

### 2-Bromo-4-(2,2-Difluoroethyl)-1-Oxophthalazin-2-yl]-N-Pyrimidin-2-Ylacetamide (99) and 2-[7-Bromo-4-(2,2-Difluoroethyl)-1-Oxophthalazin-2-yl]-N-Pyrimidin-2-Ylacetamide (100)

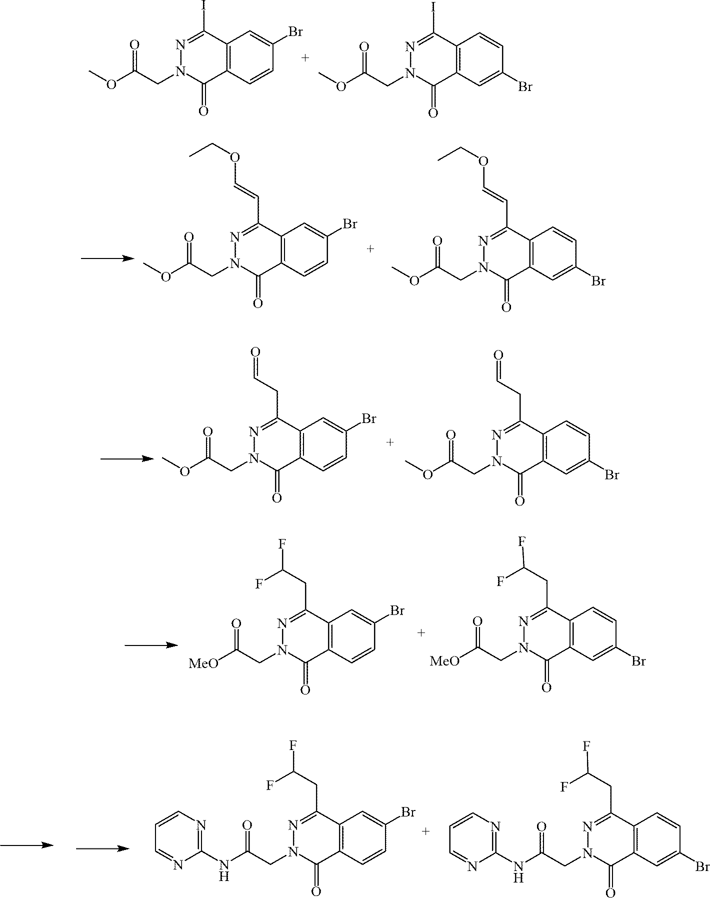

(E)-methyl 2-(6-bromo-4-(2-ethoxyvinyl)-1-oxophthalazin-2(1H)-yl)acetate and (E)-methyl 2-(7-bromo-4-(2-ethoxyvinyl)-1-oxophthalazin-2(1H)-yl)acetate mixture: To a solution of methyl 2-(6-bromo-4-iodo-1-oxophthalazin-2(1H)-yl)acetate and methyl 2-(7-bromo-4-iodo-1-oxophthalazin-2(1H)-yl)acetate (1:1 mixture, 500 mg, 1.18 mmol) in 1,4-dioxane (8 mL) were added (E)-2-(2-ethoxyvinyl)-4,4,5,5-tetramethyl-1,3,2-dioxaborolane (234 mg, 1.18 mmol), Cs₂CO₃ (1.16 g, 3.55 mmol) and Pd(dppf)Cl₂ (87 mg, 0.12 mmol). The reaction mixture was stirred at 100° C. for 6 h. The reaction mixture was diluted with water (10 mL) and extracted with EtOAc (3 × 10 mL). The combined organic layers were washed with brine (10 mL), dried over anhydrous Na₂SO₄, filtered, and concentrated under reduced pressure. The residue was purified by silica gel column chromatography to provide a 1:1 mixture of (E)-methyl 2-(6-bromo-4-(2-ethoxyvinyl)-1-oxophthalazin-2(1H)-yl)acetate and (E)-methyl 2-(7-bromo-4-(2-ethoxyvinyl)-1-oxophthalazin-2(1H)-yl)acetate. LCMS: m/z = 367.0, 369.0 [M+H]⁺.

Methyl 2-(6-bromo-1-oxo-4-(2-oxoethyl)phthalazin-2(1H)-yl)acetate and methyl 2-(7-bromo-1-oxo-4-(2-oxoethyl)phthalazin-2(1H)-yl)acetate: To a solution of (E)-methyl 2-(6-bromo-4-(2-ethoxyvinyl)-1-oxophthalazin-2(1H)-yl)acetate and (E)-methyl 2-(7-bromo-4-(2-ethoxyvinyl)-1-oxophthalazin-2(1H)-yl)acetate (1:1 mixture, 300 mg, 0.82 mmol) in THF (3.0 mL) was added aq. HCl (1 M, 10 mL). The reaction mixture was stirred at 80° C. for 1 h. The reaction mixture was diluted with water (10 mL), adjusted to pH = 6 with aq. NaHCO₃ (1 M), and extracted with EtOAc (3 × 5 mL). The combined organic layers were washed with brine (6 mL), dried over anhydrous Na₂SO₄, filtered, and concentrated under reduced pressure to provide a residue that was used directly as a 1:1 mixture of methyl 2-(6-bromo-1-oxo-4-(2-oxoethyl)phthalazin-2(1H)-yl)acetate and methyl 2-(7-bromo-1-oxo-4-(2-oxoethyl)phthalazin-2(1H)-yl)acetate. LCMS: m/z = 338.8, 340.9 [M+H]⁺.

Methyl 2-(6-bromo-4-(2,2-difluoroethyl)-1-oxophthalazin-2(1H)-yl)acetate and methyl 2-(7-bromo-4-(2,2-difluoroethyl)-1-oxophthalazin-2(1H)-yl)acetate mixture: A solution of methyl 2-(6-bromo-1-oxo-4-(2-oxoethyl)phthalazin-2(1H)-yl)acetate and methyl 2-(7-bromo-1-oxo-4-(2-oxoethyl)phthalazin-2(1H)-yl)acetate (1:1 mixture, 100 mg, 0.30 mmol) in BAST (2.02 g, 9.13 mmol) was stirred at 20° C. for 16 h. The reaction mixture was poured into sat. aq. NaHCO₃ (4 mL) and extracted with EtOAc (3 × 3 mL). The combined organic layers were washed brine (4 mL), dried over anhydrous Na₂SO₄, filtered, and concentrated under reduced pressure. The residue was purified by preparatory TLC to provide a 1:1 mixture of methyl 2-(6-bromo-4-(2,2-difluoroethyl)-1-oxophthalazin-2(1H)-yl)acetate and methyl 2-(7-bromo-4-(2,2-difluoroethyl)-1-oxophthalazin-2(1H)-yl)acetate mixture. LCMS: m/z = 360.9, 362.9 [M+H]⁺.

2-[6-bromo-4-(2,2-difluoroethyl)-1-oxophthalazin-2-yl]-N-pyrimidin-2-ylacetamide and 2-[7-bromo-4-(2,2-difluoroethyl)-1-oxophthalazin-2-yl]-N-pyrimidin-2-ylacetamide: To a solution of methyl 2-(6-bromo-4-(2,2-difluoroethyl)-1-oxophthalazin-2(1H)-yl)acetate and methyl 2-(7-bromo-4-(2,2-difluoroethyl)-1-oxophthalazin-2(1H)-yl)acetate (1:1 mixture, 70 mg, 0.19 mmol) in toluene (1 mL) and THF (1 mL) was added pyrimidin-2-amine (55 mg, 0.58 mmol) and AlMe₃ (2 M in toluene, 0.29 mL) under N₂. The reaction mixture was stirred at 100° C. for 3 h. The mixture was diluted with water (5 mL) and extracted with EtOAc (3 × 2 mL). The combined organic layers were washed with brine (5 mL), dried over Na₂SO₄, filtered, and concentrated under reduced pressure. The residue was purified by reverse-phase HPLC to provide 5:4 mixture of 2-[6-bromo-4-(2,2-difluoroethyl)-1-oxophthalazin-2-yl]-N-pyrimidin-2- and 2-[7-bromo-4-(2,2-difluoroethyl)-1-oxophthalazin-2-yl]-N-pyrimidin-2-ylacetamide. LCMS: m/z = 424.0, 426.0 [M+H]⁺.

### Examples 101 and 102

### 2-Bromo-4-(1-Hydroxyethyl)-1-Oxophthalazin-2(1H)-yl)-N-(Pyrimidin-2-yl)Acetamide (101 and 102)

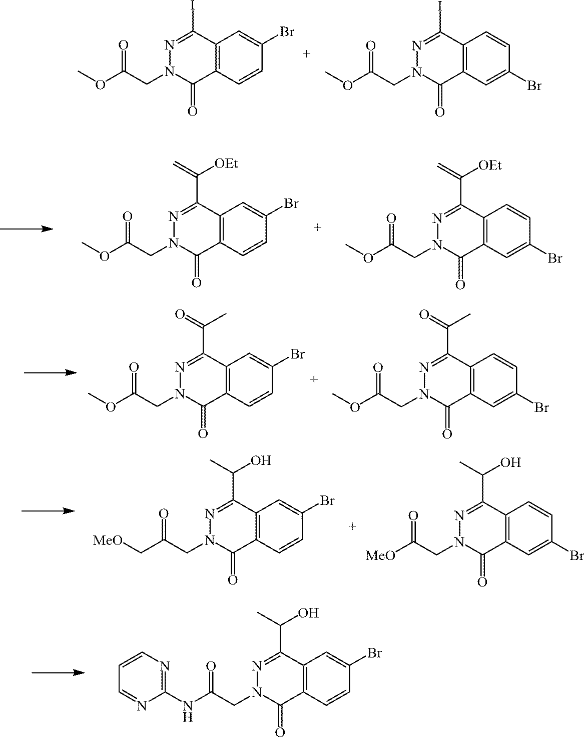

Methyl 2-(6-bromo-4-(1-ethoxyvinyl)-1-oxophthalazin-2(1H)-yl)acetate and methyl 2-(7-bromo-4-(1-ethoxyvinyl)-1-oxophthalazin-2(1H)-yl)acetate: To a solution of methyl 2-(6-bromo-4-iodo-1-oxophthalazin-2(1H)-yl)acetate and methyl 2-(7-bromo-4-iodo-1-oxophthalazin-2(1H)-yl)acetate (1:1 mixture, 1.0 g, 2.36 mmol) in 1,4-dioxane (30 mL) were added tributyl(1-ethoxyvinyl)stannane (854 mg, 2.36 mmol), Pd(PPh₃)₄ (137 mg, 0.12 mmol), LiCl (301 mg, 7.09 mmol), and CuI (1.35 g, 7.09 mmol). The reaction mixture was stirred at 50° C. for 16 h. The reaction mixture was poured into brine (50 mL) and extracted with EtOAc (3 × 30 mL). The combined organic layers were washed with brine (50 mL), dried over anhydrous Na₂SO₄, filtered, and concentrated under reduced pressure. The residue was purified by silica gel column chromatography to provide a 1:1 mixture of methyl 2-(6-bromo-4-(1-ethoxyvinyl)-1-oxophthalazin-2(1H)-yl)acetate and methyl 2-(7-bromo-4-(1-ethoxyvinyl)-1-oxophthalazin-2(1H)-yl)acetate. LCMS: m/z = 336.9, 338.9 [M+H]⁺.

Methyl 2-(4-acetyl-6-bromo-1-oxophthalazin-2(1H)-yl)acetate and methyl 2-(4-acetyl-7-bromo-1-oxophthalazin-2(1H)-yl)acetate: To a solution of methyl 2-(6-bromo-4-(1-ethoxyvinyl)-1-oxophthalazin-2(1H)-yl)acetate and methyl 2-(7-bromo-4-(1-ethoxyvinyl)-1-oxophthalazin-2(1H)-yl)acetate (1:1 mixture, 600 mg, 1.63 mmol) in 1,4-dioxane (5.0 mL) was added aq. HCl (12 N, 2.72 mL). The reaction mixture was stirred at 20° C. for 5 h. The reaction mixture was poured into sat. NaHCO₃ (30 mL) and extracted with EtOAc (3 × 10 mL). The combined organic layers were washed with brine (10 mL), dried over anhydrous Na₂SO₄, filtered, and concentrated under reduced pressure to provide a residue that was used directly as a 1:1 mixture of methyl 2-(4-acetyl-6-bromo-1-oxophthalazin-2(1H)-yl)acetate and methyl 2-(4-acetyl-7-bromo-1-oxophthalazin-2(1H)-yl)acetate.

Methyl 2-(6-bromo-4-(1-hydroxyethyl)-1-oxophthalazin-2(1H)-yl)acetate and methyl 2-(7-bromo-4-(1-hydroxyethyl)-1-oxophthalazin-2(1H)-yl)acetate: To a solution of methyl 2-(4-acetyl-6-bromo-1-oxophthalazin-2(1H)-yl)acetate and methyl 2-(4-acetyl-7-bromo-1-oxophthalazin-2(1H)-yl)acetate (1:1 mixture, 500 mg, 1.47 mmol) in THF (10 mL) at 0° C. was added NaBH₄ (56 mg, 1.47 mmol). The reaction mixture stirred at 20° C. for 5 h. The reaction mixture was poured into brine (10 mL) and extracted with EtOAc (3 × 10 mL). The combined organic layers were washed with brine (10 mL), dried over anhydrous Na₂SO₄, filtered, and concentrated under reduced pressure. The residue was purified by silica gel column chromatography to afford a 1:1 mixture of methyl 2-(6-bromo-4-(1-hydroxyethyl)-1-oxophthalazin-2(1H)-yl)acetate and methyl 2-(7-bromo-4-(1-hydroxyethyl)-1-oxophthalazin-2(1H)-yl)acetate. LCMS: m/z = 341.0, 343.0 [M+H]⁺.

2-(6-bromo-4-(1-hydroxyethyl)-1-oxophthalazin-2(1H)-yl)-N-(pyrimidin-2-yl)acetamide: To a solution of methyl 2-(6-bromo-4-(1-hydroxyethyl)-1-oxophthalazin-2(1H)-yl)acetate and methyl 2-(7-bromo-4-(1-hydroxyethyl)-1-oxophthalazin-2(1H)-yl)acetate (1:1 mixture, 150 mg, 0.44 mmol) in toluene (8.0 mL) and THF (2.0 mL) were added pyrimidin-2-amine (84 mg, 0.88 mmol) and AlMe₃ (0.66 mL, 2 M in toluene). The reaction mixture was stirred at 90° C. for 5 h. The reaction mixture was poured into sat. aq. NH₄Cl (10 mL) and extracted with EtOAc (3 × 10 mL). The combined organic layers were washed with brine (10 mL), dried over anhydrous Na₂SO₄, filtered, and concentrated under reduced pressure. The residue was purified by reverse-phase preparative HPLC followed by preparative chiral SFC (column: Daicel Chiralpak IG (50 mm x 4.6 mm, 3 µM particle size); mobile phase: A: CO₂, B: 0.05% i-PrOH in MeOH; 40% B isocratic; flow rate: 4 mL/min; column temperature: 35° C.; back pressure: 1800 psi) to provide:

2-(6-bromo-4-(1-hydroxyethyl)-1-oxophthalazin-2(1H)-yl)-N-(pyrimidin-2-yl)acetamide (first eluting isomer, 101). LCMS: m/z = 404.0, 406.0 [M+H]⁺. ¹H NMR (400 MHz, CDCl₃): δ 8.79 (s, 1H), 8.62 (d, J= 4.8 Hz, 2H), 8.37 (d, J = 8.4 Hz, 1H), 8.14 (s, 1H), 7.90 (d, J = 8.0 Hz, 1H), 7.06-7.02 (m, 1H), 5.61-5.47 (m, 2H), 5.29-5.16 (m, 1H), 3.01 (s, 1H), 1.65 (d, J = 6.4 Hz, 3H); and

2-(6-bromo-4-(1-hydroxyethyl)-1-oxophthalazin-2(1H)-yl)-N-(pyrimidin-2-yl)acetamide (second eluting isomer, 102). LCMS: m/z = 404.0, 406.0 [M+H]⁺. ¹H NMR (400 MHz, CDCl₃): δ 8.82 (br s, 1H), 8.62 (d, J = 4.8 Hz, 2H), 8.37 (d, J = 8.4 Hz, 1H), 8.14 (s, 1H), 7.90 (d, J = 8.0 Hz, 1H), 7.06-7.02 (m, 1H), 5.61-5.47 (m, 2H), 5.28-5.18 (m, 1H), 3.02 (s, 1H), 1.65 (d, J = 6.4 Hz, 3H).

### Examples 103 and 104

### 2-Bromo-4-(Difluoromethyl)-1-Oxophthalazin-2-yl]-N-(5-Fluoropyrimidin-2-yl)Acetamide (103) and 2-[7-Bromo-4-(Difluoromethyl)-1-Oxophthalazin-2-yl]-N-(5-Fluoropyrimidin-2-yl)Acetamide (104)

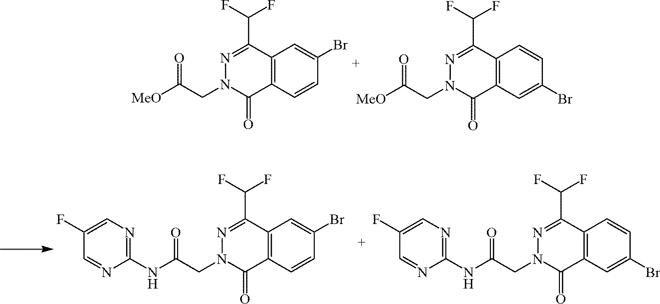

To a solution of methyl 2-(6-bromo-4-(difluoromethyl)-1-oxophthalazin-2(1H)-yl)acetate and methyl 2-(7-bromo-4-(difluoromethyl)-1-oxophthalazin-2(1H)-yl)acetate (1:1 mixture, 110 mg, 0.32 mmol) mixture in toluene (1.0 mL) and THF (1.0 mL) were added 5-fluoropyrimidin-2-amine (72 mg, 0.64 mmol) and AlMe₃ (0.48 mL, 2 M in toluene). The reaction mixture was stirred at 90° C. for 3 h. The reaction mixture was diluted with water (5 mL) and extracted with EtOAc (3 × 2 mL). The combined organic layers were washed with brine (5 mL), dried over anhydrous Na₂SO₄, filtered, and concentrated under reduced pressure. The residue was purified by reverse-phase preparative HPLC followed by preparative SFC to provide:

2-[6-bromo-4-(difluoromethyl)-1-oxophthalazin-2-yl]-N-(5-fluoropyrimidin-2-yl)acetamide: LCMS: m/z = 428.0, 430.0 [M+H]⁺. ¹H NMR (400 MHz, CDCl₃): δ 8.62 (br s, 1H), 8.49 (s, 2H), 8.36 (d, J = 8.4 Hz, 1H), 8.33 (s, 1H), 7.95 (dd, J = 1.6, 8.4 Hz, 1H), 6.61 (t, J = 53.2 Hz, 1H), 5.51 (s, 2H); and

2-[7-bromo-4-(difluoromethyl)-1-oxophthalazin-2-yl]-N-(5-fluoropyrimidin-2-yl)acetamide: LCMS: m/z = 428.0, 430.0 [M+H]⁺. ¹H NMR (400 MHz, CDCl₃): δ 8.65 (d, J = 1.6 Hz, 1H), 8.56 (br s, 1H), 8.49 (s, 2H), 8.09-8.04 (m, 1H), 8.02-7.97 (m, 1H), 6.61 (t, J = 53.2 Hz, 1H), 5.51 (s, 2H).

### Example 105

### 2-(Difluoromethyl)-5-Oxo-2-(Trifluoromethyl)Pyrido[2,3-d]Pyridazin-6-yl]-N-(5-Fluoropyrimidin-2-yl)Acetamide (105)

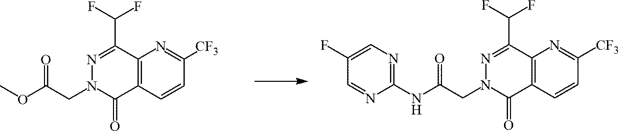 [SMILES: COC(=O)Cn1nc(C(F)F)c2nc(C(F)(F)F)ccc2c1=O ; O=C(Cn1nc(C(F)F)c2nc(C(F)(F)F)ccc2c1=O)Nc1ncc(F)cn1]

2-[8-(difluoromethyl)-5-oxo-2-(trifluoromethyl)pyrido[2,3-d]pyridazin-6-yl]-N-(5-fluoropyrimidin-2-yl)acetamide: To a solution of methyl 2-(8-(difluoromethyl)-5-oxo-2-(trifluoromethyl)pyrido[2,3-d]pyridazin-6(5H)-yl)acetate (45 mg, 0.13 mmol) and 5-fluoropyrimidin-2-amine (30 mg, 0.27 mmol) in toluene (2.0 mL) was added DABAL-Me₃ (68 mg, 0.27 mmol). The reaction mixture was stirred at 60° C. for 4 h. The reaction mixture was diluted with water (5 mL) and extracted with EtOAc (3 × 10 mL). The combined organic layers were washed with brine (3 mL), dried over anhydrous Na₂SO₄, filtered, and concentrated under reduced pressure. The residue was purified by reverse-phase preparative HPLC. LCMS: m/z = 419.0 [M+H]⁺. ¹H NMR (400 MHz, CDCl₃): δ 8.96 (d, J = 8.8 Hz, 1H), 8.76 (s, 1H), 8.54 (s, 2H), 8.14 (d, J= 2.8 Hz, 1H), 7.27 (t, J = 52.8 Hz, 1H), 5.68 (s, 2H).

### Example 106

### 2-Bromo-1-Oxo-4-Propan-2-Ylphthalazin-2-yl)-N-[(3R)-1-Cyclobutylpiperidin-3-yl] Acetamide (106)

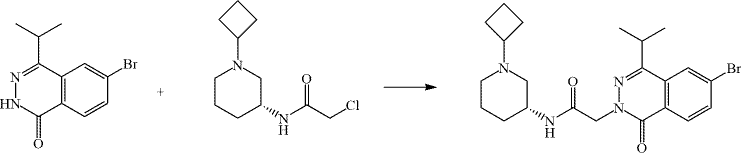

2-(6-bromo-1-oxo-4-propan-2-ylphthalazin-2-yl)-N-[(3R)-1-cyclobutylpiperidin-3-yl]acetamide: To a solution of (R)-2-chloro-N-(1-cyclobutylpiperidin-3-yl)acetamide (82 mg, 0.36 mmol) and 6-bromo-4-isopropylphthalazin-1(2H)-one (73 mg, 0.27 mmol) in DMF (3 mL) was added Cs₂CO₃ (224 mg, 0.68 mmol). The reaction mixture was stirred at 80° C. for 3 h. The reaction mixture was diluted with ice-cold water (15 mL) and extracted with EtOAc (3 × 10 mL). The combined organic layers were washed with brine (10 mL), dried over anhydrous Na₂SO₄, filtered, and concentrated under reduced pressure. The crude residue was purified by reverse-phase preparative HPLC. LCMS: m/z = 461.0, 463.0 [M+H]⁺. ¹H NMR (400 MHz, DMSO-d₆): δ 8.29 (d, J = 1.8 Hz, 1H), 8.21 (d, J = 8.5 Hz, 1H), 8.05 (dd, J = 8.5, 1.8 Hz, 1H), 7.90 (d, J = 7.7, 1H), 4.68 (d, J = 1.7 Hz, 2H), 3.72-3.66 (m, 1H), 3.63-3.58 (m, 1H), 3.29-3.26 (m, 1H), 2.68-2.57 (m, 2H), 1.94-1.88 (m, 2H), 1.78-1.76 (m, 1H), 1.71-1.54 (m, 8H), 1.45-1.38 (m, 1H), 1.24 (d, J = 6.7 Hz, 6H).

### Example 107

### Tert-Butyl 2-[[[2-(6-Bromo-4-Isopropyl-1-Oxo-Phthalazin-2-yl)Acetyl]Amino]Methyl]Pyrrolidine-1-Carboxylate (107)

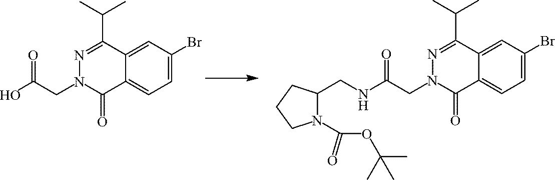 [SMILES: CC(C)c1nn(CC(=O)O)c(=O)c2ccc(Br)cc12 ; CC(C)c1nn(CC(=O)NCC2CCCN2C(=O)OC(C)(C)C)c(=O)c2ccc(Br)cc12]

tert-butyl 2-[[[2-(6-bromo-4-isopropyl-1-oxo-phthalazin-2-yl)acetyl]amino]methyl]pyrrolidine-1-carboxylate: To a mixture of 2-(6-bromo-4-isopropyl-1-oxo-phthalazin-2(1H)-yl)acetic acid (100 mg, 0.31 mmol) and tert-butyl 2-(aminomethyl)pyrrolidine-1-carboxylate (86 mg, 0.43 mmol) in THF (3.0 mL) were added DIPEA (119 mg, 0.92 mmol) and T3P (98 mg, 0.31 mmol, 50% in EtOAc). The reaction mixture was stirred at 23° C. for 1 h. The reaction mixture was poured into ice-cold water (10 mL) and extracted with EtOAc (2 × 5 mL). The organic layers were combined and washed with brine (10 mL), dried over anhydrous Na₂SO₄, filtered, and concentrated under reduced pressure. The residue was purified by reverse-phase preparative HPLC. LCMS: m/z = 507.3, 509.3 [M+H]⁺. ¹H NMR (400 MHz, DMSO-d₆): δ 8.29 (d, J = 1.8 Hz, 1H), 8.22-8.20 (m, 1H), 8.15 (d, J = 8.6, 1H), 8.04 (dd, J = 8.6, 1.8 Hz, 1H), 4.71 (s, 2H), 3.75-3.72 (m, 1H), 3.65-3.57 (m, 1H), 3.26-3.17 (m, 3H), 1.83-1.69 (m, 5H), 1.37 (d, J = 9.6 Hz, 9H), 1.24 (d, J= 6.7 Hz, 6H).

### Example 108

### 2-Bromo-1-Oxo-4-Propan-2-Ylphthalazin-2-yl)-N-(Pyrrolidin-2-Ylmethyl)Acetamide HCl Salt (108)

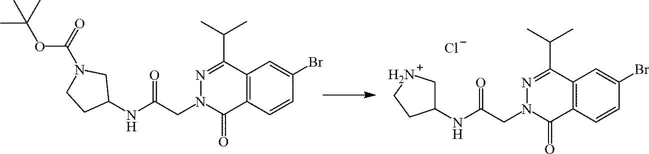 [SMILES: CC(C)c1nn(CC(=O)NC2CCN(C(=O)OC(C)(C)C)C2)c(=O)c2ccc(Br)cc12 ; CC(C)c1nn(CC(=O)NC2CC[NH2+]C2)c(=O)c2ccc(Br)cc12 ; [Cl-]]

2-(6-bromo-1-oxo-4-propan-2-ylphthalazin-2-yl)-N-(pyrrolidin-2-ylmethyl)acetamide HCl salt: A solution of tert-butyl 2-[[[2-(6-bromo-4-isopropyl-1-oxo-phthalazin-2-yl)acetyl]amino]methyl]pyrrolidine-1-carboxylate (110 mg, 0.21 mmol) in HCl (10 mL, 4 N in dioxane) was stirred at 20° C. for 1 h. The reaction mixture was concentrated under reduced pressure to provide a residue that was used directly. LCMS: m/z = 407.4, 409.4 [M+H]⁺. ¹H NMR (400 MHz, DMSO-d₆): δ 9.47-9.42 (m, 1H), 8.91-8.90 (m, 1H), 8.56-8.53 (m, 1H), 8.29 (d, J = 1.8 Hz, 1H), 8.20 (d, J = 8.5 Hz, 1H), 8.05 (dd, J = 8.5, 1.8 Hz, 1H), 4.75-4.73 (m, 2H), 3.63-3.58 (m, 2H), 3.44-3.39 (m, 2H), 3.15-3.11 (m, 2H), 2.01-1.80 (m, 3H), 1.69-1.62 (m, 1H), 1.25 (d, J = 6.7 Hz, 6H).

### Example 109

### 2-Bromo-1-Oxo-4-Propan-2-Ylphthalazin-2-yl)-N-[(1-Ethylpyrrolidin-2-yl)Methyl]Acetamide (109)

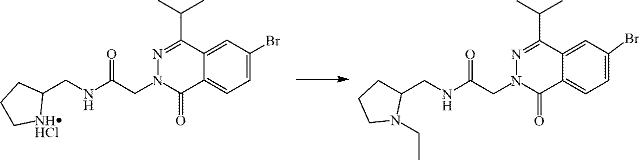

2-(6-bromo-1-oxo-4-propan-2-ylphthalazin-2-yl)-N-[(1-ethylpyrrolidin-2-yl)methyl]acetamide: To a solution of 2-(6-bromo-1-oxo-4-propan-2-ylphthalazin-2-yl)-N-(pyrrolidin-2-ylmethyl)acetamide HCl salt (100 mg, 0.23 mmol) and iodoethane (42 mg, 0.27 mmol) in MeCN (7.0 mL) was added K₂CO₃ (93 mg, 0.68 mmol). The reaction mixture was stirred at 60° C. for 18 h. The reaction mixture was poured into ice-cold water (10 mL) and extracted with EtOAc (2 × 5 mL). The organic layers were combined and washed with brine (10 mL), dried over anhydrous Na₂SO₄, filtered, and concentrated under reduced pressure. The residue was purified by reverse-phase HPLC. LCMS: m/z = 435.6, 437.6 [M+H]⁺. ¹H NMR (400 MHz, DMSO-d₆): δ 8.29 (d, J = 1.8 Hz, 1H), 8.20 (d, J= 8.2 Hz, 1H), 8.04 (dd, J = 8.5, 1.8 Hz, 2H), 4.70 (d, J = 2.7 Hz, 2H), 3.64-3.58 (m, 1H), 3.33-3.27 (m, 1H), 3.08-3.03 (m, 1H), 2.98-2.94 (m, 1H), 2.86-2.79 (m, 1H), 2.61-2.55 (m, 1H), 2.35-2.27 (m, 1H), 2.23-2.19 (m, 1H), 1.85-1.76 (m, 1H), 1.68-1.61 (m, 2H), 1.55-1.48 (m, 1H), 1.24 (d, J = 6.7 Hz, 6H), 1.02 (t, J = 7.2 Hz, 3H).

### Example 110

### 2-Bromo-1-Oxo-4-Propan-2-Ylphthalazin-2-yl)-N-[4-(Trifluoromethyl)Pyrimidin-2-yl]Acetamide (110)

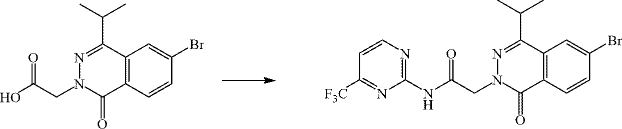 [SMILES: CC(C)c1nn(CC(=O)O)c(=O)c2ccc(Br)cc12 ; CC(C)c1nn(CC(=O)Nc2nccc(C(F)(F)F)n2)c(=O)c2ccc(Br)cc12]

2-(6-bromo-1-oxo-4-propan-2-ylphthalazin-2-yl)-N-[4-(trifluoromethyl)pyrimidin-2-yl]acetamide: To a solution of 2-(6-bromo-4-isopropyl-1-oxo-phthalazin-2-yl)acetic acid (43 mg, 0.13 mmol) in MeCN (1.5 mL) were added 4-(trifluoromethyl)pyrimidin-2-amine (28 mg, 0.17 mmol), 1-methylimidazole (44 mg, 0.53 mmol), and chloro-N,N,N′,N′-tetramethylformamidinium hexafluorophosphate (45 mg, 0.16 mmol). The reaction mixture was stirred at 23° C. for 20 h. The reaction mixture was purified directly by reverse-phase preparative HPLC. LCMS: m/z = 470.3, 472.2 [M+H]⁺. ¹H NMR (400 MHz, CDCl₃): δ 8.87 (d, J = 5.0 Hz, 1H), 8.77-8.76 (m, 1H), 8.35 (d, J = 8.5 Hz, 1H), 8.02 (d, J = 1.8 Hz, 1H), 7.86 (dd, J = 8.5, 1.8 Hz, 1H), 7.34 (d, J = 5.0 Hz, 1H), 5.46 (s, 2H), 3.46-3.38 (m, 1H), 1.36 (d, J = 6.8 Hz, 6H).

The following compound was, or can be, made via similar procedures as those described above.

| Ex. | Structure | Name | NMR | LCMS |
|---|---|---|---|---|
| 111 | 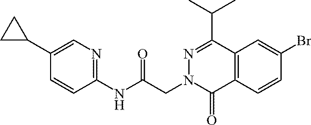 [SMILES: CC(C)c1nn(CC(=O)Nc2ccc(C3CC3)cn2)c(=O)c2ccc(Br)cc12] | 2-(6-bromo-1-oxo-4-propan-2-ylphthalazin-2-yl)-N-(5-cyclopropylpyridin-2-yl)acetamide | ¹H NMR (400 MHz, DMSO-d₆): δ 10.44 (s, 1H), 8.57 (d, J = 2.5 Hz, 1H), 8.31 (d, J = 1.8 Hz, 1H), 8.21 (d, J = 8.5 Hz, 1H), 8.06 (dd, J = 8.5, 1.8 Hz, 1H), 7.89 (dd, J = 8.5, 2.5 Hz, 1H), 7.27 (d, J = 8.3 Hz, 1H), 4.93 (s, 2H), 3.66-3.60 (m, 1H), 2.08-2.04 (m, 1H), 1.25 (t, J = 6.2 Hz, 6H), 0.95-0.91 (m, 2H), 0.89-0.85 (m, 2H) | m/z = 441.3, 443.3 [M+H]⁺ |
| 112 | 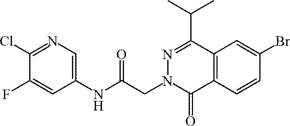 [SMILES: CC(C)c1nn(CC(=O)Nc2cnc(Cl)c(F)c2)c(=O)c2ccc(Br)cc12] | 2-(6-bromo-1-oxo-4-propan-2-ylphthalazin-2-yl)-N-(6-chloro-5-fluoropyridin-3-yl)acetamide | ¹H NMR (400 MHz, CDCl₃): δ 9.45 (s, 1H), 8.36 (d, J = 8.5 Hz, 1H), 8.14 (dd, J = 9.8, 2.3 Hz, 1H), 8.10 (d, J = 2.3 Hz, 1H), 8.06 (d, J = 1.8 Hz, 1H), 7.92 (dd, J = 8.5, 1.8 Hz, 1H), 5.06 (s, 2H), 3.51-3.40 (m, 1H), 1.36 (d, J = 6.8 Hz, 6H) | m/z = 453.3, 455.3, 457.2 [M+H]⁺ |
| 113 | 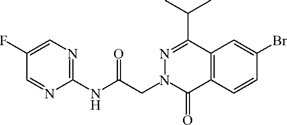 [SMILES: CC(C)c1nn(CC(=O)Nc2ncc(F)cn2)c(=O)c2ccc(Br)cc12] | 2-(6-bromo-1-oxo-4-propan-2-ylphthalazin-2-yl)-N-(5-fluoropyrimidin-2-yl)acetamide | ¹H NMR (400 MHz, CDCl₃): δ 9.02 (s, 1H), 8.48 (s, 2H), 8.37 (d, J = 8.5 Hz, 1H), 8.02 (d, J = 1.8 Hz, 1H), 7.86 (dd, J = 8.5, 1.8 Hz, 1H), 5.35 (s, 2H), 3.48-3.38 (m, 1H), 1.35 (d, J = 6.8 Hz, 6H) | m/z = 420.4, 422.3 [M+H]⁺ |
| 114 | 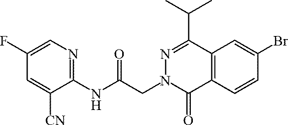 [SMILES: CC(C)c1nn(CC(=O)Nc2ncc(F)cc2C#N)c(=O)c2ccc(Br)cc12] | 2-(6-bromo-1-oxo-4-propan-2-ylphthalazin-2-yl)-N-(3-cyano-5-fluoropyridin-2-yl)acetamide | ¹H NMR (400 MHz, CDCl₃): δ 9.12 (s, 1H), 8.46 (d, J = 2.5 Hz, 1H), 8.38 (d, J = 8.5 Hz, 1H), 8.04 (d, J = 1.8 Hz, 1H), 7.89 (dd, J = 8.5, 1.8 Hz, 1H), 7.71 (dd, J = 7.0, 2.9 Hz, 1H), 5.19 (s, 2H), 3.47-3.39 (m, 1H), 1.37 (d, J = 6.8 Hz, 6H) | m/z = 444.3, 446.3 [M+H]⁺ |
| 115 | 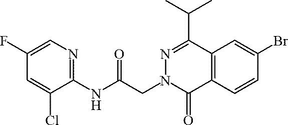 [SMILES: CC(C)c1nn(CC(=O)Nc2ncc(F)cc2Cl)c(=O)c2ccc(Br)cc12] | 2-(6-bromo-1-oxo-4-propan-2-ylphthalazin-2-yl)-N-(3-chloro-5-fluoropyridin-2-yl)acetamide | ¹H NMR (400 MHz, CDCl₃): δ 8.65 (br s, 1H), 8.35 (d, J = 8.5, 1H), 8.22 (d, J = 2.6 Hz, 1H), 8.02 (d, J = 1.8 Hz, 1H), 7.86 (dd, J = 8.5, 1.8 Hz, 1H), 7.53 (dd, J = 7.3, 2.7 Hz, 1H), 5.29 (s, 2H), 3.46-3.38 (m, 1H), 1.35 (d, J = 6.8 Hz, 6H) | m/z = 453.2, 455.3, 457.2 [M+H]⁺ |
| 116 | 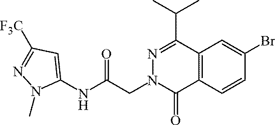 [SMILES: CC(C)c1nn(CC(=O)Nc2cc(C(F)(F)F)nn2C)c(=O)c2ccc(Br)cc12] | 2-(6-bromo-1-oxo-4-propan-2-ylphthalazin-2-yl)-N-[2-methyl-5-(trifluoromethyl)pyrazol -3-yl]acetamide | ¹H NMR (400 MHz, CDCl₃): δ 9.56 (s, 1H), 8.37 (d, J = 8.5 Hz, 1H), 8.08 (d, J = 1.7 Hz, 1H), 7.94 (s, 1H), 6.68 (s, 1H), 5.06 (s, 2H), 3.80 (s, 3H), 3.49-3.42 (m, 1H), 1.37 (d, J = 6.8 Hz, 6H) | m/z = 472.4, 474.3 [M+H]⁺ |
| 117 | 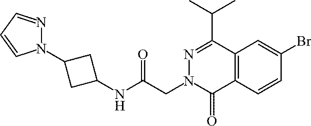 [SMILES: CC(C)c1nn(CC(=O)NC2CC(n3cccn3)C2)c(=O)c2ccc(Br)cc12] | 2-(6-bromo-1-oxo-4-propan-2-ylphthalazin-2-yl)-N-(3-pyrazol-1-ylcyclobutyl)acetamide | ¹H NMR (400 MHz, CDCl₃): δ 8.32 (d, J = 8.5 Hz, 1H), 8.01 (d, J = 1.8 Hz, 1H), 7.86 (dd, J = 8.5, 1.8 Hz, 1H), 7.54-7.54 (m, 1H), 7.45 (d, J = 2.2 Hz, 1H), 6.77-6.75 (m, 1H), 6.26 (t, J = 2.1 Hz, 1H), 4.99-4.95 (m, 1H), 4.84 (s, 2H), 4.56-4.50 (m, 1H), 3.45-3.38 (m, 1H), 2.93-2.85 (m, 2H), 2.61-2.54 (m, 2H), 1.34 (d, J = 6.8 Hz, 6H) | m/z = 444.5, 446.4 [M+H]⁺ |
| 118 | 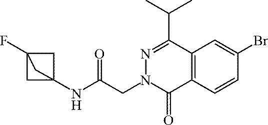 [SMILES: CC(C)c1nn(CC(=O)NC23CC(F)(C2)C3)c(=O)c2ccc(Br)cc12] | 2-(6-bromo-1-oxo-4-propan-2-ylphthalazin-2-yl)-N-(3-fluoro-1-bicyclo[1.1.1]pentanyl)a cetamide | ¹H NMR (400 MHz, CDCl₃): δ 8.33 (d, J = 8.5 Hz, 1H), 8.02 (d, J = 1.8 Hz, 1H), 7.88 (dd, J = 8.5, 1.8 Hz, 1H), 6.74 (s, 1H), 4.82 (s, 2H), 3.47-3.37 (m, 1H), 2.40 (d, J = 2.1 Hz, 6H), 1.35 (d, J = 6.8 Hz, 6H) | m/z = 408.4, 410.3 [M+H]⁺ |
| 119 | 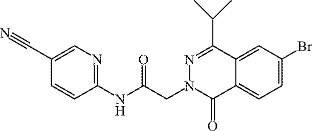 [SMILES: CC(C)c1nn(CC(=O)Nc2ccc(C#N)cn2)c(=O)c2ccc(Br)cc12] | 2-(6-bromo-1-oxo-4-propan-2-ylphthalazin-2-yl)-N-(5-cyanopyridin-2-yl)acetamide | ¹H NMR (400 MHz, CDCl₃): δ 9.14 (s, 1H), 8.54 (dd, J = 2.3, 0.8 Hz, 1H), 8.38 (d, J = 8.6 Hz, 1H), 8.33 (dd, J = 8.8, 0.7 Hz, 1H), 8.04 (d, J = 1.8 Hz, 1H), 7.94-7.89 (m, 2H), 5.06 (s, 2H), 3.50-3.42 (m, 1H), 1.38 (d, J = 6.8 Hz, 6H) | m/z = 426.5, 428.5 [M+H]⁺ |
| 120 | 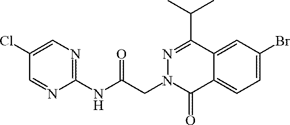 [SMILES: CC(C)c1nn(CC(=O)Nc2ncc(Cl)cn2)c(=O)c2ccc(Br)cc12] | 2-(6-bromo-1-oxo-4-propan-2-ylphthalazin-2-yl)-N-(5-chloropyrimidin-2-yl)acetamide | ¹H NMR (400 MHz, CDCl₃): δ 8.90 (s, 1H), 8.57 (s, 2H), 8.39 (dd, J = 8.5, 0.4 Hz, 1H), 8.04 (d, J = 1.8 Hz, 1H), 7.89 (dd, J = 8.5, 1.8 Hz, 1H), 5.40-5.39 (m, 2H), 3.45 (dt, J = 13.6, 6.8 Hz, 1H), 1.38 (d, J = 6.8 Hz, 6H) | m/z = 436.2, 438.2 [M+H]⁺ |

### Example 121

### 2-Bromo-1-Oxo-4-Propan-2-Ylphthalazin-2-yl)-N-Pyridazin-4-Ylacetamide: (121)

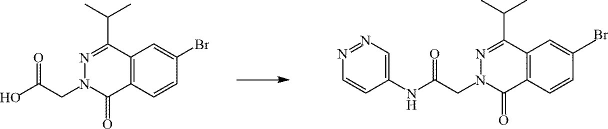 [SMILES: CC(C)c1nn(CC(=O)O)c(=O)c2ccc(Br)cc12 ; Cc1ccc2c(=O)n(CC(=O)Nc3ccnnc3)nc(C(C)C)c2c1 ; Br]

2-(6-bromo-1-oxo-4-propan-2-ylphthalazin-2-yl)-N-pyridazin-4-ylacetamide: To a solution of 2-(6-bromo-4-isopropyl-1-oxo-phthalazin-2-yl)acetic acid (23 mg, 0.07 mmol) in DCM (1.0 mL) were added bis(tetramethylene)fluoroformamidinium hexafluorophosphate (30 mg, 0.09 mmol) and DIPEA (37 mg, 0.28 mmol). The reaction mixture was stirred at 23° C. for 30 min. To the reaction mixture was added pyridazin-4-amine (6 mg, 0.06 mmol). The reaction mixture was stirred at 80° C. for 30 min. The reaction mixture was diluted with water (5 mL) and extracted with EtOAc (2 × 10 mL). The combined organic layers were dried over anhydrous Na₂SO₄, filtered, and concentrated under reduced pressure. The residue was purified by reverse-phase preparative HPLC. LCMS: m/z = 402.3, 404.2 [M+H]⁺. ¹H NMR (400 MHz, CDCl₃): δ 10.63-10.62 (m, 1H), 9.30 (d, J = 2.3 Hz, 1H), 9.00 (d, J = 5.8 Hz, 1H), 8.32 (d, J = 8.5 Hz, 1H), 8.18 (dd, J = 6.0, 2.6 Hz, 1H), 8.03 (d, J = 1.8 Hz, 1H), 7.88 (dd, J = 8.5, 1.8 Hz, 1H), 5.18 (s, 2H), 3.45-3.39 (m, 1H), 1.35 (d, J = 6.8 Hz, 6H).

The following compound was, or can be, made via similar procedures as those described above.

| Ex. | Structure | Name | NMR | LCMS |
|---|---|---|---|---|
| 122 | 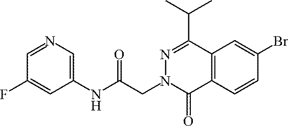 [SMILES: CC(C)c1nn(CC(=O)Nc2cncc(F)c2)c(=O)c2ccc(Br)cc12] | 2-(6-bromo-1-oxo-4-propan-2-ylphthalazin-2-yl)-N-(5-fluoropyridin-3-yl)acetamide | ¹H NMR (400 MHz, CDCl₃): δ 9.55 (s, 1H), 8.35 (s, 1H), 8.32 (d, J = 8.5 Hz, 1H), 8.13 (s, 1H), 8.09 (dd, J = 10.4, 1.6 Hz, 1H), 8.02 (d, J = 1.7 Hz, 1H), 7.87 (dd, J = 8.5, 1.6 Hz, 1H), 5.06 (s, 2H), 3.48-3.38 (m, 1H), 1.36 (d, J = 6.8 Hz, 6H) | m/z = 419.3, 421.2 [M+H]⁺ |
| 123 | 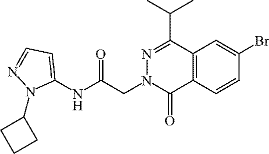 [SMILES: CC(C)c1nn(CC(=O)Nc2ccnn2C2CCC2)c(=O)c2ccc(Br)cc12] | 2-(6-bromo-1-oxo-4-propan-2-ylphthalazin-2-yl)-N-(2-cyclobutylpyrazol-3-yl)acetamide | ¹H NMR (400 MHz, CDCl₃): δ 9.24 (s, 1H), 8.38 (d, J = 8.4 Hz, 1H), 8.07 (s, 1H), 7.93 (d, J = 8.7, 1H), 7.53 (s, 1H), 6.45 (s, 1H), 5.07 (s, 2H), 4.70-4.65 (m, 1H), 3.49-3.42 (m, 1H), 2.68-2.63 (m, 2H), 2.43-2.40 (m, 2H), 1.92-1.84 (m, 2H), 1.37 (d, J = 6.8 Hz, 6H) | m/z = 444.5, 446.4 [M+H]⁺ |
| 124 | 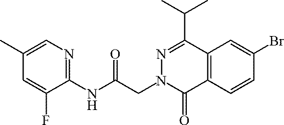 [SMILES: Cc1cnc(NC(=O)Cn2nc(C(C)C)c3cc(Br)ccc3c2=O)c(F)c1] | 2-(6-bromo-1-oxo-4-propan-2-ylphthalazin-2-yl)-N-(3-fluoro-5-methylpyridin-2-yl)acetamide | ¹H NMR (400 MHz, CDCl₃): δ 8.35 (d, J = 8.5 Hz, 1H), 8.02 (d, J = 1.6 Hz, 2H), 7.86 (dd, J = 8.5, 1.7 Hz, 1H), 7.34-7.32 (m, 1H), 5.30 (s, 2H), 5.28-5.25 (m, 1H), 3.46-3.39 (m, 1H), 2.34 (s, 3H), 1.36 (d, J = 6.8 Hz, 6H) | m/z = 433.4, 435.3 [M+H]⁺ |

### Example 125

### 2-Bromo-1-Oxo-4-Propan-2-Ylphthalazin-2-yl)-N-(Oxan-3-yl)Acetamide (125)

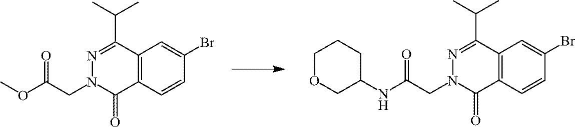 [SMILES: COC(=O)Cn1nc(C(C)C)c2cc(Br)ccc2c1=O ; CC(C)c1nn(CC(=O)NC2CCCOC2)c(=O)c2ccc(Br)cc12]

2-(6-bromo-1-oxo-4-propan-2-ylphthalazin-2-yl)-N-(oxan-3-yl)acetamide: To a solution of tetrahydro-pyran-3-ylamine HCl salt (61 mg, 0.44 mmol) in THF (1.5 mL) at 0° C. was added isopropyl magnesium chloride (2 M in THF, 0.75 mL). The reaction mixture was stirred at 0° C. for 15 min. To the reaction mixture was added methyl 2-(6-bromo-4-isopropyl-1-oxo-phthalazin-2-yl)acetate (50 mg, 0.15 mmol). The reaction mixture was stirred at 23° C. for 16 h. The reaction mixture was diluted with water (5 mL) and extracted with EtOAc (2 × 10 mL). The combined organic layers were washed with brine (5 mL), dried over anhydrous Na₂SO₄, filtered, and concentrated under reduced pressure. The residue was purified by reverse-phase preparative HPLC. LCMS: m/z = 408.4, 410.3 [M+H]⁺. ¹H NMR (400 MHz, CDCl₃): δ 8.34 (d, J = 8.5 Hz, 1H), 8.01 (d, J= 1.6 Hz, 1H), 7.88-7.86 (m, 1H), 6.48-6.42 (m, 1H), 4.86 (q, J = 13.3 Hz, 2H), 4.04-3.98 (m, 1H), 3.72 (dd, J = 11.4, 2.8 Hz, 1H), 3.67-3.61 (m, 1H), 3.59-3.53 (m, 1H), 3.47-3.39 (m, 2H), 1.83-1.78 (m, 1H), 1.74-1.71 (m, 1H), 1.70-1.66 (m, 1H), 1.56-1.51 (m, 1H), 1.36 (d, J = 6.8 Hz, 6H).

### Example 126

### 2-Bromo-1-Oxo-4-Propan-2-Ylphthalazin-2-yl)-N-Cyclobutylacetamide (126)

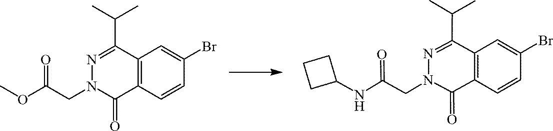 [SMILES: COC(=O)Cn1nc(C(C)C)c2cc(Br)ccc2c1=O ; CC(C)c1nn(CC(=O)NC2CCC2)c(=O)c2ccc(Br)cc12]

2-(6-bromo-1-oxo-4-propan-2-ylphthalazin-2-yl)-N-cyclobutylacetamide: To a solution of methyl 2-(6-bromo-4-(difluoromethyl)-5-fluoro-1-oxophthalazin-2(1H)-yl)acetate (34 mg, 0.10 mmol) in MeOH (1 mL) was added cyclobutylamine (21 mg, 0.30 mmol). The reaction mixture was heated at 80° C. for 72 h. The reaction mixture was concentrated under reduced pressure and purified by reverse-phase HPLC. LCMS: m/z = 378.4, 380.3 [M+H]⁺. ¹H NMR (400 MHz, DMSO-d₆): δ 8.32 (d, J= 7.9 Hz, 1H), 8.28 (d, J = 1.8 Hz, 1H), 8.19 (d, J= 8.5 Hz, 1H), 8.03 (dd, J = 8.5, 1.8 Hz, 1H), 4.64 (s, 2H), 4.20 (sextet, J = 8.1 Hz, 1H), 3.60 (dt, J = 13.5, 6.7 Hz, 1H), 2.18-2.11 (m, 2H), 1.95-1.85 (m, 2H), 1.65-1.58 (m, 2H), 1.23 (d, J = 6.7 Hz, 6H).

The following compound was, or can be, made via similar procedures as those described above.

| Ex. | Structure | Name | NMR | LCMS |
|---|---|---|---|---|
| 127 | 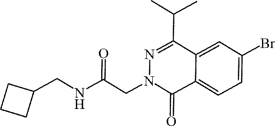 [SMILES: CC(C)c1nn(CC(=O)NCC2CCC2)c(=O)c2ccc(Br)cc12] | 2-(6-bromo-1-oxo-4-propan-2-ylphthalazin-2-yl)-N-(cyclobutylmethyl)aceta mide | ¹H NMR (400 MHz, CDCl₃): δ 8.36 (d, J = 8.4 Hz, 1H), 8.04 (d, J = 1.8 Hz, 1H), 7.89 (dd, J = 8.5, 1.8 Hz, 1H), 6.16 (s, 1H), 4.87 (s, 2H), 3.44 (7, J = 6.8 Hz, 1H), 3.31 (dd, J = 7.1, 5.8 Hz, 2H), 2.46 (dquintet, J = 15.2, 7.6 Hz, 1H), 2.05-1.97 (m, 2H), 1.91-1.80 (m, 2H), 1.70-1.62 (m, 2H), 1.37 (d, J = 6.8 Hz, 6H) | m/z = 392.4, 394.4 [M+H]⁺ |
| 128 | 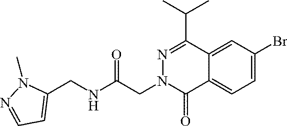 [SMILES: CC(C)c1nn(CC(=O)NCc2ccnn2C)c(=O)c2ccc(Br)cc12] | 2-(6-bromo-1-oxo-4-propan-2-ylphthalazin-2-yl)-N-[(2-methylpyrazol-3-yl)methyl]acetamide | ¹H NMR (400 MHz, DMSO-d₆): δ 8.62-8.54 (m, 1H), 8.33-8.28 (m, 1H), 8.28-8.19 (m, 1H), 8.09-8.03 (m, 1H), 7.35-7.29 (m, 1H), 6.18-6.14 (m, 1H), 4.79-4.71 (m, 2H), 4.39-4.33 (m, 2H), 3.81-3.71 (m, 3H), 3.66-3.56 (m, 1H), 1.27-1.18 (m, 6H) | m/z = 418.6, 420.5 [M+H]⁺ |
| 129 | 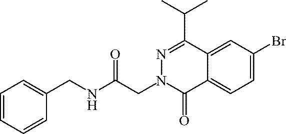 [SMILES: CC(C)c1nn(CC(=O)NCc2ccccc2)c(=O)c2ccc(Br)cc12] | N-benzyl-2-(6-bromo-1-oxo-4-propan-2-ylphthalazin-2-yl)acetamide | ¹H NMR (400 MHz, DMSO-d₆): δ 8.60-8.57 (m, 1H), 8.29 (d, J = 1.8 Hz, 1H), 8.22 (d, J = 8.5 Hz, 1H), 8.05 (dd, J = 8.5, 1.8 Hz, 1H), 7.35-7.22 (m, 5H), 4.77 (s, 2H), 4.32 (d, J = 6.0 Hz, 2H), 3.62 (dt, J = 13.6, 6.8 Hz, 1H), 1.25 (d, J = 6.7 Hz, 6H) | m/z = 414.4, 416.3 [M+H]⁺ |
| 130 | 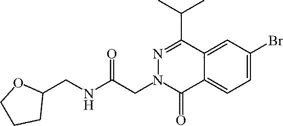 [SMILES: CC(C)c1nn(CC(=O)NCC2CCCO2)c(=O)c2ccc(Br)cc12] | 2-(6-bromo-1-oxo-4-propan-2-ylphthalazin-2-yl)-N-(oxolan-2-ylmethyl)acetamide | ¹H NMR (400 MHz, CDCl₃): δ 8.36 (d, J = 8.5 Hz, 1H), 8.03 (d, J = 1.7 Hz, 1H), 7.88 (dd, J = 8.5, 1.8 Hz, 1H), 6.39-6.36 (m, 1H), 4.89 (s, 2H), 4.00-3.94 (m, 1H), 3.81-3.68 (m, 2H), 3.60-3.54 (m, 1H), 3.47-3.41 (m, 1H), 3.30-3.23 (m, 1H), 2.00-1.92 (m, 1H), 1.91-1.83 (m, 2H), 1.59-1.54 (m, 1H), 1.37 (d, J = 6.8 Hz, 6H) | m/z = 408.4, 410.3 [M+H]⁺ |

### Examples 131 and 132

### 2-Bromo-1-Oxo-4-(Trifluoromethyl)Phthalazin-2-yl]-N-(5-Fluoropyrimidin-4-yl)Acetamide (131) and 2-[7-Bromo-1-Oxo-4-(Trifluoromethyl)Phthalazin-2-yl]-N-(5-Fluoropyrimidin-4-yl)Acetamide (132)

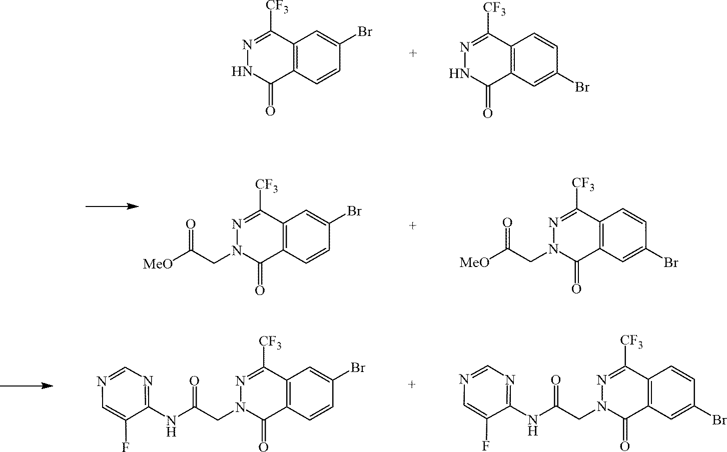

methyl 2-(6-bromo-1-oxo-4-(trifluoromethyl)phthalazin-2(1H)-yl)acetate and methyl 2-(7-bromo-1-oxo-4-(trifluoromethyl)phthalazin-2(1H)-yl)acetate: To a solution of 6-bromo-4-(trifluoromethyl)phthalazin-1(2H)-one and 7-bromo-4-(trifluoromethyl)phthalazin-1(2H)-one (1:1 mixture, 400 mg, 1.37 mmol) in DMF (5.0 mL) were added Cs₂CO₃ (890 mg, 2.73 mmol) and methyl 2-bromoacetate (251 mg, 1.64 mmol). The reaction mixture was stirred at 20° C. for 3 h. The reaction mixture was diluted with water (10 mL) and extracted with EtOAc (3 × 5 mL). The combined organic layers were dried over anhydrous Na₂SO₄, filtered, and concentrated under reduced pressure. The residue was purified by silica gel column chromatography to provide a 1:1 mixture of methyl 2-(6-bromo-1-oxo-4-(trifluoromethyl)phthalazin-2(1H)-yl)acetate and methyl 2-(7-bromo-1-oxo-4-(trifluoromethyl)phthalazin-2(1H)-yl)acetate.

2-[6-bromo-1-oxo-4-(trifluoromethyl)phthalazin-2-yl]-N-(5-fluoropyrimidin-4-yl)acetamide (131) and 2-[7-bromo-1-oxo-4-(trifluoromethyl)phthalazin-2-yl]-N-(5-fluoropyrimidin-4-yl)acetamide (132): To a solution of methyl 2-(6-bromo-1-oxo-4-(trifluoromethyl)phthalazin-2(1H)-yl)acetate and methyl 2-(7-bromo-1-oxo-4-(trifluoromethyl)phthalazin-2(1H)-yl)acetate (1:1 mixture, 100 mg, 0.27 mmol) in toluene (1.5 mL) and THF (4.0 mL) were added 5-fluoropyrimidin-4-amine (93 mg, 0.82 mmol) and AlMe₃ (0.41 mL, 2 M in toluene). The reaction mixture was stirred at 110° C. for 6 h. The reaction mixture was diluted with water (5 mL) and extracted with EtOAc (3 × 5 mL). The combined organic layers were dried over anhydrous Na₂SO₄, filtered, and concentrated under reduced pressure. The residue was purified by reverse-phase preparative HPLC followed by preparative SFC to provide:

2-[6-bromo-1-oxo-4-(trifluoromethyl)phthalazin-2-yl]-N-(5-fluoropyrimidin-4-yl)acetamide (131): LCMS: m/z = 446.0, 448.0 [M+H]⁺. ¹H NMR (400 MHz, DMSO-d₆): δ 11.27 (s, 1H), 8.85-8.80 (m, 2H), 8.52-8.48 (m, 1H), 8.31-8.27 (m, 1H), 7.98-7.92 (m, 1H), 5.26 (s, 2H); and

2-[7-bromo-1-oxo-4-(trifluoromethyl)phthalazin-2-yl]-N-(5-fluoropyrimidin-4-yl)acetamide (132): LCMS: m/z = 446.0, 448.0 [M+H]⁺. ¹H NMR (400 MHz, DMSO-d₆): δ 11.27 (s, 1H), 8.85-8.80 (m, 2H), 8.33-8.30 (m, 1H), 8.25-8.20 (m, 1H), 8.05 (s, 1H), 5.25 (s, 2H).

### Example 133

### 2-Bromo-4-(Difluoromethyl)-5-Fluoro-1-Oxophthalazin-2-yl]-N-(5-Cyanopyrimidin-2-yl)Acetamide (133)

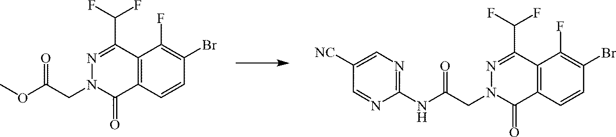

To a solution of methyl 2-(6-bromo-4-(difluoromethyl)-5-fluoro-1-oxophthalazin-2(1H)-yl)acetate (150 mg, 0.41 mmol) and 5-cyanopyrimidin-2-amine (54 mg, 0.45 mmol) in toluene (1.5 mL) and THF (1.5 mL) at 0° C. was added Al(CH₃)₃ (2 M in toluene, 0.82 mmol). The reaction mixture was stirred at 90° C. for 4 h. The reaction mixture was diluted with water (30 mL) and extracted with EtOAc (3 × 30 mL). The combined organic layers were washed with brine (30 mL), dried over anhydrous Na₂SO₄, filtered, and concentrated under reduced pressure. The residue was purified by reverse-phase preparative HPLC. LCMS: m/z = 452.9, 454.9 [M+H]⁺. ¹H NMR (400 MHz, CDCl₃) δ 8.88 (s, 2H), 8.83 (br s, 1H), 8.21 (d, J = 8.8 Hz, 1H), 8.06-8.02 (m, 1H), 6.92-6.65 (m, 1H), 5.61 (s, 2H).

### Example 134

### 2-Bromo-4-(Difluoromethyl)-1-Oxophthalazin-2-yl]-N-(5-Chloro-3-Fluoropyridin-2-yl)Acetamide (134)

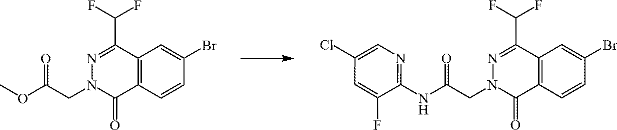 [SMILES: COC(=O)Cn1nc(C(F)F)c2cc(Br)ccc2c1=O ; O=C(Cn1nc(C(F)F)c2cc(Br)ccc2c1=O)Nc1ncc(Cl)cc1F]

To a solution of methyl 2-(6-bromo-4-(difluoromethyl)-1-oxophthalazin-2(1H)-yl)acetate (30 mg, 0.09 mmol) in DCE (2 mL) were added 5-chloro-3-fluoropyridin-2-amine (38 mg, 0.26 mmol) and AlMe₃ (1 M in n-heptane, 0.26 mL). The reaction mixture was stirred at 85° C. for 2 h. The reaction mixture was diluted with sat. aq. NH₄Cl (10 mL) and extracted with EtOAc (3 × 5 mL). The combined organic layers were washed with brine (10 mL), dried over anhydrous Na₂SO₄, filtered, and concentrated under reduced pressure. The residue was purified by reverse-phase preparative HPLC. LCMS: m/z = 460.9, 462.9, 464.9 [M+H]⁺. ¹H NMR (400 MHz, DMSO-d₆) δ 10.87 (br s, 1H), 8.37 (d, J = 2.0 Hz, 1H), 8.27 (d, J = 8.4 Hz, 1H), 8.23 (s, 1H), 8.20-8.13 (m, 2H), 7.23 (t, J = 52.4 Hz, 1H), 5.09 (s, 2H).

### Example 135

### 2-Bromo-4-(Difluoromethyl)-1-Oxophthalazin-2-yl]-N-(5-Chloropyrimidin-2-yl)Acetamide (135)

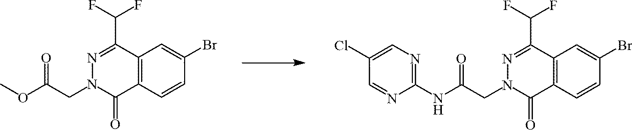 [SMILES: COC(=O)Cn1nc(C(F)F)c2cc(Br)ccc2c1=O ; O=C(Cn1nc(C(F)F)c2cc(Br)ccc2c1=O)Nc1ncc(Cl)cn1]

To a solution of methyl 2-(6-bromo-4-(difluoromethyl)-1-oxophthalazin-2(1H)-yl)acetate (50 mg, 0.14 mmol) in DCE (2.0 mL) were added 5-chloropyrimidin-2-amine (37 mg, 0.29 mmol) and AlMe₃ (2 M in n-heptane, 0.22 mL). The reaction mixture was stirred at 90° C. for 2 h. The reaction mixture was diluted with water (15 mL), filtered, and the filtrate was extracted with EtOAc (3 × 10 mL). The combined organic layers were dried over anhydrous Na₂SO₄, filtered, and concentrated under reduced pressure. The residue was purified by reverse-phase preparative HPLC. LCMS: m/z = 443.9, 445.9, 447.9 [M+H]⁺. ¹H NMR (400 MHz, DMSO-d₆) δ 11.26 (br s, 1H), 8.79 (s, 2H), 8.30-8.22 (m, 2H), 8.20-8.14 (m, 1H), 7.21 (t, J = 52.8, 1H), 5.21 (s, 2H).

### Example 136

### 2-Bromo-4-(Difluoromethyl)-1-Oxophthalazin-2-yl]-N-(5-Cyanopyrimidin-2-yl)Acetamide (136)

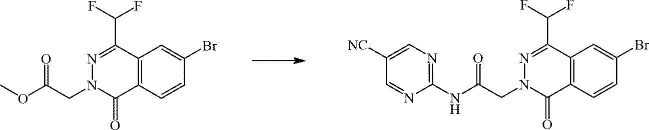

To a solution of methyl 2-(6-bromo-4-(difluoromethyl)-1-oxophthalazin-2(1H)-yl)acetate (50 mg, 0.14 mmol) and 5-cyanopyrimidin-2-amine (21 mg, 0.17 mmol) in DCE (1 mL) was added AlMe₃ (1 M in n-heptane, 0.43 mL). The reaction mixture was stirred at 60° C. for 5 h. The reaction mixture was diluted with water (5 mL) and extracted with EtOAc (3 × 5 mL). The combined organic layers were washed with brine (5 mL), dried over anhydrous Na₂SO₄, filtered, and concentrated under reduced pressure. The residue was purified by reverse-phase preparative HPLC. LCMS: m/z = 434.9, 437.0 [M+H]⁺. ¹H NMR (400 MHz, DMSO-d₆) δ 11.62 (br s, 1H), 9.15 (s, 2H), 8.26 (m, 2H), 8.18 (dd, J = 1.6, 8.4 Hz, 1H), 7.39-7.08 (t, J = 52.8 Hz, 1H), 5.28 (s, 2H).

### Example 137

### 2-Bromo-1-Oxo-4-(Fluoromethoxy)Phthalazin-2-yl]-N-(5-Fluoropyrimidin-2-yl)Acetamide (137)

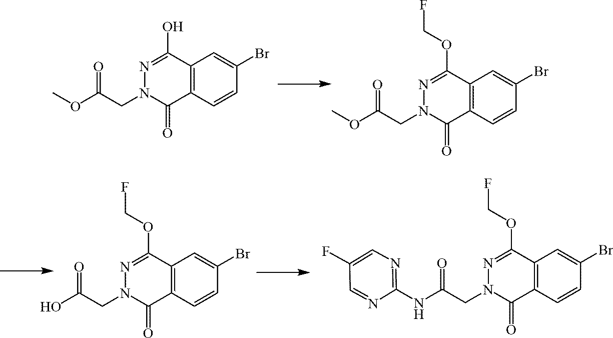 [SMILES: COC(=O)Cn1nc(O)c2cc(Br)ccc2c1=O ; COC(=O)Cn1nc(OCF)c2cc(Br)ccc2c1=O ; O=C(O)Cn1nc(OCF)c2cc(Br)ccc2c1=O ; O=C(Cn1nc(OCF)c2cc(Br)ccc2c1=O)Nc1ncc(F)cn1]

methyl 2-(6-bromo-4-(fluoromethoxy)-1-oxophthalazin-2(1H)-yl)acetate: To a mixture of methyl 2-(6-bromo-4-hydroxy-1-oxophthalazin-2(1H)-yl)acetate (500 mg, 1.60 mmol) and fluoromethyl-4-methylbenzenesulfonate (489 mg, 2.40 mmol) in DMF (5 mL) was added K₂CO₃ (441 mg, 3.19 mmol). The reaction mixture was stirred at 20° C. for 12 h. The reaction mixture was diluted with water (10 mL) and extracted with EtOAc (3 × 5 mL). The combined organic layers were washed with brine (5 mL), dried over anhydrous Na₂SO₄, filtered, and concentrated under reduced pressure. The residue was purified by silica gel column chromatography. LCMS: m/z = 344.9, 346.9 [M+H]⁺.

2-(6-bromo-4-(fluoromethoxy)-1-oxophthalazin-2(1H)-yl)acetic acid: To a mixture of methyl 2-(6-bromo-4-(fluoromethoxy)-1-oxophthalazin-2(1H)-yl)acetate (100 mg, 0.29 mmol) in THF (2.0 mL) and water (0.5 mL) was added LiOH•H₂O (24 mg, 0.58 mmol). The reaction mixture was stirred at 20° C. for 1 h. The reaction mixture was adjusted to pH = 4 with aq. HCl (1 M) and extracted with EtOAc (3 × 5 mL). The combined organic layers were washed with brine (5 mL), dried over anhydrous Na₂SO₄, filtered, and concentrated under reduced pressure to provide a residue that was used directly. LCMS: m/z = 330.9, 332.9 [M+H]⁺.

2-[6-bromo-1-oxo-4-(fluoromethoxy)phthalazin-2-yl]-N-(5-fluoropyrimidin-2-yl)acetamide: To a mixture of 2-(6-bromo-4-(fluoromethoxy)-1-oxophthalazin-2(1H)-yl)acetic acid (40 mg, 0.13 mmol) and 5-fluoropyrimidin-2-amine (27 mg, 0.24 mmol) in pyridine (1.0 mL) was added EDCI (46 mg, 0.25 mmol). The reaction mixture was stirred at 20° C. for 12 h. The reaction mixture was diluted with water (10 mL) and extracted with EtOAc (3 × 5 mL). The combined organic layers were washed with brine (5 mL), dried over anhydrous Na₂SO₄, filtered, and concentrated under reduced pressure. The residue was purified by reverse-phase preparative HPLC. LCMS: m/z = 425.9, 427.9 [M+H]⁺. ¹H NMR (400 MHz, CDCl₃) δ 8.78 (br s, 1H), 8.49 (s, 2H), 8.31 (d, J = 8.4 Hz, 1H), 8.19 (d, J = 1.8 Hz, 1H), 7.95 (dd, J = 2.0, 8.4 Hz, 1H), 6.1 (s, 1H), 5.97 (s, 1H), 5.35 (br s, 2H).

### Example 138

### 2-Bromo-1-Oxo-4-(2,2,2-Trifluoroethoxy)Phthalazin-2-yl]-N-(5-Fluoropyrimidin-2-yl)Acetamide (138)

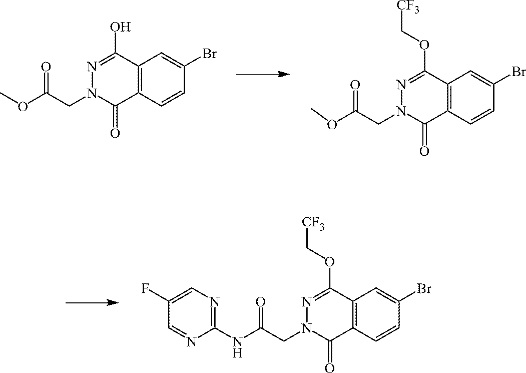 [SMILES: COC(=O)Cn1nc(O)c2cc(Br)ccc2c1=O ; COC(=O)Cn1nc(OCC(F)(F)F)c2cc(Br)ccc2c1=O ; O=C(Cn1nc(OCC(F)(F)F)c2cc(Br)ccc2c1=O)Nc1ncc(F)cn1]

methyl 2-(6-bromo-1-oxo-4-(2,2,2-trifluoroethoxy)phthalazin-2(1H)-yl)acetate: To a solution of methyl 2-(6-bromo-4-hydroxy-1-oxophthalazin-2(1H)-yl)acetate (50 mg, 0.16 mmol) and 2,2,2-trifluoroethyl trifluoromethanesulfonate (56 mg, 0.24 mmol) in DMF (1.0 mL) was added K₂CO₃ (44 mg, 0.32 mmol). The reaction mixture was stirred at 25° C. for 2 h. The reaction mixture was diluted with water (10 mL) and extracted with EtOAc (3 × 5 mL). The combined organic layers were washed with brine (5 mL), dried over anhydrous Na₂SO₄, filtered, and concentrated under reduced pressure to provide a residue that was used directly. LCMS: m/z = 394.9, 396.8 [M+H]⁺.

2-[6-bromo-1-oxo-4-(2,2,2-trifluoroethoxy)phthalazin-2-yl]-N-(5-fluoropyrimidin-2-yl)acetamide: To a solution of methyl 2-(6-bromo-1-oxo-4-(2,2,2-trifluoroethoxy)phthalazin-2(1H)-yl)acetate (45 mg, 0.11 mmol) and 5-fluoropyrimidin-2-amine (39 mg, 0.34 mmol) in DCE (1.0 mL) was added AlMe₃ (1 M in n-heptane, 0.17 mmol). The reaction mixture was stirred at 60° C. for 12 h. The reaction mixture was diluted with water (5 mL) and extracted with EtOAc (3 × 5 mL). The combined organic layers were dried over anhydrous Na₂SO₄, filtered, and concentrated under reduced pressure. The residue was purified by reverse-phase preparative HPLC. LCMS: m/z = 475.9, 477.9 [M+H]⁺. ¹H NMR (400 MHz, CDCl₃) δ 8.66 (br s, 1H), 8.49 (s, 2H), 8.31 (d, J = 8.4 Hz, 1H), 8.16 (d, J = 1.6 Hz, 1H), 7.96 (dd, J = 1.6, 8.4 Hz, 1H), 5.31 (br s, 2H), 4.70 (q, J = 8.4 Hz, 2H).

### Example 139

### 2-Bromo-4-(1,2-Difluoroethyl)-1-Oxophthalazin-2-yl]-N-(5-Fluoropyrimidin-2-yl)Acetamide (139)

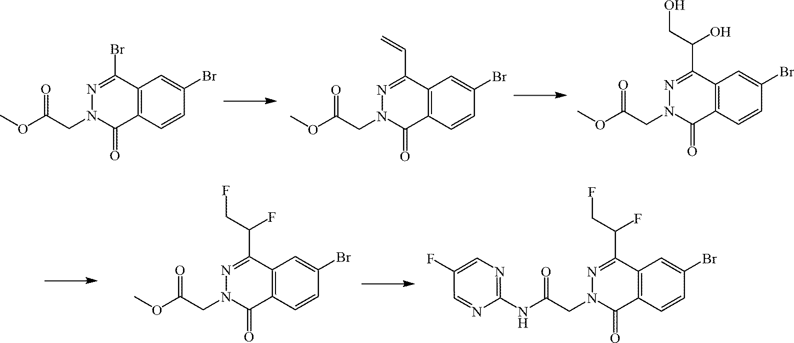 [SMILES: COC(=O)Cn1nc(Br)c2cc(Br)ccc2c1=O ; C=Cc1nn(CC(=O)OC)c(=O)c2ccc(Br)cc12 ; COC(=O)Cn1nc(C(O)CO)c2cc(Br)ccc2c1=O ; COC(=O)Cn1nc(C(F)CF)c2cc(Br)ccc2c1=O ; O=C(Cn1nc(C(F)CF)c2cc(Br)ccc2c1=O)Nc1ncc(F)cn1]

methyl 2-(6-bromo-1-oxo-4-vinylphthalazin-2(1H)-yl)acetate: To a solution of methyl 2-(4,6-dibromo-1-oxophthalazin-2(1H)-yl)acetate (2.5 g, 6.65 mmol) and tributyl(vinyl)stannane (2.11 g, 6.65 mmol) in DMF (30 mL) was added Pd(PPh₃)₄ (768 mg, 0.67 mmol). The reaction mixture was stirred at 80° C. for 8 h. The reaction mixture was quenched by addition of sat. aq. KF (30 mL) and extracted with DCM (3 × 20 mL). The combined organic layers were washed with brine (10 mL), dried over anhydrous Na₂SO₄, filtered, and concentrated under reduced pressure. The crude residue was purified by silica gel column chromatography. LCMS: m/z = 322.9, 324.9 [M+H]⁺.

methyl 2-(6-bromo-4-(1,2-dihydroxyethyl)-1-oxophthalazin-2(1H)-yl)acetate: To a solution of methyl 2-(6-bromo-1-oxo-4-vinylphthalazin-2(1H)-yl)acetate (1.0 g, 3.09 mmol) in DCM (10 mL) and water (2 mL) were added K₂OsO₄•2H₂O (114 mg, 0.31 mmol) and 4-methyl-4-oxido-morpholin-4-ium (1.09 g, 9.28 mmol). The reaction mixture was stirred at 25° C. for 12 h. The reaction mixture was diluted with water (20 mL) and extracted with DCM (3 × 10 mL). The combined organic layers were dried over anhydrous Na₂SO₄, filtered, and concentrated under reduced pressure. The residue was purified by silica gel column chromatography. LCMS: m/z = 356.9, 358.9 [M+H]⁺.

methyl 2-(6-bromo-4-(1,2-difluoroethyl)-1-oxophthalazin-2(1H)-yl)acetate: To a solution of methyl 2-(6-bromo-4-(1,2-dihydroxyethyl)-1-oxophthalazin-2(1H)-yl)acetate (80 mg, 0.22 mmol) in DCM (2.0 mL) at 0° C. was added DAST (144 mg, 0.90 mmol). The reaction mixture was stirred at 25° C. for 12 h. The reaction mixture was quenched by addition of sat. aq. NaHCO₃ (10 mL) and extracted with DCM (3 × 5 mL). The combined organic layers were dried over anhydrous Na₂SO₄, filtered, and concentrated under reduced pressure. The residue was purified by preparative TLC. LCMS: m/z = 360.9, 362.9 [M+H]⁺.

2-[6-bromo-4-(1,2-difluoroethyl)-1-oxophthalazin-2-yl]-N-(5-fluoropyrimidin-2-yl)acetamide: To a solution of methyl 2-(6-bromo-4-(1,2-difluoroethyl)-1-oxophthalazin-2(1H)-yl)acetate (18 mg, 0.05 mmol) and 5-fluoropyrimidin-2-amine (17 mg, 0.15 mmol) in DCE (1.0 mL) was added AlMe₃ (2 M in toluene, 0.15 mmol). The reaction mixture was stirred at 90° C. for 3 h. The reaction mixture was diluted with water (5 mL) and extracted with EtOAc (3 × 5 mL). The combined organic layers were dried over anhydrous Na₂SO₄, filtered, and concentrated under reduced pressure. The residue was purified by reverse-phase preparative HPLC. LCMS: m/z = 441.9, 443.9 [M+H]⁺. ¹H NMR (400 MHz, CDCl₃) δ 8.61 (br s, 1H), 8.49 (s, 2H), 8.36 (d, J = 8.8 Hz, 1H), 8.21 (s, 1H), 7.94 (dd, J = 1.6, 8.8 Hz, 1H), 6.11-5.88 (m, 1H), 5.61-5.48 (m, 1H), 5.43-5.34 (m, 1H), 5.17-5.07 (m, 1H), 5.05-4.94 (m, 1H).

### Examples 140 and 141

### 2-Bromo-5-Fluoro-4-Methoxy-1-Oxophthalazin-2-yl)-N-(5-Fluoropyrimidin-2-yl)Acetamide (140) and 2-Bromo-4-Chloro-5-Methoxy-1-Oxophthalazin-2-yl)-N-(5-Fluoropyrimidin-2-yl)Acetamide (141)

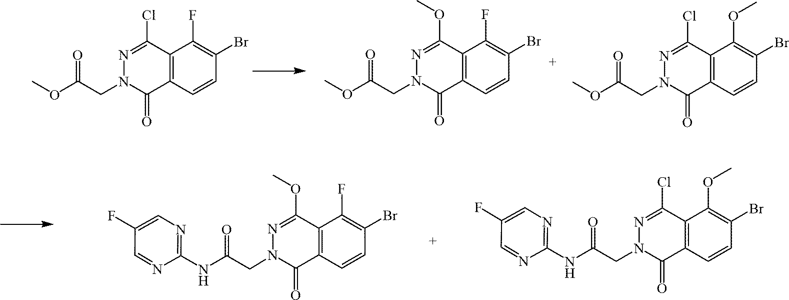

methyl 2-(6-bromo-5-fluoro-4-methoxy-1-oxophthalazin-2(1H)-yl)acetate and methyl 2-(6-bromo-4-chloro-5-methoxy-1-oxophthalazin-2(1H)-yl)acetate: To a solution of methyl 2-(6-bromo-4-chloro-5-fluoro-1-oxophthalazin-2(1H)-yl)acetate (220 mg, 0.63 mmol) in MeOH (4 mL) was added NaOMe (113 mg, 0.63 mmol, 30% purity in MeOH). The reaction mixture was stirred at 50° C. for 2 h. The reaction mixture was diluted with water (8 mL) and extracted with EtOAc (3 × 3 mL). The combined organic layers were washed with brine (5 mL), dried over anhydrous Na₂SO₄, filtered, and concentrated under reduced pressure. The residue was purified by silica gel column chromatography to give an 3:1 mixture of methyl 2-(6-bromo-5-fluoro-4-methoxy-1-oxophthalazin-2(1H)-yl)acetate and methyl 2-(6-bromo-4-chloro-5-methoxy-1-oxophthalazin-2(1H)-yl)acetate. LCMS: m/z = 344.9, 346.9 [M+H]⁺ and m/z = 360.2, 362.2, 364.2.

2-(6-bromo-5-fluoro-4-methoxy-1-oxophthalazin-2-yl)-N-(5-fluoropyrimidin-2-yl)acetamide and 2-(6-bromo-4-chloro-5-methoxy-1-oxophthalazin-2-yl)-N-(5-fluoropyrimidin-2-yl)acetamide: To a solution of the mixture of methyl 2-(6-bromo-5-fluoro-4-methoxy-1-oxophthalazin-2(1H)-yl)acetate and methyl 2-(6-bromo-4-chloro-5-methoxy-1-oxophthalazin-2(1H)-yl)acetate mixture (3:1 ratio, 90 mg, 0.26 mmol) in DCE (1.0 mL) were added 5-fluoropyrimidin-2-amine (88 mg, 0.78 mmol) and AlMe₃ (1 M in toluene, 0.78 mL). The reaction mixture was stirred at 60° C. for 3 h. The reaction mixture was diluted with water (8 mL) and extracted with EtOAc (3 × 3 mL). The combined organic layers were washed with brine (5 mL), dried over anhydrous Na₂SO₄, filtered, and concentrated under reduced pressure. The residue was purified by reverse-phase preparative HPLC to give:

2-(6-bromo-5-fluoro-4-methoxy-1-oxophthalazin-2-yl)-N-(5-fluoropyrimidin-2-yl)acetamide. LCMS: m/z = 425.9, 427.9 [M+H]⁺. ¹H NMR (400 MHz, CDCl₃) δ 8.94 (br s, 1H), 8.50 (s, 2H), 8.15 (d, J = 8.4 Hz, 1H), 7.96 (dd, J = 6.0, 8.4 Hz, 1H), 5.29 (s, 2H), 3.99 (s, 3H);

and 2-(6-bromo-4-chloro-5-methoxy-1-oxophthalazin-2-yl)-N-(5-fluoropyrimidin-2-yl)acetamide. LCMS: m/z = 441.9, 443.9, 445.9 [M+H]⁺. ¹H NMR (400 MHz, CDCl₃) δ 9.58 (br s, 1H), 8.54 (s, 2H), 8.17 (d, J = 8.8 Hz, 1H), 8.02 (d, J = 8.4 Hz, 1H), 5.51 (br s, 2H), 4.00 (s, 3H).

### Examples 142 and 143

### 2-Bromo-4-(Fluoromethyl)-1-Oxophthalazin-2-yl]-N-(5-Fluoropyrimidin-2-yl)Acetamide (142) and 2-[7-Bromo-4-(Fluoromethyl)-1-Oxophthalazin-2-yl]-N-(5-Fluoropyrimidin-2-yl)Acetamide (143)

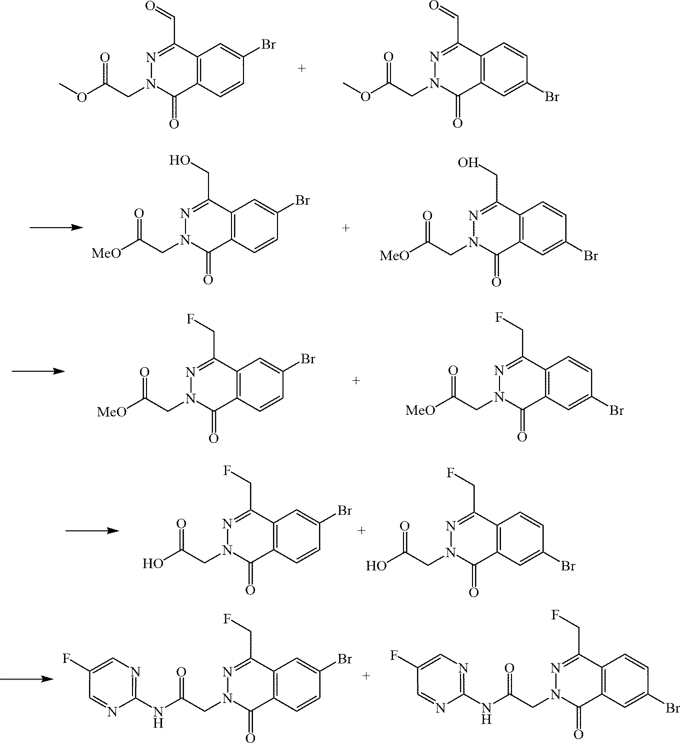

methyl 2-(6-bromo-4-(hydroxymethyl)-1-oxophthalazin-2(1H)-yl)acetate and methyl 2-(7-bromo-4-(hydroxymethyl)-1-oxophthalazin-2(1H)-yl)acetate: To a solution of methyl 2-(6-bromo-4-formyl-1-oxophthalazin-2(1H)-yl)acetate and methyl 2-(7-bromo-4-formyl-1-oxophthalazin-2(1H)-yl)acetate (1:1 mixture, 250 mg, 0.77 mmol) in MeOH (10 mL) and water (1 mL) at 0° C. was added NaBH₄ (29 mg, 0.77 mmol). The reaction mixture was stirred at 0° C. for 1 h. The reaction mixture was diluted with sat. aq. NH₄Cl (10 mL) and extracted with EtOAc (3 × 10 mL). The combined organic layers were washed with brine (50 mL), dried over anhydrous Na₂SO₄, filtered, and concentrated under reduced pressure. The residue was purified by silica gel column chromatography. LCMS: m/z = 327.0, 329.0 [M+H]⁺.

methyl 2-(6-bromo-4-(fluoromethyl)-1-oxophthalazin-2(1H)-yl)acetate and methyl 2-(7-bromo-4-(fluoromethyl)-1-oxophthalazin-2(1H)-yl)acetate: A solution of methyl 2-(6-bromo-4-(hydroxymethyl)-1-oxophthalazin-2(1H)-yl)acetate and methyl 2-(7-bromo-4-(hydroxymethyl)-1-oxophthalazin-2(1H)-yl)acetate (1:1 mixture, 250 mg, 0.76 mmol) in BAST (3.03 g, 13.70 mmol, 3 mL) was stirred at 20° C. for 16 h. The reaction mixture was poured into sat. aq. NaHCO₃ (5 mL) and extracted with EtOAc (3 × 3 mL). The combined organic layers were washed with brine (10 mL), dried over anhydrous Na₂SO₄, filtered, and concentrated under reduced pressure. The residue was purified by silica gel column chromatography. LCMS: m/z = 329.1, 331.1 [M+H]⁺.

2-(6-bromo-4-(fluoromethyl)-1-oxophthalazin-2(1H)-yl)acetic acid and 2-(7-bromo-4-(fluoromethyl)-1-oxophthalazin-2(1H)-yl)acetic acid: To a solution of methyl 2-(6-bromo-4-(fluoromethyl)-1-oxophthalazin-2(1H)-yl)acetate and methyl 2-(7-bromo-4-(fluoromethyl)-1-oxophthalazin-2(1H)-yl)acetate (1:1 mixture, 65 mg, 0.20 mmol) in THF (2.0 mL) and water (2.0 mL) was added LiOH•H₂O (21 mg, 0.50 mmol). The reaction mixture was stirred at 20° C. for 2 h. The reaction mixture was diluted with water (5 mL) and washed with MTBE (3 mL). The aqueous phase was then adjusted pH = 3 with aq. HCl (3 M) and extracted with EtOAc (3 × 3 mL). The combined organic layers were washed with brine (5 mL), dried over anhydrous Na₂SO₄, filtered, and concentrated under reduced pressure to provide a residue that was used directly. LCMS: m/z = 315.0, 317.0 [M+H]⁺.

2-[6-bromo-4-(fluoromethyl)-1-oxophthalazin-2-yl]-N-(5-fluoropyrimidin-2-yl)acetamide and 2-[7-bromo-4-(fluoromethyl)-1-oxophthalazin-2-yl]-N-(5-fluoropyrimidin-2-yl)acetamide: To a solution of 2-(6-bromo-4-(fluoromethyl)-1-oxophthalazin-2(1H)-yl)acetic acid and 2-(7-bromo-4-(fluoromethyl)-1-oxophthalazin-2(1H)-yl)acetic acid (1:1 mixture, 80 mg, 0.25 mmol) in pyridine (2.0 mL) were added 5-fluoropyrimidin-2-amine (57 mg, 0.51 mmol) and EDCI (73 mg, 0.38 mmol). The reaction mixture was stirred at 20° C. for 16 h. The reaction mixture was diluted with water (5 mL) and extracted with EtOAc (3 × 2 mL). The combined organic layers were washed with brine (5 mL), dried over anhydrous Na₂SO₄, filtered, and concentrated under reduced pressure. The residue was purified by reverse-phase preparative HPLC to provide:

2-[6-bromo-4-(fluoromethyl)-1-oxophthalazin-2-yl]-N-(5-fluoropyrimidin-2-yl)acetamide: LCMS: m/z = 409.9, 411.9 [M+H]⁺. ¹H NMR (400 MHz, DMSO-d₆) δ 11.18 (br s, 1H), 8.77 (s, 2H), 8.30 (s, 1H), 8.22 (d, J = 8.4 Hz, 1H), 8.12 (d, J = 8.4 Hz, 1H), 5.78 (s, 1H), 5.66 (s, 1H), 5.16 (s, 2H); and

2-[7-bromo-4-(fluoromethyl)-1-oxophthalazin-2-yl]-N-(5-fluoropyrimidin-2-yl)acetamide: LCMS: m/z = 409.9, 411.9 [M+H]⁺. ¹H NMR (400 MHz, DMSO-d₆) δ11.17 (br s, 1H), 8.77 (s, 2H), 8.41 (d, J = 2.0 Hz, 1H), 8.23 (dd, J = 2.0, 8.4 Hz, 1H), 8.05 (br d, J= 7.6 Hz, 1H), 5.76 (s, 1H), 5.65 (s, 1H), 5.17 (s, 2H).

### Example 144

### 2-Bromo-5-Fluoro-4-(1-Fluoroethyl)-1-Oxophthalazin-2-yl]-N-(5-Fluoropyrimidin-2-yl)Acetamide (144)

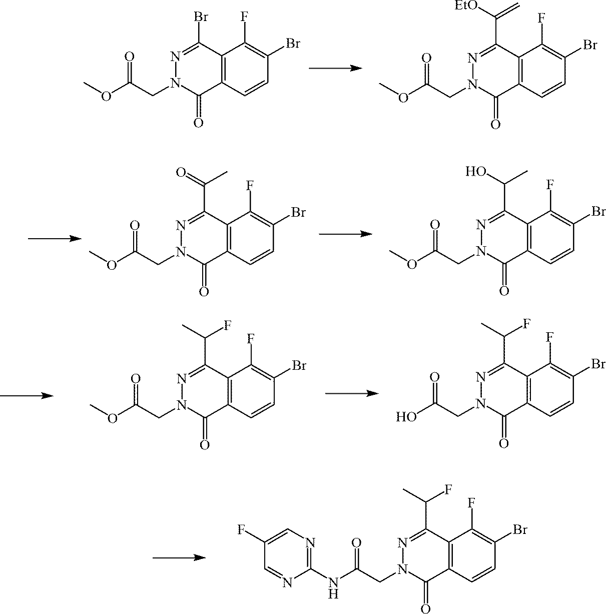

methyl 2-(6-bromo-4-(1-ethoxyvinyl)-5-fluoro-1-oxophthalazin-2(1H)-yl)acetate: To a solution of methyl 2-(4,6-dibromo-5-fluoro-1-oxophthalazin-2(1H)-yl)acetate (1.0 g, 2.54 mmol) in DMF (20 mL) were added Pd(PPh₃)₄ (293 mg, 0.25 mmol) and tributyl(1-ethoxyvinyl)stannane (917 mg, 2.54 mmol, 0.86 mL). The reaction mixture was stirred at 100° C. for 8 h. The reaction mixture was poured into sat. aq. KF (30 mL), stirred at 20° C. for 0.5 h, and then extracted with EtOAc (3 × 10 mL). The combined organic layers were washed with brine (10 mL), dried over anhydrous Na₂SO₄, filtered, and concentrated under reduced pressure. The crude residue was purified by silica gel column chromatography. LCMS: m/z = 385.2, 387.1 [M+H]⁺.

methyl 2-(4-acetyl-6-bromo-5-fluoro-1-oxophthalazin-2(1H)-yl)acetate: To a solution of methyl 2-(6-bromo-4-(1-ethoxyvinyl)-5-fluoro-1-oxophthalazin-2(1H)-yl)acetate (500 mg, 1.30 mmol) in EtOAc (5.0 mL) was added HCl (4 M in EtOAc, 0.32 mL). The reaction mixture was stirred at 20° C. for 2 h. The reaction mixture was concentrated under reduced pressure. The crude residue was purified by silica gel column chromatography. LCMS: m/z = 357.1, 359.1 [M+H]⁺.

methyl 2-(6-bromo-5-fluoro-4-(1-hydroxyethyl)-1-oxophthalazin-2(1H)-yl)acetate: To a solution of methyl 2-(4-acetyl-6-bromo-5-fluoro-1-oxophthalazin-2(1H)-yl)acetate (230 mg, 0.64 mmol) in THF (3.0 mL) at 0° C. was added NaBH₄ (17 mg, 0.45 mmol). The reaction mixture was stirred at 0° C. for 2 h. The reaction mixture was diluted with sat. aq. NH₄Cl (10 mL) and extracted with EtOAc (3 × 5 mL). The combined organic layers were washed with brine (10 mL), dried over anhydrous Na₂SO₄, filtered, and concentrated under reduced pressure to provide a residue that was used directly. LCMS: m/z = 359.1, 361.1 [M+H]⁺.

methyl 2-(6-bromo-5-fluoro-4-(1-fluoroethyl)-1-oxophthalazin-2(1H)-yl)acetate: Methyl 2-(6-bromo-5-fluoro-4-(1-hydroxyethyl)-1-oxophthalazin-2(1H)-yl)acetate (160 mg, 0.45 mmol) was suspended in BAST (3.03 g, 13.7 mmol) at 0° C. The reaction mixture was stirred at 20° C. for 3 h. The reaction mixture was poured into ice-cold sat. aq. NaHCO₃ (20 mL) and extracted with DCM (3 × 10 mL). The combined organic layers were washed with brine (10 mL), dried over anhydrous Na₂SO₄, filtered, and concentrated under reduced pressure. The crude residue was purified by silica gel column chromatography. LCMS: m/z = 361.1, 363.1 [M+H]⁺.

2-(6-bromo-5-fluoro-4-(1-fluoroethyl)-1-oxophthalazin-2(1H)-yl)acetic acid: To a solution of methyl 2-(6-bromo-5-fluoro-4-(1-fluoroethyl)-1-oxophthalazin-2(1H)-yl)acetate (70 mg, 0.19 mmol) in THF (2.0 mL) and water (0.4 mL) was added LiOH•H₂O (16 mg, 0.39 mmol). The reaction mixture was stirred at 20° C. for 1 h. The reaction mixture was diluted with water (10 mL) and washed with MTBE (3 × 5 mL). The aqueous was then cooled to 0° C., adjusted to pH = 3 with aq. HCl (3 M), and extracted with EtOAc (3 × 10 mL). The combined organic layers were washed with brine (10 mL), dried over anhydrous Na₂SO₄, filtered, and concentrated under reduced pressure to provide a residue that was used directly. LCMS: m/z = 347.0, 349.0 [M+H]⁺.

2-[6-bromo-5-fluoro-4-(1-fluoroethyl)-1-oxophthalazin-2-yl]-N-(5-fluoropyrimidin-2-yl)acetamide: To a solution of 2-(6-bromo-5-fluoro-4-(1-fluoroethyl)-1-oxophthalazin-2(1H)-yl)acetic acid (45 mg, 0.13 mmol) and 5-fluoropyrimidin-2-amine (22 mg, 0.19 mmol) in pyridine (2.0 mL) was added EDCI (37 mg, 0.19 mmol). The reaction mixture was stirred at 30° C. for 12 h and then 60° C. for a further 2 h. The reaction mixture was then cooled to 20° C. and then additional EDCI (37 mg, 0.19 mmol) was added. The reaction mixture was stirred at 60° C. for a further 12 h. The reaction mixture was diluted with water (10 mL) and extracted with EtOAc (3 × 5 mL). The combined organic layers were washed with brine (2 × 5 mL), dried over anhydrous Na₂SO₄, filtered, and concentrated under reduced pressure. The residue was purified by reverse-phase preparative HPLC. LCMS: m/z = 441.9, 443.9 [M+H]⁺. ¹H NMR (400 MHz, DMSO-d₆) δ 11.17 (s, 1H), 8.77 (s, 2H), 8.23 (dd, J = 6.4, 8.4 Hz, 1H), 8.07 (d, J = 8.4 Hz, 1H), 6.29-6.08 (m, 1H), 5.16 (s, 2H), 1.65 (dd, J = 5.6, 23.6 Hz, 3H).

### Example 145

### 2-Bromo-5-Fluoro-4-Methyl-1-Oxophthalazin-2-yl)-N-(5-Fluoropyrimidin-2-yl)Acetamide (145)

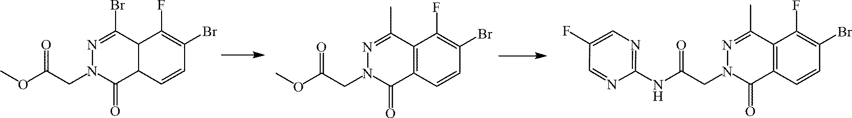 [SMILES: COC(=O)CN1N=C(Br)C2C(F)=C(Br)C=CC2C1=O ; COC(=O)Cn1nc(C)c2c(F)c(Br)ccc2c1=O ; Cc1nn(CC(=O)Nc2ncc(F)cn2)c(=O)c2ccc(Br)c(F)c12]

methyl 2-(6-bromo-5-fluoro-4-methyl-1-oxophthalazin-2(1H)-yl)acetate and methyl 2-(4,6-dibromo-5-fluoro-1-oxophthalazin-2(1H)-yl)acetate: To a solution of methyl 2-(4,6-dibromo-5-fluoro-1-oxo-phthalazin-2-yl)acetate (500 mg, 1.27 mmol) and Pd(PPh₃)₄ (147 mg, 0.13 mmol) in DMF (7 mL) was added tetramethylstannane (908 mg, 5.08 mmol). The reaction mixture was stirred at 110° C. for 2 h. The reaction mixture was quenched with aq. sat. KF (30 mL) and extracted with EtOAc (3 × 10 mL). The combined organic layers were washed with brine (10 mL), dried over anhydrous Na₂SO₄, filtered, and concentrated under reduced pressure. The residue was purified by silica gel column chromatography. LCMS: m/z = 328.9, 330.9 [M+H]⁺.

2-(6-bromo-5-fluoro-4-methyl-1-oxophthalazin-2-yl)-N-(5-fluoropyrimidin-2-yl)acetamide: To a solution of methyl 2-(6-bromo-5-fluoro-4-methyl-1-oxo-phthalazin-2-yl)acetate (80 mg, 0.24 mmol) and 5-fluoropyrimidin-2-amine (55 mg, 0.4 mmol) in DCE (3 mL) was added AlMe₃ (1 M in n-heptane, 0.53 mL). The reaction mixture was stirred at 90° C. for 2 h. The reaction mixture was diluted with water (9 mL) and extracted with EtOAc (3 × 3 mL). The combined organic layers were washed with brine (3 mL), dried over anhydrous Na₂SO₄, filtered, and concentrated under reduced pressure. The residue was purified by reverse-phase preparative HPLC. LCMS: m/z = 409.9, 411.9 [M+H]⁺. ¹H NMR (400 MHz, CDCl₃) δ 8.73 (br s, 1H), 8.49 (s, 2H), 8.19 (d, J = 8.3 Hz, 1H), 7.94 (dd, J = 6.8, 8.3 Hz, 1H), 5.42 (br s, 2H), 2.72 (d, J = 6.6 Hz, 3H).

### Example 146

### N-fluoropyrimidin-2-yl)-2-[4-methoxy-1-oxo-6-(trifluoromethyl)phthalazin-2-yl]acetamide (146)

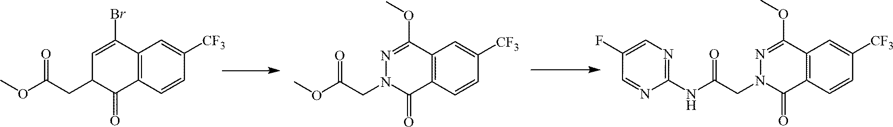 [SMILES: COC(=O)CC1C=C(Br)c2cc(C(F)(F)F)ccc2C1=O ; COC(=O)Cn1nc(OC)c2cc(C(F)(F)F)ccc2c1=O ; COc1nn(CC(=O)Nc2ncc(F)cn2)c(=O)c2ccc(C(F)(F)F)cc12]

methyl 2-(4-methoxy-1-oxo-6-(trifluoromethyl)phthalazin-2(1H)-yl)acetate: To a solution of methyl 2-(4-bromo-1-oxo-6-(trifluoromethyl)phthalazin-2(1H)-yl)acetate (200 mg, 0.55 mmol) in MeOH (5.0 mL) at 0° C. was added NaOMe (89 mg, 1.64 mmol). The reaction mixture was stirred at 40° C. for 5 h. The reaction mixture was diluted with water (10 mL) and extracted with MTBE (3 × 5 mL). The combined organic layers were washed with brine (10 mL), dried over anhydrous Na₂SO₄, filtered, and concentrated under reduced pressure to provide a residue that was used directly. LCMS: m/z = 317.2 [M+H]⁺.

N-(5-fluoropyrimidin-2-yl)-2-[4-methoxy-1-oxo-6-(trifluoromethyl)phthalazin-2-yl]acetamide: To a mixture of methyl 2-(4-methoxy-1-oxo-6-(trifluoromethyl)phthalazin-2(1H)-yl)acetate (40 mg, 0.13 mmol), 5-fluoropyrimidin-2-amine (43 mg, 0.38 mmol) in DCE (2.0 mL) was added AlMe₃ (1 M in n-heptane, 0.4 mL). The reaction mixture was stirred for at 90° C. for 90 min. The reaction mixture was cooled to 0° C., diluted with water (10 mL), and extracted with EtOAc (4 × 5 mL). The combined organic layers were washed with brine (2 × 10 mL), dried over anhydrous Na₂SO₄, filtered, and concentrated under reduced pressure. The residue was purified by reverse-phase preparative HPLC. LCMS: m/z = 398.0 [M+H]⁺. ¹H NMR (400 MHz, DMSO-d₆) δ 11.10 (br s, 1H), 8.78 (s, 2H), 8.46 (d, J = 8.0 Hz, 1H), 8.26 (d, J = 9.6 Hz, 2H), 5.06 (s, 2H), 3.96 (s, 3H).

### Example 147

### 2-(Fluoromethyl)-1-Oxo-6-(Trifluoromethyl)Phthalazin-2-yl]-N-(5-Fluoropyrimidin-2-yl)Acetamide (147)

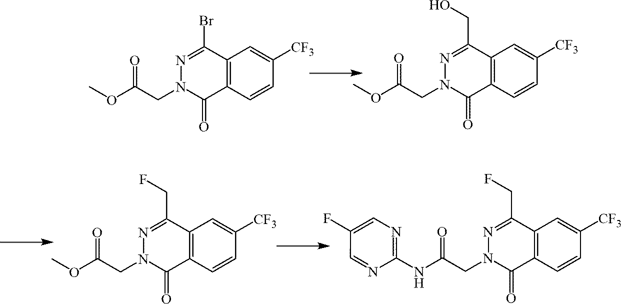 [SMILES: COC(=O)Cn1nc(Br)c2cc(C(F)(F)F)ccc2c1=O ; COC(=O)Cn1nc(CO)c2cc(C(F)(F)F)ccc2c1=O ; COC(=O)Cn1nc(CF)c2cc(C(F)(F)F)ccc2c1=O ; O=C(Cn1nc(CF)c2cc(C(F)(F)F)ccc2c1=O)Nc1ncc(F)cn1]

methyl 2-(4-(hydroxymethyl)-1-oxo-6-(trifluoromethyl)phthalazin-2(1H)-yl)acetate: A mixture of methyl 2-(4-bromo-1-oxo-6-(trifluoromethyl)phthalazin-2(1H)-yl)acetate (200 mg, 0.55 mmol), tributylstannylmethanol (264 mg, 0.82 mmol), Pd(PPh₃)₄ (63 mg, 0.054 mmol) in 1,4-dioxane (5.0 mL) was degassed and purged with N₂ 3 times. The reaction mixture was stirred at 80° C. for 16 h and then 110° C. for a further 3 h. The reaction mixture was quenched by addition of sat. aq. KF (20 mL) and extracted with EtOAc (3 × 10 mL). The combined organic layers were washed with brine (3 × 10 mL), dried over anhydrous Na₂SO₄, filtered, and concentrated under reduced pressure. The residue was purified by silica gel column chromatography. LCMS: m/z = 317.2 [M+H]⁺.

methyl 2-(4-(fluoromethyl)-1-oxo-6-(trifluoromethyl)phthalazin-2(1H)-yl)acetate: To a mixture of methyl 2-(4-(hydroxymethyl)-1-oxo-6-(trifluoromethyl)phthalazin-2(1H)-yl)acetate (80 mg, 0.25 mmol) in DCM (3.0 mL) at -78° C. were added N,N-diethylethanamine trihydrofluoride (82 mg, 0.51 mmol) and (difluoro-λ⁴-sulfanylidene)diethylammonium tetrafluoroborate (116 mg, 0.51 mmol). The reaction mixture was stirred at 20° C. for 2 h. The reaction mixture was diluted with water (10 mL) and extracted with EtOAc (3 × 10 mL). The combined organic layers were washed with brine (3 × 10 mL), dried over anhydrous Na₂SO₄, filtered, and concentrated under reduced pressure. The residue was purified by silica gel column chromatography. LCMS: m/z = 319.2 [M+H]⁺.

2-[4-(fluoromethyl)-1-oxo-6-(trifluoromethyl)phthalazin-2-yl]-N-(5-fluoropyrimidin-2-yl)acetamide: To a mixture of 5-fluoropyrimidin-2-amine (21 mg, 0.19 mmol) and methyl 2-(4-(fluoromethyl)-1-oxo-6-(trifluoromethyl)phthalazin-2(1H)-yl)acetate (20 mg, 0.062 mmol) in DCE (3.0 mL) was added AlMe₃ (1 M in toluene, 0.19 mL). The reaction mixture was stirred at 80° C. for 2 h. The reaction mixture was diluted with water (10 mL) and extracted with DCM (3 × 10 mL). The combined organic layers were washed with brine (3 × 10 mL), dried over anhydrous Na₂SO₄, filtered, and concentrated under reduced pressure. The residue was purified by reverse-phase preparative HPLC. LCMS: m/z = 400.0 [M+H]⁺. ¹H NMR (400 MHz, CDCl₃) δ 8.64 (d, J= 8.5 Hz, 1H), 8.59 (br s, 1H), 8.50 (s, 2H), 8.26 (s, 1H), 8.04 (d, J = 7.9 Hz, 1H), 5.73-5.58 (m, 2H), 5.54 (br s, 2H).

### Example 148

### 2-Bromo-5-Fluoro-4-(Fluoromethyl)-1-Oxophthalazin-2-yl]-N-(5-Fluoropyrimidin-2-yl)Acetamide (148)

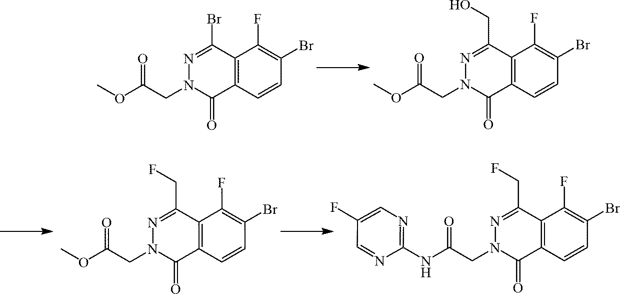 [SMILES: COC(=O)Cn1nc(Br)c2c(F)c(Br)ccc2c1=O ; COC(=O)Cn1nc(CO)c2c(F)c(Br)ccc2c1=O ; COC(=O)Cn1nc(CF)c2c(F)c(Br)ccc2c1=O ; O=C(Cn1nc(CF)c2c(F)c(Br)ccc2c1=O)Nc1ncc(F)cn1]

methyl 2-(6-bromo-5-fluoro-4-(hydroxymethyl)-1-oxophthalazin-2(1H)-yl)acetate: To a solution of methyl 2-(4,6-dibromo-5-fluoro-1-oxophthalazin-2(1H)-yl)acetate (600 mg, 1.52 mmol) and (tributylstannyl)methanol (440 mg, 1.37 mmol) in 1,4-dioxane (5.0 mL) was added Pd(PPh₃)₄ (176 mg, 0.15 mmol). The reaction mixture was stirred at 80° C. for 6 h. To the reaction mixture was add sat. aq. KF (10 mL) and the mixture was stirred for 1 h. The reaction mixture was extracted with EtOAc (3 × 10 mL). The combined organics were dried over anhydrous Na₂SO₄, filtered, and concentrated under reduced pressure. The residue was purified by silica gel column chromatography. ¹H-NMR (400 MHz, CDCl₃): δ 8.19 (dd, J = 8.4, 0.8 Hz, 1H), 7.98 (dd, J = 8.4, 6.4 Hz, 1H), 5.03 (d, J = 3.6 Hz, 2H), 4.98 (s, 2H), 3.81 (s, 3H), 3.18 (br s, 1H).

methyl 2-(6-bromo-5-fluoro-4-(fluoromethyl)-1-oxophthalazin-2(1H)-yl)acetate: To a solution of methyl 2-(6-bromo-5-fluoro-4-(hydroxymethyl)-1-oxophthalazin-2(1H)-yl)acetate (200 mg, 0.58 mmol) in DCM (10 mL) were added N,N-diethylethanamine trihydrofluoride (187 mg, 1.16 mmol) and (difluoro-λ⁴-sulfanylidene)diethylammonium tetrafluoroborate (265 mg, 1.16 mmol). The reaction mixture was stirred at 15° C. for 16 h. The reaction mixture was diluted with water (10 mL) and extracted with DCM (2 × 10 mL). The combined organics were dried over anhydrous Na₂SO₄, filtered, and concentrated under reduced pressure. The residue was purified by silica gel column chromatography. ¹H NMR (400 MHz, CDCl₃): δ 8.11-8.20 (m, 1H), 7.97 (dd, J= 8.4, 6.4 Hz, 1H), 5.64 (d, J= 3.2 Hz, 1H), 5.53 (d, J= 2.8 Hz, 1H), 4.97 (s, 2H), 3.79 (s, 3H).

2-[6-bromo-5-fluoro-4-(fluoromethyl)-1-oxophthalazin-2-yl]-N-(5-fluoropyrimidin-2-yl)acetamide: To a solution of methyl 2-(6-bromo-5-fluoro-4-(fluoromethyl)-1-oxophthalazin-2(1H)-yl)acetate (48 mg, 0.14 mmol) and 5-fluoropyrimidin-2-amine (19 mg, 0.17 mmol) in DCE (1.0 mL) was added AlMe₃ (1 M in n-heptane, 0.21 mL). The reaction mixture was stirred at 90° C. for 2 h. The reaction mixture was diluted with water (5 mL) and extracted with EtOAc (3 × 5 mL). The combined organic layers were dried over anhydrous Na₂SO₄, filtered, and concentrated under reduced pressure. The residue was purified by reverse-phase preparative HPLC. LCMS: m/z = 427.9, 429.9 [M+H]⁺. ¹H NMR (400 MHz, CDCl₃) δ 8.61 (br s, 1H), 8.49 (s, 2H), 8.24-8.15 (m, 1H), 8.04-7.93 (m, 1H), 5.67-5.50 (m, 4H).

### Example 149

### 2-Bromo-4-Methylsulfanyl-1-Oxophthalazin-2-yl)-N-(5-Fluoropyrimidin-2-yl)Acetamide (149)

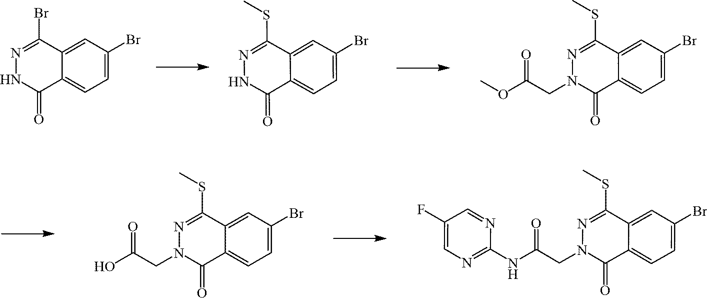 [SMILES: O=c1[nH]nc(Br)c2cc(Br)ccc12 ; CSc1n[nH]c(=O)c2ccc(Br)cc12 ; COC(=O)Cn1nc(SC)c2cc(Br)ccc2c1=O ; CSc1nn(CC(=O)O)c(=O)c2ccc(Br)cc12 ; CSc1nn(CC(=O)Nc2ncc(F)cn2)c(=O)c2ccc(Br)cc12]

6-bromo-4-methylsulfanyl-2H-phthalazin-1-one: To a solution of sodium methylthiolate (230 mg, 3.29 mmol) in DMF (6 mL) was added K₂CO₃ (900 mg, 6.5 mmol). The mixture was stirred at 25° C. for 24 h. The mixture was diluted with water (20 mL) and extracted with EtOAc (3 × 20 mL). The combined organic layers were washed with brine, dried over anhydrous Na₂SO₄, filtered, and concentrated under reduced pressure to provide a residue that was used directly. LCMS: m/z = 271.1, 273.0 [M+H]⁺.

methyl 2-(6-bromo-4-methylsulfanyl-1-oxo-phthalazin-2-yl)acetate: To a solution of 6-bromo-4-methylsulfanyl-2H-phthalazin-1-one (300 mg, 1.11 mmol) and Cs₂CO₃ (540 mg, 1.66 mmol) in DMF (3.0 mL) was added methyl bromoacetate (203 mg, 1.33 mmol). The mixture was stirred at 40° C. for 2 h. The mixture was diluted with water (10 mL) and extracted with EtOAc (3 × 15 mL). The combined organic layers were washed with brine, dried over anhydrous Na₂SO₄, filtered, and concentrated under reduced pressure to provide a residue that was used directly. LCMS: m/z = 343.1, 345.0 [M+H]⁺.

2-(6-bromo-4-methylsulfanyl-1-oxo-phthalazin-2-yl)acetic acid: To a solution of methyl 2-(6-bromo-4-methylsulfanyl-1-oxo-phthalazin-2-yl)acetate (40 mg, 0.12 mmol) in THF (0.5 mL) was added aq. LiOH (0.17 mL, 1 M). The mixture was stirred at 40° C. for 2 h followed by the addition of aq. HCl (0.34 mL, 1 M). The reaction mixture was diluted with water and extracted with EtOAc (2 × 10 mL). The combined organic layers were washed with brine (10 mL), dried over anhydrous Na₂SO₄, filtered, and concentrated under reduced pressure to provide a residue that was used directly. LCMS: m/z = 329.0, 331.0 [M+H]⁺.

2-(6-bromo-4-methylsulfanyl-1-oxophthalazin-2-yl)-N-(5-fluoropyrimidin-2-yl)acetamide: To a solution of 2-(6-bromo-4-methylsulfanyl-1-oxo-phthalazin-2-yl)acetic acid (30 mg, 0.36 mmol), 5-fluoropyrimidin-2-amine (24 mg, 0.21 mmol), and 1-methylimidazole (30 mg, 0.91 mmol) in MeCN (1.0 mL) was added TCFH (46 mg, 0.17 mmol). The mixture was stirred at 40° C. for 2 h. A second portion of TCFH was added (46 mg, 0.17 mmol) at 40° C. and the mixture was stirred for an additional 2 h. The mixture was diluted with MeCN (5 mL) and water (3 mL), filtered, and directly purified by reverse-phase preparative HPLC. LCMS: m/z = 424.1, 426.1 [M+H]⁺. 1H NMR (400 MHz, DMSO-d₆): δ 11.19 (s, 1H), 8.79 (s, 2H), 8.18 (dd, J= 8.5, 0.4 Hz, 1H), 7.85 (dd, J= 8.5, 1.9 Hz, 1H), 7.62 (d, J = 1.6 Hz, 1H), 5.13-5.12 (m, 2H), 2.68 (s, 3H).

### Example 150

### 2-Bromo-4-Cyclopropyloxy-1-Oxophthalazin-2-yl)-N-(5-Fluoropyrimidin-2-yl)Acetamide (150)

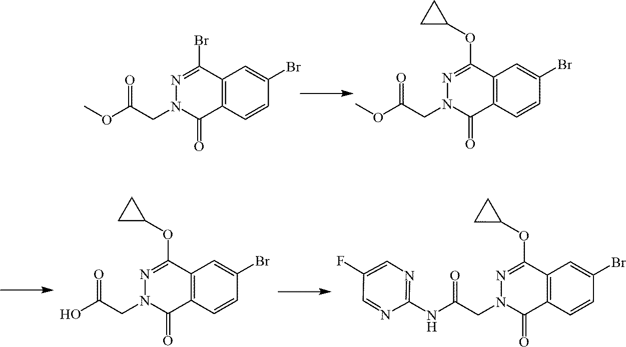 [SMILES: COC(=O)Cn1nc(Br)c2cc(Br)ccc2c1=O ; COC(=O)Cn1nc(OC2CC2)c2cc(Br)ccc2c1=O ; O=C(O)Cn1nc(OC2CC2)c2cc(Br)ccc2c1=O ; O=C(Cn1nc(OC2CC2)c2cc(Br)ccc2c1=O)Nc1ncc(F)cn1]

methyl 2-[6-bromo-4-(cyclopropoxy)-1-oxo-phthalazin-2-yl]acetate: To a solution of NaH (36 mg, 1.5 mmol) in DMF (2 mL) at 0° C. was added cyclopropanol (92 mg, 1.6 mmol). The mixture was stirred at 0° C. for 45 min. Methyl 2-(4,6-dibromo-1-oxo-phthalazin-2-yl)acetate (200 mg, 0.53 mmol) was added as a solid in one portion at 0° C. The mixture was stirred for 2 h at 0° C. The reaction mixture was diluted with aq. HCl (2 mL, 1 M) and extracted with EtOAc (2 × 10 mL). The combined organic layers were washed with brine (10 mL), dried over anhydrous Na₂SO₄, filtered, and concentrated under reduced pressure to provide a residue that was used directly. LCMS: m/z = 353.2, 355.1 [M+H]⁺.

2-[6-bromo-4-(cyclopropoxy)-1-oxo-phthalazin-2-yl]acetic acid: To a solution of methyl 2-[6-bromo-4-(cyclopropoxy)-1-oxo-phthalazin-2-yl]acetate (490 mg, 1.4 mmol) in THF (5.7 mL) was added aq. LiOH (2.7 mL, 1 M). The mixture was stirred at 40° C. for 2 h. The mixture was diluted with aq. HCl (3.5 mL, 1 M) and extracted with EtOAc (2 × 10 mL). The combined organic layers were washed with brine (10 mL), dried over anhydrous Na₂SO₄, filtered, and concentrated under reduced pressure to provide a residue that was used directly. LCMS: m/z = 339.1, 341.1 [M+H]⁺.

2-(6-bromo-4-cyclopropyloxy-1-oxophthalazin-2-yl)-N-(5-fluoropyrimidin-2-yl)acetamide: To a solution of 2-[6-bromo-4-(cyclopropoxy)-1-oxo-phthalazin-2-yl]acetic acid (500 mg, 1.47 mmol), 5-fluoropyrimidin-2-amine (300 mg, 2.6 mmol), and 1-methylimidazole (484 mg, 5.9 mmol) in MeCN (5.0 mL) was added TCFH (580 mg, 2.1 mmol). The mixture was stirred at 40° C. for 2 h. A second portion of TCFH was added (580 mg, 2.1 mmol) at 40° C. and the mixture was stirred for an additional 2 h. The mixture was diluted with MeCN (5 mL) and water (3 mL), filtered, and directly purified by reverse-phase preparative HPLC. LCMS: m/z = 434.2, 436.2 [M+H]⁺. ¹H NMR (400 MHz, CDCl₃): δ 9.37 (s, 1H), 8.52 (s, 2H), 8.30 (d, J = 8.5 Hz, 1H), 8.06 (s, 1H), 7.90 (dd, J = 8.5, 1.8 Hz, 1H), 5.30 (s, 2H), 4.30-4.25 (m, 1H), 0.88-0.83 (m, 4H).

### Example 151

### 2-Bromo-4-Cyclopropyloxy-1-Oxophthalazin-2-yl)-N-(5-Cyano-3-Fluoropyridin-2-yl)Acetamide (151)

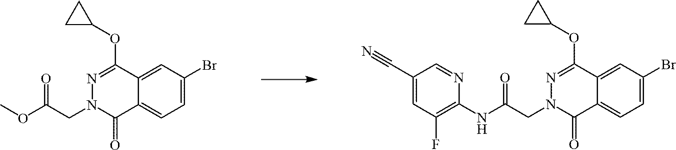 [SMILES: COC(=O)Cn1nc(OC2CC2)c2cc(Br)ccc2c1=O ; N#Cc1cnc(NC(=O)Cn2nc(OC3CC3)c3cc(Br)ccc3c2=O)c(F)c1]

To a solution of methyl 2-(6-bromo-4-cyclopropoxy-1-oxophthalazin-2(1H)-yl)acetate (50 mg, 0.14 mmol) and 6-amino-5-fluoronicotinonitrile (58 mg, 0.43 mmol) in DCE (1.0 mL) at 0° C. was added AlMe₃ (1 M in n-heptane, 0.43 mL). The mixture was stirred at 60° C. for 2 h. The reaction mixture cooled to 0° C., diluted with water (3 mL), and extracted with EtOAc (3 × 4 mL). The combined organic layers were washed with brine (6 mL), dried over anhydrous Na₂SO₄, filtered, concentrated under reduced pressure, and purified by reverse-phase preparative HPLC. LCMS: m/z = 457.9, 459.9 [M+H]⁺. ¹H NMR (400 MHz, CDCl₃): δ 9.00 (br s, 1H), 8.51 (s, 1H), 8.29 (d, J= 8.4 Hz, 1H), 8.07 (s, 1H), 7.92 (dd, J= 1.6, 8.4 Hz, 1H), 7.68 (br d, J= 9.6 Hz, 1H), 5.25 (s, 2H), 4.34-4.19 (m, 1H), 0.89-0.82 (m, 4H).

### Example 152

### 2-Bromo-4-Cyclopropyloxy-1-Oxophthalazin-2-yl)-N-(5-Methylpyrimidin-2-yl)Acetamide (152)

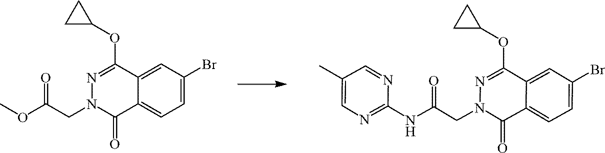 [SMILES: COC(=O)Cn1nc(OC2CC2)c2cc(Br)ccc2c1=O ; Cc1cnc(NC(=O)Cn2nc(OC3CC3)c3cc(Br)ccc3c2=O)nc1]

To a solution of methyl 2-(6-bromo-4-cyclopropoxy-1-oxophthalazin-2(1H)-yl)acetate (50 mg, 0.14 mmol) and 5-methylpyrimidin-2-amine (31 mg, 0.28 mmol) in DCE (1.0 mL) was added AlMe₃ (1 M in n-heptane, 0.28 mL). The reaction mixture was stirred at 60° C. for 2 h. The reaction mixture was diluted with water (3 mL) and extracted with EtOAc (3 × 3 mL). The combined organic layers were washed with brine (3 mL), dried over anhydrous Na₂SO₄, filtered, and concentrated under reduced pressure. The residue was purified by reverse-phase preparative HPLC. LCMS: m/z = 430.1, 432.1 [M+H]⁺. ¹H NMR (400 MHz, CDCl₃): δ 9.23 (s, 1H), 8.45 (s, 2H), 8.28 (d, J= 8.4 Hz, 1H), 8.05 (d, J= 1.6 Hz, 1H), 7.88 (dd, J = 1.6, 8.4 Hz, 1H), 5.35 (s, 2H), 4.24-4.29 (m, 1H), 2.27 (s, 3H), 0.91-0.74 (m, 4H).

### Example 153

### 2-Bromo-4-Cyclobutyloxy-1-Oxophthalazin-2-yl)-N-(5-Fluoropyrimidin-2-yl)Acetamide (153)

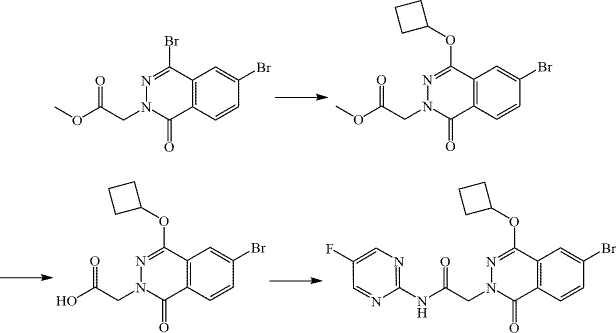 [SMILES: COC(=O)Cn1nc(Br)c2cc(Br)ccc2c1=O ; COC(=O)Cn1nc(OC2CCC2)c2cc(Br)ccc2c1=O ; O=C(O)Cn1nc(OC2CCC2)c2cc(Br)ccc2c1=O ; O=C(Cn1nc(OC2CCC2)c2cc(Br)ccc2c1=O)Nc1ncc(F)cn1]

methyl 2-[6-bromo-4-(cyclobutoxy)-1-oxo-phthalazin-2-yl]acetate: To a solution of NaH (36 mg, 1.5 mmol) in DMF (1.6 mL) at 0° C. was added cyclobutanol (72 mg, 0.79 mmol). The mixture wasstirred at 0° C. for 45 min. Methyl 2-(4,6-dibromo-1-oxo-phthalazin-2-yl)acetate (200 mg, 0.53 mmol) was added as a solid in one portion at 0° C. The mixture was stirred for 2 h at 0° C. The reaction mixture was diluted with aq. HCl (2 mL, 1 M) and extracted with EtOAc (2 × 10 mL). The combined organic layers were washed with brine (10 mL), dried over anhydrous Na₂SO₄, filtered, and concentrated under reduced pressure to provide a residue that was used directly. LCMS: m/z = 367.1, 369.1 [M+H]⁺.

2-[6-bromo-4-(cyclobutoxy)-1-oxo-phthalazin-2-yl]acetic acid: To a solution of methyl 2-[6-bromo-4-(cyclobutoxy)-1-oxo-phthalazin-2-yl]acetate (250 mg, 0.68 mmol) in THF (2.9 mL) was added aq. LiOH (1.3 mL, 1 M). The reaction mixture was stirred at 40° C. for 2 h. The reaction mixture was diluted with aq. HCl (2.6 mL, 1 M) and extracted with EtOAc (2 × 10 mL). The combined organic layers were washed with brine (10 mL), dried over anhydrous Na₂SO₄, filtered, and concentrated under reduced pressure to provide a residue that was used directly. LCMS: m/z = 353.1, 355.1 [M+H]⁺.

2-(6-bromo-4-cyclobutyloxy-1-oxophthalazin-2-yl)-N-(5-fluoropyrimidin-2-yl)acetamide: To a solution of 2-[6-bromo-4-(cyclobutoxy)-1-oxo-phthalazin-2-yl]acetic acid (200 mg, 0.56 mmol), 5-fluoropyrimidin-2-amine (115 mg, 1.0 mmol), and 1-methylimidazole (185 mg, 2.2 mmol) in MeCN (5 mL) was added TCFH (222 mg, 0.79 mmol). The reaction mixture was stirred at 40° C. for 2 h. A second portion of TCFH was added (222 mg, 0.79 mmol) at 40° C. and the reaction mixture was stirred for an additional 2 h. The reaction mixture was diluted with MeCN (5 mL) and water (3 mL), filtered, and directly purified by reverse-phase preparative HPLC. LCMS: m/z = 448.2, 450.1 [M+H]⁺. ¹H NMR (400 MHz, DMSO-d₆) δ 11.09 (s, 1H), 8.79 (d, J= 0.6 Hz, 2H), 8.17-8.09 (m, 3H), 5.06-4.97 (m, 3H), 2.44-2.38 (m, 2H), 2.22-2.12 (m, 2H), 1.85-1.76 (m, 1H), 1.70-1.60 (m, 1H).

### Example 154

### 2-Bromo-4-Ethoxy-1-Oxophthalazin-2-yl)-N-(5-Fluoropyrimidin-2-yl)Acetamide (154)

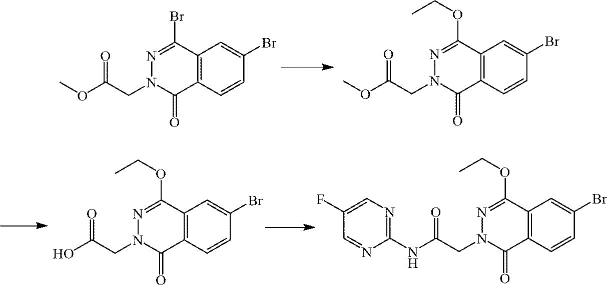 [SMILES: COC(=O)Cn1nc(Br)c2cc(Br)ccc2c1=O ; CCOc1nn(CC(=O)OC)c(=O)c2ccc(Br)cc12 ; CCOc1nn(CC(=O)O)c(=O)c2ccc(Br)cc12 ; CCOc1nn(CC(=O)Nc2ncc(F)cn2)c(=O)c2ccc(Br)cc12]

methyl 2-(6-bromo-4-ethoxy-1-oxo-phthalazin-2-yl)acetate: To a solution of NaH (36 mg, 1.5 mmol) in DMF (1.6 mL) at 0° C. was added ethanol (72 mg, 1.5 mmol). The mixture was stirred at 0° C. for 45 min. Methyl 2-(4,6-dibromo-1-oxo-phthalazin-2-yl)acetate (200 mg, 0.53 mmol) was added as a solid in one portion at 0° C. The mixture was stirred for 2 h at 0° C. The reaction was diluted with aq. HCl (2 mL, 1 M) and extracted with EtOAc (2 × 10 mL). The combined organic layers were washed with brine (10 mL), dried over anhydrous Na₂SO₄, filtered, and concentrated under reduced pressure to provide a residue that was used directly. LCMS: m/z = 341.1, 343.1 [M+H]⁺.

2-(6-bromo-4-ethoxy-1-oxo-phthalazin-2-yl)acetic acid: To a solution of methyl 2-(6-bromo-4-ethoxy-1-oxo-phthalazin-2-yl)acetate (240 mg, 0.67 mmol) in THF (2.9 mL) was added aq. LiOH (0.95 mL, 1 M). The mixture was stirred at 40° C. for 2 h. The mixture was diluted with aq. HCl (1.5 mL, 1 M) and extracted with EtOAc (2 × 10 mL). The combined organic layers were washed with brine (10 mL), dried over anhydrous Na₂SO₄, filtered, and concentrated under reduced pressure to provide a residue that was used directly. LCMS: m/z = 327.1, 329.1 [M+H]⁺.

2-(6-bromo-4-ethoxy-1-oxophthalazin-2-yl)-N-(5-fluoropyrimidin-2-yl)acetamide: To a solution of 2-(6-bromo-4-ethoxy-1-oxo-phthalazin-2-yl)acetic acid (150 mg, 0.45 mmol), 5-fluoropyrimidin-2-amine (93 mg, 0.82 mmol), and 1-methylimidazole (150 mg, 1.81 mmol) in MeCN (3 mL) was added TCFH (180 mg, 0.64 mmol). The mixture was stirred at 40° C. for 2 h. A second portion of TCFH was added (180 mg, 0.64 mmol) at 40° C. and the mixture was stirred for an additional 2 h. The mixture was diluted with MeCN (5 mL) and water (3 mL), filtered, and directly purified by reverse-phase preparative HPLC. LCMS: m/z = 422.1, 424.1 [M+H]⁺. ¹H NMR (400 MHz, DMSO-d₆): δ 11.10 (s, 1H), 8.79 (s, 2H), 8.18-8.15 (m, 1H), 8.12-8.10 (m, 2H), 5.01 (s, 2H), 4.33-4.28 (m, 2H), 1.40 (t, J= 7.0 Hz, 3H).

### Example 155

### 2-Bromo-1-Oxo-4-Propan-2-Yloxyphthalazin-2-yl)-N-(5-Fluoropyrimidin-2-yl)Acetamide (155)

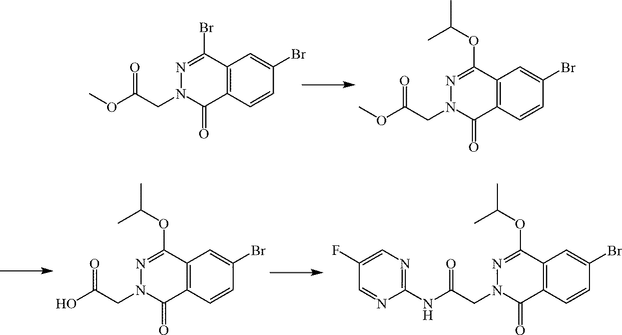 [SMILES: COC(=O)Cn1nc(Br)c2cc(Br)ccc2c1=O ; COC(=O)Cn1nc(OC(C)C)c2cc(Br)ccc2c1=O ; CC(C)Oc1nn(CC(=O)O)c(=O)c2ccc(Br)cc12 ; CC(C)Oc1nn(CC(=O)Nc2ncc(F)cn2)c(=O)c2ccc(Br)cc12]

methyl 2-(6-bromo-4-isopropoxy-1-oxo-phthalazin-2-yl)acetate: To a solution of NaH (36 mg, 1.5 mmol) in DMF (1.6 mL) at 0° C. was added isopropanol (72 mg, 1.5 mmol). The reaction mixture was stirred at 0° C. for 45 min. Methyl 2-(4,6-dibromo-1-oxo-phthalazin-2-yl)acetate (200 mg, 0.53 mmol) was then added as a solid in one portion. The reaction mixture was stirred for 0° C. for a further 2h. The reaction mixture was diluted with aq. HCl (2 mL, 1 M) and extracted with EtOAc (2 × 10 mL). The combined organic layers were washed with brine (10 mL), dried over anhydrous Na₂SO₄, filtered, and concentrated under reduced pressure to provide a residue that was used directly. LCMS: m/z = 355.1, 357.1 [M+H]⁺.

2-(6-bromo-4-isopropoxy-1-oxo-phthalazin-2-yl)acetic acid: To a solution of methyl 2-(6-bromo-4-isopropoxy-1-oxo-phthalazin-2-yl)acetate (230 mg, 0.67 mmol) in THF (2.0 mL) was added aq. LiOH (1.3 mL, 1 M). The mixture was stirred at 40° C. for 2 h. The mixture was diluted with aq. HCl (2 mL, 1 M) and extracted with EtOAc (2 × 10 mL). The combined organic layers were washed with brine (10 mL), dried over anhydrous Na₂SO₄, filtered, and concentrated under reduced pressure to provide a residue that was used directly. LCMS: m/z = 341.1, 343.1 [M+H]⁺.

2-(6-bromo-1-oxo-4-propan-2-yloxyphthalazin-2-yl)-N-(5-fluoropyrimidin-2-yl)acetamide: To a solution of 2-(6-bromo-4-isopropoxy-1-oxo-phthalazin-2-yl)acetic acid (200 mg, 0.58 mmol), 5-fluoropyrimidin-2-amine (119 mg, 1.05 mmol), and 1-methylimidazole (192 mg, 2.34 mmol) in MeCN (3 mL) was added TCFH (230 mg, 0.82 mmol). The mixture was stirred at 40° C. for 2 h. A second portion of TCFH was added (230 mg, 0.82 mmol) at 40° C. and the mixture was stirred for an additional 2 h. The mixture was diluted with MeCN (5 mL) and water (3 mL), filtered, and directly purified by reverse-phase preparative HPLC. LCMS: m/z = 436.1, 438.1 [M+H]⁺. ¹H NMR (400 MHz, CDCl₃) δ 8.99 (s, 1H), 8.49 (s, 2H), 8.30 (d, J= 8.6 Hz, 1H), 8.17-8.17 (m, 1H), 7.92-7.90 (m, 1H), 5.25-5.18 (m, 3H), 1.43 (d, J = 6.2 Hz, 6H).

### Example 156

### 2-Bromo-4-(3,3-Dimethylcyclobutoxy)-1-Oxo-Phthalazin-2-yl]-N-(5-Fluoropyrimidin-2-yl)Acetamide (156)

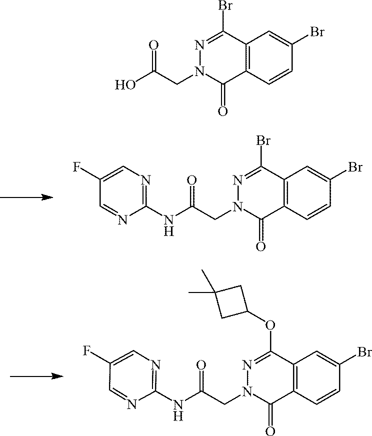 [SMILES: O=C(O)Cn1nc(Br)c2cc(Br)ccc2c1=O ; O=C(Cn1nc(Br)c2cc(Br)ccc2c1=O)Nc1ncc(F)cn1 ; CC1(C)CC(Oc2nn(CC(=O)Nc3ncc(F)cn3)c(=O)c3ccc(Br)cc23)C1]

2-(4,6-dibromo-1-oxo-phthalazin-2-yl)-N-(5-fluoropyrimidin-2-yl)acetamide: To a solution of 2-(4,6-dibromo-1-oxo-phthalazin-2-yl)acetic acid (1.3 g, 3.6 mmol), 5-fluoropyrimidin-2-amine (736 mg, 6.5 mmol), and 1-methylimidazole (1.18 g, 14.4 mmol) in MeCN (13 mL) was added TCFH (1.4 g, 5.0 mmol). The reaction mixture was stirred at 40° C. for 2 h. A second portion of TCFH was added (1.4 g, 5.0 mmol) at 40° C. and the mixture was stirred for an additional 2 h. The reaction mixture was diluted with water (20 mL), filtered, and the filter cake was dried under reduced pressure to provide a residue that was used directly. LCMS: m/z = 456.1, 458.1, 460.1 [M+H]⁺.

2-[6-bromo-4-(3,3-dimethylcyclobutoxy)-1-oxo-phthalazin-2-yl]-N-(5-fluoropyrimidin-2-yl)acetamide: To a solution of NaH (12 mg, 0.52 mmol) in DMF (0.5 mL) at 0° C. was added 3,3-dimethylcyclobutanol (39 mg, 0.39 mmol). The reaction mixture was stirred at 0° C. for 45 min. 2-(4,6-dibromo-1-oxo-phthalazin-2-yl)-N-(5-fluoropyrimidin-2-yl)acetamide (60 mg, 0.13 mmol) was added as a solid in one portion at 0° C. The mixture was stirred for 2 h at 0° C. The reaction mixture was diluted with aq. HCl (1 mL, 1 M) and extracted with EtOAc (2 × 10 mL). The combined organic layers were washed with brine (5 mL), dried over anhydrous Na₂SO₄, filtered, and concentrated under reduced pressure. The residue was purified by silica gel column chromatography. LCMS: m/z = 476.2, 478.2 [M+H]⁺. ¹H NMR (400 MHz, CDCl₃): δ 9.31 (s, 1H), 8.52 (s, 2H), 8.29 (dd, J= 8.5, 0.5 Hz, 1H), 8.17 (dd, J= 2.0, 0.5 Hz, 1H), 7.91 (dd, J= 8.5, 2.0 Hz, 1H), 5.23 (s, 2H), 5.16-5.09 (m, 1H), 2.40-2.35 (m, 2H), 2.02 (ddd, J= 11.3, 5.8, 2.7 Hz, 2H), 1.21 (d, J= 6.5 Hz, 6H).

### Example 157 and 158

### 2-Bromo-4-(3-Cis-Methylcyclobutoxy)-1-Oxo-Phthalazin-2-yl]-N-(5-Fluoropyrimidin-2-yl)Acetamide (157) and 2-Bromo-4-(3-Trans-Methylcyclobutoxy)-1-Oxo-Phthalazin-2-yl]-N-(5-Fluoropyrimidin-2-yl)Acetamide (158)

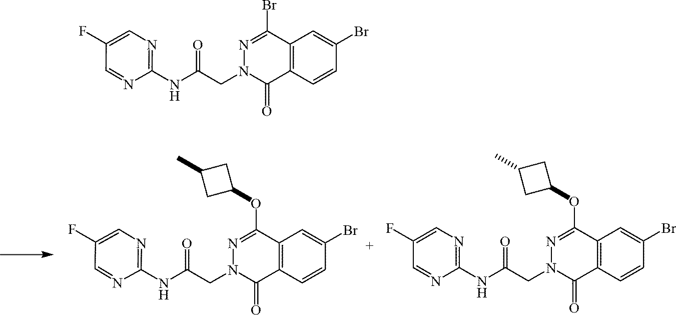

To a solution of NaH (12 mg, 0.52 mmol) in DMF (0.5 mL) at 0° C. was added 3-methylcyclobutanol (34 mg, 0.39 mmol). The mixture was stirred at 0° C. for 45 min. 2-(4,6-dibromo-1-oxo-phthalazin-2-yl)-N-(5-fluoropyrimidin-2-yl)acetamide (60 mg, 0.13 mmol) was added as a solid in one portion at 0° C. The mixture was stirred for 2 h at 0° C. The reaction mixture was diluted with aq. HCl (1 mL, 1 M) and extracted with EtOAc (2 × 10 mL). The combined organic layers were washed withbrine (5 mL), dried over anhydrous Na₂SO₄, filtered, and concentrated under reduced pressure. The residue was purified by silica gel column chromatography and further purified by preparative SFC to provide:

2-[6-bromo-4-(3-cis-methylcyclobutoxy)-1-oxo-phthalazin-2-yl]-N-(5-fluoropyrimidin-2-yl)acetamide (157). LCMS: m/z = 461.9, 464.0 [M+H]⁺. ¹H NMR (400 MHz, DMSO-d₆): δ 11.06 (br s, 1H), 8.74 (m, 2H), 8.17-8.06 (m, 3H), 4.96 (s, 2H), 4.90-4.80 (m, 1H), 2.63-2.54 (m, 2H), 2.07-1.88 (m, 1H), 1.79-1.64 (m, 2H), 1.13 (d, J= 6.4 Hz, 3H); and 2-[6-bromo-4-(3-trans-methylcyclobutoxy)-1-oxo-phthalazin-2-yl]-N-(5-fluoropyrimidin-2-yl)acetamide (158). LCMS: m/z = 461.9, 463.9 [M+H]⁺. ¹H NMR (400 MHz, DMSO-d₆): δ 11.06 (br s, 1H), 8.75 (s, 2H), 8.17-8.12 (m, 2H), 8.12-8.07 (m, 1H), 5.28-5.10 (m, 1H), 4.96 (s, 2H), 2.41-2.29 (m, 3H), 2.14-2.03 (m, 2H), 1.09 (d, J= 6.8 Hz, 3H).

### Example 159

### 2-Bromo-1-Oxo-4-Propan-2-Ylphthalazin-2-yl)-N-(4-Ethyl-1,4-Oxazepan-6-yl)Acetamide (159)

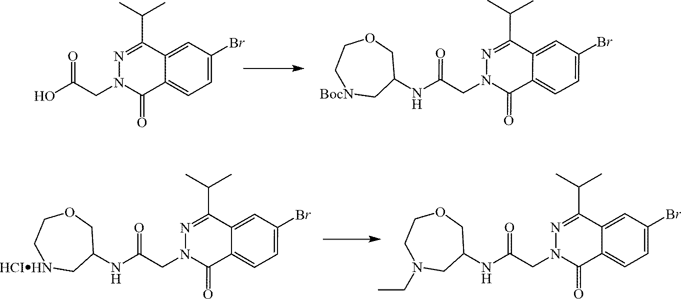

tert-butyl 6-[[2-(6-bromo-4-isopropyl-1-oxo-phthalazin-2-yl)acetyl]amino]-1,4-oxazepane-4-carboxylate: To a solution of 2-(6-bromo-4-isopropyl-1-oxo-phthalazin-2-yl)acetic acid (114 mg, 0.35 mmol) and Et₃N (0.24 mL, 1.75 mmol) in DMF (1.0 mL) was added HATU (200 mg, 0.53 mmol). The reaction mixture was stirred at 25° C. for 5 min followed by the addition of tert-butyl 6-amino-1,4-oxazepane-4-carboxylate (114 mg, 0.53 mmol). The mixture was stirred at 25° C. for 4 h. The mixture was diluted with water (5 mL) and filtered. The filter cake was washed with water (10 mL), Et₂O (10 mL), dried under reduced pressure, and purified by reverse-phase preparative HPLC. LCMS: m/z = 523.5, 525.5 [M+H]⁺.

2-(6-bromo-4-isopropyl-1-oxo-phthalazin-2-yl)-N-(1,4-oxazepan-6-yl)acetamide HCl salt: To a solution of tert-butyl 6-[[2-(6-bromo-4-isopropyl-1-oxo-phthalazin-2-yl)acetyl]amino]-1,4-oxazepane-4-carboxylate (226 mg, 0.41 mmol) in EtOAc (1.6 mL) was added HCl (2 mL, 1 M in dioxane). The reaction mixture was stirred at 25° C. for 2 h. The mixture was concentrated under reduced pressure to provide a residue that was used directly. LCMS: m/z = 423.3, 425.3 [M+H]⁺.

2-(6-bromo-1-oxo-4-propan-2-ylphthalazin-2-yl)-N-(4-ethyl-1,4-oxazepan-6-yl)acetamide: To a solution of 2-(6-bromo-4-isopropyl-1-oxo-phthalazin-2-yl)-N-(1,4-oxazepan-6-yl)acetamide HCl salt (165 mg, 0.36 mmol) and AcOH (1.0 mL) in DCM (1.0 mL) was added acetaldehyde (316 mg, 7.18 mmol). The reaction mixture was stirred for 5 min at 25° C. followed by the addition of Na(OAc)₃BH (380 mg, 1.79 mmol). The mixture was stirred for 15 min at 25° C. The mixture was diluted with water (10 mL) and extracted with DCM (3 × 10 mL). The combined organic layers were washed with brine (10 mL), dried over anhydrous Na₂SO₄, filtered, concentrated under reduced pressure, and purified by reverse-phase preparative HPLC. LCMS: m/z = 451.5, 453.4 [M+H]⁺. ¹H NMR (400 MHz, CDCl₃) δ 8.31 (dd, J= 8.5, 5.4 Hz, 1H), 8.04-8.01 (m, 2H), 7.85 (dd, J= 8.5, 1.8 Hz, 1H), 4.96-4.82 (m, 2H), 4.64 (dt, J= 3.9, 2.1 Hz, 1H), 4.18 (dd, J= 13.1, 7.4 Hz, 1H), 4.00-3.95 (m, 1H), 3.83 (ddd, J= 13.5, 10.4, 3.1 Hz, 1H), 3.62 (dd, J= 13.1, 7.0 Hz, 1H), 3.46-3.39 (m, 1H), 3.26-3.19 (m, 2H), 3.16 (dd, J= 13.6, 3.6 Hz, 1H), 3.05-3.00 (m, 1H), 2.95-2.84 (m, 2H), 1.37 (dt, J= 10.8, 5.1 Hz, 6H), 1.21 (t, J = 7.2 Hz, 3H).

### Example 160

### 2-Bromo-4-(3-Trans-Methoxycyclobutoxy)-1-Oxo-Phthalazin-2-yl]-N-(5-Fluoropyrimidin-2-yl)Acetamide (160)

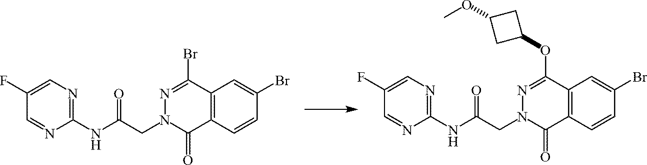 [SMILES: O=C(Cn1nc(Br)c2cc(Br)ccc2c1=O)Nc1ncc(F)cn1 ; CO[C@H]1C[C@H](Oc2nn(CC(=O)Nc3ncc(F)cn3)c(=O)c3ccc(Br)cc23)C1]

To a solution of NaH (7.8 mg, 0.33 mmol) in DMF (0.4 mL) was added 3-trans-methoxycyclobutanol (28 mg, 0.27 mmol) at 0° C. The mixture was stirred at 0° C. for 45 min. 2-(4,6-dibromo-1-oxo-phthalazin-2-yl)-N-(5-fluoropyrimidin-2-yl)acetamide (50 mg, 0.11 mmol) was added as a solid in one portion at 0° C. The mixture was stirred for 2 h at 0° C. The reaction was diluted with aq. HCl (1 mL, 1 M) and extracted with EtOAc (2 × 10 mL). The combined organic layers were washed with brine (5 mL), dried over anhydrous Na₂SO₄, filtered, concentrated under reduced pressure, and purified by silica gel column chromatography. LCMS: m/z = 478.2, 480.2 [M+H]⁺. ¹H NMR (400 MHz, DMSO-d₆): δ 11.09 (s, 1H), 8.79 (s, 2H), 8.17-8.15 (m, 2H), 8.11 (dd, J = 8.5, 1.9 Hz, 1H), 5.19-5.13 (m, 1H), 5.02-4.96 (m, 2H), 4.13-4.07 (m, 1H), 3.16 (s, 3H), 2.45-2.39 (m, 4H).

### Example 161

### 2-Bromo-4-(Oxetan-3-Yloxy)-1-Oxo-Phthalazin-2-yl]-N-(5-Fluoropyrimidin-2-yl)Acetamide (161)

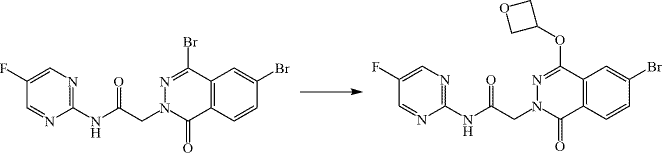 [SMILES: O=C(Cn1nc(Br)c2cc(Br)ccc2c1=O)Nc1ncc(F)cn1 ; O=C(Cn1nc(OC2COC2)c2cc(Br)ccc2c1=O)Nc1ncc(F)cn1]

To a solution of NaH (7.8 mg, 0.33 mmol) in DMF (0.4 mL) was added oxetan-3-ol (20 mg, 0.27 mmol) at 0° C. The mixture was stirred at 0° C. for 45 min. 2-(4,6-dibromo-1-oxo-phthalazin-2-yl)-N-(5-fluoropyrimidin-2-yl)acetamide (50 mg, 0.11 mmol) was added as a solid in one portion at 0° C. The mixture was stirred for 2 h at 0° C. The reaction was diluted with aq. HCl (1 mL, 1 M) and extracted with EtOAc (2 × 10 mL). The combined organic layers were washed with brine (5 mL), dried over anhydrous Na₂SO₄, filtered, concentrated under reduced pressure, and purified by silica gel column chromatography. LCMS: m/z = 450.2, 452.1 [M+H]⁺. ¹H NMR (400 MHz, DMSO-d₆): δ 11.09 (s, 1H), 8.79 (d, J= 0.7 Hz, 2H), 8.26 (dd, J= 1.9, 0.6 Hz, 1H), 8.19-8.12 (m, 2H), 5.55-5.49 (m, 1H), 4.96 (s, 2H), 4.90-4.86 (m, 2H), 4.69 (ddd, J= 7.7, 5.1, 0.7 Hz, 2H).

### Example 162

### 2-Bromo-4-(2-Fluoropropoxy)-1-Oxo-Phthalazin-2-yl]-N-(5-Fluoropyrimidin-2-yl)Acetamide (162)

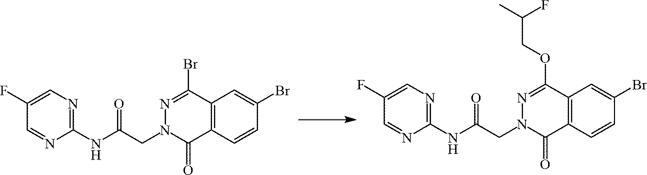 [SMILES: O=C(Cn1nc(Br)c2cc(Br)ccc2c1=O)Nc1ncc(F)cn1 ; CC(F)COc1nn(CC(=O)Nc2ncc(F)cn2)c(=O)c2ccc(Br)cc12]

To a solution of NaH (7.8 mg, 0.33 mmol) in DMF (0.5 mL) was added 2-fluoropropan-1-ol (21 mg, 0.39 mmol) at 0° C. The mixture was stirred at 0° C. for 45 min. 2-(4,6-dibromo-1-oxo-phthalazin-2-yl)-N-(5-fluoropyrimidin-2-yl)acetamide (50 mg, 0.11 mmol) was added as a solid in one portion at 0° C. The mixture was stirred for 2 h at 0° C. The reaction was diluted with aq. HCl (1 mL, 1 M) and extracted with EtOAc (2 × 10 mL). The combined organic layers were washed with brine (5 mL), dried over anhydrous Na₂SO₄, filtered, concentrated under reduced pressure, and purified by silica gel column chromatography. LCMS: m/z = 454.2, 456.2 [M+H]⁺. ¹H NMR (400 MHz, CDCl₃): δ 8.85-8.84 (m, 1H), 8.50 (s, 2H), 8.32 (d, J= 8.5 Hz, 1H), 8.22-8.21 (m, 1H), 7.94 (dd, J= 8.5, 2.0 Hz, 1H), 5.28-5.23 (m, 2H), 5.20-5.01 (m, 1H), 4.45-4.36 (m, 2H), 1.50 (dd, J= 23.4, 6.5 Hz, 3H).

### Example 163

### 2-Bromo-4-(2-Methoxyethoxy)-1-Oxophthalazin-2-yl]-N-(5-Fluoropyrimidin-2-yl)Acetamide (163)

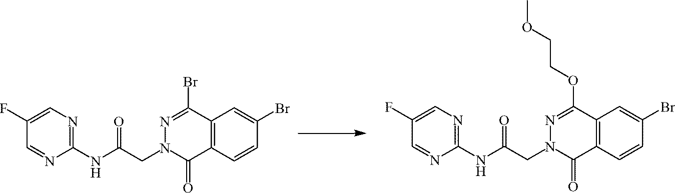 [SMILES: O=C(Cn1nc(Br)c2cc(Br)ccc2c1=O)Nc1ncc(F)cn1 ; COCCOc1nn(CC(=O)Nc2ncc(F)cn2)c(=O)c2ccc(Br)cc12]

To a solution of NaH (7.8 mg, 0.33 mmol) in DMF (0.4 mL) was added 2-methoxyethanol (20 mg, 0.27 mmol) at 0° C. The mixture was stirred at 0° C. for 45 min. 2-(4,6-dibromo-1-oxo-phthalazin-2-yl)-N-(5-fluoropyrimidin-2-yl)acetamide (50 mg, 0.11 mmol) was added as a solid in one portion at 0° C. The mixture was stirred for 2 h at 0° C. The reaction was diluted with aq. HCl (1 mL, 1 M) and extracted with EtOAc (2 × 10 mL). The combined organic layers were washed with brine (5 mL), dried over anhydrous Na₂SO₄, filtered, concentrated under reduced pressure, and purified by silica gel column chromatography. LCMS: m/z = 452.2, 454.2 [M+H]⁺. ¹H NMR (400 MHz; CDCl₃): δ 9.00 (s, 1H), 8.50 (s, 2H), 8.30 (dd, J= 8.5, 0.5 Hz, 1H), 8.22 (dd, J= 2.0, 0.5 Hz, 1H), 7.92 (dd, J= 8.5, 1.9 Hz, 1H), 5.25 (s, 2H), 4.50-4.47 (m, 2H), 3.83-3.81 (m, 2H), 3.48 (s, 3H).

### Example 164

### 2-Bromo-4-Cyclopropyl-1-Oxophthalazin-2-yl)-N-(5-Fluoropyrimidin-2-yl)Acetamide (164)

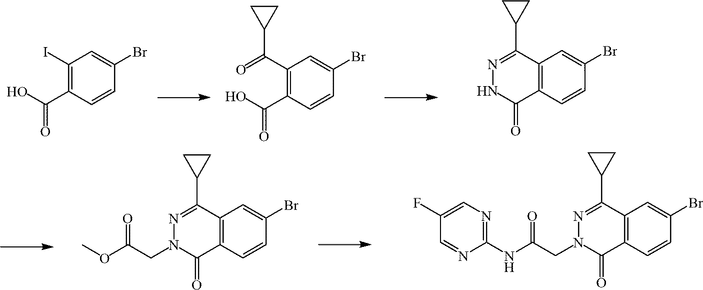 [SMILES: O=C(O)c1ccc(Br)cc1I ; O=C(O)c1ccc(Br)cc1C(=O)C1CC1 ; O=c1[nH]nc(C2CC2)c2cc(Br)ccc12 ; COC(=O)Cn1nc(C2CC2)c2cc(Br)ccc2c1=O ; O=C(Cn1nc(C2CC2)c2cc(Br)ccc2c1=O)Nc1ncc(F)cn1]

4-bromo-2-(cyclopropanecarbonyl)benzoic acid: To a solution of 4-bromo-2-iodobenzoic acid (500 mg, 1.53 mmol) in THF (5.0 mL) at -78° C. was added n-BuLi (2.5 M in hexane, 1.22 mL). The reaction mixture was stirred at -78° C. for 0.5 h followed by that addition of a solution of N-methoxy-N-methylcyclopropanecarboxamide (217 mg, 1.68 mmol) in THF (3.0 mL). The reaction mixture was allowed to warm to 15° C. and stirred for a further 2.5 h. The reaction mixture was quenched by the addition of sat. aq. NH₄Cl (100 mL), adjusted to pH=3 using aq. HCl (3 M), and extracted with EtOAc (3 × 200 mL). The combined organics were dried over anhydrous Na₂SO₄, filtered, and concentrated under reduced pressure to provide a residue that was used directly. LCMS: m/z = 266.9, 268.9 [M-H]⁻.

6-bromo-4-cyclopropylphthalazin-1(2H)-one: To a solution of 4-bromo-2-(cyclopropanecarbonyl)benzoic acid (400 mg, 1.49 mmol) in toluene (10 mL) was added NH₂NH₂·H₂O (759 mg, 14.9 mmol). The reaction mixture was stirred at 100° C. for 16 h. The reaction mixture was concentrated under reduced pressure to provide a residue that was used directly. LCMS: m/z = 265.0, 267.0 [M+H]⁺.

methyl 2-(6-bromo-4-cyclopropyl-1-oxophthalazin-2(1H)-yl)acetate: To a solution of 6-bromo-4-cyclopropylphthalazin-1(2H)-one (250 mg, 0.94 mmol) in DMF (5.0 mL) were added Cs₂CO₃ (615 mg, 1.89 mmol) and methyl 2-bromoacetate (173 mg, 1.13 mmol). The reaction mixture was stirred at 90° C. for 2 h. The reaction mixture was diluted with water (15 mL) and extracted with EtOAc (3 × 8 mL). The combined organics were washed with brine (10 mL), dried over anhydrous Na₂SO₄, filtered, and concentrated under reduced pressure. The residue was purified by silica gel column chromatography. LCMS: m/z = 337.0, 339.0 [M+H]⁺.

2-(6-bromo-4-cyclopropyl-1-oxophthalazin-2-yl)-N-(5-fluoropyrimidin-2-yl)acetamide: To a solution of methyl 2-(6-bromo-4-cyclopropyl-1-oxophthalazin-2(1H)-yl)acetate (30 mg, 0.09 mmol) in DCE (2.0 mL) were added 5-fluoropyrimidin-2-amine (12 mg, 0.11 mmol) and trimethyl-(4-trimethylalumanuidyl-1,4-diazoniabicyclo[2.2.2]octan-1-yl)alumanuide (30 mg, 0.12 mmol). The reaction mixture was stirred at 60° C. for 12 h. The reaction mixture was diluted with water (15 mL), filtered, and the filtrate was extracted with EtOAc (3 × 10 mL). The combined organic layers were dried over anhydrous Na₂SO₄, filtered, and concentrated under reduced pressure. The residue was purified by reverse-phase preparative HPLC. LCMS: m/z = 418.1, 420.1 [M+H]⁺. ¹H NMR (400 MHz, CDCl₃) δ 8.80 (br s, 1H), 8.48 (s, 2H), 8.35 (d, J= 8.4 Hz, 1H), 8.28 (d, J= 1.6 Hz, 1H), 7.90 (dd, J = 8.4, 1.6 Hz, 1H), 5.32 (s, 2H), 2.26-2.16 (m, 1H), 1.08-1.02 (m, 4H).

### Examples 165 and 166

### 2-Bromo-4-[2-Cis-Fluorocyclopropyl]-1-Oxophthalazin-2-yl]-N-(5-Fluoropyrimidin-2-yl)Acetamide (165 and 166)

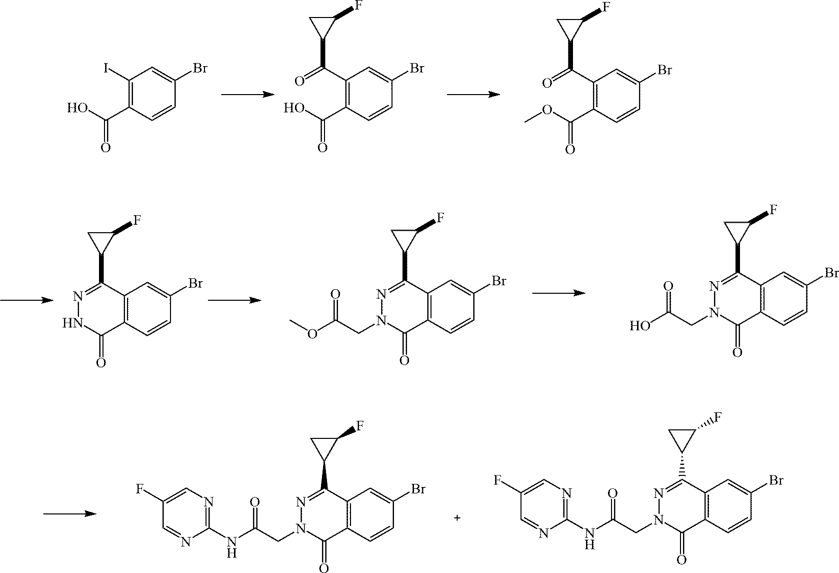

4-bromo-2-(2-cis-fluorocyclopropanecarbonyl)benzoic acid: To a solution of 4-bromo-2-iodobenzoic acid (4.0 g, 12.2 mmol) in THF (40 mL) at -78° C. was added n-BuLi (9.79 mL, 2.5 M in hexanes). The reaction mixture was stirred at -78° C. for 0.5 h followed by the addition of a solution of 2-cis-fluoro-N-methoxy-N-methylcyclopropanecarboxamide (1.98 g, 13.5 mmol) in THF (3.0 mL). The reaction mixture was stirred at -78° C. for a further 1 h. The reaction mixture was quenched by the addition of sat. aq. NH₄Cl (40 mL) and allowed to warm to ambient temperature. The reaction mixture was then adjusted to pH=9 using sat. aq. Na₂CO₃ and washed with MBTE (20 mL). The aqueous layer was then adjusted pH=3 using aq. HCl (3 M) and extracted with EtOAc (3 × 20 mL). The combined organic layers were washed with brine (20 mL), dried over anhydrous Na₂SO₄, filtered, and concentrated under reduced pressure to provide a residue that was used directly. LCMS: m/z = 286.9, 288.9 [M+H]⁺.

methyl 4-bromo-2-(2-cis-fluorocyclopropanecarbonyl)benzoate: To a solution of 4-bromo-2-(2-cis-fluorocyclopropanecarbonyl)benzoic acid (1.0 g, 3.48 mmol) and K₂CO₃ (1.44 g, 10.5 mmol) in DMF (10 mL) at 0° C. was added CH₃I (494 mg, 3.48 mmol). The reaction mixture was stirred at 20° C. for 2 h. The reaction mixture was diluted with water (50 mL) and extracted with EtOAc (3 × 20 mL). The combined organic layers were washed with brine (20 mL), dried over anhydrous Na₂SO₄, filtered, and concentrated under reduced pressure The residue was purified by silica gel column chromatography. LCMS: m/z = 300.9, 302.9 [M+H]⁺.

6-bromo-4-(2-cis-fluorocyclopropyl)phthalazin-1(2H)-one: To a solution of methyl 4-bromo-2-(2-cis-fluorocyclopropanecarbonyl)benzoate (250 mg, 0.83 mmol) in EtOH (10 mL) was added N₂H₄·H₂O (42 mg, 0.83 mmol). The reaction mixture was stirred at 20° C. for 16 h. The reaction mixture was concentrated under reduced pressure to provide a residue that was used directly. LCMS: m/z = 282.9, 284.9 [M+H]⁺.

methyl 2-(6-bromo-4-(2-cis-fluorocyclopropyl)-1-oxophthalazin-2(1H)-yl)acetate: To a solution of 6-bromo-4-(2-cis-fluorocyclopropyl)phthalazin-1(2H)-one mixture (230 mg, 0.81 mmol) and methyl 2-bromoacetate (248 mg, 1.62 mmol) in DMF (5 mL) was added Cs₂CO₃ (794 mg, 2.44 mmol). The reaction mixture was stirred at 20° C. for 16 h. The reaction mixture was diluted with water (30 mL) and extracted with EtOAc (3 × 10 mL). The combined organic layers were washed with brine (10 mL), dried over anhydrous Na₂SO₄, filtered, and concentrated under reduced pressure. The residue was purified by silica gel column chromatography. LCMS: m/z = 354.9, 356.9 [M+H]⁺.

2-(6-bromo-4-(2-cis-fluorocyclopropyl)-1-oxophthalazin-2(1H)-yl)acetic acid: To a solution of methyl 2-(6-bromo-4-(2-cis-fluorocyclopropyl)-1-oxophthalazin-2(1H)-yl)acetate (80 mg, 0.23 mmol) in THF (1.0 mL) and water (1.0 mL) was added LiOH·H₂O (23.63 mg, 0.56 mmol). The reaction mixture was stirred at 20° C. for 2 h. The reaction mixture was washed with MBTE (1 mL) and then the aqueous layer was adjusted to pH=3 using aq. HCl (3 M). The aqueous layer was extracted with EtOAc (3 × 3 mL). The combined organic layers were washed with brine (3 mL), dried over anhydrous Na₂SO₄, filtered, and concentrated under reduced pressure to provide a residue that was used directly. LCMS: m/z = 341.0, 343.1 [M+H]⁺.

2-[6-bromo-4-[2-cis-fluorocyclopropyl]-1-oxophthalazin-2-yl]-N-(5-fluoropyrimidin-2-yl)acetamide: To a solution of 2-(6-bromo-4-(2-cis-fluorocyclopropyl)-1-oxophthalazin-2(1H)-yl)acetic acid (50 mg, 0.15 mmol) and 5-fluoropyrimidin-2-amine (74 mg, 0.66 mmol) in pyridine (1.0 mL) was added EDCI (140 mg, 0.73 mmol). The reaction mixture was stirred at 80° C. for 2 h. The reaction mixture was diluted with water (1 mL) and extracted with EtOAc (3 × 10 mL). The combined organic layers were washed with brine (3 mL), dried over anhydrous Na₂SO₄, filtered, and concentrated under reduced pressure. The residue was purified by reverse-phase preparative HPLC followed by preparative chiral SFC (column: Chiralpak IC-3 (50 mm × 4.6 mm, 3 µM particle size); mobile phase: A: CO₂, B: 0.1% iPrOH in EtOH; 50% B isocratic; flow rate: 3.4 mL/min; column temperature: 35° C.; back pressure: 1800 psi) to provide:

2-[6-bromo-4-[2-cis-fluorocyclopropyl]-1-oxophthalazin-2-yl]-N-(5-fluoropyrimidin-2-yl)acetamide (first eluting isomer, 165). LCMS: m/z = 435.9, 437.9 [M+H]⁺. ¹H NMR (400 MHz, CDCl₃) δ 9.04 (br s, 1H), 8.49 (s, 2H), 8.35 (d, J= 8.4 Hz, 1H), 8.21 (d, J= 1.6 Hz, 1H), 7.90 (dd, J = 2.0, 8.8 Hz, 1H), 5.59-5.51 (m, 1H), 5.29-5.12 (m, 1H), 5.15-4.90 (m, 1H), 2.41-2.30 (m, 1H), 2.06-1.92 (m, 1H), 1.31-1.18 (m, 1H); and

2-[6-bromo-4-[2-cis-fluorocyclopropyl]-1-oxophthalazin-2-yl]-N-(5-fluoropyrimidin-2-yl)acetamide (second eluting isomer, 166). LCMS: m/z = 435.9, 437.9 [M+H]⁺. ¹H NMR (400 MHz, CDCl₃) δ 9.20 (br s, 1H), 8.50 (s, 2H), 8.34 (d, J= 8.8 Hz, 1H), 8.20 (d, J= 1.2 Hz, 1H), 7.90 (dd, J= 2.0, 8.4 Hz, 1H), 5.59-5.51 (m, 1H), 5.29-5.12 (m, 1H), 5.14-4.89 (m, 1H), 2.41-2.27 (m, 1H), 2.05-1.91 (m, 1H), 1.33-1.16 (m, 1H).

### Example 167

### 2-Bromo-4-[2-Trans-Fluorocyclopropyl]-1-Oxophthalazin-2-yl]-N-(5-Fluoropyrimidin-2-yl)Acetamide (167)

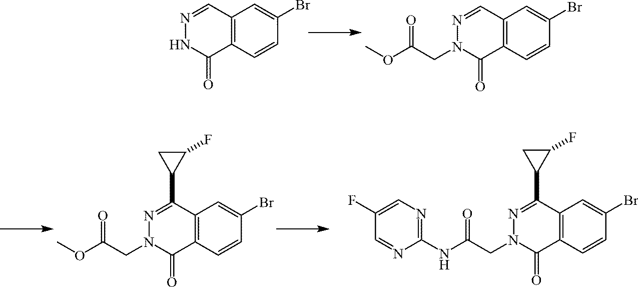 [SMILES: O=c1[nH]ncc2cc(Br)ccc12 ; COC(=O)Cn1ncc2cc(Br)ccc2c1=O ; COC(=O)Cn1nc([C@H]2C[C@@H]2F)c2cc(Br)ccc2c1=O ; O=C(Cn1nc([C@H]2C[C@@H]2F)c2cc(Br)ccc2c1=O)Nc1ncc(F)cn1]

methyl 2-(6-bromo-1-oxophthalazin-2(1H)-yl)acetate: To a solution of 6-bromophthalazin-1(2H)-one (3.0 g, 13.3 mmol) in DMF (30 mL) were added Cs₂CO₃ (8.69 g, 26.7 mmol) and methyl 2-bromoacetate (4.08 g, 26.7 mmol). The reaction mixture was stirred at 25° C. for 12 h. The reaction mixture was diluted with water (30 mL). The solid was collected by filtration, washed with water (3 × 10 mL), and dried under reduced pressure to give a residue that was used directly. LCMS: m/z = 296.9, 298.9 [M+H]⁺.

methyl 2-(6-bromo-4-(2-trans-fluorocyclopropyl)-1-oxophthalazin-2(1H)-yl)acetate: To a solution of methyl 2-(6-bromo-1-oxophthalazin-2(1H)-yl)acetate (2.4 g, 8.08 mmol) and 2-cis-fluorocyclopropanecarboxylic acid (2.5 g, 24.2 mmol) in MeCN (30 mL) and water (5 mL) were added a solution of AgNO₃ (5.49 g, 32.3 mmol) in water (5.0 mL) and TFA (184 mg, 1.62 mmol) followed by a solution of ammonia sulfooxy hydrogen sulfate (9.22 g, 40.4 mmol) in water (5 mL). The reaction mixture was stirred at 90° C. for 12 h. The reaction mixture was diluted with water (50 mL) and extracted with EtOAc (3 × 10 mL). The combined organics were washed with brine (10 mL), dried over anhydrous Na₂SO₄, filtered, and concentrated under reduced pressure. The residue was purified by reverse-phase preparative HPLC. LCMS: m/z = 354.9, 356.9 [M+H]⁺.

2-[6-bromo-4-[2-trans-fluorocyclopropyl]-1-oxophthalazin-2-yl]-N-(5-fluoropyrimidin-2-yl)acetamide: To a solution of 2-(6-bromo-4-(2-trans-fluorocyclopropyl)-1-oxophthalazin-2(1H)-yl)acetate (70 mg, 0.20 mmol) and 5-fluoropyrimidin-2-amine (67 mg, 0.59 mmol) in DCE (2.0 mL) was added AlMe₃ (1 M in n-heptane, 0.59 mmol). The reaction mixture was stirred at 60° C. for 12 h. The reaction mixture was diluted with water (5 mL) and extracted with EtOAc (3 × 5 mL). The combined organic layers were dried over anhydrous Na₂SO₄, filtered, and concentrated under reduced pressure. The residue was purified by reverse-phase preparative HPLC. LCMS: m/z = 435.9, 437.9 [M+H]⁺. ¹H NMR (400 MHz, CDCl₃) δ 8.77 (s, 1H), 8.65 (br s, 1H), 8.49 (s, 2H), 8.23 (d, J = 8.5 Hz, 1H), 8.00-7.88 (m, 1H), 5.57-5.35 (s, 2H), 4.93-4.63 (m, 1H), 2.65-2.45 (m, 1H), 2.08-1.85 (m, 1H), 1.29-1.17 (m, 1H).

### Example 168

### 2-Bromo-4-[Cyclopropyl(Fluoro)Methoxy]-1-Oxophthalazin-2-yl]-N-(5-Fluoropyrimidin-2-yl)Acetamide (168)

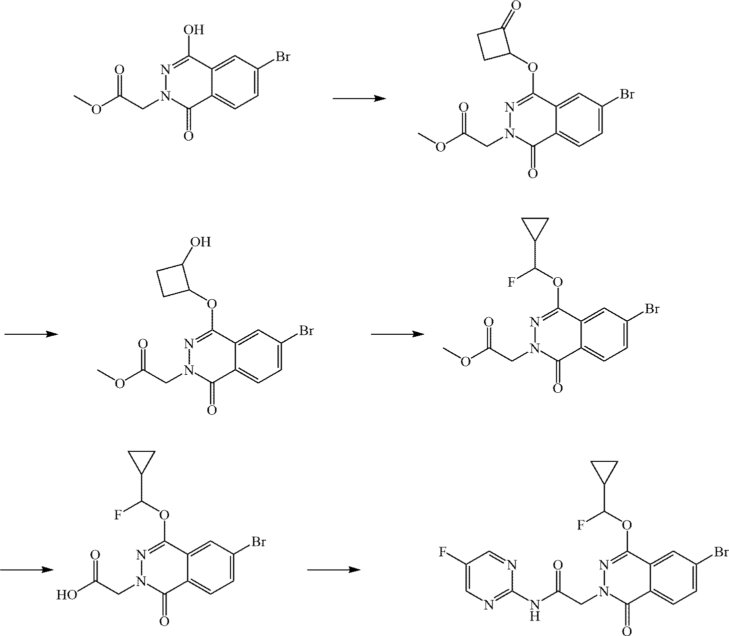 [SMILES: COC(=O)Cn1nc(O)c2cc(Br)ccc2c1=O ; COC(=O)Cn1nc(OC2CCC2=O)c2cc(Br)ccc2c1=O ; COC(=O)Cn1nc(OC2CCC2O)c2cc(Br)ccc2c1=O ; COC(=O)Cn1nc(OC(F)C2CC2)c2cc(Br)ccc2c1=O ; O=C(O)Cn1nc(OC(F)C2CC2)c2cc(Br)ccc2c1=O ; O=C(Cn1nc(OC(F)C2CC2)c2cc(Br)ccc2c1=O)Nc1ncc(F)cn1]

methyl 2-(6-bromo-1-oxo-4-(2-oxocyclobutoxy)phthalazin-2(1H)-yl)acetate: To a solution of methyl 2-(6-bromo-4-hydroxy-1-oxophthalazin-2(1H)-yl)acetate (200 mg, 0.64 mmol) and 2-bromocyclobutanone (190 mg, 1.3 mmol) in MeCN (3 mL) was added K₂CO₃ (176 mg, 1.3 mmol). The mixture was stirred at 25° C. for 1 h. The reaction mixture was poured into water (10 mL) and extracted with EtOAc (3 × 10 mL). The combined organic layers were dried over anhydrous Na₂SO₄, filtered, and concentrated under reduced pressure and purified by silica gel column chromatography. LCMS: m/z = 380.9, 382.9 [M+H]⁺.

methyl 2-(6-bromo-4-(2-hydroxycyclobutoxy)-1-oxophthalazin-2(1H)-yl)acetate: To a solution of methyl 2-(6-bromo-1-oxo-4-(2-oxocyclobutoxy)phthalazin-2(1H)-yl)acetate (130 mg, 0.34 mmol) in MeOH (2 mL) at 0° C. was added NaBH₄ (256 mg, 0.68 mmol). The mixture was stirred at 0° C. for 1 h. The reaction mixture was quenched by addition of aq. sat. NH₄Cl (10 mL) and extracted with EtOAc (3 × 10 mL). The combined organic layers were dried over anhydrous Na₂SO₄, filtered, and concentrated under reduced pressure to provide a residue that was used directly. LCMS: m/z = 382.9, 384.9 [M+H]⁺.

methyl 2-(6-bromo-4-(cyclopropylfluoromethoxy)-1-oxophthalazin-2(1H)-yl)acetate: To a solution of methyl 2-(6-bromo-4-(2-hydroxycyclobutoxy)-1-oxophthalazin-2(1H)-yl)acetate (380 mg, 0.99 mmol) in DCM (2 mL) at -78° C. were added N,N-diethylethanamine;trihydrofluoride (1.60 g, 9.9 mmol) and (diethylamino)difluorosulfonium tetrafluoroborate (2.27 g, 9.9 mmol). The reaction mixture was stirred at 15° C. for 2 h. The reaction mixture was quenched by addition of water 10 (mL) and extracted with EtOAc (3 × 10 mL). The combined organic layers were dried over anhydrous Na₂SO₄, filtered, concentrated under reduced pressure, and purified by silica gel column chromatography. LCMS: m/z = 384.9, 386.9 [M+H]⁺.

2-(6-bromo-4-(cyclopropylfluoromethoxy)-1-oxophthalazin-2(1H)-yl)acetic acid: To a solution of methyl 2-(6-bromo-4-(cyclopropylfluoromethoxy)-1-oxophthalazin-2(1H)-yl)acetate (70 mg, 0.18 mmol) in THF (1.0 mL) and water (0.2 mL) at 0° C. was added LiOH·H₂O (15 mg, 0.36 mmol). The reaction mixture was stirred at 0° C. for 1 h. The reaction mixture was diluted with water (5 mL). The aqueous phase was acidified to pH = 3 using aq. HCl (3 M) and extracted with EtOAc (3 × 10 mL). The combined organic layers were dried over anhydrous Na₂SO₄, filtered, and concentrated under reduced pressure to provide a residue that was used directly. LCMS: m/z = 370.9, 372.9 [M+H]⁺.

2-[6-bromo-4-[cyclopropyl(fluoro)methoxy]-1-oxophthalazin-2-yl]-N-(5-fluoropyrimidin-2-yl)acetamide: To a solution of 2-(6-bromo-4-(cyclopropylfluoromethoxy)-1-oxophthalazin-2(1H)-yl)acetic acid (60 mg, 0.16 mmol) and 5-fluoropyrimidin-2-amine (21 mg, 0.20 mmol) in pyridine (1 mL) was added EDCI (46 mg, 0.24 mmol). The mixture was stirred at 25° C. for 12 h. The reaction mixture was poured into water (10 mL) and extracted with EtOAc (2 × 10 mL). The combined organic layers were dried over anhydrous Na₂SO₄, filtered, concentrated under reduced pressure, and purified by reverse-phase preparative HPLC. LCMS: m/z = 465.9, 467.9 [M+H]⁺. ¹H NMR (400 MHz, DMSO-d₆): δ 11.11 (s, 1H), 8.77 (s, 2H), 8.28-8.03 (m, 3H), 6.10 (dd, J= 6.4, 57.2 Hz, 1H), 5.01 (s, 2H), 1.68-1.50 (m, 1H), 0.75-0.58 (m, 4H).

### Example 169

### 2-Bromo-4-(1-Cyclopropylethoxy)-1-Oxophthalazin-2-yl]-N-(5-Fluoropyrimidin-2-yl)Acetamide (169)

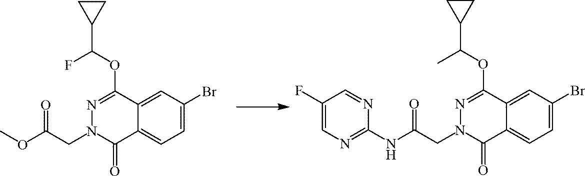 [SMILES: COC(=O)Cn1nc(OC(F)C2CC2)c2cc(Br)ccc2c1=O ; CC(Oc1nn(CC(=O)Nc2ncc(F)cn2)c(=O)c2ccc(Br)cc12)C1CC1]

To a solution of methyl 2-(6-bromo-4-(cyclopropylfluoromethoxy)-1-oxophthalazin-2(1H)-yl)acetate (80 mg, 0.20 mmol) and 5-fluoropyrimidin-2-amine (47 mg, 0.42 mmol) in DCE (2 mL) was added AlMe₃ (1 M in n-heptane, 0.30 mmol). The mixture was stirred at 60° C. for 12 h. The mixture was diluted with water (5 mL) and extracted with EtOAc (3 × 5 mL). The combined organic layers were dried over anhydrous Na₂SO₄, filtered, concentrated under reduced pressure, and purified by reverse-phase preparative HPLC. LCMS: m/z = 461.9, 463.9 [M+H]⁺. ¹H NMR (400 MHz, CDCl₃): δ 8.98 (br s, 1H), 8.48 (br s, 2H), 8.29 (d, J= 8.4 Hz, 1H), 8.20 (d, J= 1.6 Hz, 1H), 7.90 (dd, J = 2.0, 8.4 Hz, 1H), 5.18 (br s, 2H), 4.61-4.47 (m, 1H), 1.45 (d, J = 6.4 Hz, 3H), 1.28-1.13 (m, 1H), 0.64-0.56 (m, 2H), 0.53-0.45 (m, 1H), 0.37-0.25 (m, 1H).

### Example 170

### 2-Bromo-1-Oxo-4-[1-(2,2,2-Trifluoroethyl)Azetidin-3-yl]Oxyphthalazin-2-yl]-N-(5-Fluoropyrimidin-2-yl)Acetamide (170)

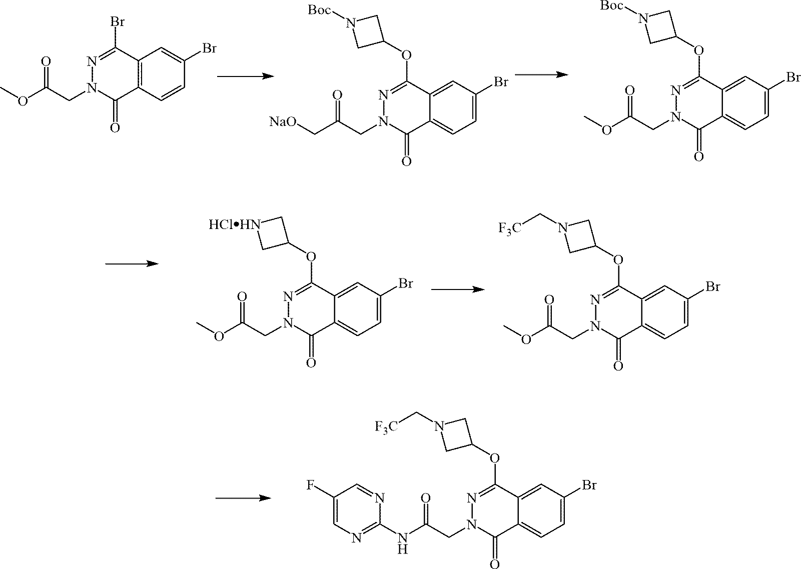

sodium 2-(6-bromo-4-((1-(tert-butoxycarbonyl)azetidin-3-yl)oxy)-1-oxophthalazin-2(1H)-yl)acetate: DMF (50 mL) was added to NaH (1.63 g, 41.0 mmol, 60% purity) at 0° C. followed by tert-butyl 3-hydroxyazetidine-1-carboxylate (4.4 g, 25.5 mmol). The reaction mixture was stirred at 0° C. for 30 min. Methyl 2-(4,6-dibromo-1-oxophthalazin-2(1H)-yl)acetate (4.8 g, 12.8 mmol) was then added and the mixture was stirred at 0° C. for 2 h. The reaction mixture was poured into ice-cold water (10 mL) and filtered. The filter cake was dried under reduced pressure to provide a residue that was used directly.

tert-butyl 3-((7-bromo-3-(2-methoxy-2-oxoethyl)-4-oxo-3,4-dihydrophthalazin-1-yl)oxy)azetidine-1-carboxylate: To a solution of sodium 2-(6-bromo-4-((1-(tert-butoxycarbonyl)azetidin-3-yl)oxy)-1-oxophthalazin-2(1H)-yl)acetate (500 mg, 1.05 mmol) in DMF (10 mL) was added methyl iodide (223 mg, 1.57 mmol). The mixture was stirred at 25° C. for 6 h. The reaction mixture was quenched by addition of water (10 mL) and extracted with EtOAc (3 × 15 mL). The combined organic layers were washed with brine (15 mL), dried over anhydrous Na₂SO₄, filtered, concentrated under reduced pressure, and purified by silica gel column chromatography.

methyl 2-(4-(azetidin-3-yloxy)-6-bromo-1-oxophthalazin-2(1H)-yl)acetate HCl salt: To a solution of tert-butyl 3-((7-bromo-3-(2-methoxy-2-oxoethyl)-4-oxo-3,4-dihydrophthalazin-1-yl)oxy)azetidine-1-carboxylate (1.1 g, 2.35 mmol) in EtOAc (5 mL) was added HCl (4 M in EtOAc, 20 mL). The mixture was stirred at 20° C. for 1 h. The reaction mixture was concentrated under reduced pressure to provide a residue that was used directly. LCMS: m/z = 367.9, 369.8 [M+H]⁺.

methyl 2-(6-bromo-1-oxo-4-((1-(2,2,2-trifluoroethyl)azetidin-3-yl)oxy)phthalazin-2(1H)-yl)acetate: To a solution of methyl 2-(4-(azetidin-3-yloxy)-6-bromo-1-oxophthalazin-2(1H)-yl)acetate HCl salt (100 mg, 0.24 mmol) in DMF (2 mL) was added DIPEA (96 mg, 0.74 mmol). The mixture was stirred at 25° C. for 10 min before a solution of 2,2,2-trifluoroethyl trifluoromethanesulfonate (57 mg, 0.24 mmol) in DMF (0.5 mL) was added. The mixture was stirred at 25° C. for 6 h. The reaction mixture was cooled to 0° C., diluted with water (10 mL), and extracted with EtOAc (3 × 5 mL). The combined organic layers were washed with brine (20 mL), dried over anhydrous Na₂SO₄, filtered, concentrated under reduced pressure, and purified by preparative TLC. LCMS: m/z = 450.2, 452.1 [M+H]⁺.

2-[6-bromo-1-oxo-4-[1-(2,2,2-trifluoroethyl)azetidin-3-yl]oxyphthalazin-2-yl]-N-(5-fluoropyrimidin-2-yl)acetamide: To a solution of methyl 2-(6-bromo-1-oxo-4-((1-(2,2,2-trifluoroethyl)azetidin-3-yl)oxy)phthalazin-2(1H)-yl)acetate (50 mg, 0.11 mmol) and 5-fluoropyrimidin-2-amine (25 mg, 0.22 mmol) in DCE (2.0 mL) was added AlMe₃ (1 M in n-heptane, 0.33 mL). The reaction mixture was stirred at 60° C. for 8 h. The reaction mixture cooled to 0° C., diluted with water (8 mL), and extracted with EtOAc (3 × 5 mL). The combined organic layers were washed with brine (15 mL), dried over anhydrous Na₂SO₄, filtered, concentrated under reduced pressure, and purified by reverse-phase preparative HPLC. LCMS: m/z = 531.0, 533.0 [M+H]⁺. ¹H NMR (400 MHz, CDCl₂): δ 9.22 (s, 1H), 8.50 (s, 2H), 8.28 (d, J= 8.8 Hz, 1H), 8.15 (d, J= 1.6 Hz, 1H), 7.91 (dd, J= 2.0, 8.6 Hz, 1H), 5.28-5.21 (m, 3H), 4.08-3.92 (m, 2H), 3.54-3.34 (m, 2H), 3.10 (q, J= 9.2 Hz, 2H).

### Example 171

### 2-Bromo-4-(3-Cis-Cyanocyclobutyl)Oxy-1-Oxophthalazin-2-yl]-N-(5-Fluoropyrimidin-2-yl)Acetamide (171)

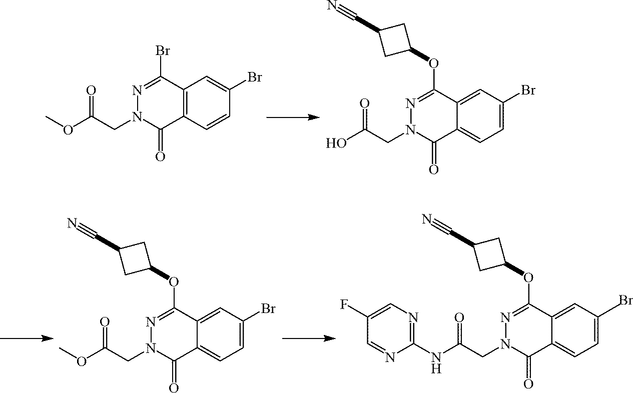 [SMILES: COC(=O)Cn1nc(Br)c2cc(Br)ccc2c1=O ; N#C[C@H]1C[C@@H](Oc2nn(CC(=O)O)c(=O)c3ccc(Br)cc23)C1 ; COC(=O)Cn1nc(O[C@H]2C[C@@H](C#N)C2)c2cc(Br)ccc2c1=O ; Cc1ccc2c(=O)n(CC(=O)Nc3ncc(F)cn3)nc(O[C@H]3C[C@@H](C#N)C3)c2c1 ; Br]

2-(6-bromo-4-(3-cis-cyanocyclobutoxy)-1-oxophthalazin-2(1H)-yl)acetic acid: To a solution of NaH (63 mg, 1.60 mmol, 60% purity) in DMF (2.0 mL) at -10° C. was added 3-cis-hydroxycyclobutanecarbonitrile (103 mg, 1.06 mmol). The reaction was stirred at -10° C. for 30 min. Methyl 2-(4,6-dibromo-1-oxophthalazin-2(1H)-yl)acetate (200 mg, 0.53 mmol) was then added. The reaction mixture was stirred at 20° C. for a further 1 h. The reaction mixture was quenched by addition sat. aq. NH₄Cl (6 mL). The aqueous was adjusted to pH = 4 by addition of citric acid and then extracted with EtOAc (3 × 2 mL). The combined organic layers were washed with brine (3 mL), dried over anhydrous Na₂SO₄, filtered, concentrated under reduced pressure to provide a residue that was used directly. LCMS: m/z = 377.9, 379.9 [M+H]⁺.

methyl 2-(6-bromo-4-(3-cis-cyanocyclobutoxy)-1-oxophthalazin-2(1H)-yl)acetate: To a mixture of 2-(6-bromo-4-(3-cis-cyanocyclobutoxy)-1-oxophthalazin-2(1H)-yl)acetic acid (350 mg, 0.93 mmol) and K₂CO₃ (191 mg, 1.39 mmol) in DMF (4 mL) at 0° C. was added iodomethane (197 mg, 1.39 mmol). The mixture was stirred at 20° C. for 5 h. The reaction mixture was quenched by addition of water (8 mL) and extracted with EtOAc (4 × 2 mL). The combined organic layers were washed with brine (5 mL), dried over anhydrous Na₂SO₄, filtered, concentrated under reduced pressure, and purified by silica gel column chromatography. LCMS: m/z = 391.9, 393.9 [M+H]⁺.

2-[6-bromo-4-(3-cis-cyanocyclobutyl)oxy-1-oxophthalazin-2-yl]-N-(5-fluoropyrimidin-2-yl)acetamide: To a solution of 5-fluoropyrimidin-2-amine (40 mg, 0.35 mmol) and methyl 2-(6-bromo-4-(3-cis-cyanocyclobutoxy)-1-oxophthalazin-2(1H)-yl)acetate (70 mg, 0.18 mmol) in DCE (2 mL) at 0° C. was added AlMe₃ (1 M in n-heptane, 0.392 mL). The mixture was stirred at 60° C. for 5 h. The reaction mixture was quenched by addition of water (6 mL) and extracted with EtOAc (4 × 3 mL). The combined organic layers were washed with brine (5 mL), dried over anhydrous Na₂SO₄, filtered, concentrated under reduced pressure, and purified by reverse-phase preparative HPLC. LCMS: m/z = 473.0, 474.9 [M+H]⁺. ¹H NMR (400 MHz, CDCl₃): δ 8.86 (br s, 1H), 8.49 (s, 2H), 8.30 (d, J= 8.4 Hz, 1H), 8.14 (d, J= 1.2 Hz, 1H), 7.93 (dd, J = 1.6, 8.4 Hz, 1H), 5.20 (s, 2H), 5.16-5.03 (m, 1H), 3.06-2.94 (m, 2H), 2.89-2.76 (m, 1H), 2.73-2.59 (m, 2H).

### Example 172

### 2-Bromo-4-(3-Methoxycyclobutyl)Oxy-1-Oxophthalazin-2-yl]-N-(5-Fluoropyrimidin-2-yl)Acetamide (172)

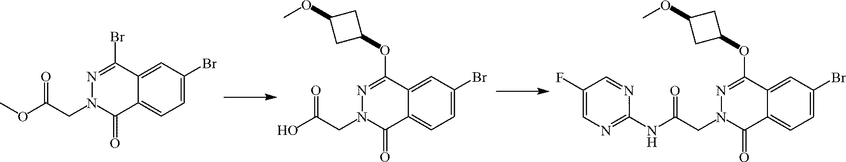 [SMILES: COC(=O)Cn1nc(Br)c2cc(Br)ccc2c1=O ; CO[C@H]1C[C@@H](Oc2nn(CC(=O)O)c(=O)c3ccc(Br)cc23)C1 ; CO[C@H]1C[C@@H](Oc2nn(CC(=O)Nc3ncc(F)cn3)c(=O)c3ccc(Br)cc23)C1]

2-(6-bromo-4-(3-cis-methoxycyclobutoxy)-1-oxophthalazin-2(1H)-yl)acetic acid: To a mixture of 3-cis-methoxycyclobutanol (104 mg, 1.01 mmol) in DMF (2.0 mL) at 0° C. was added NaH (61 mg, 1.52 mmol). The mixture was stirred at 0° C. for 0.5 h followed by the addition of methyl 2-(4,6-dibromo-1-oxo-phthalazin-2-yl)acetate (200 mg, 0.52 mmol). The mixture was stirred at 20° C. for a further 50 min. The reaction mixture was diluted with water (10 mL) and the aqueous layer was acidified to pH = 4 with aq. HCl (3 M). The mixture was extracted with EtOAc (3 × 5 mL). The combined organic layers were washed with brine (5 mL), dried over anhydrous Na₂SO₄, filtered, and concentrated under reduced pressure to provide a residue that was used directly.

2-[6-bromo-4-(3-cis-methoxycyclobutyl)oxy-1-oxophthalazin-2-yl]-N-(5-fluoropyrimidin-2-yl)acetamide: To a mixture of 2-(6-bromo-4-(3-cis-methoxycyclobutoxy)-1-oxophthalazin-2(1H)-yl)acetic acid (100 mg, 0.26 mmol) and 5-fluoropyrimidin-2-amine (59 mg, 0.52 mmol) in pyridine (3 mL) was added EDCI (100 mg, 0.53 mmol). The mixture was stirred at 20° C. for 12 h. The reaction mixture was diluted with water (10 mL) and extracted with EtOAc (3 × 5 mL). The combined organic layers were washed with brine (5 mL), dried over anhydrous Na₂SO₄, filtered, concentrated under reduced pressure, and purified by reverse-phase preparative HPLC. LCMS: m/z = 477.9, 479.9 [M+H]⁺. ¹H NMR (400 MHz, CDCl₃): δ 11.09 (s, 1H), 8.78 (s, 2H), 8.21-8.05 (m, 3H), 4.98 (s, 2H), 4.75-4.68 (m, 1H), 3.68-3.59 (m, 1H), 3.15 (s, 3H), 2.85-2.82 (m, 2H), 2.07-2.02 (m, 2H).

### Example 173

### 2-Cyclopropyl-4-Cyclopropyloxy-1-Oxophthalazin-2-yl)-N-(5-Fluoropyrimidin-2-yl)Acetamide (173)

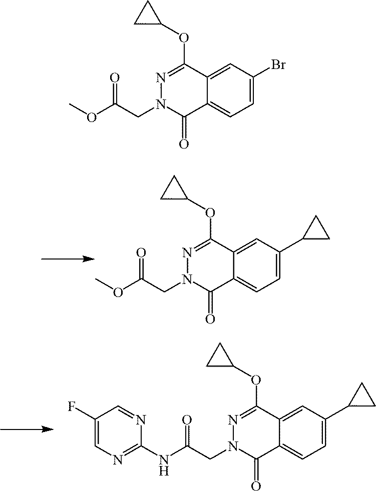

methyl 2-(4-cyclopropoxy-6-cyclopropyl-1-oxophthalazin-2(1H)-yl)acetate: To a solution of methyl 2-(6-bromo-4-cyclopropoxy-1-oxophthalazin-2(1H)-yl)acetate (150 mg, 0.42 mmol) and cyclopropylboronic acid (109 mg, 1.27 mmol) in 1,4-dioxane (2 mL) were added Pd(dppf)Cl₂ (31 mg, 0.04 mmol) and CsF (194 mg, 1.27 mmol). The mixture was stirred at 100° C. for 3 h. The reaction mixture was diluted with water (5 mL) and extracted with EtOAc (3 × 5 mL). The combined organic layers were washed with brine (5 mL), dried over anhydrous Na₂SO₄, filtered, concentrated under reduced pressure, and purified by silica gel column chromatography. ¹H NMR (400 MHz, CDCl₃): δ 8.26 (d, J= 8.4 Hz, 1H), 7.52 (d, J= 1.6 Hz, 1H), 7.43 (dd, J= 1.6, 8.4 Hz, 1H), 4.88 (s, 2H), 4.24-4.20 (m, 1H), 3.78 (s, 3H), 2.14-1.94 (m, 1H), 1.21-1.04 (m, 2H), 0.94-0.73 (m, 6H).

2-(6-cyclopropyl-4-cyclopropyloxy-1-oxophthalazin-2-yl)-N-(5-fluoropyrimidin-2-yl)acetamide: To a solution of methyl 2-(4-cyclopropoxy-6-cyclopropyl-1-oxophthalazin-2(1H)-yl)acetate (80 mg, 0.25 mmol) and 5-fluoropyrimidin-2-amine (86 mg, 0.75 mmol) in DCE (1.0 mL) was added AlMe₃ (1 M in n-heptane, 0.76 mL). The mixture was stirred at 60° C. for 2 h. The mixture was diluted with water (5 mL) and extracted with EtOAc (3 x 2 mL). The combined organic layers were washed with brine (2 mL), dried over anhydrous Na₂SO₄, filtered, concentrated under reduced pressure, and purified by reverse-phase preparative HPLC. LCMS: m/z = 396.2 [M+H]⁺. ¹H NMR (400 MHz, CDCl₃): δ 11.06 (br s, 1H), 8.77 (s, 2H), 8.10 (d, J = 8.8 Hz, 1H), 7.63-7.46 (m, 2H), 5.01 (s, 2H), 4.23-4.20 (m, 1H), 2.24-2.14 (m, 1H), 1.14-1.08 (m, 2H), 0.87-0.83 (m, 2H), 0.82-0.76 (m, 4H).

### Example 174

### 2-(Cyclopropoxy)-1-Oxo-6-(Trifluoromethyl)Phthalazin-2-yl]-N-(5-Fluoropyrimidin-2-yl)Acetamide (174)

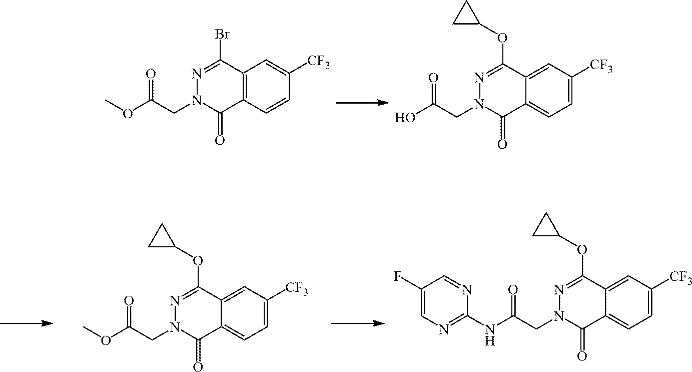 [SMILES: COC(=O)Cn1nc(Br)c2cc(C(F)(F)F)ccc2c1=O ; O=C(O)Cn1nc(OC2CC2)c2cc(C(F)(F)F)ccc2c1=O ; COC(=O)Cn1nc(OC2CC2)c2cc(C(F)(F)F)ccc2c1=O ; O=C(Cn1nc(OC2CC2)c2cc(C(F)(F)F)ccc2c1=O)Nc1ncc(F)cn1]

2-(4-cyclopropoxy-1-oxo-6-(trifluoromethyl)phthalazin-2(1H)-yl)acetic acid: To a mixture of NaH (329 mg, 8.22 mmol, 60% purity) in DMA (5 mL) at 0° C. was added cyclopropanol (318 mg, 5.48 mmol). The reaction mixture was stirred at 0° C. for 30 min followed by addition of 2-(4-bromo-1-oxo-6-(trifluoromethyl)phthalazin-2(1H)-yl)acetate (1.0 g, 2.74 mmol). The reaction mixture was stirred at 0° C. for 1 h. The reaction mixture was quenched by the addition of sat. aq. NH₄Cl (20 mL) and extracted with EtOAc (3 × 20 mL). The combined organic layers were washed with brine (20 mL), dried over anhydrous Na₂SO₄, filtered, and concentrated under reduced pressure. The crude product was triturated with PE:MTBE (3:1, 30 mL) and dried under reduced pressure to provide a residue that was used directly. LCMS: m/z = 329.2 [M+H]⁺.

methyl 2-(4-cyclopropoxy-1-oxo-6-(trifluoromethyl)phthalazin-2(1H)-yl)acetate: To a solution of 2-(4-cyclopropoxy-1-oxo-6-(trifluoromethyl)phthalazin-2(1H)-yl)acetic acid (200 mg, 0.61 mmol) in DMF (5 mL) at 0° C. were added MeI (95 mg, 0.67 mmol) and K₂CO₃ (101 mg, 0.73 mmol). The reaction mixture was stirred at 23° C. for 3 h. The reaction mixture was quenched by addition water (15 mL) and extracted with EtOAc (3 × 10 mL). The combined organic layers were washed with brine (2 × 10 mL), dried over anhydrous Na₂SO₄, filtered, and concentrated under reduced pressure. The residue was purified by silica gel column chromatography. LCMS: m/z = 343.2 [M+H]⁺.

2-(4-cyclopropoxy-1-oxo-6-(trifluoromethyl)phthalazin-2(1H)-yl)-N-(5-fluoropyrimidin-2-yl)acetamide: To a solution of methyl 2-(4-cyclopropoxy-1-oxo-6-(trifluoromethyl)phthalazin-2(1H)-yl)acetate (50 mg, 0.15 mmol) in DCE (5.0 mL) were added 5-fluoropyrimidin-2-amine (20 mg, 0.18 mmol) and AlMe₃ (1 M in n-heptane, 0.23 mL). The reaction mixture was stirred at 90° C. for 2 h. The reaction mixture was cooled to 23° C., diluted with water (15 mL), filtered, and extracted with EtOAc (3 × 10 mL). The combined organics was dried over anhydrous Na₂SO₄, filtered, and concentrated under reduced pressure. The residue was purified by reverse-phase preparative HPLC. LCMS: m/z = 424.0 [M+H]⁺. ¹H NMR (400 MHz, CDCl₃): δ 8.80 (br s, 1H), 8.57 (d, J = 8.4 Hz, 1H), 8.56 (s, 2H), 8.23-8.17 (m, 1H), 8.02 (d, J= 8.4 Hz, 1H), 5.32 (s, 2H), 4.34-4.26 (m, 1H), 0.91-0.83 (m, 4H).

### Example 175

### Tert-Butyl (3R)-3-[[2-(6-Bromo-4-Cyclopropyloxy-1-Oxophthalazin-2-yl)Acetyl]Amino]Piperidine-1-Carboxylate (175)

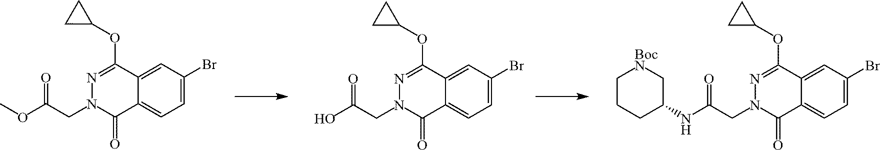 [SMILES: COC(=O)Cn1nc(OC2CC2)c2cc(Br)ccc2c1=O ; O=C(O)Cn1nc(OC2CC2)c2cc(Br)ccc2c1=O ; CC(C)(C)OC(=O)N1CCC[C@@H](NC(=O)Cn2nc(OC3CC3)c3cc(Br)ccc3c2=O)C1]

2-(6-bromo-4-cyclopropoxy-1-oxophthalazin-2(1H)-yl)acetic acid: To a solution of methyl 2-(6-bromo-4-cyclopropoxy-1-oxophthalazin-2(1H)-yl)acetate (500 mg, 1.42 mmol) in THF (5 mL) and water (5 mL) was added LiOH•H₂O (148 mg, 3.54 mmol). The reaction mixture was stirred at 25° C. for 2 h. The reaction mixture was poured into water (5 mL) and washed with MTBE (5 mL). The aqueous phase was adjusted pH = 3 with aq. HCl (3 M) and extracted with EtOAc (3 × 5 mL). The combined organic layers were washed with brine (10 mL), dried over anhydrous Na₂SO₄, filtered, and concentrated under reduced pressure to provide a residue that was used directly. LCMS: m/z = 339.0, 341.0 [M+H]⁺.

tert-butyl (3R)-3-[[2-(6-bromo-4-cyclopropyloxy-1-oxophthalazin-2-yl)acetyl]amino]piperidine-1-carboxylate: To a solution of 2-(6-bromo-4-cyclopropoxy-1-oxophthalazin-2(1H)-yl)acetic acid (350 mg, 1.03 mmol) and tert-butyl (3R)-aminopiperidine-1-carboxylate (227 mg, 1.14 mmol) in DMF (5 mL) were added DIPEA (533 mg, 4.13 mmol) and HATU (784 mg, 2.06 mmol). The reaction mixture was stirred at 20° C. for 2 h. The reaction mixture was poured into water (30 mL) and extracted with EtOAc (3 × 10 mL). The combined organic layers were washed with brine (10 mL), dried over anhydrous Na₂SO₄, filtered, and concentrated under reduced pressure. The residue was purified by silica gel column chromatography. LCMS: m/z = 421.0, 423.0 [M-99]⁺. ¹H NMR (400 MHz, CDCl₃): δ 8.25 (br d, J = 8.4 Hz, 1H), 8.04 (s, 1H), 7.89 (br d, J = 8.8 Hz, 1H), 6.37 (br s, 1H), 4.78 (br s, 2H), 4.27 (br s, 1H), 3.96 (br s, 1H), 3.64-3.19 (m, 4H), 1.84-1.50 (m, 4H), 1.38 (s, 9H), 0.86 (br s, 4H).

### Example 176

### 2-Bromo-4-Cyclopropyloxy-1-Oxophthalazin-2-yl)-N-[(3R)-Piperidin-3-yl]Acetamide HCl Salt (176)

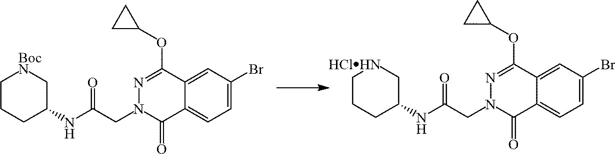

A solution of tert-butyl (3R)-3-[[2-(6-bromo-4-cyclopropyloxy-1-oxophthalazin-2-yl)acetyl]amino]piperidine-1-carboxylate (300 mg, 0.57 mmol) in HCl (4 M in EtOAc, 10 mL) was stirred at 25° C. for 2 h. The reaction mixture was concentrated under reduced pressure to give a residue that was resuspended in THF (2 mL), stirred for 30 min, and filtered. The filter cake was dried under reduced pressure to give a solid that was used directly. LCMS: m/z = 421.1, 423.1 [M+H]⁺. ¹H NMR (400 MHz, DMSO-d₆) δ 8.91 (br s, 2H), 8.29 (d, J= 7.6 Hz, 1H), 8.16-8.12 (m, 1H), 8.11-8.06 (m, 1H), 8.03 (d, J = 1.6 Hz, 1H), 4.64 (s, 2H), 4.19-4.24 (m, 1H), 4.03-3.89 (m, 1H), 3.25-3.10 (m, 2H), 2.85-2.63 (m, 2H), 1.90-1.78 (m, 2H), 1.73-1.41 (m, 2H), 0.87-0.77 (m, 4H).

### Example 177

### 2-Bromo-4-Cyclopropyloxy-1-Oxophthalazin-2-yl)-N-[(3R)-1-Cyclobutylpiperidin-3-yl]Acetamide (177)

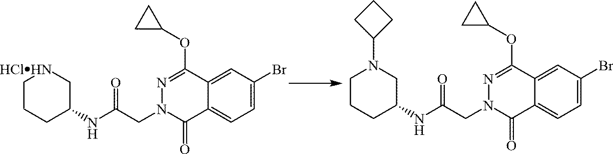

To a solution of 2-(6-bromo-4-cyclopropyloxy-1-oxophthalazin-2-yl)-N-[(3R)-piperidin-3-yl]acetamide HCl salt (100 mg, 0.22 mmol), cyclobutanone (15 mg, 0.22 mmol), and Et₃N (44 mg, 0.44 mmol) in DCM (1 mL) was added AcOH (26 mg, 0.44 mmol). The reaction mixture was stirred at 20° C. for 30 min, followed by addition of NaBH₃CN (41 mg, 0.65 mmol). The reaction mixture was stirred at 20° C. for a further 1 h. The reaction mixture was diluted with sat. aq. Na₂CO₃ (3 mL) and extracted with EtOAc (3 × 3 mL). The combined organic layers were washed with brine (3 mL), dried over anhydrous Na₂SO₄, filtered, and concentrated under reduced pressure. The residue was purified by reverse-phase preparative HPLC. LCMS: m/z = 475.0, 477.0 [M+H]⁺. ¹H NMR (400 MHz, CDCl₃): δ 8.28 (d, J= 8.4 Hz, 1H), 8.06 (s, 1H), 7.90 (br d, J = 7.2 Hz, 1H), 6.68 (br s, 1H), 4.91-4.71 (m, 2H), 4.30-4.28 (m, 1H), 4.06 (br s, 1H), 2.50-2.07 (m, 4H), 1.90-1.78 (m, 3H), 1.74-1.65 (m, 1H), 1.52-1.43 (m, 7H), 0.87-0.83 (m, 4H).

### Example 178

### 2-Bromo-4-Cyclopropyloxy-1-Oxophthalazin-2-yl)-N-[(3R)-1-Cyclopropylpiperidin-3-yl]Acetamide (178)

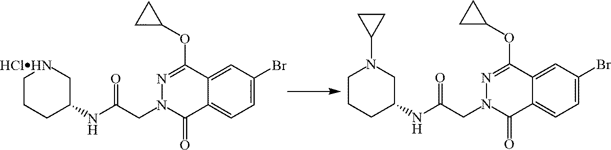

To a solution of 2-(6-bromo-4-cyclopropyloxy-1-oxophthalazin-2-yl)-N-[(3R)-piperidin-3-yl]acetamide HCl salt (50 mg, 0.11 mmol), (1-ethoxycyclopropoxy)trimethylsilane (19 mg, 0.11 mmol), and Et₃N (22 mg, 0.22 mmol) in MeOH (2.0 mL) was added AcOH (13 mg, 0.22 mmol). The reaction mixture was stirred at 20° C. for 30 min followed by addition of NaBH₃CN (21 mg, 0.33 mmol). The reaction mixture was stirred at 60° C. for a further 16 h. The reaction mixture was diluted with sat. aq. Na₂CO₃ (3 mL) and extracted with EtOAc (3 × 3 mL). The combined organic layers were washed with brine (3 mL), dried over anhydrous Na₂SO₄, filtered, and concentrated under reduced pressure. The residue was purified by reverse-phase preparative HPLC. LCMS: m/z = 461.0, 463.0 [M+H]⁺. ¹H NMR (400 MHz, CDCl₃): δ 8.27 (d, J= 8.4 Hz, 1H), 8.06 (d, J = 1.6 Hz, 1H), 7.91 (dd, J= 2.0, 8.4 Hz, 1H), 6.52 (br s, 1H), 4.86-4.69 (m, 2H), 4.31-4.22 (m, 1H), 4.03 (br s, 1H), 2.64-2.28 (m, 4H), 1.55-1.43 (m, 5H), 0.91-0.81 (m, 4H), 0.32-0.24 (m, 2H), (-0.02)-(-0.06) (m, 2H).

### Example 179

### 2-Bromo-4-(Difluoromethyl)-1-Sulfanylidenephthalazin-2-yl]-N-(5-Fluoropyrimidin-2-yl)Acetamide (179)

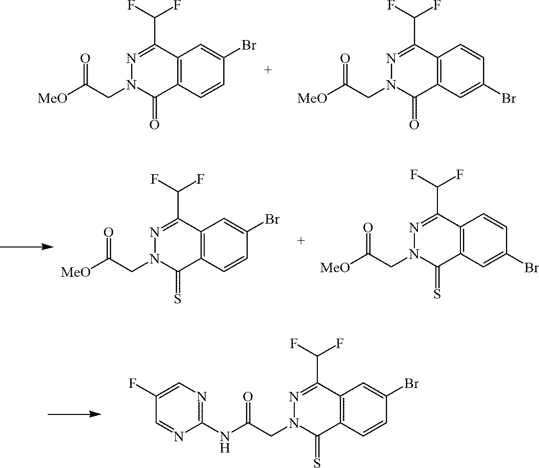

methyl 2-(6-bromo-4-(difluoromethyl)-1-thioxophthalazin-2(1H)-yl)acetate and 2-(7-bromo-4-(difluoromethyl)-1-thioxophthalazin-2(1H)-yl)acetate: To a solution of methyl 2-(6-bromo-4-(difluoromethyl)-1-oxophthalazin-2(1H)-yl)acetate and methyl 2-(7-bromo-4-(difluoromethyl)-1-oxophthalazin-2(1H)-yl)acetate (1:1 mixture, 250 mg, 0.72 mmol) in toluene (3.0 mL) was added 2,4-bis-(4-methoxyphenyl)-1,3-dithia-2,4-diphosphetane 2,4-disulfide (350 mg, 0.87 mmol). The reaction mixture was stirred at 90° C. for 40 h. The reaction mixture was diluted with water (2 mL) and extractedwith EtOAc (3 × 8 mL). The combined organic layers were washed with brine (5 mL), dried over anhydrous Na₂SO₄, filtered, and concentrated under reduced pressure. The residue was purified by preparative silica gel TLC. LCMS: m/z = 362.9, 364.9 [M+H]⁺.

2-[6-bromo-4-(difluoromethyl)-1-thioxophthalazin-2-yl]-N-(5-fluoropyrimidin-2-yl)acetamide: To a solution of methyl 2-(6-bromo-4-(difluoromethyl)-1-thioxophthalazin-2(1H)-yl)acetate and methyl 2-(7-bromo-4-(difluoromethyl)-1-thioxophthalazin-2(1H)-yl)acetate (1:1 mixture, 160 mg, 0.44 mmol) and 5-fluoropyrimidin-2-amine (65 mg, 0.57 mmol) in toluene (4.0 mL) was added trimethyl-(4-trimethylalumanuidyl-1,4-diazoniabicyclo[2.2.2]octan-1-yl)alumanuide (147 mg, 0.57 mmol). The reaction mixture was stirred at 90° C. for 48 h. The reaction mixture was concentrated under reduced pressure. The residue was purified by reverse-phase preparative HPLC followed by preparative SFC. LCMS: m/z = 443.8, 445.8 [M+H]⁺. ¹H NMR (400 MHz, CDCl₃): δ 8.84 (d, J= 8.8 Hz, 1H), 8.53 (br s, 1H), 8.50 (s, 2H), 8.33 (d, J= 1.2 Hz, 1H), 7.95 (dd, J= 2.0, 8.8 Hz, 1H), 6.66 (t, J= 53.2 Hz, 1H), 6.03 (s, 2H).

### Example 180

### 2-Bromo-6-(Dimethylamino)-1-Oxophthalazin-2-yl]-N-(5-Fluoropyrimidin-2-yl)Acetamide (180)

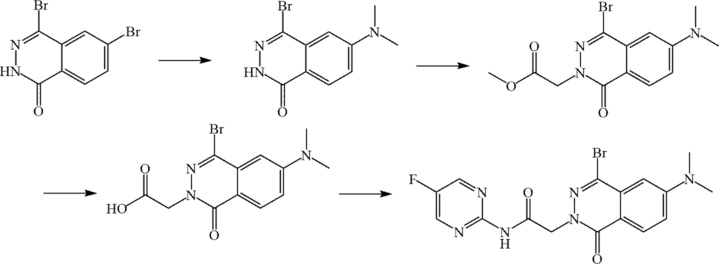

4-bromo-6-(dimethylamino)-2H-phthalazin-1-one: To a solution of 4,6-dibromo-2H-phthalazin-1-one (500 mg, 1.65 mmol) in water (8 mL) was added dimethylamine (8.23 mL, 2 M in THF). The mixture was stirred at 100° C. for 24 h. The mixture was diluted with water and extracted with EtOAc (3 × 10 mL). The combined organic layers were washed with brine (10 mL), dried over anhydrous Na₂SO₄, filtered, and concentrated under reduced pressure to provide a residue that was used directly. LCMS: m/z = 268.0, 270.0 [M+H]⁺.

methyl 2-[4-bromo-6-(dimethylamino)-1-oxo-phthalazin-2-yl]acetate: To a solution of 4-bromo-6-(dimethylamino)-2H-phthalazin-1-one (170 mg, 0.63 mmol) and K₂CO₃ (306 mg, 2.21 mmol) in DMF (3.0 mL) was added methyl bromoacetate (290 mg, 1.9 mmol). The mixture was stirred at 40° C. for 2 h. The mixture was diluted with water (10 mL) and extracted with EtOAc (3 × 15 mL). The combined organic layers were washed with brine, dried over anhydrous Na₂SO₄, filtered, and concentrated under reduced pressure to provide a residue that was used directly. LCMS: m/z = 340.2, 342.1 [M+H]⁺.

2-[4-bromo-6-(dimethylamino)-1-oxo-phthalazin-2-yl]acetic acid: To a solution of methyl 2-[4-bromo-6-(dimethylamino)-1-oxo-phthalazin-2-yl]acetate (180 mg, 0.52 mmol) in THF (2.0 mL) was added aq. LiOH (1.05 mL, 1 M). The mixture was stirred at 40° C. for 2 h. The mixture was diluted with aq. HCl (1.5 mL, 1 M) and extracted with EtOAc (2 × 10 mL). The combined organic layers were washed with brine (10 mL), dried over anhydrous Na₂SO₄, filtered, and concentrated under reduced pressure to provide a residue that was used directly. LCMS: m/z = 324.1, 326.1 [M-H]⁻.

2-[4-bromo-6-(dimethylamino)-1-oxophthalazin-2-yl]-N-(5-fluoropyrimidin-2-yl)acetamide: To a solution of 2-[4-bromo-6-(dimethylamino)-1-oxo-phthalazin-2-yl]acetic acid (250 mg, 0.76 mmol), 5-fluoropyrimidin-2-amine (104 mg, 0.91 mmol), and 1-methylimidazole (251 mg, 3.06 mmol) in MeCN (2.5 mL) was added TCFH (230 mg, 0.82 mmol) at 25° C. The mixture was stirred at 40° C. for 2 h. A second portion of TCFH was added (301 mg, 1.07 mmol) at 40° C. and the mixture was stirred for an additional 2 h. The mixture was diluted with MeCN (5 mL) and water (3 mL), filtered, and directly purified by reverse-phase preparative HPLC. LCMS: m/z = 421.1, 423.1 [M+H]⁺. ¹H NMR (400 MHz, DMSO-d₆) δ 11.14 (s, 1H), 8.78 (s, 2H), 8.05 (d, J= 9.0 Hz, 1H), 7.34-7.31 (m, 1H), 6.81-6.81 (m, 1H), 5.06 (s, 2H), 3.15-3.13 (m, 6H).

### Example 181

### 2-(4,6-Dibromo-1-Oxophthalazin-2-yl)-N-(5-Fluoropyrimidin-2-yl)Acetamide (181)

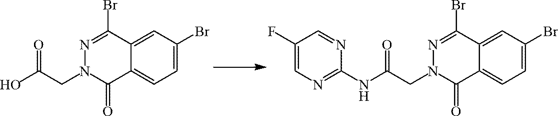 [SMILES: O=C(O)Cn1nc(Br)c2cc(Br)ccc2c1=O ; O=C(Cn1nc(Br)c2cc(Br)ccc2c1=O)Nc1ncc(F)cn1]

To a solution of 2-(4,6-dibromo-1-oxo-phthalazin-2-yl)acetic acid (1.3 g, 3.6 mmol), 5-fluoropyrimidin-2-amine (736 mg, 6.5 mmol), and 1-methylimidazole (1.18 g, 14.4 mmol) in MeCN (13 mL) was added TCFH (1.4 g, 5.0 mmol) at 25° C. The mixture was stirred at 40° C. for 2 h. A second portion of TCFH was added (1.4 g, 5.0 mmol) at 40° C. and the mixture was stirred for an additional 2 h. The mixture was diluted with water (20 mL) and filtered. The collected solid was purified by silica gel column chromatography. LCMS: m/z = 456.1, 458.1, 460.1 [M+H]⁺. ¹H NMR (400 MHz, DMSO-d₆) δ 11.20 (s, 1H), 8.79 (s, 2H), 8.23-8.16 (m, 2H), 8.11-8.10 (m, 1H), 5.14 (s, 2H).

### Example 182

### 2-Bromo-4-(Difluoromethyl)-5-Fluoro-1-Oxophthalazin-2-yl]-N-(5-Chloropyrimidin-2-yl)Acetamide (182)

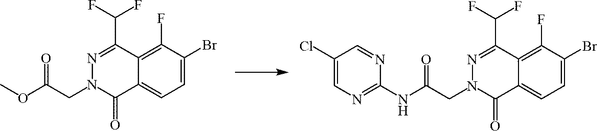 [SMILES: COC(=O)Cn1nc(C(F)F)c2c(F)c(Br)ccc2c1=O ; O=C(Cn1nc(C(F)F)c2c(F)c(Br)ccc2c1=O)Nc1ncc(Cl)cn1]

To a solution of methyl 2-(6-bromo-4-(difluoromethyl)-5-fluoro-1-oxophthalazin-2(1H)-yl)acetate (52 mg, 0.14 mmol) and 5-chloropyrimidin-2-amine (27 mg, 0.21 mmol) in toluene (1.0 mL) and THF (0.5 mL) was added AlMe₃ (2 M in toluene, 0.43 mmol). The reaction mixture was stirred at 90° C. for 6 h. The reaction mixture was diluted with water (5 mL) and extracted with EtOAc (3 × 5 mL). The combined organic layers were dried over anhydrous Na₂SO₄, filtered, and concentrated under reduced pressure. The residue was purified by reverse-phase preparative HPLC. LCMS: m/z = 462.1, 464.1, 466.1 [M+H]⁺. ¹H NMR (400 MHz, CDCl₃) δ 11.30 (s, 1H), 8.81 (s, 2H), 8.31 (dd, J= 8.6, 6.4 Hz, 1H), 8.12 (dd, J= 8.6, 0.8 Hz, 1H), 7.35-7.08 (m, 1H), 5.25 (s, 2H).

### Example 183

### 2-Bromo-4-(Difluoromethyl)-5-Fluoro-1-Oxophthalazin-2-yl]-N-(1-Ethylpiperidin-3-yl)Acetamide (183)

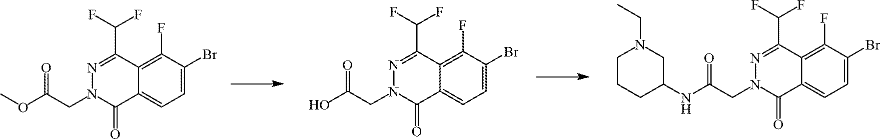 [SMILES: COC(=O)Cn1nc(C(F)F)c2c(F)c(Br)ccc2c1=O ; O=C(O)Cn1nc(C(F)F)c2c(F)c(Br)ccc2c1=O ; CCN1CCCC(NC(=O)Cn2nc(C(F)F)c3c(F)c(Br)ccc3c2=O)C1]

2-[6-bromo-4-(difluoromethyl)-5-fluoro-1-oxo-phthalazin-2-yl]acetic acid: To a solution of methyl 2-(6-bromo-4-(difluoromethyl)-5-fluoro-1-oxophthalazin-2(1H)-yl)acetate (40 mg, 0.11 mmol) in THF (0.7 mL) was added aq. LiOH (0.21 mL, 1 M). The mixture was stirred at 40° C. for 2 h. The mixture was diluted with aq. HCl (0.40 mL, 1 M) and extracted with EtOAc (2 × 5 mL). The combined organic layers were washed with brine (10 mL), dried over anhydrous Na₂SO₄, filtered, and concentrated under reduced pressure to provide a residue that was used directly. LCMS: m/z = 349.1, 351.1 [M-H]⁻.

2-[6-bromo-4-(difluoromethyl)-5-fluoro-1-oxophthalazin-2-yl]-N-(1-ethylpiperidin-3-yl)acetamide: To a solution of 2-[6-bromo-4-(difluoromethyl)-5-fluoro-1-oxo-phthalazin-2-yl]acetic acid (48 mg, 0.14 mmol), 1-ethyl-3-piperidinamine (31 mg, 0.26 mmol), and 1-methylimidazole (44 mg, 0.54 mmol) in MeCN (0.64 mL) was added TCFH (48 mg, 0.54 mmol). The mixture was stirred at 40° C. for 2 h. A second portion of TCFH was added (48 mg, 0.54 mmol) at 40° C. and the mixture was stirred for an additional 2 h. The mixture was diluted with water (3 mL), filtered, and the filter cake was purified by silica gel column chromatography. LCMS: m/z = 461.2, 463.2 [M+H]⁺. ¹H NMR (400 MHz, DMSO-d₆) δ 9.26 (s, 1H), 8.51 (d, J= 7.5 Hz, 1H), 8.30 (dd, J= 8.6, 6.4 Hz, 1H), 8.10 (dd, J= 8.6, 0.8 Hz, 1H), 7.34-7.07 (m, 1H), 4.91-4.77 (m, 2H), 4.01-3.91 (m, 1H), 3.48-3.43 (m, 2H), 3.20-3.11 (m, 2H), 2.86-2.76 (m, 1H), 2.68-2.57 (m, 1H), 1.98-1.86 (m, 2H), 1.73-1.60 (m, 1H), 1.49-1.37 (m, 1H), 1.21 (t, J= 7.3 Hz, 3H).

### Example 184

### 2-Cyclopropyl-4-(Difluoromethyl)-5-Fluoro-1-Oxophthalazin-2-yl]-N-(5-Fluoropyrimidin-2-yl)Acetamide (184)

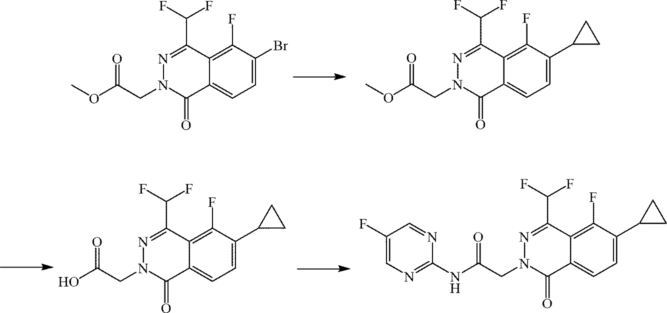 [SMILES: COC(=O)Cn1nc(C(F)F)c2c(F)c(Br)ccc2c1=O ; COC(=O)Cn1nc(C(F)F)c2c(F)c(C3CC3)ccc2c1=O ; O=C(O)Cn1nc(C(F)F)c2c(F)c(C3CC3)ccc2c1=O ; O=C(Cn1nc(C(F)F)c2c(F)c(C3CC3)ccc2c1=O)Nc1ncc(F)cn1]

methyl 2-[6-cyclopropyl-4-(difluoromethyl)-5-fluoro-1-oxo-phthalazin-2-yl]acetate: To a solution of methyl 2-(6-bromo-4-(difluoromethyl)-5-fluoro-1-oxophthalazin-2(1H)-yl)acetate (80 mg, 0.21 mmol), CsF (99 mg, 0.66 mmol), and Pd(dppf)Cl₂ (16 mg, 0.02 mmol) in 1,4-dioxane (1.0 mL) was added cyclopropylboronic acid (56 mg, 0.66 mmol). The mixture was stirred at 100° C. for 2 h. The mixture was diluted with water (2 mL) and extracted with EtOAc (2 × 5 mL). The combined organic layers were washed with brine (10 mL), dried over anhydrous Na₂SO₄, filtered, and concentrated under reduced pressure to provide a residue that was used directly. LCMS: m/z = 327.2 [M+H]⁺.

2-[6-cyclopropyl-4-(difluoromethyl)-5-fluoro-1-oxo-phthalazin-2-yl]acetic acid: To a solution of methyl 2-[6-cyclopropyl-4-(difluoromethyl)-5-fluoro-1-oxo-phthalazin-2-yl]acetate (80 mg, 0.25 mmol) in THF (2.0 mL) was added aq. LiOH (0.49 mL, 1 M). The mixture was stirred at 40° C. for 2 h. The mixture was diluted with aq. HCl (0.75 mL, 1 M) and extracted with EtOAc (2 × 5 mL). The combined organic layers were washed with brine (10 mL), dried over anhydrous Na₂SO₄, filtered, and concentrated under reduced pressure to provide a residue that was used directly. LCMS: m/z = 313.2 [M+H]⁺.

2-[6-cyclopropyl-4-(difluoromethyl)-5-fluoro-1-oxophthalazin-2-yl]-N-(5-fluoropyrimidin-2-yl)acetamide: To a solution of 2-[6-cyclopropyl-4-(difluoromethyl)-5-fluoro-1-oxo-phthalazin-2-yl]acetic acid (80 mg, 0.25 mmol), 5-fluoropyrimidin-2-amine (52 mg, 0.46 mmol), and 1-methylimidazole (84 mg, 1.02 mmol) in MeCN (1.5 mL) was added TCFH (89 mg, 0.32 mmol) at 25° C. The mixture was stirred at 40° C. for 2 h. A second portion of TCFH was added (89 mg, 0.32 mmol) at 40° C. and the mixture was stirred for an additional 2 h. The mixture was diluted with MeCN (5 mL) and water (3 mL), filtered, and directly purified by reverse-phase preparative HPLC. LCMS: m/z = 408.2 [M+H]⁺. ¹H NMR (400 MHz, DMSO-d₆) δ 11.21 (s, 1H), 8.79 (d, J = 0.7 Hz, 2H), 8.08 (d, J= 8.3 Hz, 1H), 7.58 (ddd, J= 8.2, 6.9, 0.5 Hz, 1H), 7.35-7.08 (m, 1H), 5.20-5.19 (m, 2H), 2.34-2.32 (m, 1H), 1.20-1.16 (m, 2H), 0.97-0.93 (m, 2H).

### Example 185

### 2-Cyclobutyl-4-(Difluoromethyl)-5-Fluoro-1-Oxophthalazin-2-yl]-N-(5-Fluoropyrimidin-2-yl)Acetamide (185)

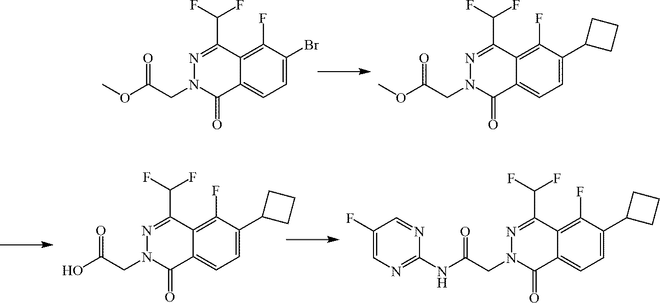 [SMILES: COC(=O)Cn1nc(C(F)F)c2c(F)c(Br)ccc2c1=O ; COC(=O)Cn1nc(C(F)F)c2c(F)c(C3CCC3)ccc2c1=O ; O=C(O)Cn1nc(C(F)F)c2c(F)c(C3CCC3)ccc2c1=O ; O=C(Cn1nc(C(F)F)c2c(F)c(C3CCC3)ccc2c1=O)Nc1ncc(F)cn1]

methyl 2-[6-cyclobutyl-4-(difluoromethyl)-5-fluoro-1-oxo-phthalazin-2-yl] acetate: To a solution of methyl 2-(6-bromo-4-(difluoromethyl)-5-fluoro-1-oxophthalazin-2(1H)-yl)acetate (80 mg, 0.21 mmol), CsF (99 mg, 0.66 mmol), and Pd(dppf)Cl₂ (16 mg, 0.02 mmol) in 1,4-dioxane (1.0 mL) was added cyclobutylboronic acid (66 mg, 0.66 mmol). The mixture was stirred at 100° C. for 6 h. The mixture was diluted with water (2 mL) and extracted with EtOAc (2 × 5 mL). The combined organic layers were washed with brine (10 mL), dried over anhydrous Na₂SO₄, filtered, and concentrated under reduced pressure to provide a residue that was used directly. LCMS: m/z = 341.2 [M+H]⁺.

2-[6-cyclobutyl-4-(difluoromethyl)-5-fluoro-1-oxo-phthalazin-2-yl]acetic acid: To a solution of methyl 2-[6-cyclobutyl-4-(difluoromethyl)-5-fluoro-1-oxo-phthalazin-2-yl]acetate (60 mg, 0.17 mmol) in THF (1.5 mL) was added aq. LiOH (0.35 mL, 1 M). The mixture was stirred at 40° C. for 2 h. The mixture was diluted with aq. HCl (0.50 mL, 1 M) and extracted with EtOAc (2 × 5 mL). The combined organic layers were washed with brine (10 mL), dried over anhydrous Na₂SO₄, filtered, and concentrated under reduced pressure to provide a residue that was used directly. LCMS: m/z = 325.0 [M-H]⁻.

2-[6-cyclobutyl-4-(difluoromethyl)-5-fluoro-1-oxophthalazin-2-yl]-N-(5-fluoropyrimidin-2-yl)acetamide: To a solution of 2-[6-cyclobutyl-4-(difluoromethyl)-5-fluoro-1-oxo-phthalazin-2-yl]acetic acid (60 mg, 0.18 mmol), 5-fluoropyrimidin-2-amine (37 mg, 0.33 mmol), and 1-methylimidazole (60 mg, 0.73 mmol) in MeCN (1.5 mL) was added TCFH (64 mg, 0.22 mmol). The mixture was stirred at 40° C. for 2 h. A second portion of TCFH was added (64 mg, 0.22 mmol) at 40° C. and the mixture was stirred for an additional 2 h. The mixture was diluted with MeCN (5 mL) and water (3 mL), filtered, and directly purified by reverse-phase preparative HPLC. LCMS: m/z = 422.2 [M+H]⁺. ¹H NMR (400 MHz, DMSO-d₆) δ 11.22 (s, 1H), 8.79 (s, 2H), 8.18 (d, J= 8.2 Hz, 1H), 8.02-7.98 (m, 1H), 7.32-7.05 (m, 1H), 5.20-5.20 (m, 2H), 3.98-3.89 (m, 1H), 2.43-2.40 (m, 2H), 2.29-2.23 (m, 2H), 2.16-2.06 (m, 1H), 1.92-1.86 (m, 1H).

### BIOLOGICAL EXAMPLE 1

### Biochemical Assay of the Compounds

### Procedure for Culturing THP-1 Cells

Compounds as provided herein were tested in the following assay. Cell culture medium employed contained RPMI 1640 medium (89%), FBS (10%), Pen/Strep (1%), and 2-mercaptoethanol (0.05 mM). Freezing medium was made up of 90% FBS and 10% DMSO. THP-1 cells were removed from the liquid nitrogen and placed into a 37° C. water bath to thaw, until signs of ice dissipated. The cells were then added to 9 mL of warm cell culture medium and centrifuged for 5 minutes at 1000 rpm. The supernatant was discarded, and the cells were resuspended in new cell culture medium. THP-1 cells were then split and cultured in the cell culture medium, being passaged every 2-3 days with the cell density will be maintained between 5×10⁵ and 1.5×10⁶ viable cells/mL.

To freeze, cells were resuspended with fresh freezing medium, adjusting the cell density to 5×10⁶ cells/mL. The cell suspension was partitioned into 1 mL aliquots per vial, and the vials were transferred to a -80° C. freezer. After one day at -80° C., the cell vials were transferred to liquid nitrogen freezer for storage.

### Procedure for IL-1β Secretion Assay in 384-Well Plates

PMA was dissolved in DMSO to make a stock solution at 5 mg/ml and stored in 10 µl aliquots at -20° C. for single use. PMA is added to normal growth medium. LPS was diluted with 1 mL of water solution to provide a 1 mg/mL stock solution and stored in 15 µl aliquots at -20° C. for single use. Nigericin is diluted in ice cold 100% ethanol to 5 mg/ml (6.7 mM) and stored in 75 µL aliquots at -20° C. for single use. Serum-free media contains RPMI 1640 medium (99%), Pen/Strep (1%), and 2-mercaptoethanol (0.05 mM). The two control conditions used to qualify and normalize test compound dose-response curves were as follows: High Control = 25 ng/mL LPS, 5 µM Nigericin, 0.5% DMSO, Low Control = 25 ng/mL, LPS, 0.5% DMSO.

### Day 1: Differentiation With PMA

THP-1 cells were diluted to provide a suspension at a concentration of 1.0×10⁶ cells/mL with the total volume of suspension required to enable the desired number of assay plates. The growth media was supplemented with PMA (5 ng/mL final concentration) and the cells were incubated at 37° C. under a humidified atmosphere of 5% CO₂ for 40 h.

### Day 3: Plating With Sequential LPS and Nigericin Stimulation

All media was carefully aspirated from each culture flask. The cells were washed carefully with 1x DPBS. The cells were then briefly digested with trypsin LE for 5 minutes at 23° C. and immediately resuspended in cell growth media. After resuspension, the cells were centrifuged at 1000 rpm for 3 minutes and the supernatant was discarded. The cells were resuspended in DPBS and once again centrifuged at 1000 rpm for 5 minutes. The supernatant was discarded and the cell pellet was resuspended in serum-free media supplemented with LPS (25 ng/mL final concentration) to enable the distribution of 30 K THP-1 cells within 45 µL of media into each well of 384-well PDL-coated plates. The 384-well plates were then incubated at 37° C. under a humidified atmosphere of 5% CO₂ for 2 h. Following this period, test compounds were dispensed by Tecan across the desired concentration range with all wells normalized to a final 0.5 % DMSO concentration. The plates were then then incubated at 37° C. under a humidified atmosphere of 5% CO₂ for 1 h. Following this period, 5 µL of the 5 mg/mL nigericin stock solution was added to each of the appropriate wells and plates were centrifuged at 1000 rpm for 30 seconds. The plates were the immediately reintroduced to the incubator at 37° C. under a humidified atmosphere of 5% CO₂ for 2 h. After this time, 35 µL/well of supernatant was collected and transferred into v-bottom plate and centrifuged at 1000 rpm for 1 minute. These supernatant aliquots were analyzed using an IL-Iβ detection kit as described below. If needed, the test samples could be snap frozen and stored at -80° C. until analyzed.

### IL-Iβ Detection

To prepare each ELISA plate, capture antibody (mAb Mt175) was diluted with PBS to a final concentration of 2 µg/mL and then 20 µL of this solution was added to each well of the ELISA plate. Each plate was allowed to incubate overnight at 4° C. The next day, the capture antibody solution was removed and discarded. Each ELISA plate was washed 4 times with PBST followed by the addition of 25 µL/well of blocking buffer (Licor-927-40010) supplemented with 0.1% Tween 20. Each ELISA plate was then allowed to incubate for 1 hour at 23° C. After this time, the blocking buffer was removed and discarded. Each ELISA plate was washed 4 times with PBST. During this time, the v-bottomed plates containing the supernatant aliquots from the assay run were centrifuged at 300 g for 5 minutes before transferring 15 µL/well of the supernatant sample to each ELISA plate. Each ELISA plate was then allowed to incubate for 2 h at 23° C. After this time, the supernatant samples were removed and discarded. Each ELISA plate was washed 4 times with PBST. To each ELISA plate was added 15 µL/well of mAb7P10-biotin at 0.5 µg/mL (1:1000 diluted in blocking buffer). Each ELISA plate was then allowed to incubate for 1 h at 23° C. After this time, the antibody solution was removed and discarded. Each ELISA plate was washed 4 times with PBST. To each ELISA plate was added 20 µL/well of streptavidin-HRP (1:2000 diluted in blocking buffer). Each ELISA plate was then allowed to incubate for 1 h at 23° C. After this time, the buffer was removed and discarded. Each ELISA plate was washed 4 times with PBST. To each ELISA plate was added 20 µL/well of HRP substrate. Each ELISA plate was then allowed to incubate for 2 minutes at 23° C. After this time, to each ELISA plate was added 40 µL/well of stop solution. Each ELISA plate was centrifuged at 1200 rpm for 30 seconds.

The plate was then read at 450 nm in a microplate reader. Percent inhibition was calculated as follows:

$$\begin{matrix}\text{\% inhibition rate =} \\ \left(\text{treated samples-high control}\right) / \left(\text{low control-high control}\right) \times 100\end{matrix}$$

Activity of the tested compounds is provided in Table 3 below as follows: +++ = IC₅₀ < 10 µM; ++ = IC₅₀ 10-15 µM; + = 1C₅₀> 15 µM.

TABLE 3

| Ex. | Activity | Activity |
|---|---|---|
| 1 | 0.030 | +++ |
| 2 | 2.11 | +++ |
| 3 | 0.159 | +++ |
| 4 | 1.83 | +++ |
| 5 | 0.046 | +++ |
| 6 | 0.121 | +++ |
| 7 | 1.03 | +++ |
| 8 | 0.235 | +++ |
| 9 | 0.144 | +++ |
| 10 | 3.05 | +++ |
| 11 | 3.15 | +++ |
| 12 | 2.59 | +++ |
| 13 | 2.32 | +++ |
| 14 | 3.66 | +++ |
| 15 | 6.27 | +++ |
| 16 | 0.787 | +++ |
| 17 | 0.876 | +++ |
| 18 | 10.7 | ++ |
| 19 | 0.334 | +++ |
| 20 | 0.018 | +++ |
| 21 | 1.06 | +++ |
| 22 | 0.398 | +++ |
| 23 | 2.07 | +++ |
| 24 | 0.138 | +++ |
| 25 | 0.689 | +++ |
| 26 | 0.078 | +++ |
| 27 | 0.137 | +++ |
| 28 | 0.307 | +++ |
| 29 | 0.286 | +++ |
| 30 | 0.519 | +++ |
| 31 | 1.32 | +++ |
| 32 | 0.128 | +++ |
| 33 | 0.176 | +++ |
| 34 | 1.89 | +++ |
| 35 | 0.529 | +++ |
| 36 | 2.80 | +++ |
| 37 | 4.95 | +++ |
| 38 | 0.913 | +++ |
| 39 | 0.054 | +++ |
| 40 | 1.06 | +++ |
| 41 | 0.163 | +++ |
| 42 | 3.35 | +++ |
| 43 | 4.23 | +++ |
| 44 | 12.3 | ++ |
| 45 | 1.19 | +++ |
| 46 | 1.17 | +++ |
| 47 | 2.55 | +++ |
| 48 | 4.09 | +++ |
| 49 | 1.70 | +++ |
| 50 | 27.5 | + |
| 51 | 0.863 | +++ |
| 52 | 2.53 | +++ |
| 53 | 1.13 | +++ |
| 54 | 0.349 | +++ |
| 55 | 0.710 | +++ |
| 56 | 3.23 | +++ |
| 57 | 4.40 | +++ |
| 58 | 3.99 | +++ |
| 59 | 0.457 | +++ |
| 60 | 1.41 | +++ |
| 61 | 0.943 | +++ |
| 62 | 10.3 | ++ |
| 63 | 12.8 | ++ |
| 64 | 0.392 | +++ |
| 65 | 22.7 | + |
| 66 | 2.44 | +++ |
| 67 | 1.03 | +++ |
| 68 | 0.110 | +++ |
| 69 | 1.71 | +++ |
| 70 | 0.952 | +++ |
| 71 | 0.499 | +++ |
| 72 | 9.04 | +++ |
| 73 | 9.63 | +++ |
| 74 | 3.74 | +++ |
| 75 | 2.85 | +++ |
| 76 | 8.06 | +++ |
| 77 | 3.85 | +++ |
| 78 | 1.51 | +++ |
| 79 | 0.610 | +++ |
| 80 | 0.197 | +++ |
| 81 | 32.0 | + |
| 82 | 1.48 | +++ |
| 83 | >50 | + |
| 84 | 3.71 | +++ |
| 85 | 0.382 | +++ |
| 86 | >50 | + |
| 87 | 0.180 | +++ |
| 88 | >50 | + |
| 89 | 0.764 | +++ |
| 90 | 6.74 | +++ |
| 91 | 0.489 | +++ |
| 92 | 0.741 | +++ |
| 93 | 0.14 | +++ |
| 94 | 6.05 | +++ |
| mixture of 95 and 96 | 4.35 | +++ |
| mixture of 97 and 98 | 0.369 | +++ |
| mixture of 99 and 100 | 0.838 | +++ |
| 101 | 1.45 | +++ |
| 102 | 2.44 | +++ |
| 103 | 0.265 | +++ |
| 104 | 6.99 | +++ |
| 105 | 0.614 | +++ |
| 106 | 0.058 | +++ |
| 107 | >50 | + |
| 108 | 8.23 | +++ |
| 109 | 5.81 | +++ |
| 110 | 1.35 | +++ |
| 111 | 0.796 | +++ |
| 112 | 1.12 | +++ |
| 113 | 0.035 | +++ |
| 114 | 1.77 | +++ |
| 115 | 1.53 | +++ |
| 116 | 4.60 | +++ |
| 117 | 6.45 | +++ |
| 118 | 0.737 | +++ |
| 119 | 0.163 | +++ |
| 120 | 0.025 | +++ |
| 121 | 0.423 | +++ |
| 122 | 1.28 | +++ |
| 123 | 2.13 | +++ |
| 124 | 1.03 | +++ |
| 125 | 3.06 | +++ |
| 126 | 1.78 | +++ |
| 127 | 1.05 | +++ |
| 128 | 5.26 | +++ |
| 129 | 20.1 | +++ |
| 130 | 2.92 | +++ |
| 131 | 1.27 | +++ |
| 132 | >50 | + |
| 133 | 0.109 | +++ |
| 134 | 1.21 | +++ |
| 135 | 0.424 | +++ |
| 136 | 0.181 | +++ |
| 137 | 0.186 | +++ |
| 138 | 11.1 | ++ |
| 139 | 0.318 | +++ |
| 140 | 0.087 | +++ |
| 141 | 1.20 | +++ |
| 142 | 0.091 | +++ |
| 143 | 2.47 | +++ |
| 144 | 0.191 | +++ |
| 145 | 0.174 | +++ |
| 146 | 0.461 | +++ |
| 147 | 0.279 | +++ |
| 148 | 0.097 | +++ |
| 149 | 0.782 | +++ |
| 150 | 0.127 | +++ |
| 151 | 0.491 | +++ |
| 152 | 0.173 | +++ |
| 153 | 0.147 | +++ |
| 154 | 0.680 | +++ |
| 155 | 5.95 | +++ |
| 156 | 0.070 | +++ |
| 157 | 0.139 | +++ |
| 158 | 0.309 | +++ |
| 159 | 4.76 | +++ |
| 160 | 1.32 | +++ |
| 161 | 3.63 | +++ |
| 162 | 0.455 | +++ |
| 163 | 3.77 | +++ |
| 164 | 0.149 | +++ |
| 165 | 0.613 | +++ |
| 166 | 0.237 | +++ |
| 167 | 16.1 | + |
| 168 | 17.1 | + |
| 169 | 2.29 | +++ |
| 170 | 0.493 | +++ |
| 171 | 5.85 | +++ |
| 172 | 0.403 | +++ |
| 173 | 0.12 | +++ |
| 174 | 0.515 | +++ |
| 175 | 8.65 | +++ |
| 176 | 4.27 | +++ |
| 177 | 0.213 | +++ |
| 178 | 0.140 | +++ |
| 179 | 0.486 | +++ |
| 180 | 4.86 | +++ |
| 181 | 0.230 | +++ |
| 182 | 0.068 | +++ |
| 183 | 0.485 | +++ |
| 184 | 0.179 | +++ |
| 185 | 2.09 | +++ |

Unless otherwise defined, all technical and scientific terms used herein have the same meaning as commonly understood by one of ordinary skill in the art to which this disclosure belongs.

The embodiments illustratively described herein may suitably be practiced in the absence of any element or elements, limitation or limitations, not specifically disclosed herein. Thus, for example, the terms “comprising,” “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the embodiments claimed.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound of Formula I: 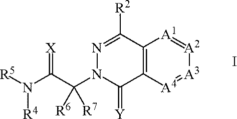
or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, –NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, —NO₂, —SF₅, -OR¹¹, -C(O)R¹², -C(O)OR¹¹, –SR¹¹, -NR¹¹S(O)₀₋₂-R¹¹, -NR¹¹S(O)₀₋₂N(R¹¹)₂, -NR¹¹C(O)N(R¹¹)₂, -NR¹¹C(O)OR¹¹, -OC(O)R¹¹, –OC(O)N(R¹¹)₂, halo, or cyano; wherein the C₁₋₆ alkyl, C₂₋₆ alkenyl, C₂₋₆ alkynyl, or C₁₋₆ haloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹;
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{1a};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, –NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, –NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)R¹², -C(O)OR¹¹, -NR¹¹S(O)₀₋₂-R¹¹, –NR¹¹S(O)₀₋₂N(R¹¹)₂, -NR¹¹C(O)N(R¹¹)₂, -NR¹¹C(O)R¹¹, -OC(O)N(R¹¹)₂, or -NR¹¹C(O)OR¹¹;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
R¹² is C₁₋₆ alkyl, C₂₋₆ alkenyl, C₂₋₆ alkynyl, or C₁₋₆ haloalkyl;
each Z1a is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)₀₋₂R¹³, –NR¹³S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂, –NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, –L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, –N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, —C(O)—,C(O)O—, —C(O)NH—, -C(O)N(C₁₋₆ alkyl)-, -C(O)N(C₂₋₆ alkenyl)-, -C(O)N(C₂₋₆ alkynyl)-, –C(O)N(C₁₋₆ haloalkyl)-, -C(O)N(C₃₋₁₀ cycloalkyl)-, -C(O)N(heterocyclyl)-, -C(O)N(aryl)-, –C(O)N(heteroaryl)-,NHC(O)-, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl, provided that:
1) when one of R⁴ and R⁵ is H, the other of R⁴ and R⁵ is not C₃-alkyl substituted with an optionally substituted piperazinyl ring;
2) when R² is unsubstituted C₁₋₆ alkyl, or unsubstituted C₂₋₆ alkenyl and one R¹ is unsubstituted C₁₋₆ alkyl, unsubstituted C₂₋₆ alkenyl, unsubstituted C₅₋₇ cycloalkyl, unsubstituted C₁₋₆ alkoxy, halo, benzyl, or hydroxy; then:
R⁴ and R⁵ are not independently hydrogen, unsubstituted C₁₋₆ alkyl, unsubstituted C₂₋₆ alkenyl, unsubstituted C₅₋₇ cycloalkyl, unsubstituted aryl or aryl substituted with one Z¹; and
R⁴ and R⁵, together with the nitrogen to which they are attached, are not unsubstituted piperidinyl, unsubstituted morpholinyl, or piperazinyl substituted with C₁₋₆ alkyl or aryl; and
3) when R² is -CH₂-C(O)OR¹¹; then R⁴ and R⁵, together with the nitrogen to which they are attached, are not unsubstituted morpholinyl.

2. The compound of claim 1, or a pharmaceutically acceptable salt, isotopically enriched analog, stereoisomer, mixture of stereoisomers, or prodrug thereof, wherein each of A¹, A², A³, and A⁴ is independently CH or CR¹; provided at least one of A¹, A², A³, and A⁴ is CR¹.

3. The compound of claim 1, or a pharmaceutically acceptable salt, isotopically enriched analog, stereoisomer, mixture of stereoisomers, or prodrug thereof, wherein one of A¹, A², A³, and A⁴ is N; one of A¹, A², A³, and A⁴ is CR¹; and the remaining A¹, A², A³, and A⁴ are independently CH or CR¹.

4. The compound of claim 1, or a pharmaceutically acceptable salt, isotopically enriched analog, stereoisomer, mixture of stereoisomers, or prodrug thereof, wherein two of A¹, A², A³, and A⁴ are N; one of A¹, A², A³, and A⁴ is CR¹; and the remaining A¹, A², A³, and A⁴ is CH or CR¹.

5. The compound of any one of claims 1-4, or a pharmaceutically acceptable salt, isotopically enriched analog, stereoisomer, mixture of stereoisomers, or prodrug thereof, wherein each R¹ is independently halo, cyano, C₁₋₆ alkyl, C₃₋₁₀ cycloalkyl, -N(R¹¹)₂, -OR¹¹, or -SR¹¹; wherein each C₁₋₆ alkyl or C₃₋₁₀ cycloalkyl is independently optionally substituted with one to eight Z¹.

6. The compound of any one of claims 1-5, or a pharmaceutically acceptable salt, isotopically enriched analog, stereoisomer, mixture of stereoisomers, or prodrug thereof, wherein R⁴ is hydrogen.

7. The compound of any one of claims 1-6, or a pharmaceutically acceptable salt, isotopically enriched analog, stereoisomer, mixture of stereoisomers, or prodrug thereof, wherein R⁶ is hydrogen or C₁₋₆ alkyl.

8. The compound of any one of claims 1-7, or a pharmaceutically acceptable salt, isotopically enriched analog, stereoisomer, mixture of stereoisomers, or prodrug thereof, wherein R⁷ is hydrogen.

9. The compound of any one of claims 1-8, or a pharmaceutically acceptable salt, isotopically enriched analog, stereoisomer, mixture of stereoisomers, or prodrug thereof, wherein
R⁵ is C₁₋₆ alkyl,C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or
R⁴ and R⁵ together form a heterocyclyl ring optionally substituted with one to eight Z¹.

10. The compound of any one of claims 1-9, or a pharmaceutically acceptable salt, isotopically enriched analog, stereoisomer, mixture of stereoisomers, or prodrug thereof, wherein R² is C₁₋₆ alkyl, C₁₋₆ haloalkyl, -SR¹¹, -OR¹¹, or halo, wherein each R¹¹ is independently C₁₋₆ alkyl optionally substituted with one to five Z^{1a}.

11. The compound of claim 1, or a pharmaceutically acceptable salt, isotopically enriched analog, stereoisomer, mixture of stereoisomers, or prodrug thereof, wherein:
each of A¹, A², A³, and A⁴ is independently CH or CR¹; provided at least one of A¹, A², A³, and A⁴ is CR¹;
each R¹ is independently halo, cyano, C₁₋₆ alkyl, C₁₋₆ alkoxy, C₁₋₆ haloalkyl, C₁₋₆ haloalkoxy, N(R¹¹)₂, -SR¹¹, or C₃₋₁₀ cycloalkyl;
R² is C₁₋₆ alkyl, C₁₋₆ haloalkyl, -SR¹¹, -OR¹¹, or halo; wherein the C₁₋₆ alkyl is optionally substituted with one to eight Z²;
R⁴ is hydrogen;
R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or
R⁴ and R⁵ together form a heterocyclyl ring optionally substituted with one to eight Z¹;
R⁶ is hydrogen or C₁₋₆ alkyl; and
R⁷ is hydrogen.

12. The compound of claim 1, or a pharmaceutically acceptable salt, isotopically enriched analog, stereoisomer, mixture of stereoisomers, or prodrug thereof, wherein:
one of A¹, A², A³, and A⁴ is N; one of A¹, A², A³, and A⁴ is CR¹; and the remaining A¹, A², A³, and A⁴ are independently CH or CR¹;
each R¹ is independently halo, cyano, C₁₋₆ alkyl, C₁₋₆ alkoxy, C₁₋₆ haloalkyl, C₁₋₆ haloalkoxy, N(R¹¹)₂, -SR¹¹, or C₃₋₁₀ cycloalkyl;
R² is C₁₋₆ alkyl, C₁₋₆ haloalkyl, -SR¹¹, -OR¹¹, or halo; wherein the C₁₋₆ alkyl is optionally substituted with one to eight Z²;
R⁴ is hydrogen;
R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or
R⁴ and R⁵ together form a heterocyclyl ring optionally substituted with one to eight Z¹;
R⁶ is hydrogen or C₁₋₆ alkyl; and
R⁷ is hydrogen.

13. The compound of claim 1, or a pharmaceutically acceptable salt, isotopically enriched analog, stereoisomer, mixture of stereoisomers, or prodrug thereof, wherein:
two of A¹, A², A³, and A⁴ are N; one of A¹, A², A³, and A⁴ is CR¹; and the remaining A¹, A², A³, and A⁴ is CH or CR¹;
each R¹ is independently halo, cyano, C₁₋₆ alkyl, C₁₋₆ alkoxy, C₁₋₆ haloalkyl, C₁₋₆ haloalkoxy, N(R¹¹)₂, -SR¹¹, or C₃₋₁₀ cycloalkyl;
R² is C₁₋₆ alkyl, C₁₋₆ haloalkyl, -SR¹¹, -OR¹¹, or halo; wherein the C₁₋₆ alkyl is optionally substituted with one to eight Z²;
R⁴ is hydrogen;
R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or
R⁴ and R⁵ together form a heterocyclyl ring optionally substituted with one to eight Z¹;
R⁶ is hydrogen or C₁₋₆ alkyl; and
R⁷ is hydrogen.

14. The compound of claim 1, or a pharmaceutically acceptable salt, isotopically enriched analog, stereoisomer, mixture of stereoisomers, or prodrug thereof, wherein:
A² is CR¹ and A¹, A ³ and A⁴ are each independently N, CH, or CR¹;
each R¹ is independently halo, cyano, C₁₋₆ alkyl, C₁₋₆ alkoxy, C₁₋₆ haloalkyl, C₁₋₆ haloalkoxy, N(R¹¹)₂, -SR¹¹, or C₃₋₁₀ cycloalkyl;
R² is C₁₋₆ alkyl, C₁₋₆ haloalkyl, -SR¹¹, -OR¹¹, or halo; wherein the C₁₋₆ alkyl is optionally substituted with one to eight Z²;
R⁴ is hydrogen;
R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or
R⁴ and R⁵ together form a heterocyclyl ring optionally substituted with one to eight Z¹;
R⁶ is hydrogen or C₁₋₆ alkyl; and
R⁷ is hydrogen.

15. The compound of claim 1, or a pharmaceutically acceptable salt, isotopically enriched analog, stereoisomer, mixture of stereoisomers, or prodrug thereof, wherein:
A³ is CR¹ and A¹, A², and A⁴ are each independently N, CH, or CR¹;
each R¹ is independently halo, cyano, C₁₋₆ alkyl, C₁₋₆ alkoxy, C₁₋₆ haloalkyl, C₁₋₆ haloalkoxy, N(R¹¹)₂, -SR¹¹, or C₃₋₁₀ cycloalkyl;
R² is C₁₋₆ alkyl, C₁₋₆ haloalkyl, -SR¹¹, -OR¹¹, or halo; wherein the C₁₋₆ alkyl is optionally substituted with one to eight Z²;
R⁴ is hydrogen;
R⁵ is C₁₋₆ alkyl, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹; or
R⁴ and R⁵ together form a heterocyclyl ring optionally substituted with one to eight Z¹;
R⁶ is hydrogen or C₁₋₆ alkyl; and
R⁷ is hydrogen.

16. A compound or a pharmaceutically acceptable salt, isotopically enriched analog, stereoisomer, mixture of stereoisomers, or prodrug thereof, selected from Table 1.

17. A compound or a pharmaceutically acceptable salt, isotopically enriched analog, stereoisomer, mixture of stereoisomers, or prodrug thereof, selected from Table 2.

18. A pharmaceutical composition comprising a compound of any preceding claim, or a pharmaceutically acceptable salt, stereoisomer, mixture of stereoisomers, or prodrug thereof, and a pharmaceutically acceptable carrier.

19. A method for treating a disease or condition mediated, at least in part, by NLRP3, the method comprising administering an effective amount of the pharmaceutical composition of claim 16, or a compound of Formula I: 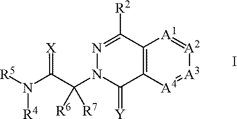
or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, —NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, C₃₋₁₀ cycloalkyl, —NO₂, —SF₅, -OR¹¹, —C(O)R¹¹, -C(O)OR¹¹, -SR¹¹, -NR¹¹S(O)₀₋₂-R¹¹, -NR¹¹S(O)₀₋₂N(R¹¹)₂, -NR¹¹C(O)N(R¹¹)₂, —NR¹¹C(O)OR¹¹, -OC(O)R¹¹, -OC(O)N(R¹¹)₂, halo, cyano, -NR¹¹C(O)R¹¹, -S(O)R¹¹, or -S(O)₂R¹¹; wherein the C₁₋₆ alkyl, C₂₋₆ alkenyl, C₂₋₆ alkynyl, C₁₋₆ haloalkyl, or C₃₋₁₀ cycloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹;
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{1a};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, —S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, —NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)R¹¹, -C(O)OR¹¹, -NR¹¹S(O)₀₋₂-R¹¹, —NR¹¹S(O)₀₋₂N(R¹¹)₂, -NR¹¹C(O)N(R¹¹)₂, -NR¹¹C(O)R¹¹, -OC(O)N(R¹¹)₂, -NR¹¹C(O)OR¹¹, -N(R¹¹)₂, —C(O)N(R¹¹)₂, -S(O)₀₋₂R¹¹, or -S(O)₀₋₂N(R¹¹)₂;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)₀₋₂R¹³, —NR¹³S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂ —NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, C(O)—,C(O)O—, —C(O)NH—, -C(O)N(C₁₋₆ alkyl)-, -C(O)N(C₂₋₆ alkenyl)-, -C(O)N(C₂₋₆ alkynyl)-, C(O)N(C₁₋₆ haloalkyl)-, -C(O)N(C₃₋₁₀ cycloalkyl)-, -C(O)N(heterocyclyl)-, -C(O)N(aryl)-, C(O)N(heteroaryl)-,NHC(O)-, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;
to a subject in need thereof.

20. The method of claim 19, wherein the disease or condition is Alzheimer disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury.

21. The method of claim 20, wherein the disease is nonalcoholic fatty liver disease (NAFLD) or nonalcoholic steatohepatitis (NASH).

22. The method of claim 20, wherein the disease is Alzheimer’s disease.

23. Use of a compound of any one of claims 1-17, or a pharmaceutically acceptable salt, stereoisomer, mixture of stereoisomers, or prodrug thereof, or a compound of Formula I: 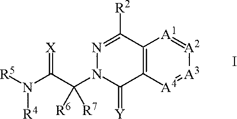
or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR11, -C(O)R¹¹, -C(O)OR¹¹, -S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, —NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋ ₁₀cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, C₃₋₁₀ cycloalkyl, —NO₂, —SF₅, -OR¹¹, —C(O)R¹¹, -C(O)OR¹¹, -SR¹¹, -NR¹¹S(O)₀₋₂-R¹¹, -NR¹¹S(O)₀₋₂N(R¹¹)₂, -NR¹¹C(O)N(R¹¹)₂, —NR¹¹C(O)OR¹¹, -OC(O)R¹¹, -OC(O)N(R¹¹)₂, halo, cyano, -NR¹¹C(O)R¹¹, -S(O)R¹¹, or -S(O)₂R¹¹; wherein the C₁₋₆ alkyl, C₂₋₆ alkenyl, C₂₋₆ alkynyl, C₁₋₆ haloalkyl, or C₃₋₁₀ cycloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹;
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{1a};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, —NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)R¹¹, -C(O)OR¹¹, -NR¹¹S(O)₀₋₂-R¹¹, —NR¹¹S(O)₀₋₂N(R¹¹)₂, -NR¹¹C(O)N(R¹¹)₂, -NR¹¹C(O)R¹¹, -OC(O)N(R¹¹)₂, -NR¹¹C(O)OR¹¹, -N(R¹¹)₂, —C(O)N(R¹¹)₂, -S(O)₀₋₂R¹¹, or -S(O)₀₋₂N(R¹¹)₂;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR1, -S(O)₀₋₂R¹³, —NR ¹³ S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂ —NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, C(O)—,C(O)O—, —C(O)NH—, -C(O)N(C₁₋₆ alkyl)-, -C(O)N(C₂₋₆ alkenyl)-, -C(O)N(C₂₋₆ alkynyl)-, C(O)N(C₁₋₆ haloalkyl)-, -C(O)N(C₃₋₁₀ cycloalkyl)-, -C(O)N(heterocyclyl)-, -C(O)N(aryl)-, C(O)N(heteroaryl)-,NHC(O)-, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;
for treating a disease or condition mediated, at least in part, by NLRP3.

24. The use of claim 23, wherein the disease or condition is Alzheimer disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury.

25. A compound of any one of claims 1-17, or a pharmaceutically acceptable salt, stereoisomer, mixture of stereoisomers, or prodrug thereof, for use in therapy.

26. A compound of any one of claims 1-17, or a pharmaceutically acceptable salt, stereoisomer, mixture of stereoisomers, or prodrug thereof, or a compound of Formula I: 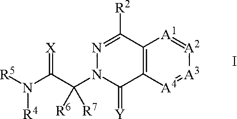
or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂,- NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, C₃₋₁₀ cycloalkyl, —NO₂, —SF₅, -OR¹¹, —C(O)R¹¹, -C(O)OR¹¹, -SR¹¹, -NR¹¹S(O)₀₋₂-R¹¹, -NR¹¹S(O)₀₋₂N(R¹¹)₂, -NR¹¹C(O)N(R¹¹)₂, —NR¹¹C(O)OR¹¹, -OC(O)R¹¹, -OC(O)N(R¹¹)₂, halo, cyano, -NR¹¹C(O)R¹¹, -S(O)R¹¹, or —S(O)₂R¹¹; wherein the C₁₋₆ alkyl, C₂₋₆ alkenyl, C₂₋₆ alkynyl, C₁₋₆ haloalkyl, or C₃₋₁₀ cycloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{1a};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, —NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)R¹¹, -C(O)OR¹¹, -NR¹¹S(O)₀₋₂-R¹¹, —NR¹¹S(O)₀₋₂N(R¹¹)₂, -NR¹¹C(O)N(R¹¹)₂, -NR¹¹C(O)R¹¹, -OC(O)N(R¹¹)₂, -NR¹¹C(O)OR¹¹, -N(R¹¹)₂, C(O)N(R¹¹)₂, -S(O)₀₋₂R¹¹, or -S(O)₀₋₂N(R¹¹)₂;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)₀₋₂R¹³, —NR¹³ S(O)₀₋₂-R ¹³, -S(O)₀₋₂N(R¹³)₂, -NR¹³S(O)₀₋₂N(R¹³)₂, -NR¹³C(O)N(R¹³)₂, -C(O)N(R¹³)₂ —NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, C(O)—,C(O)O—, —C(O)NH—, -C(O)N(C₁₋₆ alkyl)-, -C(O)N(C₂₋₆ alkenyl)-, -C(O)N(C₂₋₆ alkynyl)-, C(O)N(C₁₋₆ haloalkyl)-, -C(O)N(C₃₋₁₀ cycloalkyl)-, -C(O)N(heterocyclyl)-, -C(O)N(aryl)-, C(O)N(heteroaryl)-,NHC(O)-, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;
for use in treating Alzheimer’s disease.

27. A compound of any one of claims 1-17, or a pharmaceutically acceptable salt, stereoisomer, mixture of stereoisomers, or prodrug thereof, or a compound of Formula I: 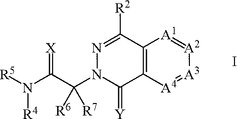
or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, —NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, C₃₋₁₀ cycloalkyl, —NO₂, —SF₅, -OR¹¹, —C(O)R¹¹, -C(O)OR¹¹, -SR¹¹, -NR¹¹S(O)₀₋₂-R¹¹, -NR¹¹S(O)₀₋₂N(R¹¹)₂, -NR¹¹C(O)N(R¹¹)₂, —NR¹¹C(O)OR¹¹, -OC(O)R¹¹, -OC(O)N(R¹¹)₂, halo, cyano, -NR¹¹C(O)R¹¹, -S(O)R¹¹, or -S(O)₂R¹¹; wherein the C₁₋₆ alkyl, C₂₋₆ alkenyl, C₂₋₆ alkynyl, C₁₋₆ haloalkyl, or C₃₋₁₀ cycloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹;
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{1a};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, —NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)R¹¹, -C(O)OR¹¹, -NR¹¹S(O)₀₋₂-R¹¹, —NR¹¹S(O)₀₋₂N(R¹¹)₂, -NR¹¹C(O)N(R¹¹)₂, -NR¹¹C(O)R¹¹, -OC(O)N(R¹¹)₂, -NR¹¹C(O)OR¹¹, -N(R¹¹)₂, —C(O)N(R¹¹)₂, -S(O)₀₋₂R¹¹, or -S(O)₀₋₂N(R¹¹)₂;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)₀₋₂R¹³, —NR¹³S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂, —NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, C(O)—,C(O)O—, —C(O)NH—, -C(O)N(C₁₋₆ alkyl)-, -C(O)N(C₂₋₆ alkenyl)-, -C(O)N(C₂₋₆ alkynyl)-, C(O)N(C₁₋₆ haloalkyl)-, -C(O)N(C₃₋₁₀ cycloalkyl)-, -C(O)N(heterocyclyl)-, -C(O)N(aryl)-, C(O)N(heteroaryl)-,NHC(O)-, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;
for use in treating nonalcoholic fatty liver disease (NAFLD) or nonalcoholic steatohepatitis (NASH).

28. The use of a compound of claims 1-27, or a pharmaceutically acceptable salt, stereoisomer, mixture of stereoisomers, or prodrug thereof, or a compound of Formula I: 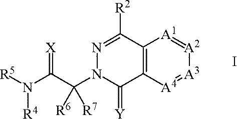
or a pharmaceutically acceptable salt, isotopically enriched analog, stereoisomer, mixture of stereoisomers, or prodrug thereof, wherein:
X is O or S;
Y is O or S;
A¹, A², A³, and A⁴ are each independently N, CH, or CR¹; provided at least one of A¹, A², A³, and A⁴ is CR¹;
each R¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, —NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹;
R² is C₁₋₆ alkyl, C₂₋₆ alkenyl, C₂₋₆ alkynyl, C₁₋₆ haloalkyl, C₃₋₁₀ cycloalkyl, —NO₂, —SF₅, -OR¹¹, —C(O)R¹¹, -C(O)OR¹¹, -SR¹¹, -NR¹¹S(O)₀₋₂-R¹¹, -NR¹¹S(O)₀₋₂N(R¹¹)₂, -NR¹¹C(O)N(R¹¹)₂, —NR¹¹C(O)OR¹¹, -OC(O)R¹¹, -OC(O)N(R¹¹)₂, halo, cyano, -NR¹¹C(O)R¹¹, -S(O)R¹¹, or -S(O)₂R¹¹; wherein the C₁₋₆ alkyl, C₂₋₆ alkenyl, C₂₋₆ alkynyl, C₁₋₆ haloalkyl, or C₃₋₁₀ cycloalkyl is independently optionally substituted with one to eight Z²;
R⁴ and R⁵ are independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to eight Z¹; or
R⁴ and R⁵ together form a heterocyclyl or heteroaryl ring optionally substituted with one to eight Z¹;
R⁶ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
R⁷ is hydrogen, halo, cyano, hydroxy, C₁₋₆ alkyl, C₂₋₆ alkenyl, C₂₋₆ alkynyl, C₁₋₆ haloalkyl, C₁₋₆ alkoxy, C₁₋₆ haloalkoxy, C₂₋₆ heteroalkyl, C₃₋₁₀ cycloalkyl, or heterocyclyl;
or R⁶ and R⁷ join to form a C₃₋₁₀ cycloalkyl or heterocyclyl ring, wherein the C₃₋₁₀ cycloalkyl or heterocyclyl ring may further be independently optionally substituted with one to five Z^{1a};
each Z¹ is independently halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹¹)₂, -OR¹¹, -C(O)R¹¹, -C(O)OR¹¹, -S(O)₀₋₂R¹¹, —NR¹¹S(O)₀₋₂-R¹¹, -S(O)₀₋₂N(R¹¹)₂, -NR¹¹S(O)₀₋₂N(R¹¹)₂, -NR¹¹C(O)N(R¹¹)₂, -C(O)N(R¹¹)₂, —NR¹¹C(O)R¹¹, -OC(O)N(R¹¹)₂, or -NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z² is independently halo, cyano, —NO₂, —SF₅, -OR¹¹, -C(O)R¹¹, -C(O)OR¹¹, -NR¹¹S(O)₀₋₂-R¹¹, —NR¹¹S(O)₀₋₂N(R¹¹)₂, -NR¹¹C(O)N(R¹¹)₂, -NR¹¹C(O)R¹¹, -OC(O)N(R¹¹)₂, -NR¹¹C(O)OR¹¹, -N(R¹¹)₂, —C(O)N(R¹¹)₂, -S(O)₀₋₂R¹¹, or -S(O)₀₋₂N(R¹¹)₂;
each R¹¹ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹¹ is independently optionally substituted with one to five Z^{1a};
each Z^{1a} is independently hydroxy, halo, cyano, —NO₂, —SF₅, C₁₋₆ alkyl, C₂₋₆ alkenyl, C₂₋₆ alkynyl, C₃₋₁₀ cycloalkyl, heterocyclyl, aryl, heteroaryl, -N(R¹³)₂, -OR¹³, -C(O)R¹³, -C(O)OR¹³, -S(O)₀₋₂R¹³, —NR¹³S(O)₀₋₂-R¹³, -S(O)₀₋₂N(R¹³)₂, -NR¹³S(O)₀₋₂N(R¹³)₂, -NR¹³C(O)N(R¹³)₂, -C(O)N(R¹³)₂, —NR¹³C(O)R¹³, -OC(O)N(R¹³)₂, or -NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl of R¹³ is independently optionally substituted with one to five Z^{1b};
each Z^{1b} is independently halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, -L-C₁₋₆ alkyl, -L-C₂₋₆ alkenyl, L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, -N(C₁₋₆ alkyl)-, -N(C₂₋₆ alkenyl)-, N(C₂₋₆ alkynyl)-, -N(C₁₋₆ haloalkyl)-, -N(C₃₋₁₀ cycloalkyl)-, -N(heterocyclyl)-, -N(aryl)-, -N(heteroaryl)-, C(O)—,C(O)O—, —C(O)NH—, -C(O)N(C₁₋₆ alkyl)-, -C(O)N(C₂₋₆ alkenyl)-, -C(O)N(C₂₋₆ alkynyl)-, C(O)N(C₁₋₆ haloalkyl)-, -C(O)N(C₃₋₁₀ cycloalkyl)-, -C(O)N(heterocyclyl)-, -C(O)N(aryl)-, C(O)N(heteroaryl)-,NHC(O)-, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five hydroxy, halo, cyano, hydroxy, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;
for the manufacture of a medicament for treating a neurodegenerative disease, treating Alzheimer’s disease, atherosclerosis, asthma, allergic airway inflammation, cryopyrin-associated periodic syndromes, gout, inflammatory bowel disease and related disorders, nonalcoholic fatty liver disease (NAFLD), nonalcoholic steatohepatitis (NASH), hypertension, myocardial infarction, multiple sclerosis, experimental autoimmune encephalitis, oxalate-induced nephropathy, hyperinflammation following influenza infection, graft-versus-host disease, stroke, silicosis, type 1 diabetes, obesity-induced inflammation or insulin resistance, rheumatoid arthritis, myelodysplastic syndrome, contact hypersensitivity, joint inflammation triggered by chikungunya virus, or traumatic brain injury.
